(19) United States
(12) Patent Application Publication
(10) Pub. No.: US 2010/0317646 A1
(43) Pub. Date: Dec. 16, 2010

(54) COMPOUNDS

(75) Inventors: Edward Giles Mciver, London (GB); Ela Smiljanic, London (GB); Denise Jamilla Harding, London (GB); Joanne Hough, London (GB)

Correspondence Address: LAHIVE & COCKFIELD, LLP;FLOOR 30, SUITE 3000, ONE POST OFFICE SQUARE, BOSTON, MA, 02109, US

(73) Assignee: Medical Research Council Technology, London (GB)

(21) Appl. No.: 12/727,754

(22) Filed: Mar. 19, 2010

Related U.S. Application Data
(60) Provisional application No. 61/162,024, filed on Mar. 20, 2009.

(30) Foreign Application Priority Data
Mar. 19, 2009 (GB) 0904746.5
Jul. 14, 2009 (GB) 0912238.3
Jan. 28, 2010 (GB) 1001418.1

(51) Int. Cl. A61K 31/437; C07D 471/04; A61K 31/496; A61K 31/5377; A61K 31/4725; A61K 31/497; A61K 31/538; A61K 31/498; A61K 31/506; A61P 25/28; A61P 35/00; A61P 25/16

(52) U.S. Cl. 514/210.18; 546/119; 514/303; 544/362; 514/253.04; 544/127; 514/234.2; 544/405; 514/255.05; 544/105; 514/230.5; 544/353; 514/249; 544/333; 514/256

(57) ABSTRACT

A compound of formula I, or a pharmaceutically acceptable salt or ester thereof,

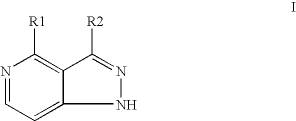

wherein R¹ is selected from: aryl; heteroaryl; —NHR³; fused aryl-C₄₋₇-heterocycloalkyl; —CONR⁴R⁵; —NHCOR⁶; —C₃₋₇-cycloalkyl; —O—C₃₋₇-cycloalkyl; —NR³R⁶; and optionally substituted —C₁₋₆ alkyl; wherein said aryl, heteroaryl, fused aryl-C₄₋₇-heterocycloalkyl and C₄₋₇-heterocycloalkyl are each optionally substituted;
R² is selected from hydrogen, aryl, C₁₋₆-alkyl, C₂₋₆-alkenyl, C₃₋₇-cycloalkyl, heteroaryl, C₄₋₇ heterocycloalkyl and halogen, wherein said C₁₋₆-alkyl, C₂₋₆-alkenyl, aryl, heteroaryl and C₄₋₇-heterocycloalkyl are each optionally substituted;
R³ is selected from aryl, heteroaryl, C₄₋₇-heterocycloalkyl, C₃₋₇-cycloalkyl, fused aryl-C₄₋₇-heterocycloalkyl and C₁₋₆-alkyl, each of which is optionally substituted;
R⁴ and R⁵ are each independently hydrogen, or optionally substituted C₃₋₇-cycloalkyl, aryl, heteroaryl, C₁₋₆-alkyl or C₃₋₆-heterocycloalkyl; or R⁴ and R⁵ together with the N to which they are attached form a C₃₋₆-heterocycloalkyl ring;
each R⁶ is independently selected from C₁₋₆-alkyl, C₃₋₇ cycloalkyl, C₄₋₇-heterocycloalkyl, aryl and heteroaryl, each of which is optionally substituted
each R⁷ is selected from hydrogen, optionally substituted C₁₋₆-alkyl and C₃₋₇-cycloalkyl;
each of R⁸ and R⁹ is independently hydrogen or optionally substituted C₁₋₆-alkyl; or
R⁸ and R⁹ together with the N to which they are attached form a C₄₋₆-heterocycloalkyl;
each R¹⁰ is selected from C₃₋₇-cycloalkyl and optionally substituted C₁₋₆-alkyl;
each R¹¹ is independently selected from C₁₋₆-alkyl, C₃₋₇-cycloalkyl, C₁₋₆ alkyl-C₃₋₇-cycloalkyl, C₄₋₇-heterocycloalkyl, aryl and heteroaryl, each of which is optionally substituted;
A is selected from halogen, —NR⁴SO₂R⁵, —CN, —OR⁶, —NR⁴R⁵, —NR⁷R¹¹, hydroxyl, —CF₃, —CONR⁴R⁵, —NR⁴COR⁵, —NR⁷(CO)NR⁴R⁵, —NO₂, —CO₂H, —CO₂R⁶, —SO₂R⁶, —SO₂NR⁴R⁵, —NR⁴COR⁵, —NR⁴COOR⁵, C₁₋₆-alkyl and —COR⁶.

Further aspects relate to pharmaceutical compositions, therapeutic uses and process for preparing compounds of formula I.

### RELATED APPLICATIONS

This application claims priority to Great Britain Application No. 0904746.5, filed Mar. 19, 2009; Great Britain Application No. 0912238.3, filed Jul. 14, 2009; Great Britain Application No. 1001418.1, filed Jan. 28, 2010; and U.S. Provisional Application No. 61/162,024, filed Mar. 20, 2009, which are incorporated herein by reference in their entirety. Additionally, the contents of any patents, patent applications, and references cited throughout this specification are hereby incorporated by reference in their entireties.

### TECHNICAL FIELD

The present invention relates to pyrazolopyridine compounds that are capable of inhibiting one or more kinases, more particularly, LRRK2. The compounds find applications in the treatment of a variety of disorders, including cancer and neurodegenerative diseases such as Parkinson's disease.

### BACKGROUND TO THE INVENTION

There has been much interest raised by the recent discovery that different autosomal dominant point mutations within the gene encoding for LRRK2 predispose humans to develop late-onset PD (OMIM accession number 609007), with a clinical appearance indistinguishable from idiopathic PD [1-3]. The genetic analysis undertaken to data indicates that mutations in LRRK2 are relatively frequent, not only accounting for 5-10% of familial PD, but also being found in a significant proportion of sporadic PD cases [4, 5]. Little is known about how LRRK2 is regulated in cells, what its physiological substrates are and how mutations cause or increase risk of PD.

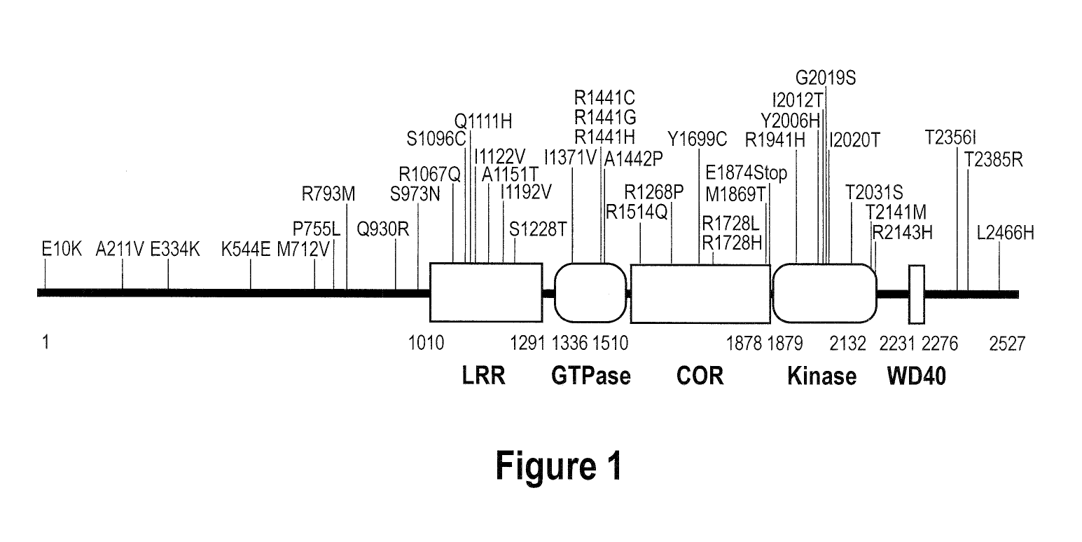

The domain structure of LRRK2 is shown in FIG. 1, which also depicts the mutations that have thus far been reported in patients with PD. The defining feature of the LRRK2 enzyme is a Leucine Rich Repeat (LRR) motif (residues 1010-1291), a Ras-like small GTPase (residues 1336-1510), a region of high amino acid conservation that has been termed the C-terminal Of Ras of complex (COR) domain (residues 1511-1878), a protein kinase catalytic domain (residues 1879-2132) and a C-terminal WD40 motif (2231-2276) [6, 7]. The protein kinase domain of LRRK2 belongs to the tyrosine-like serine/threonine protein kinases and is most similar to the kinase RIP (Receptor Interacting Protein), which play key roles in innate immunity signalling pathways [8]. To date, almost 40 single amino acid substitution mutations have been linked to autosomal-dominant PD and the location of these mutations is illustrated in FIG. 1A ([2, 3]). The most prevalent mutant form of LRRK2 accounting for approximately 6% of familial PD and 3% of sporadic PD cases in Europe, comprises an amino acid substitution of Gly2019 to a Ser residue. Gly2019 is located within the conserved DYG-Mg²⁺-binding motif, in subdomain-VII of the kinase domain [2]. Recent reports suggest that this mutation enhances the autophosphorylation of LRRK2, as well as its ability to phosphorylate myelin basic protein 2-3-fold [9, 10], a finding confirmed by the Applicant [11]. These observations suggest that over-activation of LRRK2 predisposes humans to develop PD, implying that drugs which inhibited LRRK2, could be utilised to halt progression or even perhaps reverse symptoms of some forms of PD.

The study of LRRK2 has been hampered by the difficulty in expressing active recombinant enzyme and by the lack of a robust quantitative assay. In work undertaken by the Applicant, an active recombinant fragment of LRRK2 containing the GTPase-COR and kinase domains encompassing residues 1326-2527 was expressed in 293 cells [11]. The more active G2019S mutant of this LRRK2 fragment was utilised in a KinasE Substrate TRacking and ELucidation (KESTREL) screen in an initial attempt to identify physiological substrates (reviewed in [14]). This led to the identification of a protein termed moesin, which was efficiently phosphorylated by LRRK2 in vitro [11]. Moesin is a member of the Ezrin/Radixin/Moesin (ERM) family of proteins which functions to anchor the actin cytoskeleton to the plasma membrane and plays an important role in regulating membrane structure and organization [15, 16]. It was found that LRRK2 phosphorylated moesin at Thr558 [11], a previously characterised physiologically relevant phosphorylation site [15, 16]. LRRK2 also phosphorylated ezrin and radixin at the equivalent Thr residue. Phosphorylation of ERM proteins at the residue equivalent to Thr558, opens up the structures of these proteins and enables them to interact with actin microfilaments at their C-terminal residues and phosphoinositides and plasma membrane proteins through an N-terminal FERM domain. These findings were utilised to develop a robust and quantitative assay for LRRK2, based upon the phosphorylation of moesin or a short peptide that encompasses the Thr558 residue of moesin which is also efficiently phosphorylated by LRRK2 [11]. These assays were further adapted to develop an improved assay based on the use of the Nictide peptide [17].

The present invention seeks to provide compounds that are capable of inhibiting one or more kinases, more particularly, LRRK, even more preferably LRRK2.

### STATEMENT OF INVENTION

A first aspect of the invention relates to a compound of formula I, or a pharmaceutically acceptable salt or ester thereof,

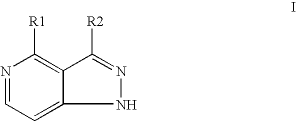

wherein:
R¹ is selected from:
aryl;
heteroaryl;
C₄₋₇-heterocycloalkyl;

### —NHR³;

fused aryl-C₄₋₇-heterocycloalkyl;

### —CONR⁴R⁵;

### —NHCOR⁶;

—C₃₋₇-cycloalkyl;

### —NR³R⁶;

### OR³;

### OH;

### NR⁴R⁵; and

—C₁₋₆ alkyl optionally substituted with a substituent selected from R¹¹ and a group A;
wherein said aryl, heteroaryl, fused aryl-C₄₋₇-heterocycloalkyl and C₄₋₇-heterocycloalkyl are each optionally substituted with one or more substituents selected from C₁₋₆-alkyl, C₃₋₇-cycloalkyl, heteroaryl, C₄₋₇-heterocycloalkyl, aryl and a group A, and said C₁₋₆-alkyl, C₃₋₇-cycloalkyl, heteroaryl, C₄₋₇-heterocycloalkyl, and aryl substituents are in turn each optionally substituted with one or more groups selected from R¹¹ and a group A;
R² is selected from hydrogen, aryl, C₁₋₆-alkyl, C₂₋₆-alkenyl, C₃₋₇-cycloalkyl, heteroaryl, C₄₋₇ heterocycloalkyl, fused aryl-C₄₋₇-heterocycloalkyl and halogen, wherein said C₁₋₆-alkyl, C₂₋₆-alkenyl, aryl, heteroaryl, fused aryl-C₄₋₇-heterocycloalkyl and C₄₋₇-heterocycloalkyl are each optionally substituted with one or more substituents selected from R¹¹ and A;
each R³ is selected from aryl, heteroaryl, C₄₋₇-heterocycloalkyl, C₃₋₇-cycloalkyl, fused aryl-C₄₋₇-heterocycloalkyl and C₁₋₆-alkyl, each of which is optionally substituted with one or more substituents selected from R¹¹ and A;
R⁴ and R⁵ are each independently selected from hydrogen, C₃₋₇-cycloalkyl, C₁₋₆-alkyl-C₃₋₇-cycloalkyl, aryl, heteroaryl, C₁₋₆-alkyl and a C₃₋₆-heterocycloalkyl ring optionally further containing one or more groups selected from oxygen, sulfur, nitrogen and CO, and optionally substituted by one or more R¹⁰ groups, wherein each C₁₋₆-alkyl, heteroaryl and aryl is optionally substituted by one or more substituents selected from C₁₋₆-alkyl, halogen, cyano, hydroxyl, aryl, halo-substituted aryl, heteroaryl, —NR⁸R⁹, —NR⁶R⁷, NR⁷(CO)R⁶, —NR⁷COOR⁶, —NR⁷(SO₂)R⁶, —COOR⁶, —CONR⁸R⁹, OR⁶, —SO₂R⁶ and a C₃₋₆-heterocycloalkyl ring optionally further containing one or more groups selected from oxygen, sulfur, nitrogen and CO and optionally substituted by one or more or R¹⁰ groups; or
R⁴ and R⁵ together with the N to which they are attached form a C₃₋₆-heterocycloalkyl ring optionally further containing one or more groups selected from oxygen, sulfur, nitrogen and CO, wherein said C₃₋₆-heterocycloalkyl ring is saturated or unsaturated and is optionally substituted with one or more groups selected from A, NR⁸R⁹ and R¹⁰;
each R⁶ is independently selected from C₁₋₆-alkyl, C₃₋₇ cycloalkyl, C₄₋₇-heterocycloalkyl, aryl and heteroaryl, each of which is optionally substituted by one or more substituents selected from R¹⁰, R¹¹ and A;
each R⁷ is selected from hydrogen, C₁₋₆-alkyl and C₃₋₇-cycloalkyl, wherein said C₁₋₆-alkyl is optionally substituted by one or more halogens;
each of R⁸ and R⁹ is independently selected from hydrogen and C₁₋₆-alkyl, wherein said C₁₋₆-alkyl group is optionally substituted by one or more halogens; or
R⁸ and R⁹ together with the N to which they are attached form a C₄₋₆-heterocycloalkyl ring optionally further containing one or more heteroatoms selected from oxygen and sulfur, wherein said C₄₋₆-heterocycloalkyl ring is optionally substituted by one or more R¹⁰ groups; and
each R¹⁰ is selected from C₃₋₇-cycloalkyl, aryl, heteroaryl, O-heteroaryl, aralkyl and C₁₋₆-alkyl, each of which is optionally substituted by one or more A groups, wherein where R¹⁰ is C₁₋₆-alkyl and two or more R¹⁰ groups are attached to the same carbon atom, the R¹⁰ groups may be linked to form a spiroalkyl group; and
each R¹¹ is independently selected from C₁₋₆-alkyl, C₃₋₇-cycloalkyl, C₁₋₆-alkyl-C₃₋₇-cycloalkyl, C₁₋₆-alkyl-heteroaryl, C₄₋₇-heterocycloalkyl, aryl and heteroaryl, each of which is optionally substituted with one or more substituents selected from A; and
A is selected from halogen, —NR⁴SO₂R⁵, —CN, —OR⁶, —NR⁴R⁵, —NR⁷R¹¹, hydroxyl, —CF₃, —CONR⁴R⁵, —NR⁴COR⁵, —NR⁷(CO)NR⁴R⁵, —NO₂, —CO₂H, —CO₂R⁶, —SO₂R⁶, —SO₂NR⁴R⁵, —NR⁴COR⁵, —NR⁴COOR⁵, C₁₋₆-alkyl, aryl and —COR⁶.

A second aspect of the invention relates to a pharmaceutical composition comprising at least one compound as described above and a pharmaceutically acceptable carrier, diluent or excipient.

A third aspect of the invention relates to a compound as described above for use in medicine.

A fourth aspect of the invention relates to a compound as described above for use in treating a disorder selected from cancer and neurodegenerative diseases such as Parkinson's Disease.

A fifth aspect of the invention relates to the use of a compound as described above in the preparation of a medicament for treating or preventing a disorder selected from cancer and neurodegenerative diseases such as Parkinson's Disease.

A sixth aspect of the invention relates to the use of a compound as described above in the preparation of a medicament for the prevention or treatment of a disorder caused by, associated with or accompanied by any abnormal kinase activity wherein the kinase is preferably LRRK, more preferably LRRK2.

A seventh aspect of the invention relates to a method of treating a mammal having a disease state alleviated by inhibition of a kinase (preferably LRRK, more preferably LRRK2), wherein the method comprises administering to a mammal a therapeutically effective amount of a compound as described above.

An eighth aspect of the invention relates to the use of a compound as described above in an assay for identifying further candidate compounds capable of inhibition of a kinase, preferably LRRK, more preferably LRRK2.

A ninth aspect of the invention relates to a process for preparing a compound of formula I, said process comprising converting a compound of formula II into a compound of formula I:

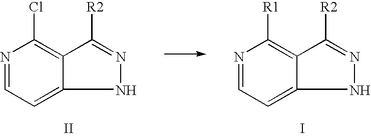

### DETAILED DESCRIPTION

The present invention relates to pyrazolopyridine compounds that are capable of inhibiting one or more kinases, more particularly LRRK, even more particularly LRRK2. Specifically, the invention relates to substituted pyrazolo[4,3-c]pyridine derivatives.

“Alkyl” is defined herein as a straight-chain or branched alkyl radical, for example, methyl, ethyl, propyl, isopropyl, butyl, isobutyl, tert-butyl, pentyl, hexyl.

“Cycloalkyl” is defined herein as a monocyclic alkyl ring, such as, cyclopropyl, cyclobutyl, cyclopentyl, cyclohexyl or cycloheptyl, or a fused bicyclic ring system such as norbornane.

“Halogen” is defined herein as chloro, fluoro, bromo or iodo.

As used herein, the term “aryl” refers to a C₆₋₁₂ aromatic group, which may be benzocondensed, for example, phenyl or naphthyl.

“Heteroaryl” is defined herein as a monocyclic or bicyclic C₂₋₁₂ aromatic ring comprising one or more heteroatoms (that may be the same or different), such as oxygen, nitrogen or sulphur. Examples of suitable heteroaryl groups include thienyl, furanyl, pyrrolyl, pyridinyl, oxazolyl, thiazolyl, imidazolyl, pyrazolyl, isoxazolyl, isothiazolyl, oxadiazolyl, triazolyl, thiadiazolyl etc. and benzo derivatives thereof, such as benzofuranyl, benzothienyl, benzimidazolyl, indolyl, isoindolyl, indazolyl etc.; or pyridyl, pyrazinyl, pyrimidinyl, pyridazinyl, triazinyl etc. and benzo derivatives thereof, such as quinolinyl, isoquinolinyl, cinnolinyl, phthalazinyl, quinazolinyl, quinoxalinyl, naphthyridinyl etc.

“Heterocycloalkyl” refers to a cyclic aliphatic group containing one or more heteroatoms selected from nitrogen, oxygen and sulphur, which is optionally interrupted by one or more —(CO)— groups in the ring and/or which optionally contains one or more double bonds in the ring. Preferably, the heterocycloalkyl group is a C₃₋₇-heterocycloalkyl, more preferably a C₃₋₆-heterocycloalkyl. Alternatively, the heterocycloalkyl group is a C₄₋₇-heterocycloalkyl, more preferably a C₄₋₆-heterocycloalkyl. Preferred heterocycloalkyl groups include, but are not limited to, piperazinyl, piperidinyl, morpholinyl, thiomorpholinyl, pyrrolidinyl, tetrahydrofuranyl and tetrahydropyranyl.

In one preferred embodiment of the invention, R² is selected from:

hydrogen;
halogen, more preferably bromine;
aryl optionally substituted by one or more substituents selected from R¹¹ and A;
C₁₋₆-alkyl optionally substituted by one or more substituents selected from R¹¹ and A;
C₂₋₆-alkenyl optionally substituted by one or more A substituents;
C₃₋₇-cycloalkyl;
heteroaryl optionally substituted by one or more substituents selected from R¹¹ and A;
C₄₋₇-heterocycloalkyl; and
fused aryl-C₄₋₇-heterocycloalkyl.

In one preferred embodiment of the invention, R² is selected from:

aryl optionally substituted by one or more substituents selected from —NR⁴COR⁵, —CONR⁴R⁵, OR⁶, halogen, optionally substituted C₁₋₆-alkyl, CN, C₄₋₇-heterocycloalkyl and heteroaryl;
C₁₋₆-alkyl optionally substituted by one or more substituents selected from —NR⁴COR⁵, —CONR⁴R⁵, —NR⁴R⁵, OR⁶, optionally substituted aryl, optionally substituted heteroaryl and C₄₋₇-heterocycloalkyl;
C₂₋₆-alkenyl optionally substituted by one or more —CONR⁴R⁵ substituents;
C₃₋₇-cycloalkyl;
heteroaryl optionally substituted by one or more substituents selected from C₄₋₇-heterocycloalkyl, C₁₋₆-alkyl, C₃₋₇-cycloalkyl, C₁₋₆-alkyl-C₃₋₇-cycloalkyl and OR⁶;
C₄₋₇-heterocycloalkyl; and
fused aryl-C₄₋₇-heterocycloalkyl.

In one preferred embodiment of the invention, R² is selected from:

a phenyl group optionally substituted by one or more substituents selected from —NHCO—C₁₋₆-alkyl, —CONHC₁₋₆-alkyl, CO—(N-morpholinyl), Cl, F, —OC₁₋₆-alkyl, —CONMe₂, OCF₃, CN, CF₃, C₁₋₆-alkyl-(A), N-morpholinyl and pyrazolyl;
a heteroaryl group selected from pyridinyl, quinolinyl, pyrazoyl, furanyl and pyrimidinyl, each of which may be optionally substituted by one or more substituents selected from C₁₋₆-alkyl, aralkyl, OC₁₋₆-alkyl, N-morpholinyl;
a C₁₋₆-alkyl group optionally substituted by one or more substituents selected from —CONR⁴R⁵, phenyl, pyridinyl and oxadiazolyl and piperidinyl, wherein said phenyl, pyridinyl and oxadiazolyl and piperidinyl groups are each optionally further substituted by one or more —NR⁴COR⁵, —CONR⁴R⁵, COR⁶, SO₂R⁶ or aryl groups.

In a more preferred embodiment of the invention, each —CONR⁴R⁵ group is independently selected from:

—CO(N-morpholinyl), —CO(N-piperidinyl), —CO(N-pyrrolidinyl), —CO—(N-piperazinyl), each of which may be optionally further substituted by one or more substituents selected from aryl, heteroaryl, —OR⁶, CF₃, aralkyl, —NR⁴COR⁵—CONR⁴R⁵, —NR⁴R⁵, halogen, C₁₋₆-alkyl; and —CON(C₁₋₆-alkyl)₂, CONH(C₁₋₆-alkyl), CON(C₁₋₆-alkyl)(aralkyl), CONH(C₃₋₇-cycloalkyl), —CONH(aryl), —CONH(heteroaryl), wherein said C₁₋₆-alkyl, aralkyl, aryl and heteroaryl groups are each optionally further substituted by one or more R¹¹ or A groups.

In one preferred embodiment of the invention, R² is a C₁₋₆-alkyl group optionally substituted by one or more substituents selected from —NR⁴COR⁵, —CONR⁴R⁵, —NR⁴R⁵, OR⁶, C₄₋₇-heterocycloalkyl, heteroaryl and aryl, wherein said aryl group is optionally substituted by one or more substituents selected from —NR⁴COR⁵ and —CONR⁴R⁵.

In one preferred embodiment of the invention, R² is selected from —CH₂CH₂CO—NR⁴R⁵, C₁₋₆-alkyl, C₃₋₇ cycloalkyl and a heteroaryl selected from furanyl and pyrazolyl, wherein said furanyl and pyrazolyl groups may be optionally substituted by one or more substituents selected from C₁₋₆-alkyl, C₃₋₇-cycloalkyl and C₁₋₆-alkyl-C₃₋₇-cycloalkyl.

In one preferred embodiment of the invention, R² is selected from Me,

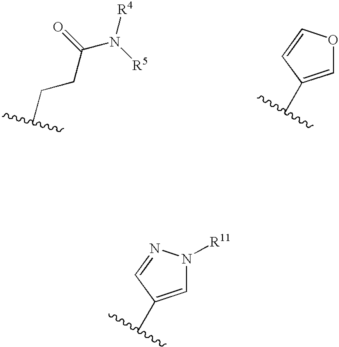

wherein R⁴ and R⁵ together with the N to which they are attached form a C₃₋₆-heterocycloalkyl ring optionally further containing one or more groups selected from oxygen, sulfur, nitrogen and CO, wherein said C₃₋₆-heterocycloalkyl ring is saturated or unsaturated and is optionally substituted with one or more groups selected from A, NR⁸R⁹ and R¹⁰. Even more preferably, R⁴ and R⁵ together with the N to which they are attached form a 6-membered heterocycloalkyl ring that is optionally substituted with one or more groups selected from A, NR⁸R⁹ and R¹⁰. More preferably still, R⁴ and R⁵ together with the N to which they are attached form a saturated 6-membered ring (more preferably, a piperidinyl ring) that is optionally substituted with one or more groups selected from A, NR⁸R⁹ and R¹⁰.

In one highly preferred embodiment of the invention, R² is selected from Me,

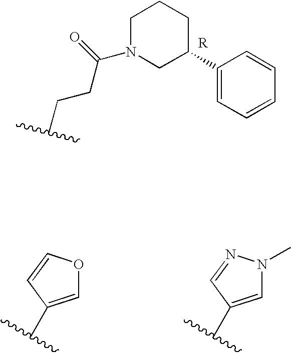

In one preferred embodiment of the invention, R² is an unsubstituted C₁₋₆-alkyl group, more preferably methyl.

In one preferred embodiment of the invention, R¹ is selected from:

### —NHR³;

aryl;
heteroaryl;
C₄₋₇-heterocycloalkyl;
fused aryl-C₄₋₇-heterocycloalkyl;
—C₃₋₇-cycloalkyl;

### —NR³R⁶;

### OR³;

### NR⁴R⁵; and

—C₁₋₆ alkyl optionally substituted with a substituent selected from R¹¹ and a group A;
wherein said aryl, heteroaryl, fused aryl-C₄₋₇-heterocycloalkyl and C₄₋₇-heterocycloalkyl are each optionally substituted with one or more substituents selected from C₁₋₆-alkyl, C₃₋₇-cycloalkyl, heteroaryl, C₄₋₇-heterocycloalkyl, aryl and a group A, and said C₁₋₆-alkyl, C₃₋₇-cycloalkyl, heteroaryl, C₄₋₇-heterocycloalkyl, and aryl substituents are in turn each optionally substituted with one or more groups selected from R¹¹ and a group A.

In one preferred embodiment of the invention, R¹ is —NHR³ and R³ is selected from:

C₁₋₆-alkyl, optionally substituted by one or more —OR⁶, NR⁴COR⁵, heteroaryl, aryl, C₄₋₇-heterocycloalkyl, and C₃₋₇-cycloalkyl groups, wherein said aryl and heteroaryl groups are each independently optionally further substituted by one or more groups selected from CF₃, halogen, C₁₋₆-alkyl, —OR⁶ and —NR⁴R⁵;
a phenyl group optionally substituted by one or more substituents selected from —OR⁶, NR⁴COR⁵, —CONR⁴R⁵, aryl, —NR⁴R⁵, C₁₋₆-alkyl-heteroaryl, heteroaryl, halogen, —SO₂R⁶, CN, CF₃, C₁₋₆-alkyl, —SO₂NR⁴R⁵, —NR⁴SO₂R⁵, wherein said C₁₋₆-alkyl, heteroaryl and aryl groups are each independently optionally further substituted by one or more groups selected from CN, CF₃, halogen, C₁₋₆-alkyl, —OR⁶ and —NR⁴R⁵;
a heteroaryl group optionally substituted by one or more substituents selected from aryl, C₁₋₆-alkyl, and —NR⁴R⁵, wherein said aryl group is optionally further substituted by one or more A groups;
a C₄₋₇-heterocycloalkyl optionally substituted by one or more —COR⁶ groups;
a C₃₋₇-cycloalkyl group optionally substituted by one or more halogen or C₁₋₆-alkyl groups.

In one preferred embodiment of the invention, R¹ is —NHR³, wherein R³ is selected from C₁₋₆-alkyl, C₃₋₇-cycloalkyl, C₄₋₇-heterocycloalkyl and aryl, each of which may be optionally substituted by one or more with one or more substituents selected from R¹¹ and A.

In one preferred embodiment of the invention, R¹ is —OR³, wherein R³ is selected from C₁₋₆-alkyl, C₃₋₇-cycloalkyl, C₄₋₇-heterocycloalkyl and aryl, each of which may be optionally substituted by one or more with one or more substituents selected from R¹¹ and A.

In one preferred embodiment of the invention, R¹ is —OR³, wherein R³ is C₁₋₆-alkyl, C₃₋₇-cycloalkyl or C₄₋₇-heterocycloalkyl, each of which may be optionally substituted by one or more A substituents. In one particularly preferred embodiment of the invention, R¹ is —O—C₃₋₇-cycloalkyl, more preferably, —O-cyclohexyl.

In one preferred embodiment of the invention. R¹ is aryl or heteroaryl, each of which may be optionally substituted by one or more with one or more substituents selected from R¹¹ and A.

In one preferred embodiment of the invention. R¹ is —NH—C₃₋₇-cycloalkyl or NH—C₄₋₇-heterocycloalkyl, each of which may be optionally substituted by one or more A substituents. Preferably, A is halogen or C₁₋₆-alkyl.

In one preferred embodiment of the invention, R³ is cyclohexyl or tetrahydropyranyl, each of which may be optionally substituted by one or more A substituents.

In one preferred embodiment of the invention. R¹ is selected from the following:

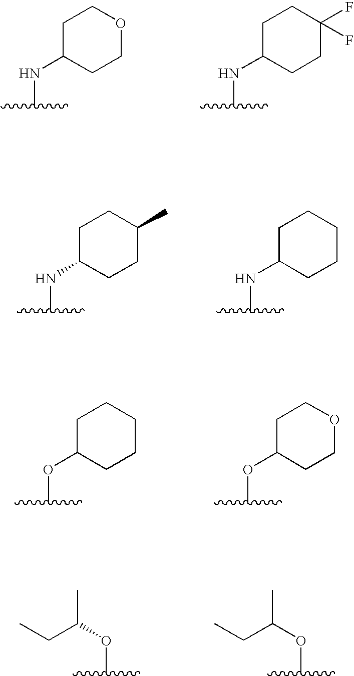

In one preferred embodiment of the invention, R¹ is —NH-cyclohexyl.

In one preferred embodiment of the invention, R¹ is —NHR³ and R² is an unsubstituted C₁₋₆-alkyl group, more preferably methyl.

In one preferred embodiment of the invention, R¹ is —NHR³ and R² is a C₁₋₆-alkyl group substituted by one or more —CONR⁴R⁵ groups.

In one preferred embodiment of the invention, R¹ is —NHR³ and R² is an aryl or heteroaryl group, each of which may be optionally substituted by one or more substituents selected from C₄₋₇-heterocycloalkyl, C₁₋₆-alkyl, C₃₋₇-cycloalkyl, C₁₋₆-alkyl-C₃₋₇-cycloalkyl and OR⁶.

In one preferred embodiment of the invention. R¹ is —OR³ and R² is a C₁₋₆-alkyl group, more preferably methyl.

In one preferred embodiment of the invention, R¹ is selected from:

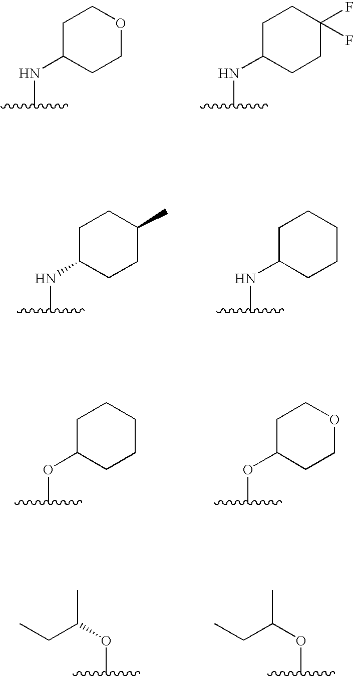

and R² is selected from

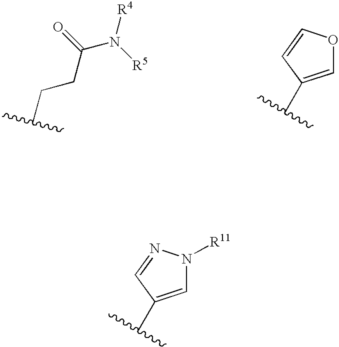

wherein R⁴ and R⁵ together with the N to which they are attached form a C₃₋₆-heterocycloalkyl ring optionally further containing one or more groups selected from oxygen, sulfur, nitrogen and CO, wherein said C₃₋₆-heterocycloalkyl ring is saturated or unsaturated and is optionally substituted with one or more groups selected from A, NR⁸R⁹ and R¹⁰. Even more preferably, R⁴ and R⁵ together with the N to which they are attached form a 6-membered heterocycloalkyl ring that is optionally substituted with one or more groups selected from A, NR⁸R⁹ and R¹⁰. More preferably still. R⁴ and R⁵ together with the N to which they are attached form a saturated 6-membered ring (more preferably, a piperidinyl ring) that is optionally substituted with one or more groups selected from A, NR⁸R⁹ and R¹⁰.

More preferably, R¹ is as defined above, and R² is selected from Me,

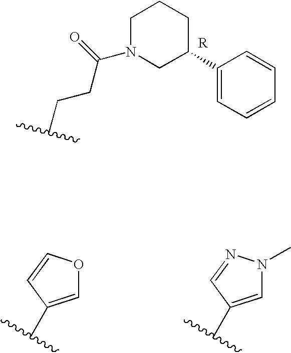

In one preferred embodiment of the invention:

R¹ is selected from aryl, heteroaryl, C₄₋₇-heterocycloalkyl, fused aryl-C₄₋₇-heterocycloalkyl and —NHR³, wherein said aryl, heteroaryl, fused aryl-C₄₋₇-heterocycloalkyl and C₄₋₇-heterocycloalkyl are each optionally substituted with one or more substituents selected from C₁₋₆-alkyl, C₃₋₇-cycloalkyl, heteroaryl, C₄₋₇-heterocycloalkyl, aryl and a group A, and said C₁₋₆-alkyl, C₃₋₇-cycloalkyl, heteroaryl, C₄₋₇-heterocycloalkyl, and aryl substituents are in turn each optionally substituted with one or more groups selected from R¹¹ and a group A; and
R² is selected from hydrogen, aryl, C₁₋₆-alkyl, C₃₋₇-cycloalkyl, heteroaryl, C₄₋₇ heterocycloalkyl and halogen, wherein said C₁₋₆-alkyl, aryl, heteroaryl and C₄₋₇-heterocycloalkyl are each optionally substituted with one or more substituents selected from R¹¹ and A.

In another preferred embodiment of the invention R² is a C₁₋₆-alkyl group optionally substituted with one or more substituents selected from R¹¹ and A.

In one preferred embodiment of the invention R¹ is selected from:

NH—R³, where R³ is selected from C₁₋₆-alkyl, morpholinyl, C₃₋₇-cycloalkyl, fused aryl-C₄₋₇-heterocycloalkyl, piperidinyl, tetrahydropyranyl, piperazinyl, phenyl, pyridinyl, indazolyl and pyrazolyl, each of which is optionally substituted by one or more substituents selected from R¹¹ and A; and
furyl, pyrazolyl and phenyl, each of which is optionally substituted by one or more substituents selected from R¹¹ and A.

In one preferred embodiment of the invention R¹ is selected from:

NH—C₁₋₆-alkyl, wherein said C₁₋₆-alkyl is optionally substituted by one or more substituents selected from OR⁶, OH, C₄₋₇ heterocycloalkyl, NR⁴R⁵, heteroaryl, C₃₋₇-cycloalkyl, phenyl, wherein said phenyl group is optionally substituted by one or more halo groups, and said C₄₋₇ heterocycloalkyl group is optionally substituted by one or more C₁₋₆-alkyl groups;
NH-piperazinyl, wherein said piperazinyl is optionally substituted by one or more substituents selected from C₁₋₆-alkyl, aryl, C₁₋₆-alkyl-aryl and heteroaryl, each of which is optionally further substituted by one or more halo groups;

### NH-morpholinyl;

NH—C₃₋₇-cycloalkyl, wherein said C₃₋₇-cycloalkyl is optionally substituted by one or more substituents selected from OH and halo;
NH-fused aryl-C₄₋₇-heterocycloalkyl, wherein said fused aryl-C₄₋₇-heterocycloalkyl is optionally substituted by one or more C₁₋₆-alkyl groups;
NH-piperidinyl, wherein said piperidinyl is optionally substituted by one or more C₁₋₆-alkyl groups;

### NH-tetrahydropyranyl;

a furyl group;
a pyrazolyl group, optionally substituted by one or more C₁₋₆-alkyl groups;
NH-phenyl, wherein said phenyl is optionally substituted by one or more substituents selected from halo, CF₃, OH, OR⁶, NR⁴SO₂R⁵, NR⁴R⁵, C₄₋₇ heterocycloalkyl, CONR⁴R⁵ and —NR⁴COR⁵;
NH-pyridinyl, wherein said pyridinyl is optionally substituted by one or more substituents selected from C₄₋₇ heterocycloalkyl and aryl, wherein said aryl group is optionally further substituted with one or more halo groups;
phenyl, optionally substituted by one or more substituents selected from halo, OR⁶, —NR⁴SO₂R⁵, CN, C₄₋₇ heterocycloalkyl and C₁₋₆-alkyl-NR⁴SO₂R⁵;

NH-indazolyl, wherein said indazolyl is optionally substituted by one or more C₁₋₆-alkyl groups; and

### NH-pyrazolyl.

In one preferred embodiment of the invention R¹ is selected from:

NH—C₁₋₆-alkyl, wherein said C₁₋₆-alkyl is optionally substituted by one or more substituents selected from OMe, OH, tetrahydropyranyl, pyrrolidinyl, NEt₂, imidazolyl, cyclopropyl, cyclobutyl, cyclopentyl, cyclohexyl, phenyl, wherein said phenyl group is optionally substituted by one or more chloro groups, and said pyrrolidinyl group is optionally substituted by one or more methyl groups;
NH-piperazinyl, wherein said piperazinyl is optionally substituted by one or more substituents selected from methyl, phenyl, CH₂-phenyl and pyridinyl, wherein the phenyl group is optionally further substituted by one or more F or Cl groups;

### NH-morpholinyl;

NH-cyclopropyl, NH-cyclobutyl, NH-cyclopentyl and NH-cyclohexyl, wherein said cyclopropyl, cyclobutyl, cyclopentyl and cyclohexyl groups are optionally substituted by one or more substituents selected from OH and F;
NH-(1,2,3,4-tetrahydroisoquinolinyl), wherein said 1,2,3,4-tetrahydroisoquinolinyl group is optionally substituted by one or more methyl groups;
NH-piperidinyl, wherein said piperidinyl is optionally substituted by one or more methyl groups;

### NH-tetrahydropyranyl;

a furyl group;
a pyrazolyl group, optionally substituted by one or more methyl groups;
NH-phenyl, wherein said phenyl is optionally substituted by one or more substituents selected from F, Cl, Br, CF₃, OH, OEt, NHSO₂Me, NMe₂, morpholinyl, CONMe₂, CONH₂ and —NHCOMe;
NH-pyridinyl, wherein said pyridinyl is optionally substituted by one or more substituents selected from morpholinyl and phenyl wherein said phenyl group is optionally further substituted with one or more CN groups;
phenyl, optionally substituted by one or more substituents selected from F, Cl, OMe, —NHSO₂Me, CN, morpholinyl and CH₂—NHSO₂Me;
NH-indazolyl, wherein said indazolyl is optionally substituted by one or more methyl groups; and

### NH-pyrazolyl.

In one highly preferred embodiment of the invention the compound of formula I is selected from the following:

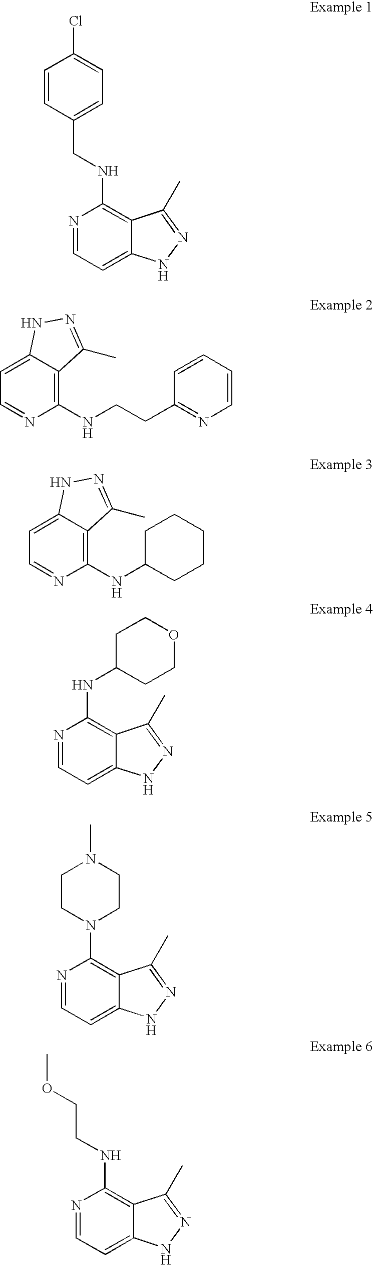

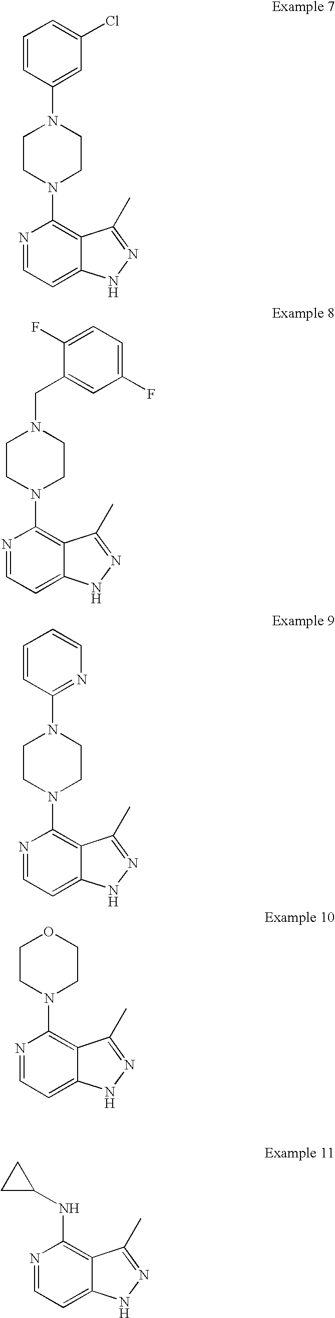

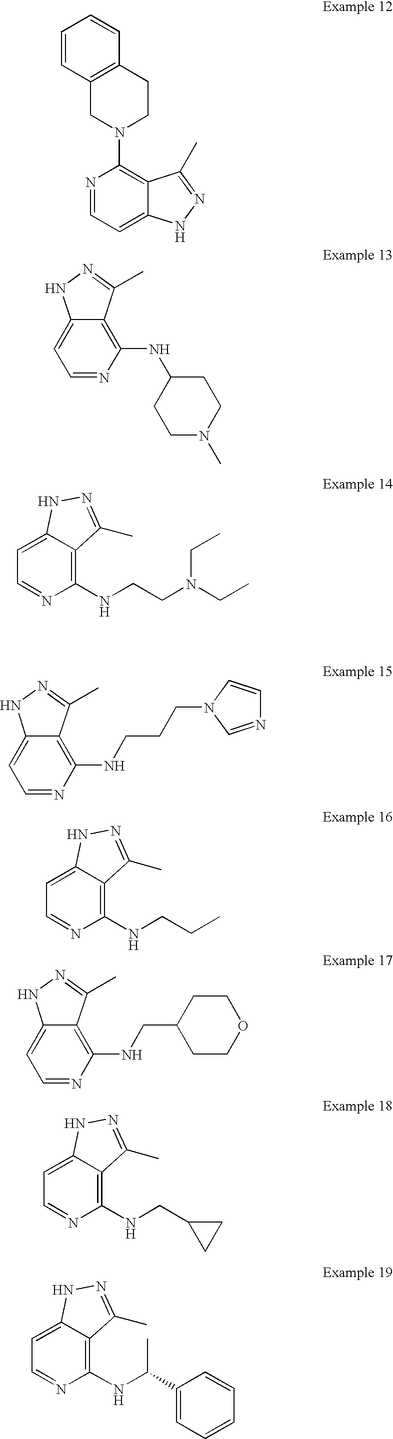

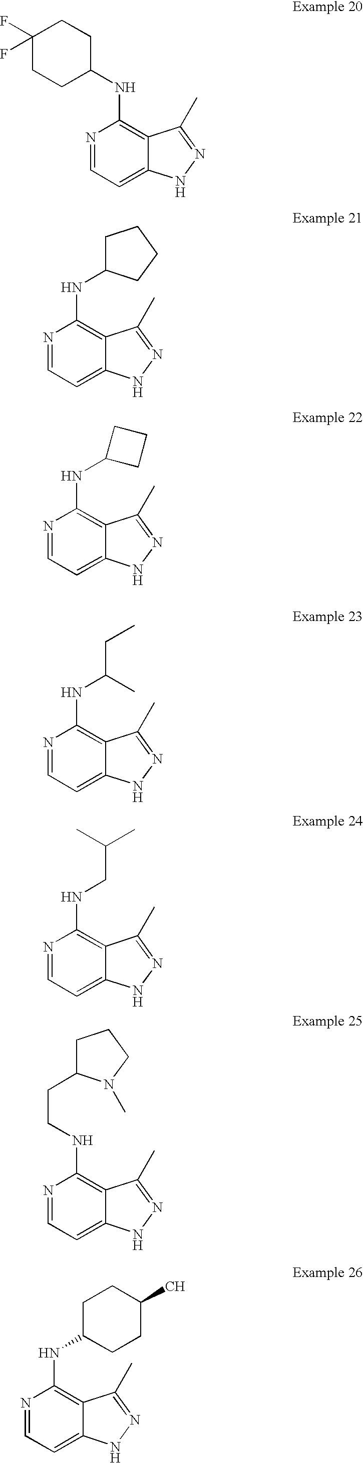

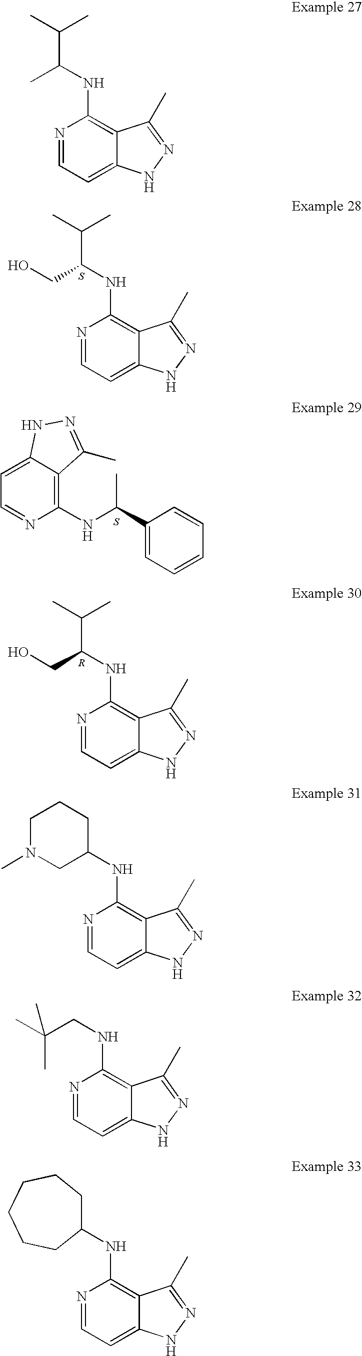

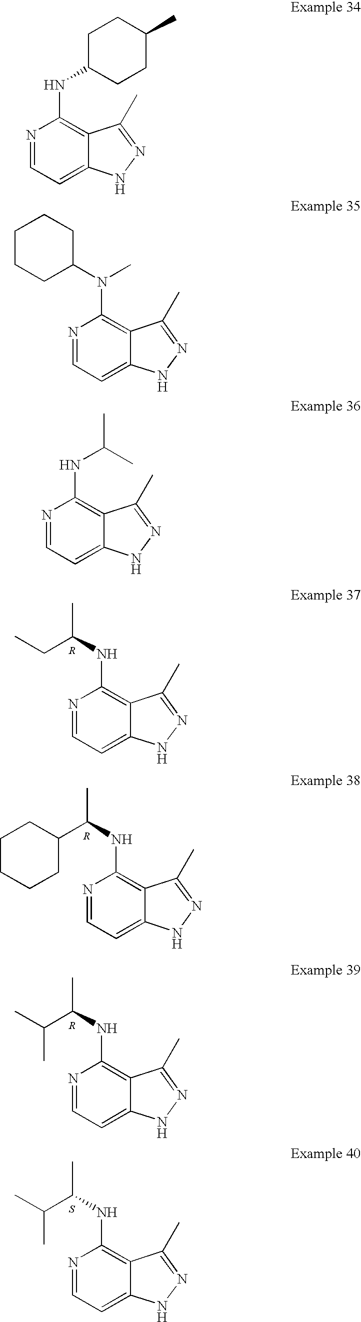

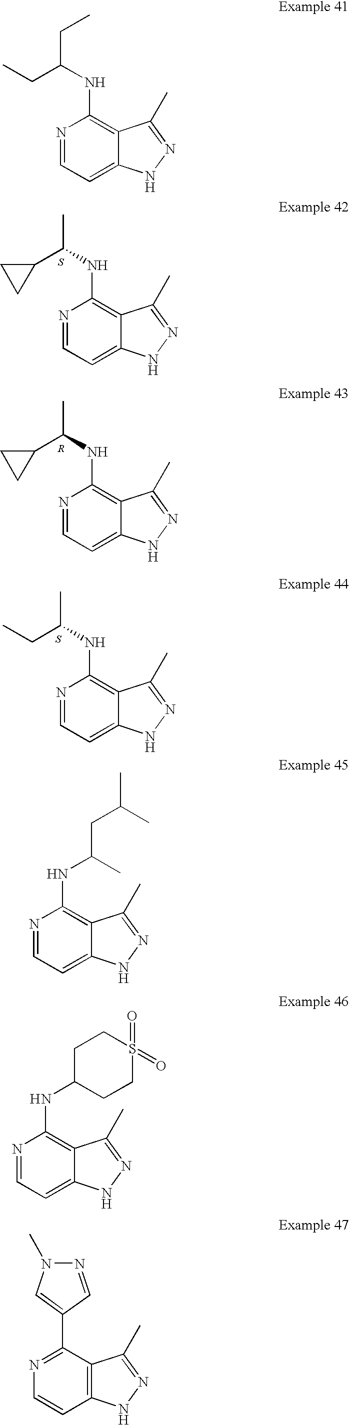

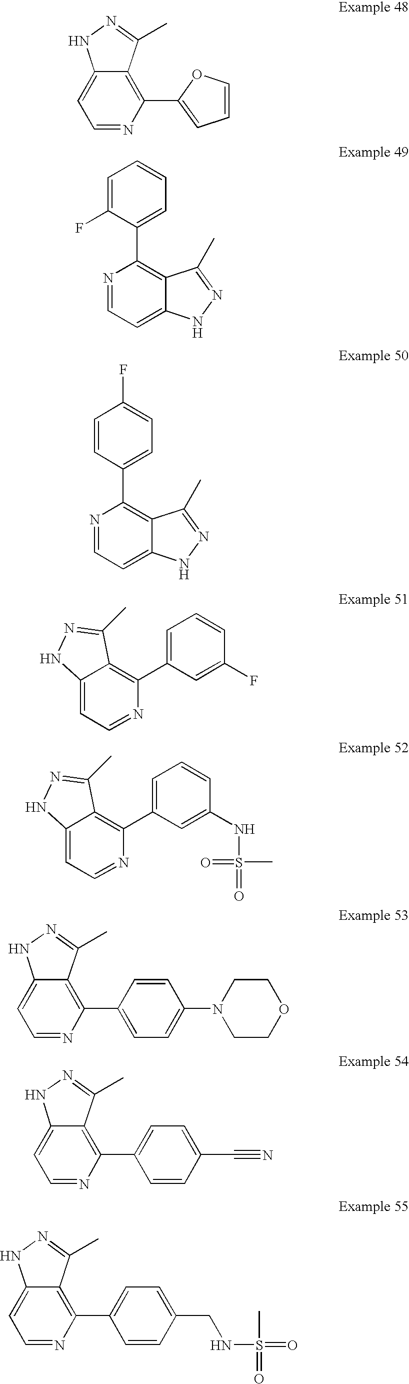

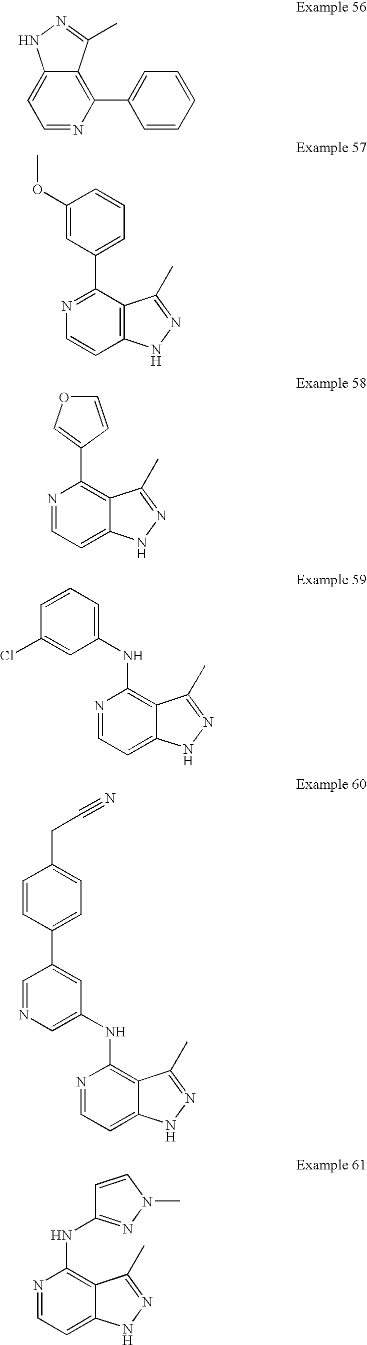

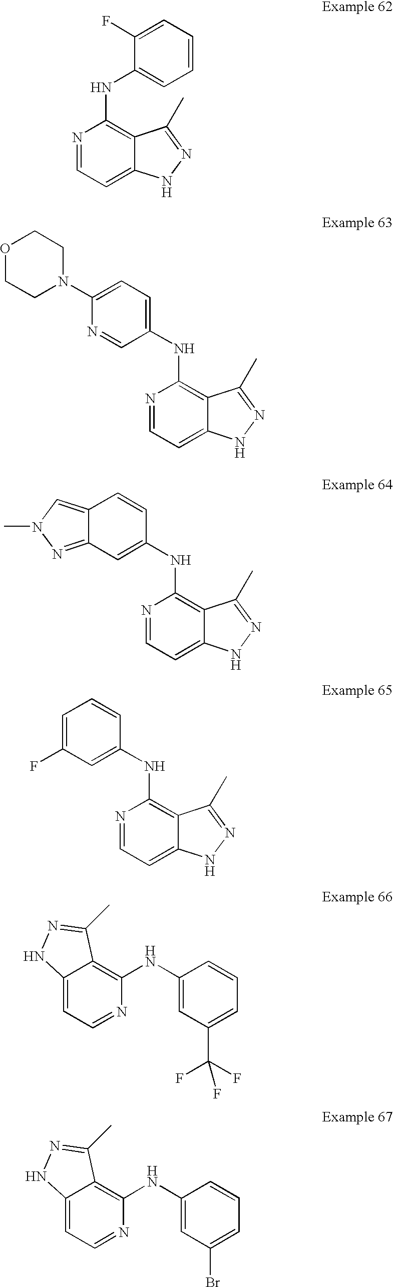

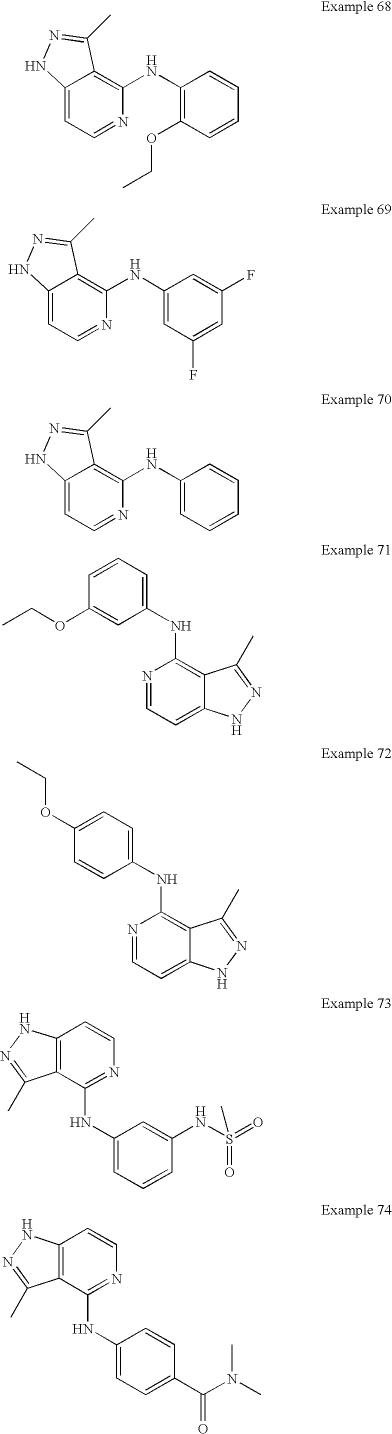

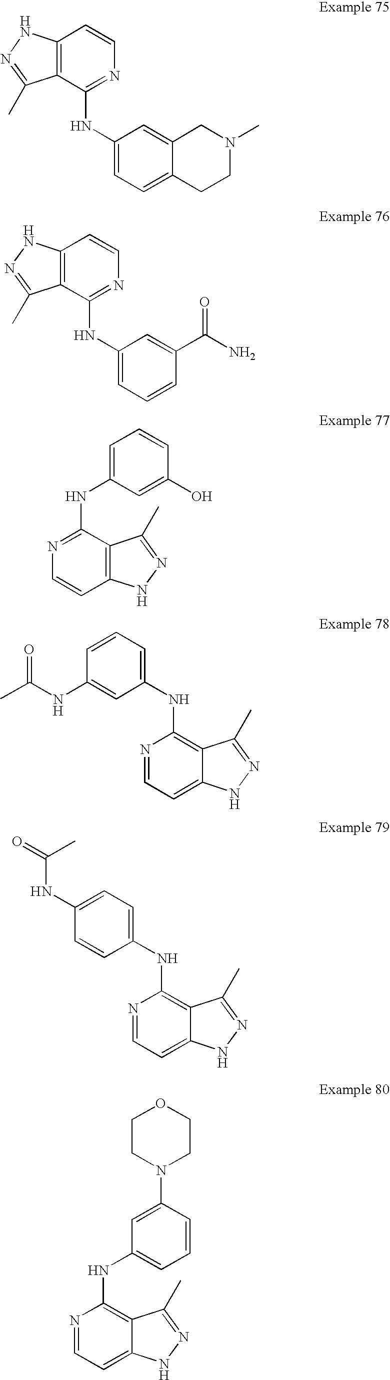

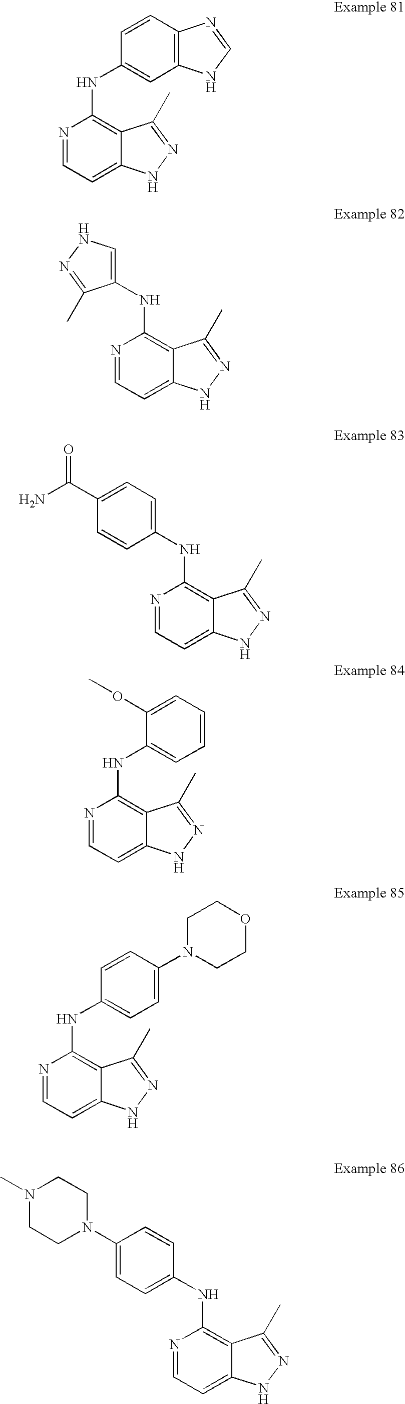

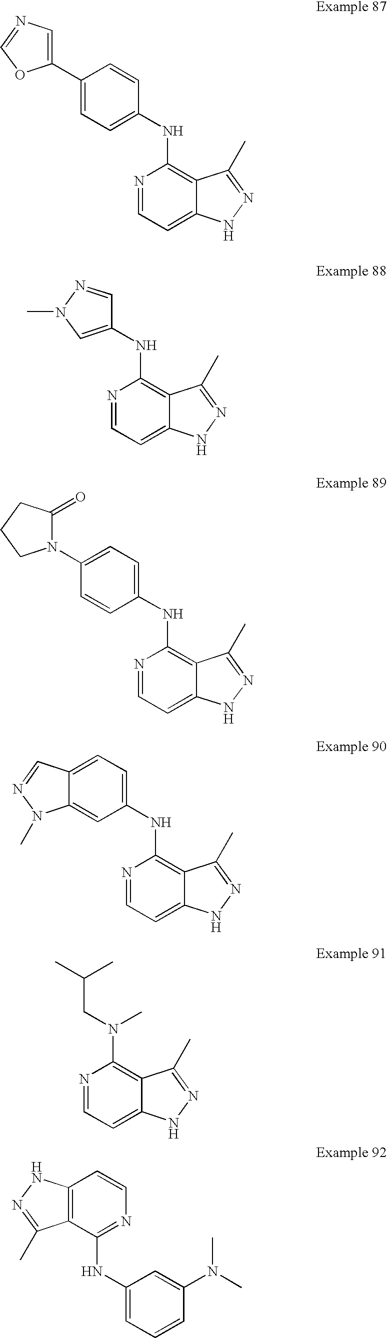

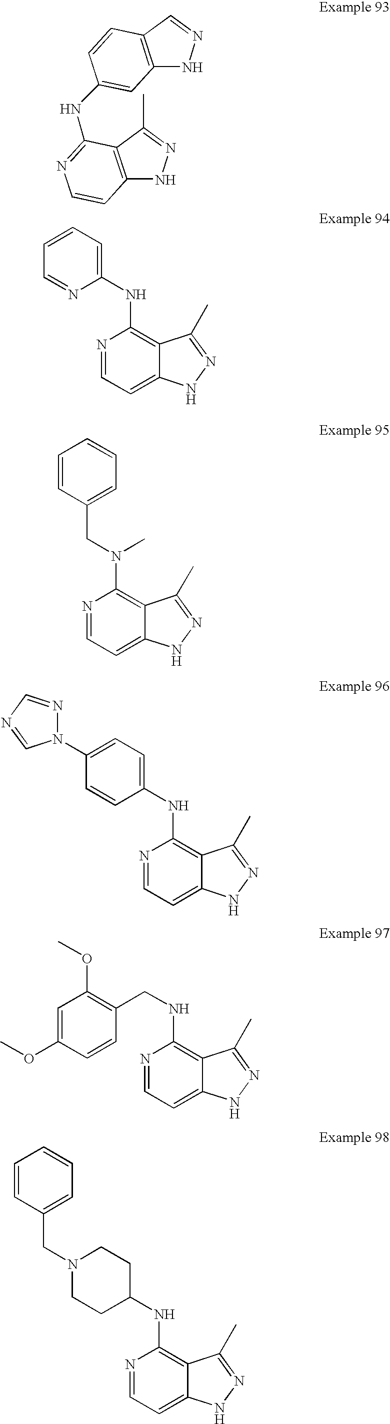

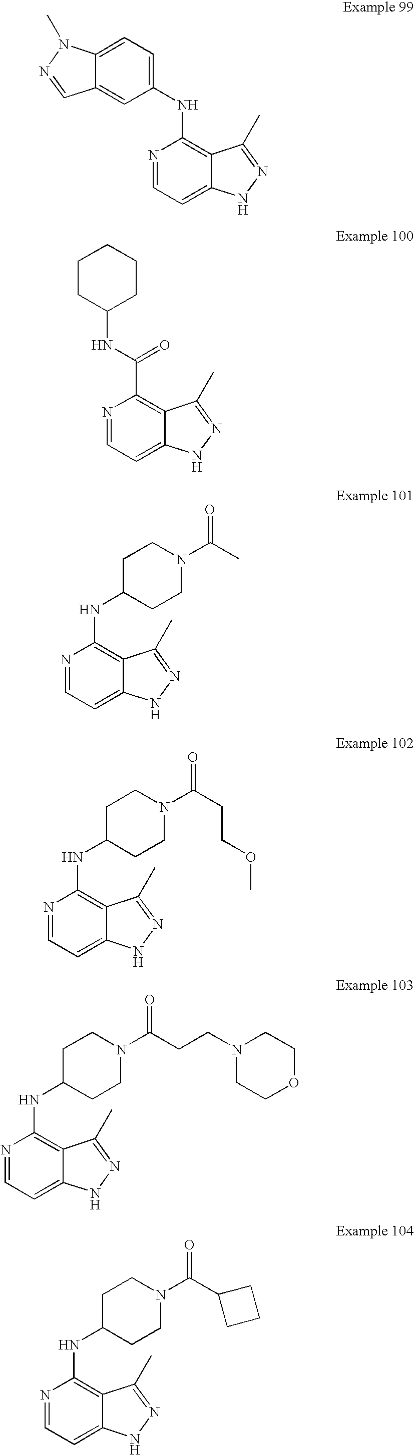

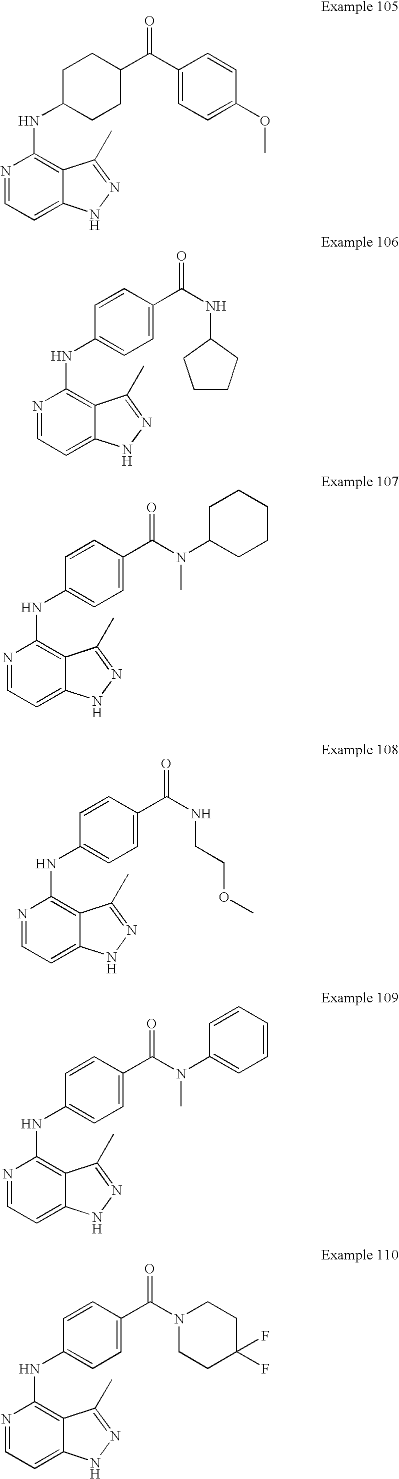

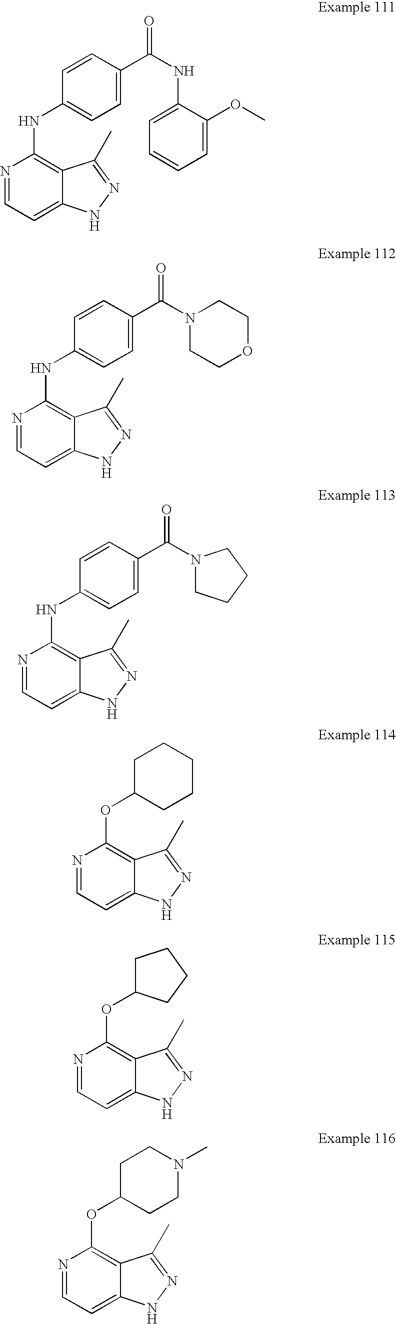

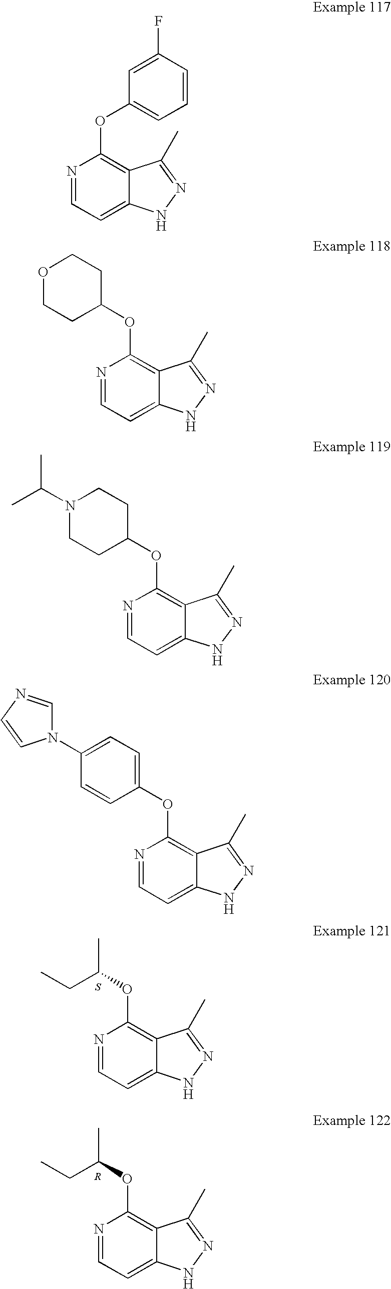

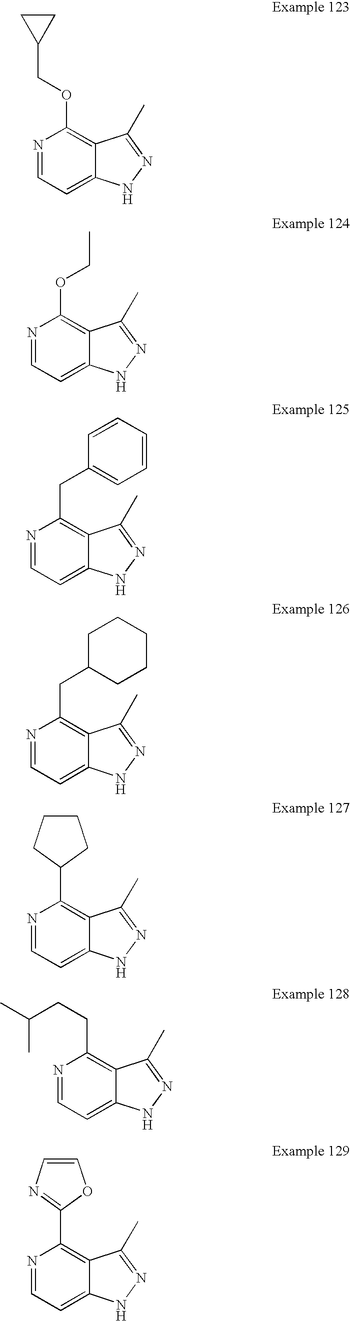

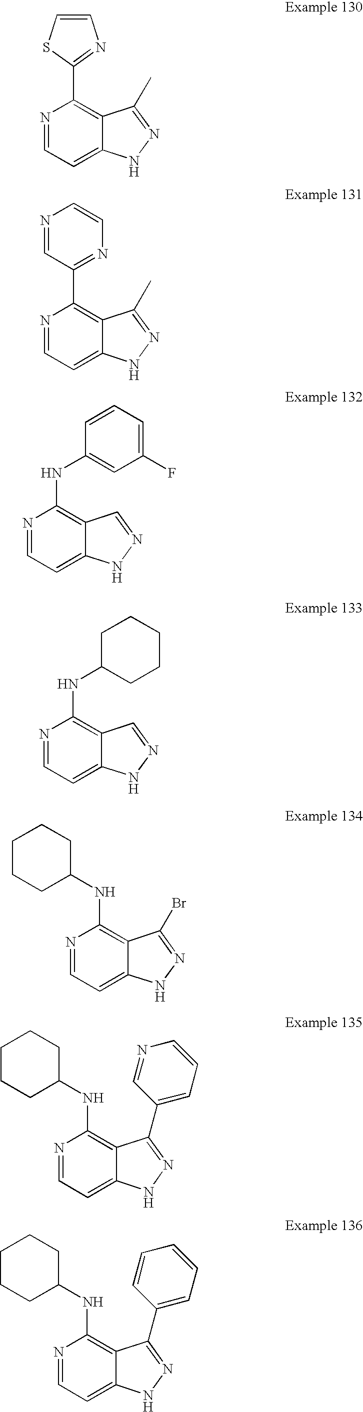

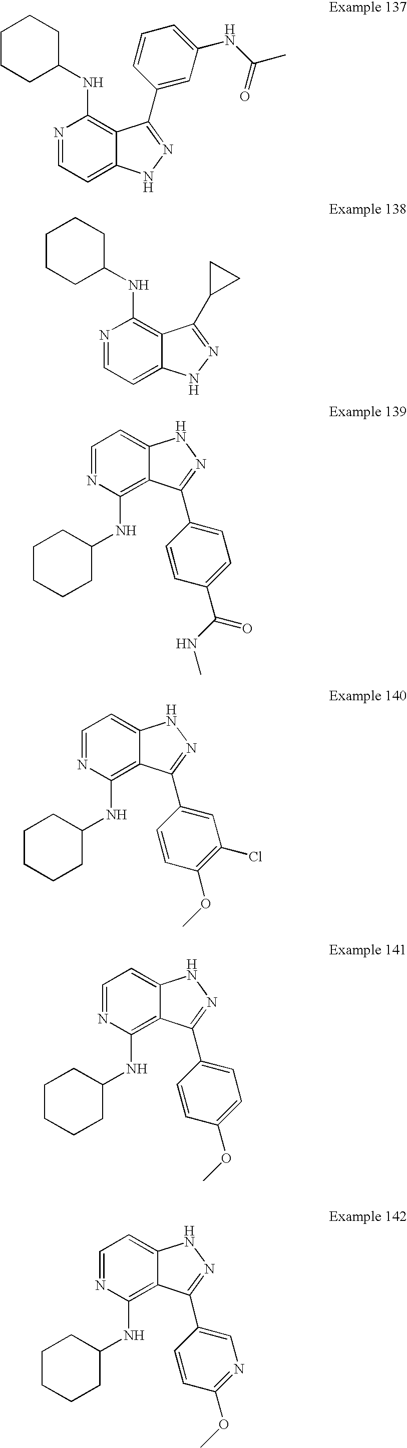

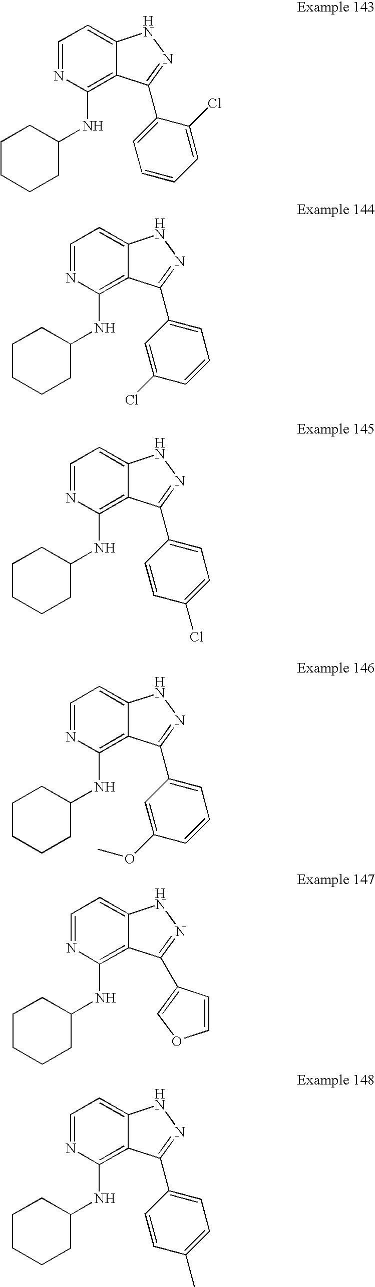

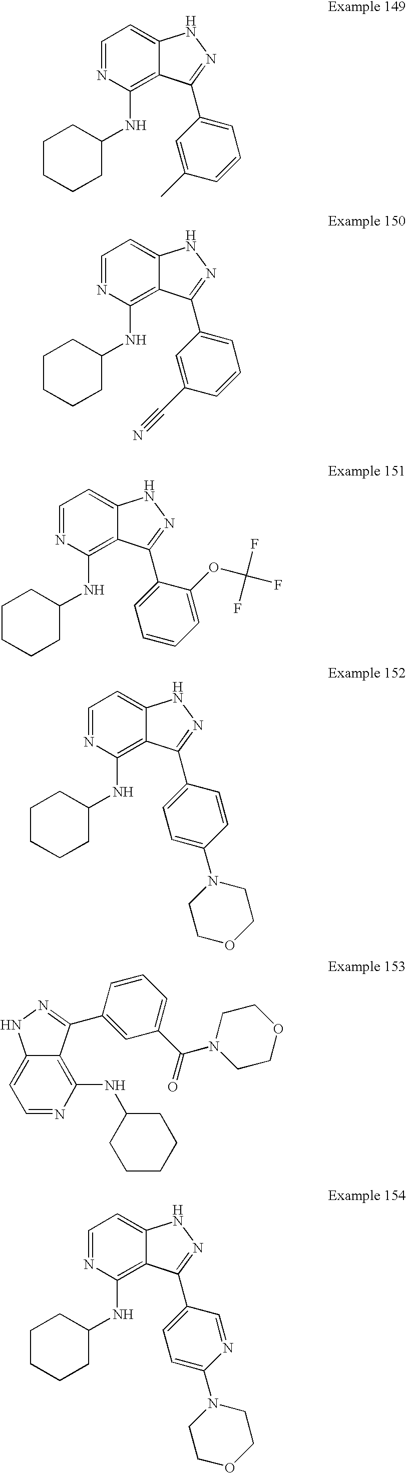

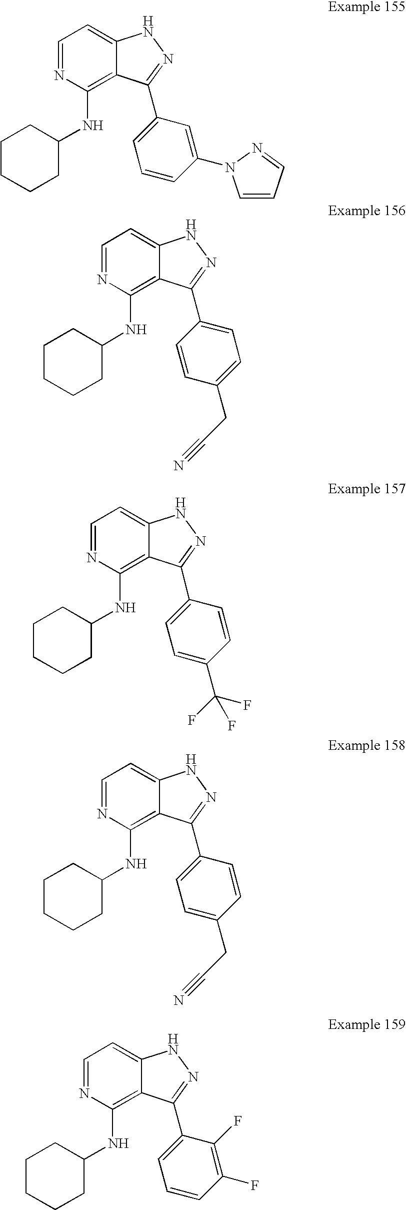

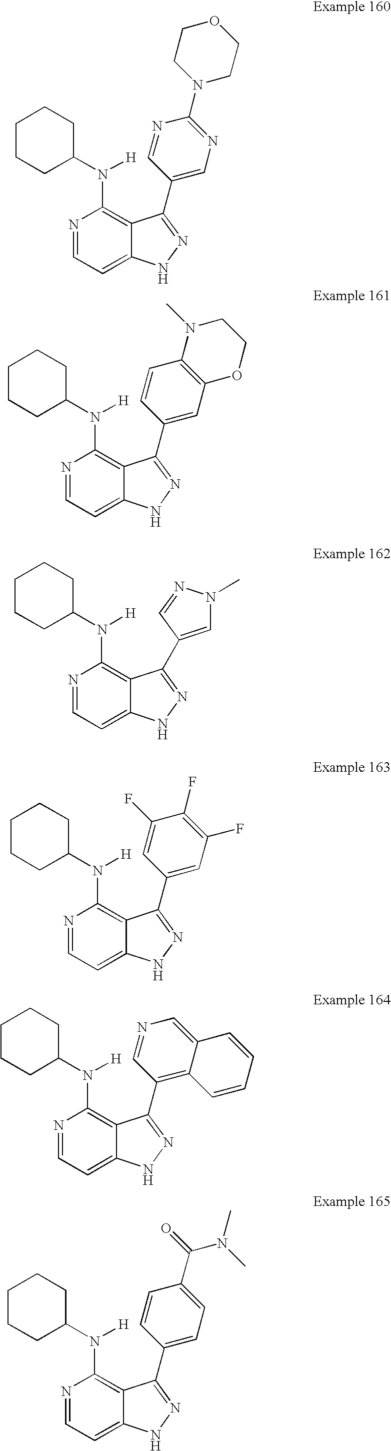

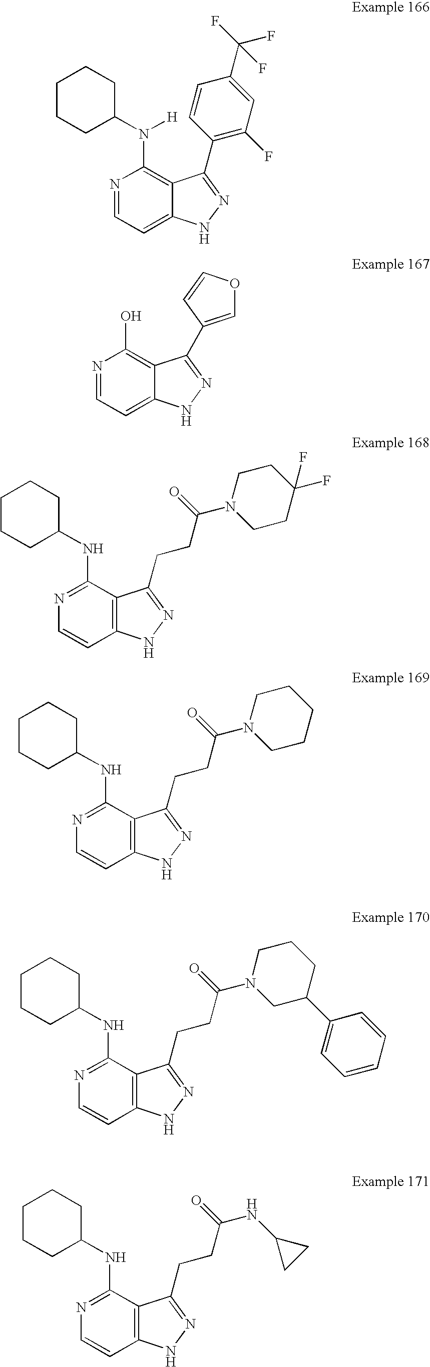

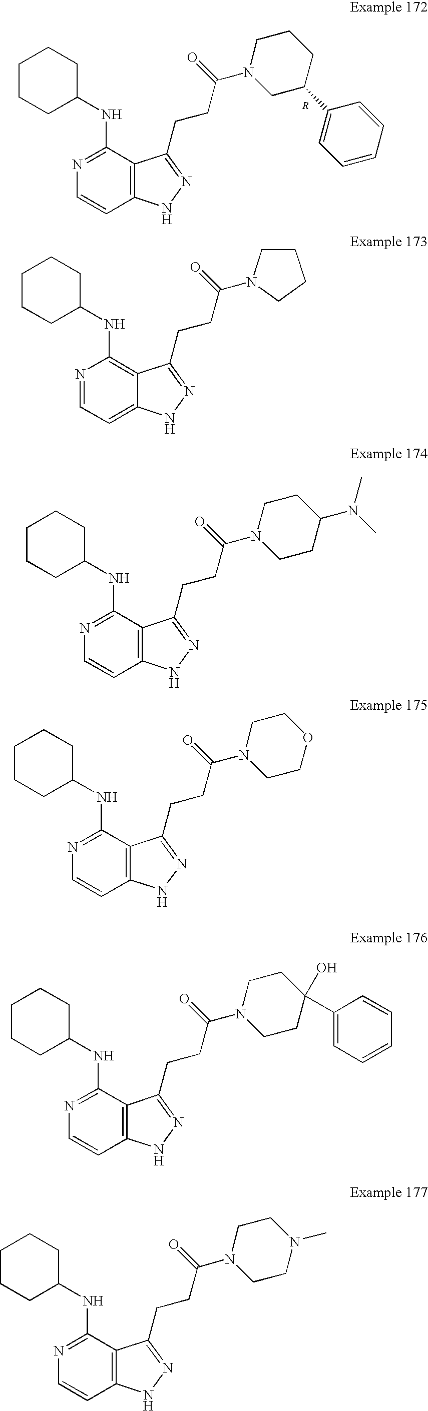

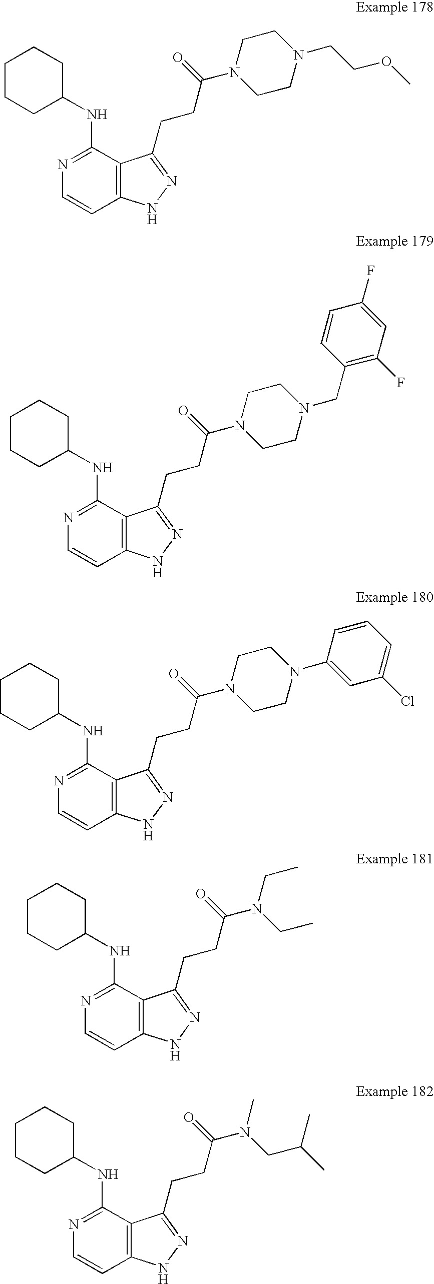

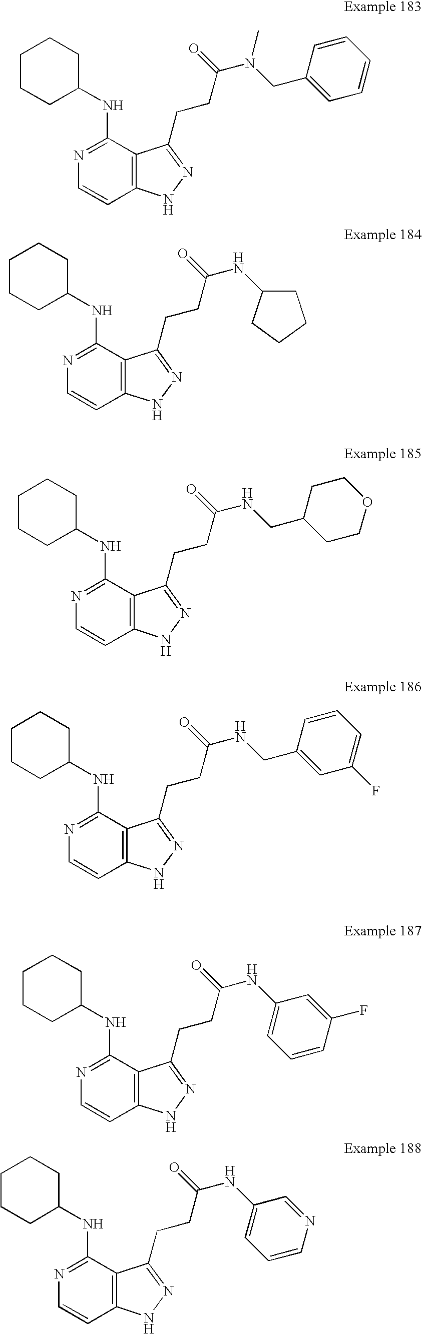

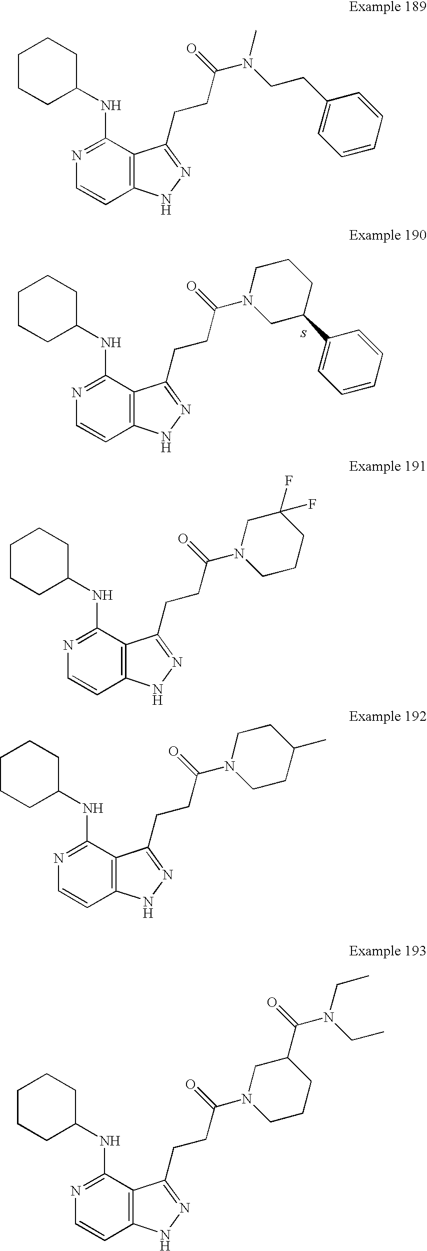

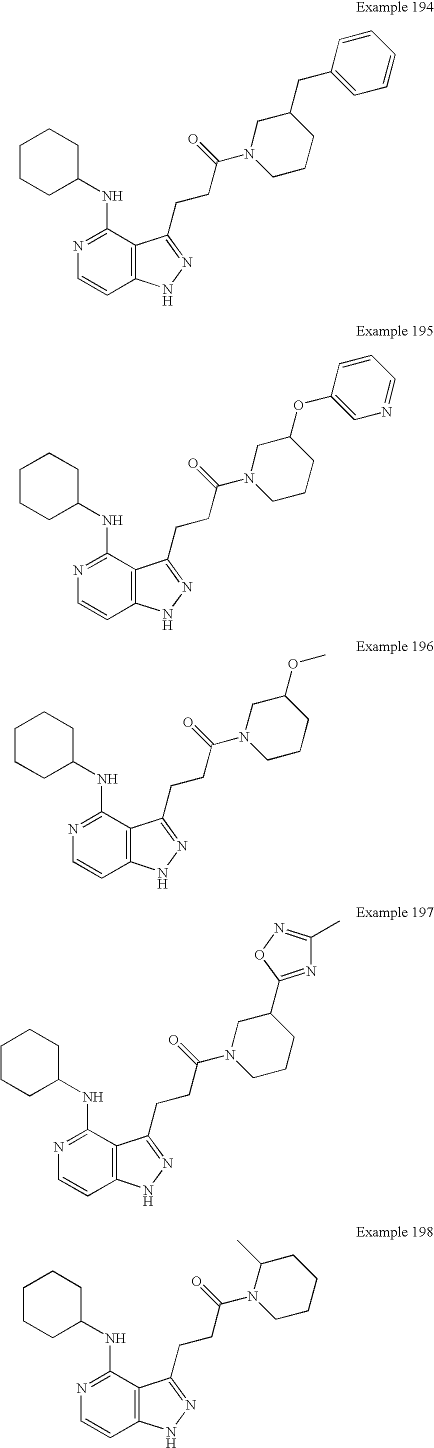

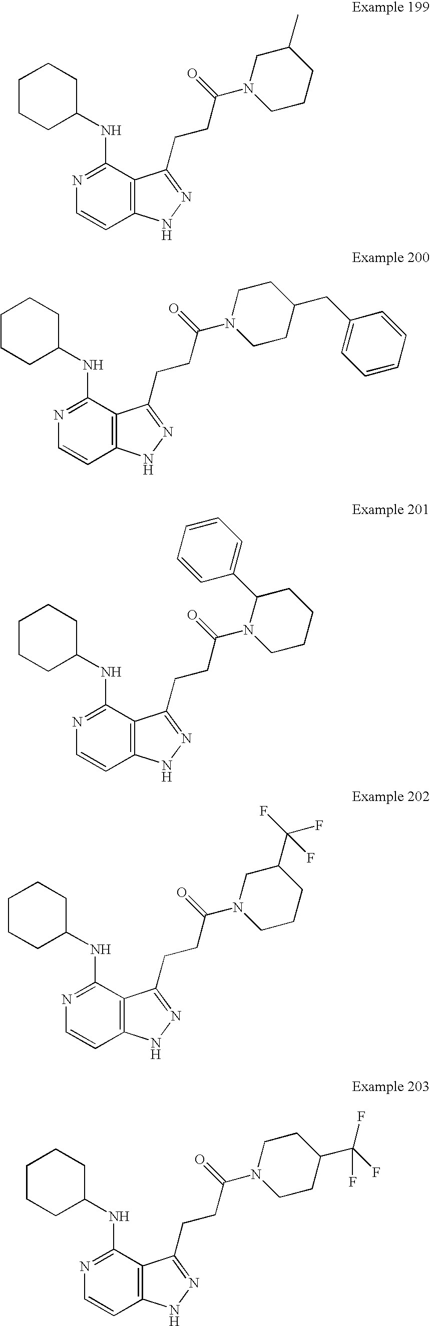

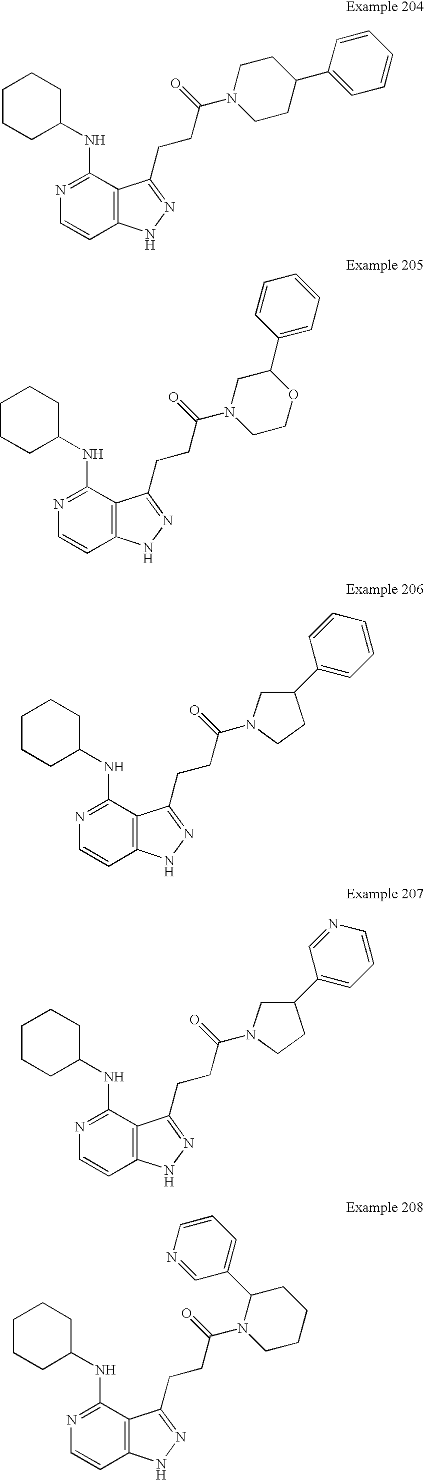

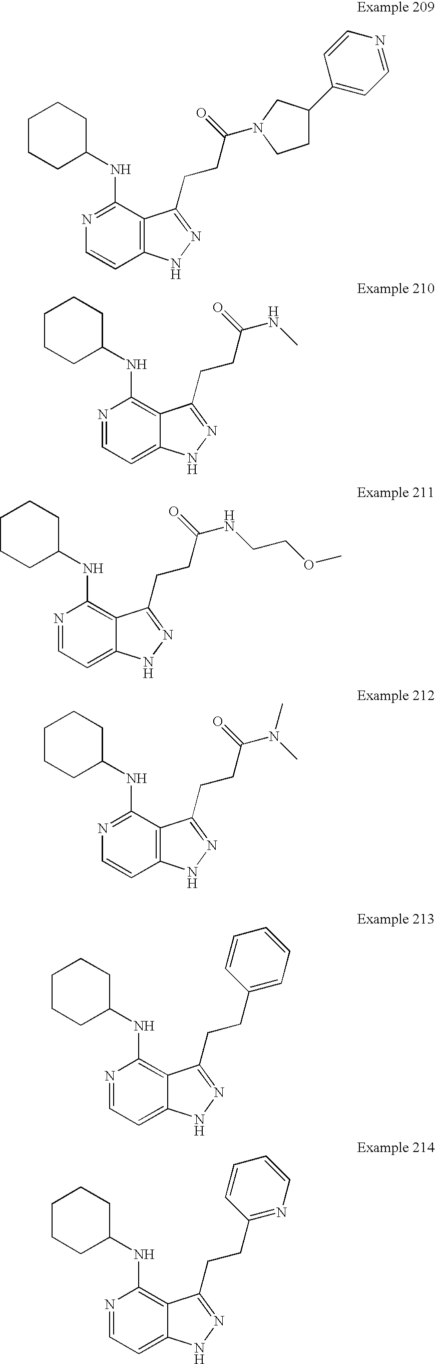

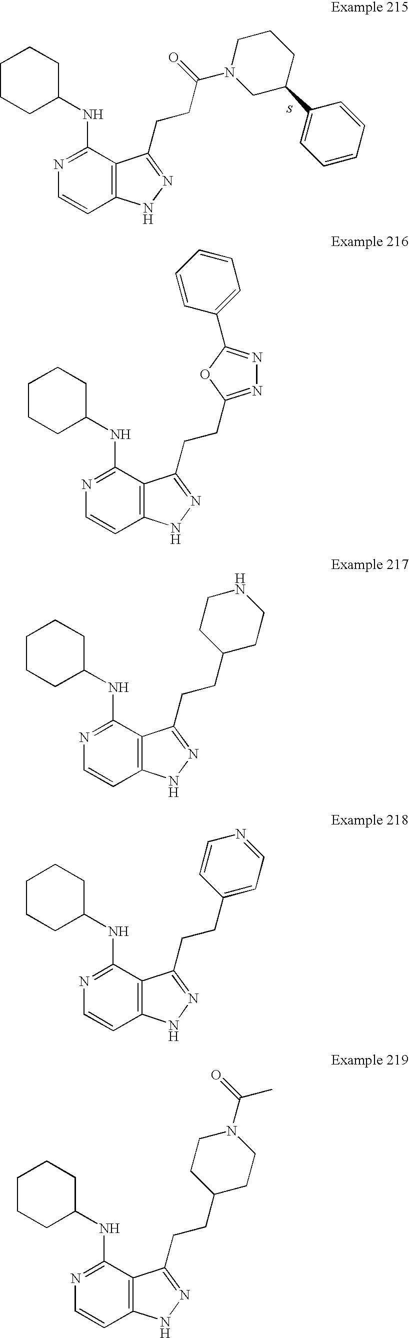

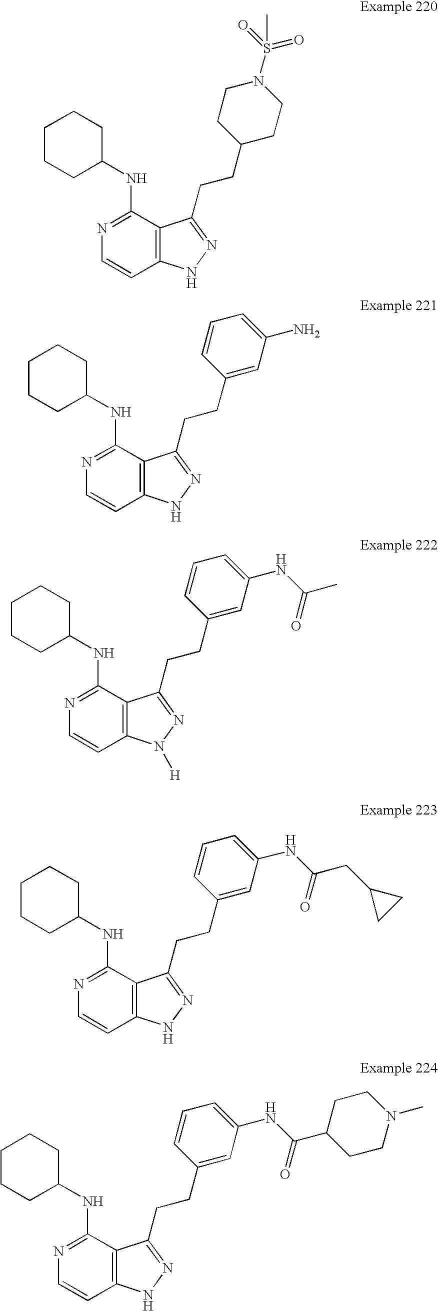

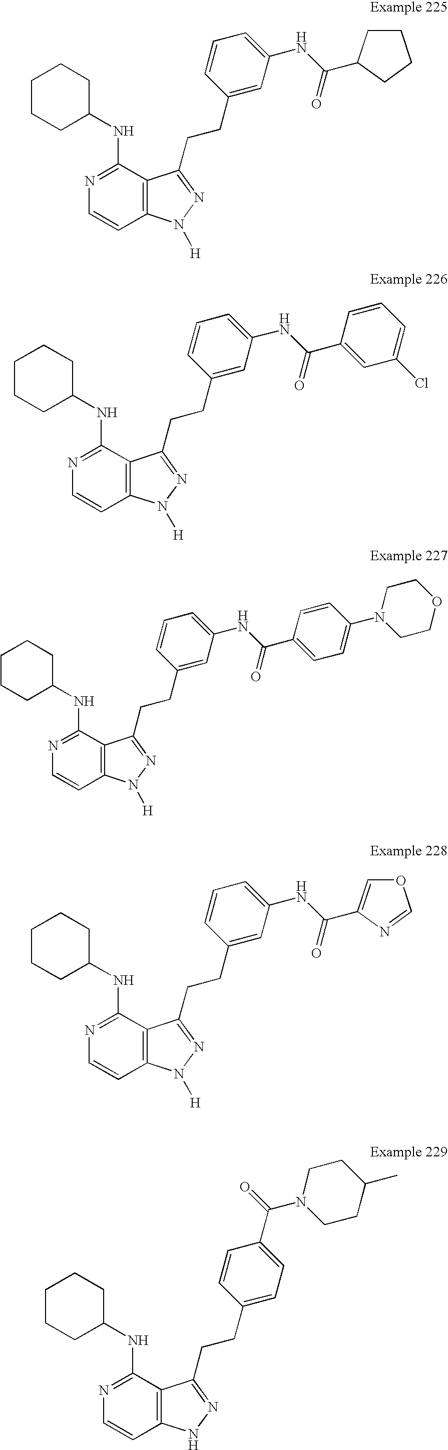

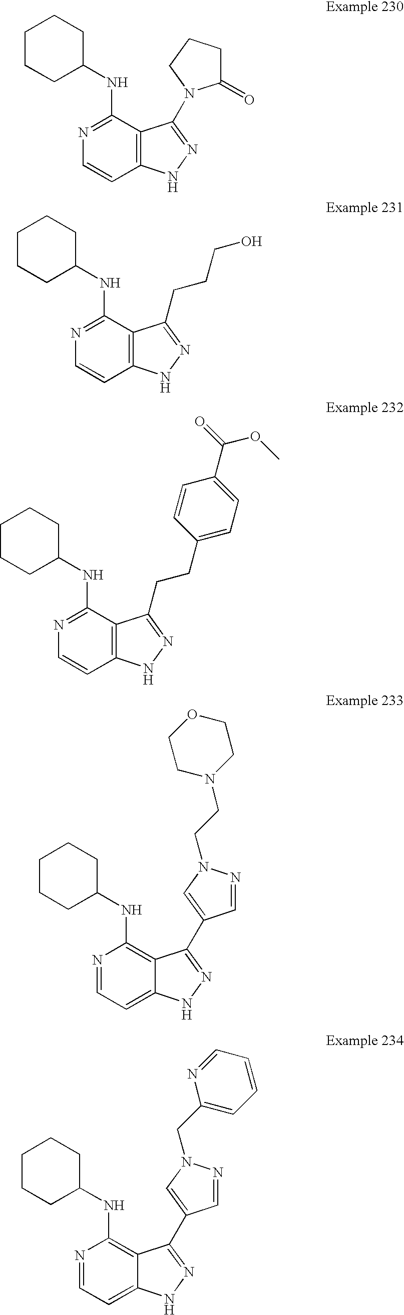

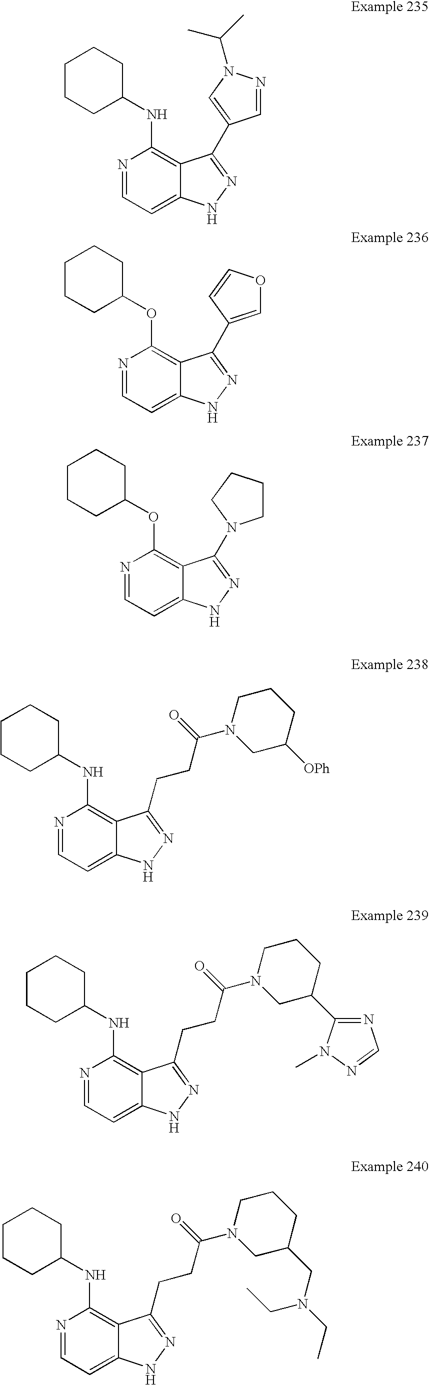

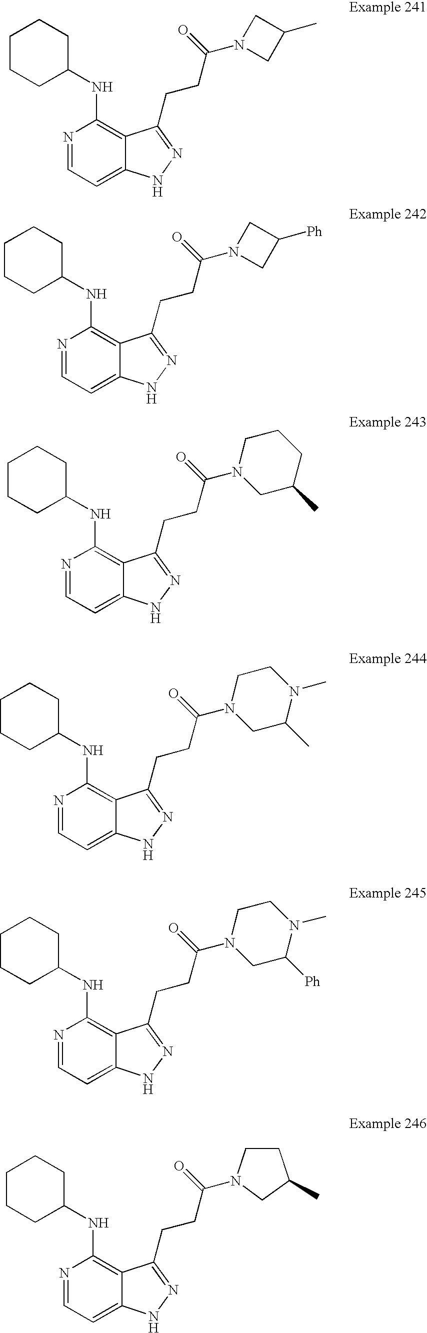

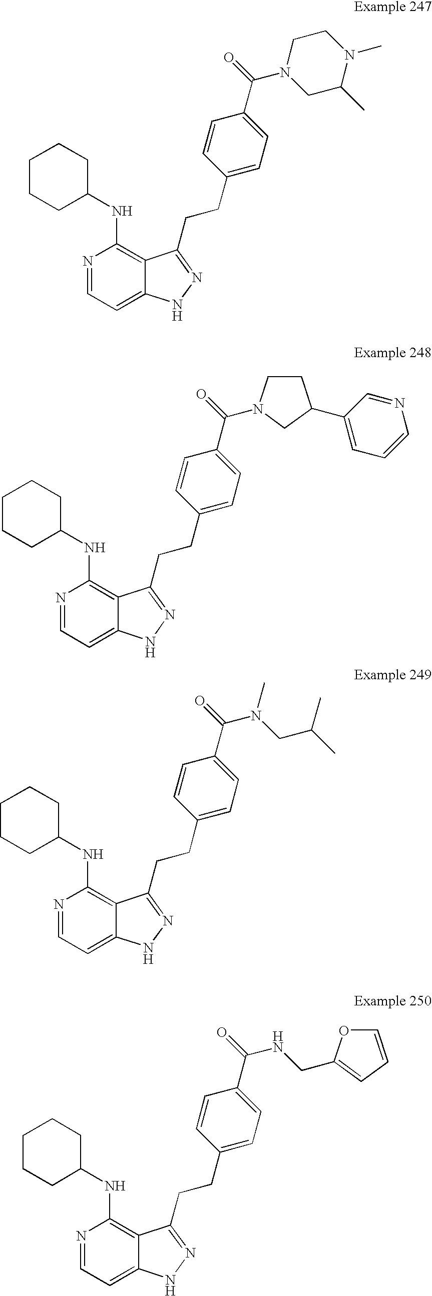

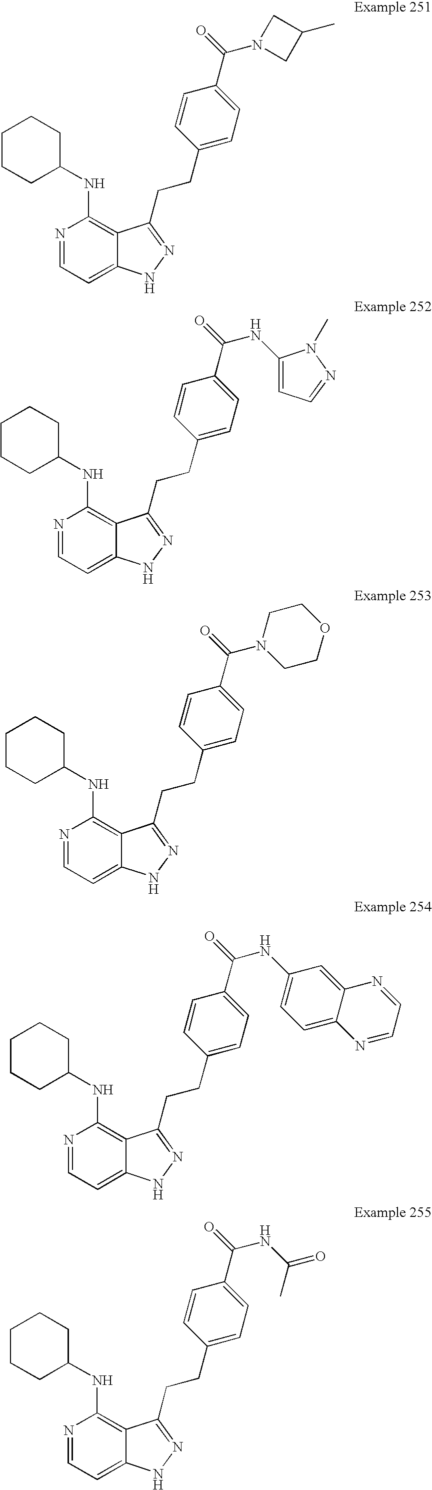

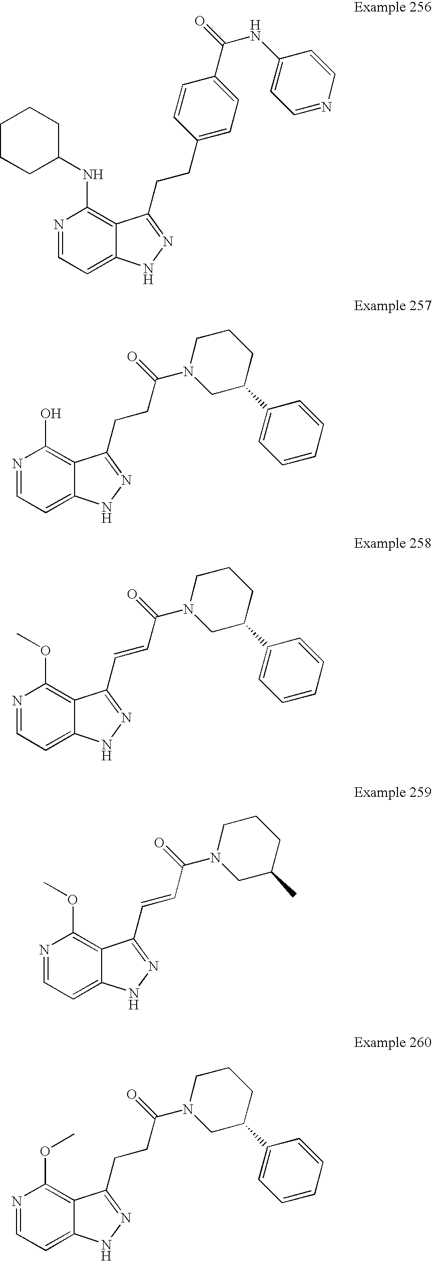

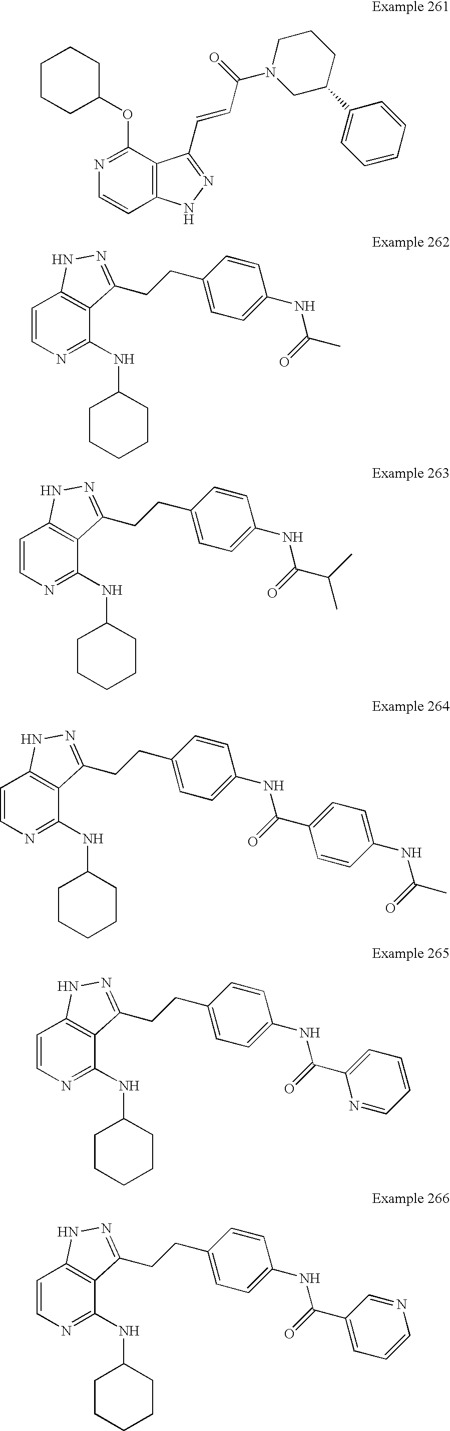

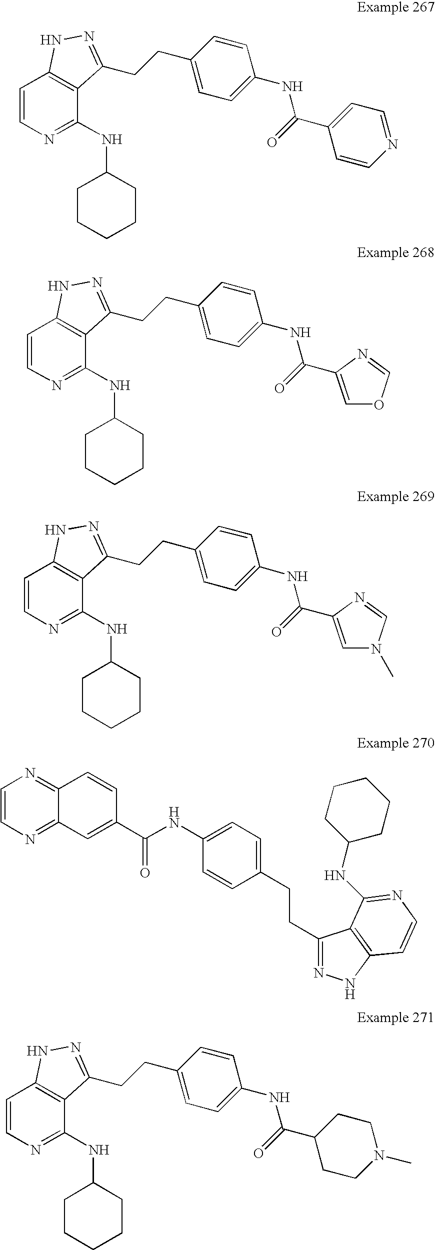

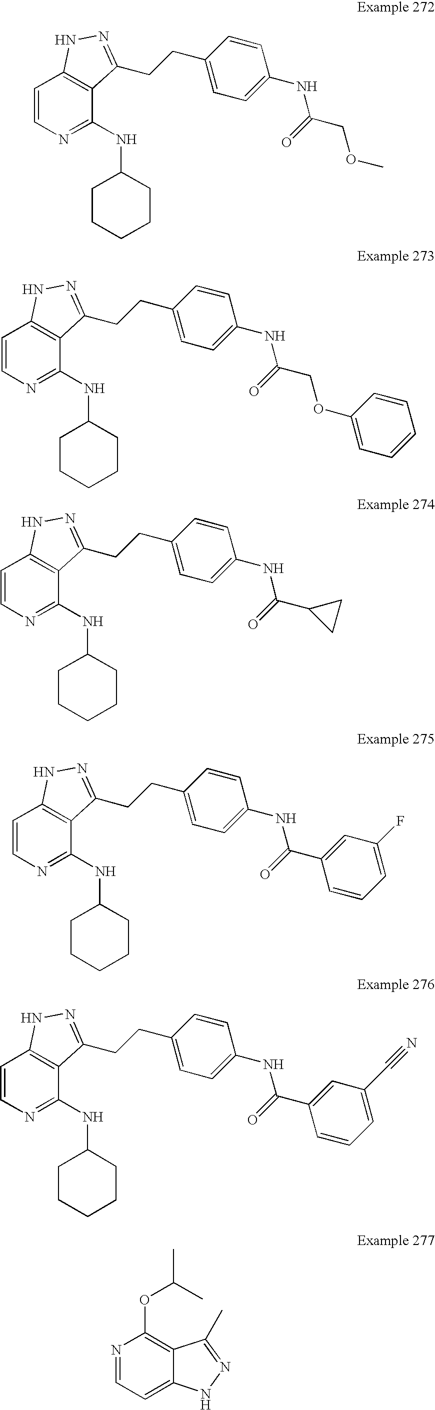

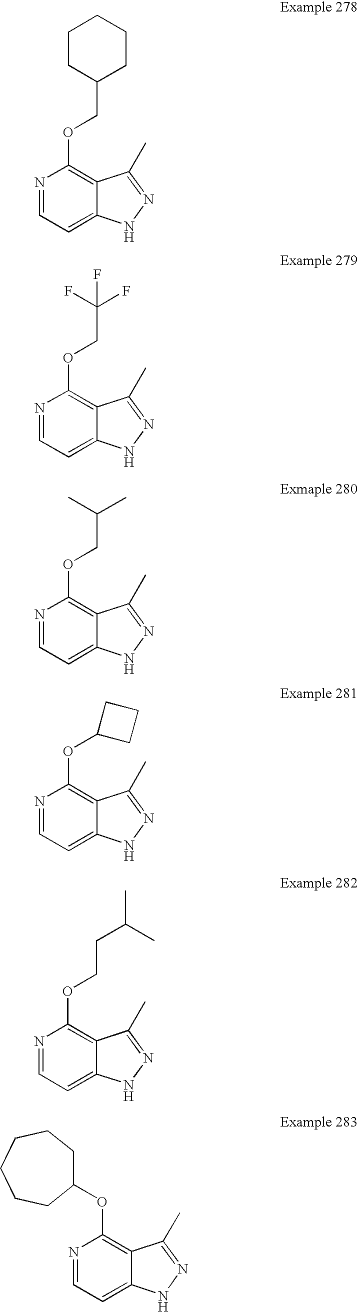

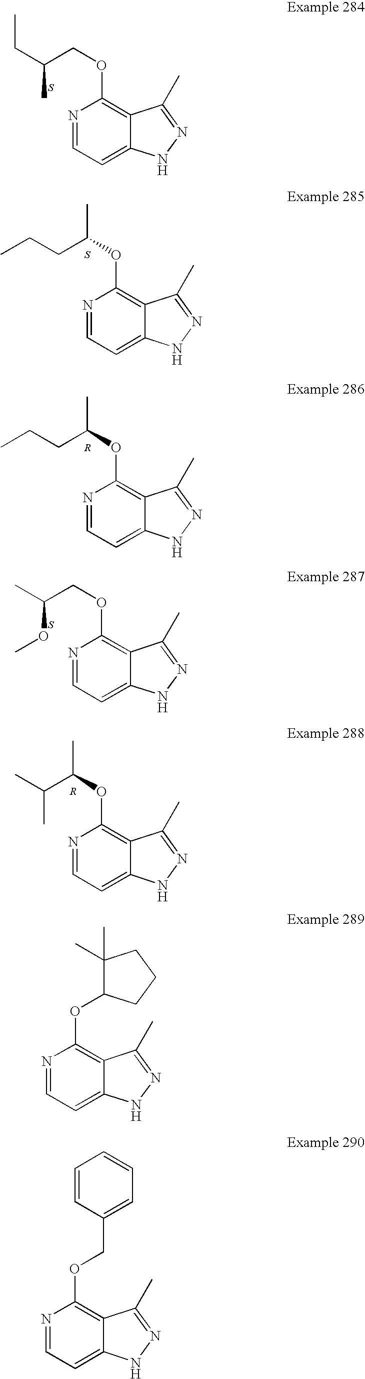

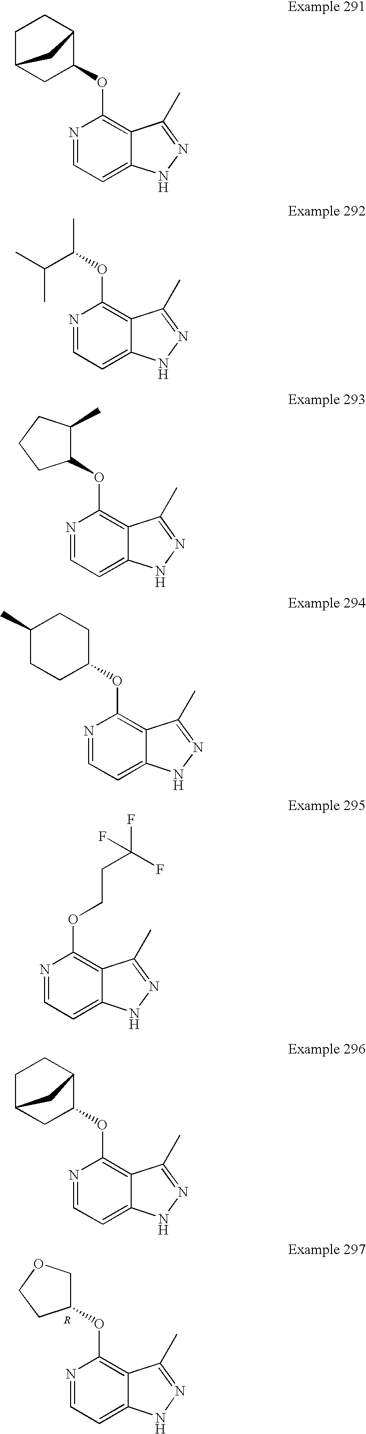

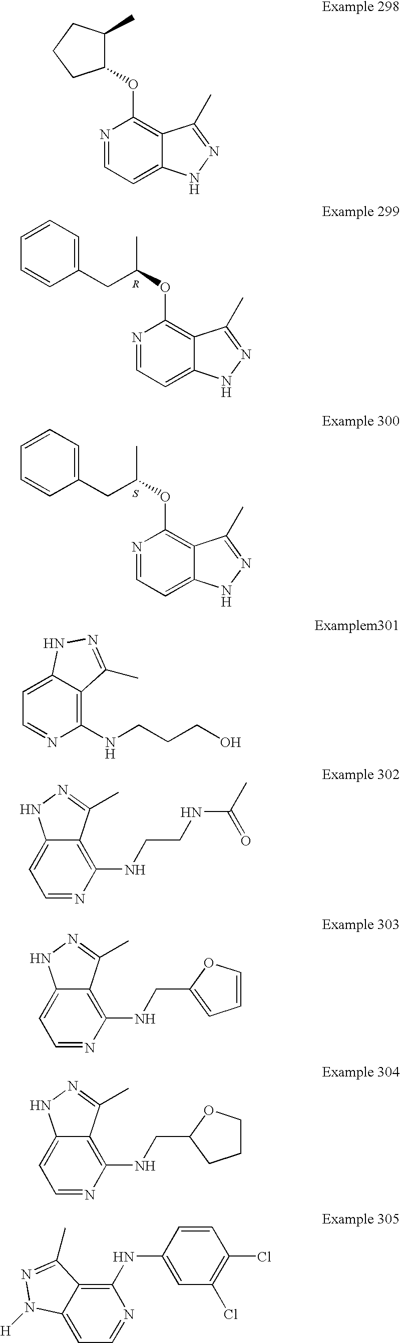

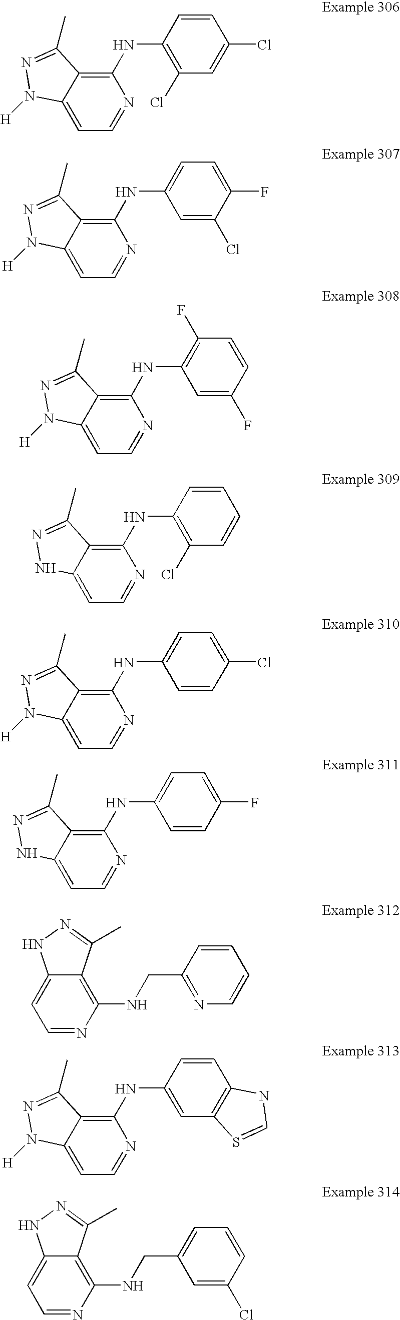

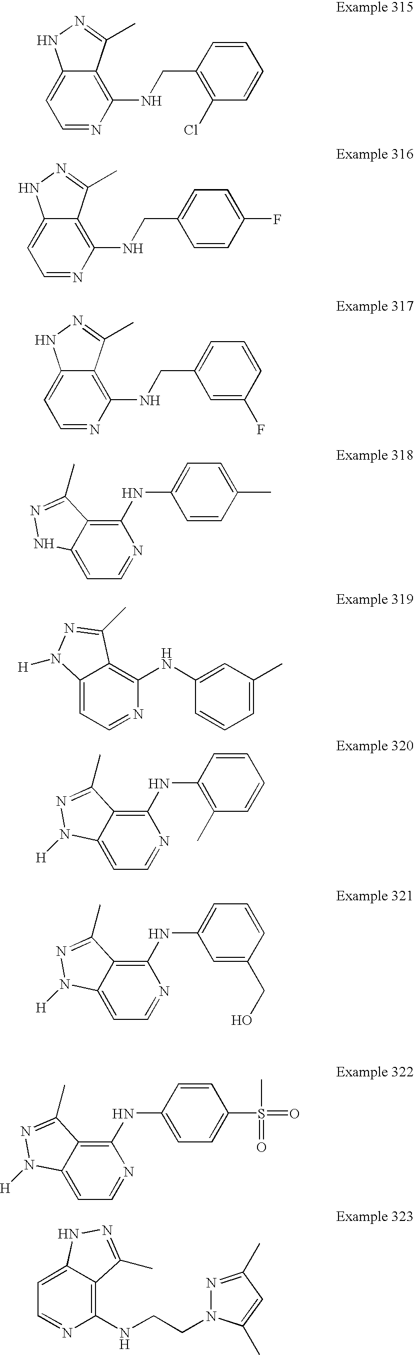

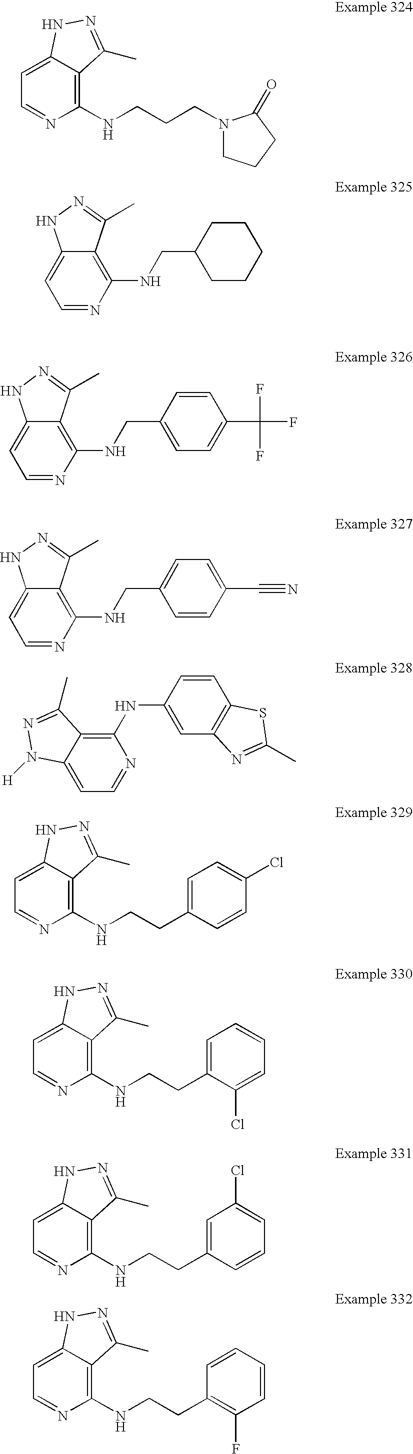

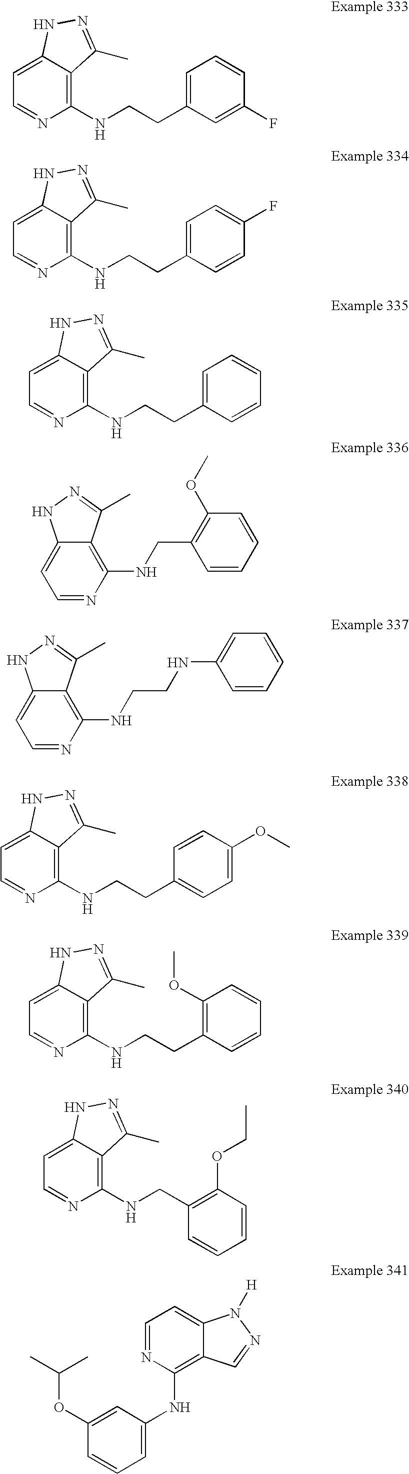

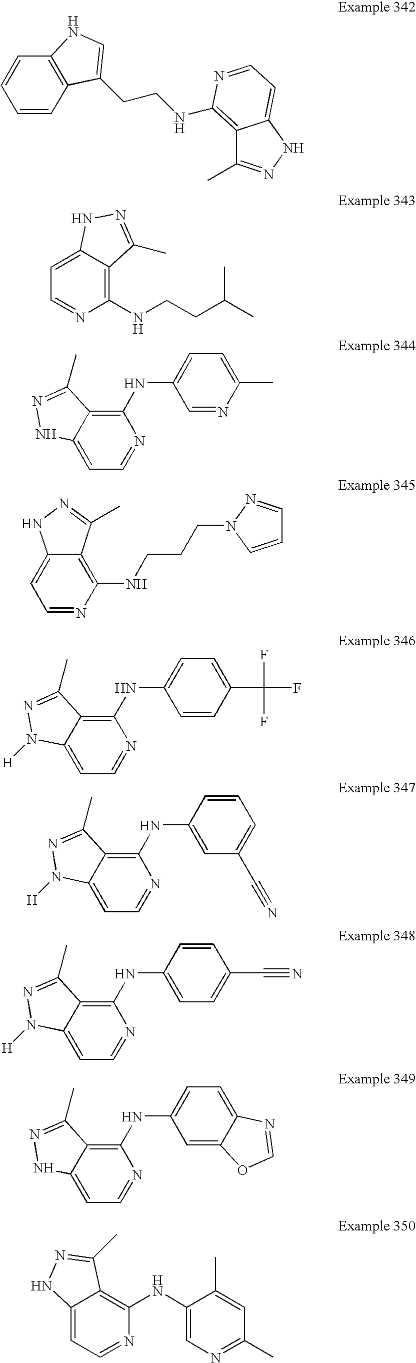

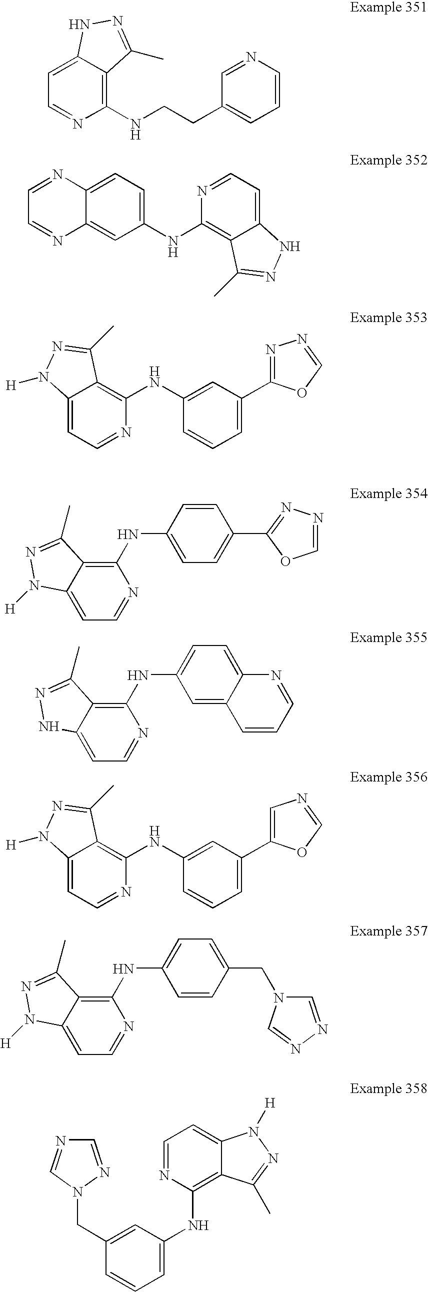

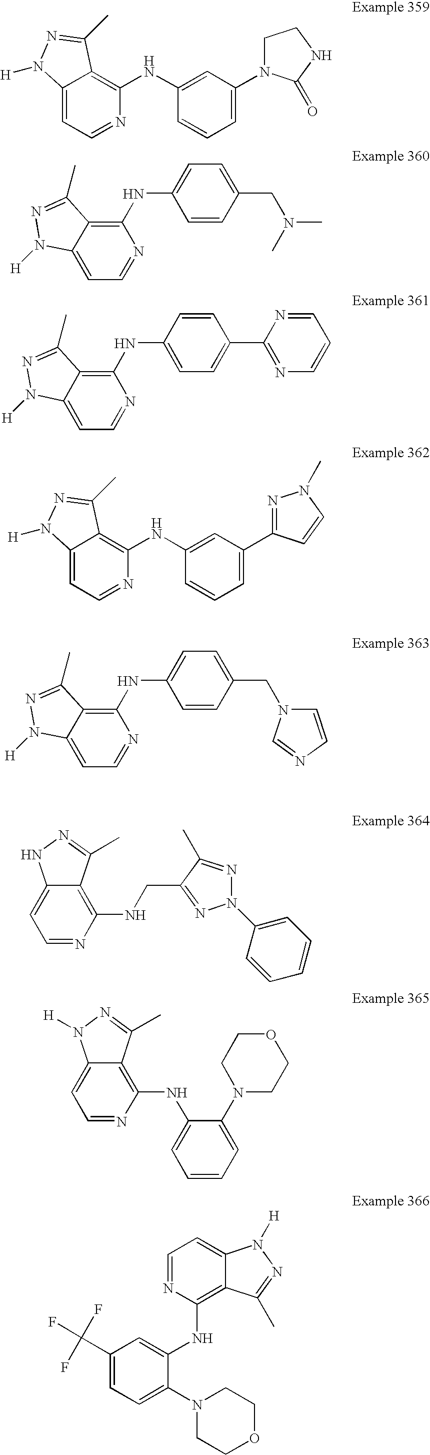

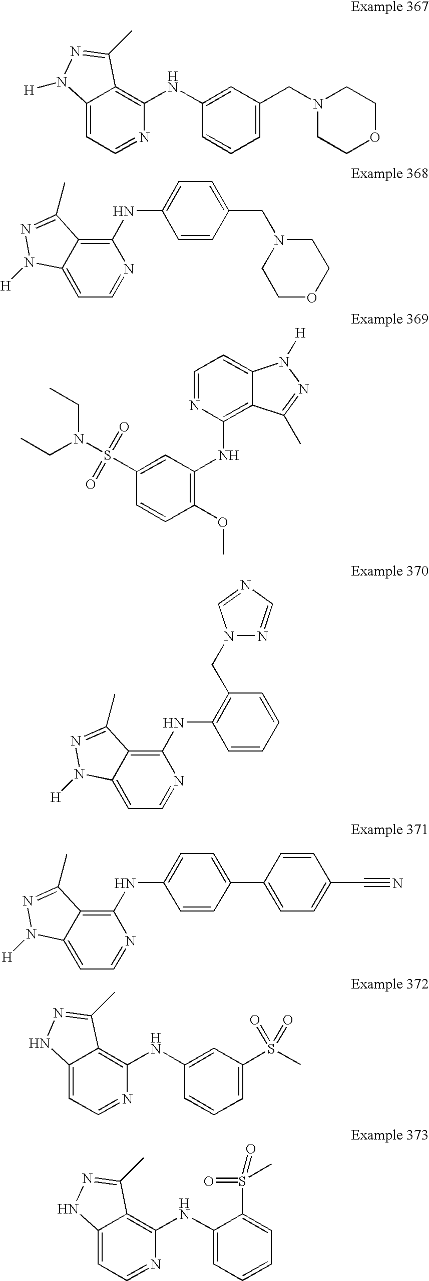

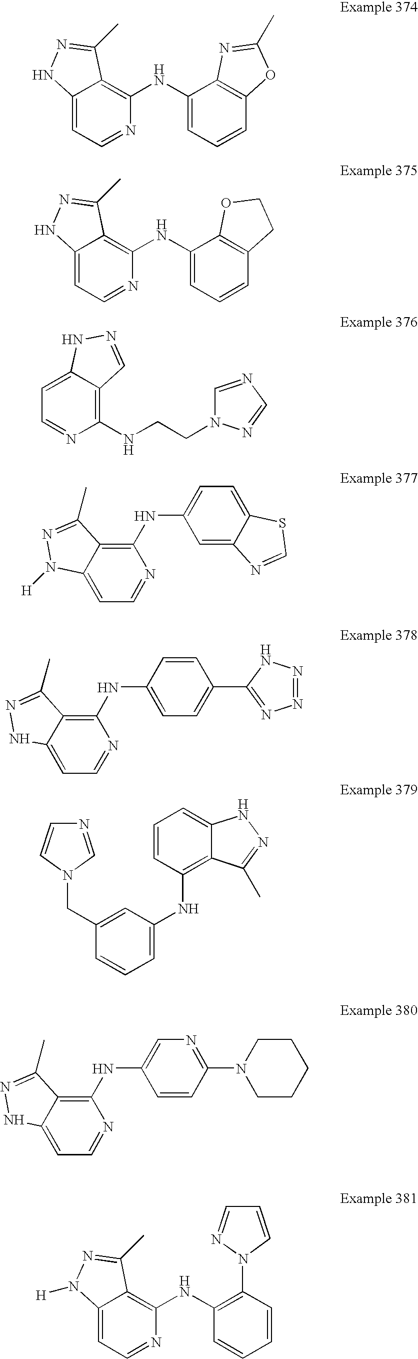

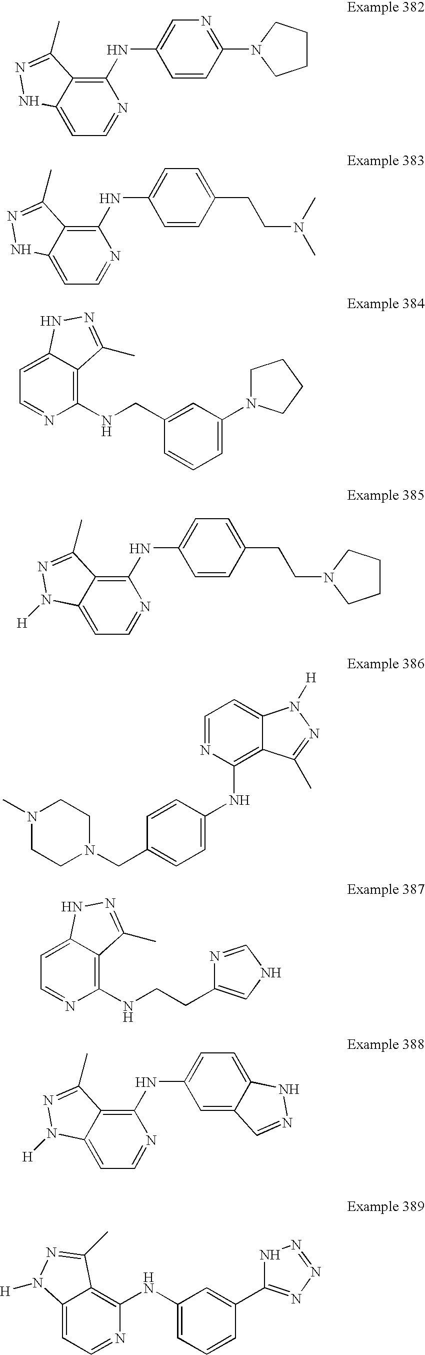

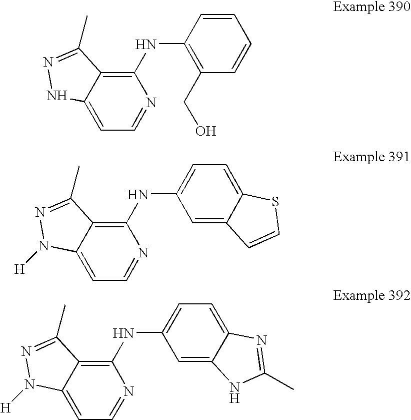

### Therapeutic Applications

A further aspect of the invention relates to a compound as described above for use in medicine.

Another aspect of the invention relates to a compound as described above for use in treating cancer or a neurodegenerative disorder.

Another aspect relates to the use of a compound as described above in the preparation of a medicament for treating or preventing a neurodegenerative disorder. Preferably, the neurodegenerative disorder is Parkinson's Disease.

Another aspect relates to the use of a compound as described above in the preparation of a medicament for treating or preventing a proliferative disorder, for example, cancer.

Preferably, the compound is administered in an amount sufficient to inhibit one or more kinases, preferably LRRK, even more preferably LRRK2.

Yet another aspect relates to the use of a compound of the invention in the preparation of a medicament for the prevention or treatment of a disorder caused by, associated with or accompanied by any abnormal activity against a biological target, wherein the target is a kinase, more preferably LRRK, even more preferably LRRK2.

Preferably, the disorder is Parkinson's Disease.

Another aspect of the invention relates to a method of treating a protein kinase related disease or disorder. The method according to this aspect of the present invention is effected by administering to a subject in need thereof a therapeutically effective amount of a compound of the present invention, as described hereinabove, either per se, or, more preferably, as a part of a pharmaceutical composition, mixed with, for example, a pharmaceutically acceptable carrier, as is detailed hereinafter.

Yet another aspect of the invention relates to a method of treating a mammal having a disease state alleviated by inhibition of a protein kinase, wherein the method comprises administering to a mammal a therapeutically effective amount of a compound according to the invention.

Preferably, the disease state is alleviated by the inhibition of the protein kinase LRRK, more preferably LRRK2.

Preferably, the mammal is a human.

The term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

The term “administering” as used herein refers to a method for bringing a compound of the present invention and a protein kinase together in such a manner that the compound can affect the enzyme activity of the protein kinase either directly; i.e., by interacting with the protein kinase itself or indirectly; i.e., by interacting with another molecule on which the catalytic activity of the protein kinase is dependent. As used herein, administration can be accomplished either in vitro, i.e. in a test tube, or in vivo, i.e., in cells or tissues of a living organism.

Herein, the term “treating” includes abrogating, substantially inhibiting, slowing or reversing the progression of a disease or disorder, substantially ameliorating clinical symptoms of a disease or disorder or substantially preventing the appearance of clinical symptoms of a disease or disorder.

Herein, the term “preventing” refers to a method for barring an organism from acquiring a disorder or disease in the first place.

The term “therapeutically effective amount” refers to that amount of the compound being administered which will relieve to some extent one or more of the symptoms of the disease or disorder being treated.

For any compound used in this invention, a therapeutically effective amount, also referred to herein as a therapeutically effective dose, can be estimated initially from cell culture assays. For example, a dose can be formulated in animal models to achieve a circulating concentration range that includes the IC₅₀ or the IC₁₀₀ as determined in cell culture. Such information can be used to more accurately determine useful doses in humans. Initial dosages can also be estimated from in vivo data. Using these initial guidelines one of ordinary skill in the art could determine an effective dosage in humans.

Moreover, toxicity and therapeutic efficacy of the compounds described herein can be determined by standard pharmaceutical procedures in cell cultures or experimental animals, e.g., by determining the LD₅₀ and the ED₅₀. The dose ratio between toxic and therapeutic effect is the therapeutic index and can be expressed as the ratio between LD₅₀ and ED₅₀. Compounds which exhibit high therapeutic indices are preferred. The data obtained from these cell cultures assays and animal studies can be used in formulating a dosage range that is not toxic for use in human. The dosage of such compounds lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition. (see, e.g., Fingl et al, 1975, In: The Pharmacological Basis of Therapeutics, chapter 1, page 1).

Dosage amount and interval may be adjusted individually to provide plasma levels of the active compound which are sufficient to maintain therapeutic effect. Usual patient dosages for oral administration range from about 50-2000 mg/kg/day, commonly from about 100-1000 mg/kg/day, preferably from about 150-700 mg/kg/day and most preferably from about 250-500 mg/kg/day. Preferably, therapeutically effective serum levels will be achieved by administering multiple doses each day. In cases of local administration or selective uptake, the effective local concentration of the drug may not be related to plasma concentration. One skilled in the art will be able to optimize therapeutically effective local dosages without undue experimentation.

As used herein, “kinase related disease or disorder” refers to a disease or disorder characterized by inappropriate kinase activity or over-activity of a kinase as defined herein. Inappropriate activity refers to either; (i) kinase expression in cells which normally do not express said kinase; (ii) increased kinase expression leading to unwanted cell proliferation, differentiation and/or growth; or, (iii) decreased kinase expression leading to unwanted reductions in cell proliferation, differentiation and/or growth. Over-activity of kinase refers to either amplification of the gene encoding a particular kinase or production of a level of kinase activity, which can correlate with a cell proliferation, differentiation and/or growth disorder (that is, as the level of the kinase increases, the severity of one or more of the symptoms of the cellular disorder increases). Over activity can also be the result of ligand independent or constitutive activation as a result of mutations such as deletions of a fragment of a kinase responsible for ligand binding.

Preferred diseases or disorders that the compounds described herein may be useful in preventing, include cancer and neurodegenerative disorders such as Parkinson's Disease.

Thus, the present invention further provides use of compounds as defined herein for the manufacture of medicaments for the treatment of diseases where it is desirable to inhibit LRRK2. Such diseases include Parkinson's Disease.

### Pharmaceutical Compostions

For use according to the present invention, the compounds or physiologically acceptable salt, ester or other physiologically functional derivative thereof, described herein, may be presented as a pharmaceutical formulation, comprising the compounds or physiologically acceptable salt, ester or other physiologically functional derivative thereof, together with one or more pharmaceutically acceptable carriers therefore and optionally other therapeutic and/or prophylactic ingredients. The carrier(s) must be acceptable in the sense of being compatible with the other ingredients of the formulation and not deleterious to the recipient thereof. The pharmaceutical compositions may be for human or animal usage in human and veterinary medicine.

Examples of such suitable excipients for the various different forms of pharmaceutical compositions described herein may be found in the “Handbook of Pharmaceutical Excipients, 2^{nd} Edition, (1994), Edited by A Wade and P J Weller.

Acceptable carriers or diluents for therapeutic use are well known in the pharmaceutical art, and are described, for example, in Remington's Pharmaceutical Sciences, Mack Publishing Co. (A. R. Gennaro edit. 1985).

Examples of suitable carriers include lactose, starch, glucose, methyl cellulose, magnesium stearate, mannitol, sorbitol and the like. Examples of suitable diluents include ethanol, glycerol and water.

The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s), buffer(s), flavouring agent(s), surface active agent(s), thickener(s), preservative(s) (including anti-oxidants) and the like, and substances included for the purpose of rendering the formulation isotonic with the blood of the intended recipient.

Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol.

Examples of suitable lubricants include sodium oleate, sodium stearate, magnesium stearate, sodium benzoate, sodium acetate, sodium chloride and the like.

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

Pharmaceutical formulations include those suitable for oral, topical (including dermal, buccal and sublingual), rectal or parenteral (including subcutaneous, intradermal, intramuscular and intravenous), nasal and pulmonary administration e.g., by inhalation. The formulation may, where appropriate, be conveniently presented in discrete dosage units and may be prepared by any of the methods well known in the art of pharmacy. All methods include the step of bringing into association an active compound with liquid carriers or finely divided solid carriers or both and then, if necessary, shaping the product into the desired formulation.

Pharmaceutical formulations suitable for oral administration wherein the carrier is a solid are most preferably presented as unit dose formulations such as boluses, capsules or tablets each containing a predetermined amount of active compound. A tablet may be made by compression or moulding, optionally with one or more accessory ingredients. Compressed tablets may be prepared by compressing in a suitable machine an active compound in a free-flowing form such as a powder or granules optionally mixed with a binder, lubricant, inert diluent, lubricating agent, surface-active agent or dispersing agent. Moulded tablets may be made by moulding an active compound with an inert liquid diluent. Tablets may be optionally coated and, if uncoated, may optionally be scored. Capsules may be prepared by filling an active compound, either alone or in admixture with one or more accessory ingredients, into the capsule shells and then sealing them in the usual manner. Cachets are analogous to capsules wherein an active compound together with any accessory ingredient(s) is sealed in a rice paper envelope. An active compound may also be formulated as dispersible granules, which may for example be suspended in water before administration, or sprinkled on food. The granules may be packaged, e.g., in a sachet. Formulations suitable for oral administration wherein the carrier is a liquid may be presented as a solution or a suspension in an aqueous or non-aqueous liquid, or as an oil-in-water liquid emulsion.

Formulations for oral administration include controlled release dosage forms, e.g., tablets wherein an active compound is formulated in an appropriate release—controlling matrix, or is coated with a suitable release—controlling film. Such formulations may be particularly convenient for prophylactic use.

Pharmaceutical formulations suitable for rectal administration wherein the carrier is a solid are most preferably presented as unit dose suppositories. Suitable carriers include cocoa butter and other materials commonly used in the art. The suppositories may be conveniently formed by admixture of an active compound with the softened or melted carrier(s) followed by chilling and shaping in moulds. Pharmaceutical formulations suitable for parenteral administration include sterile solutions or suspensions of an active compound in aqueous or oleaginous vehicles.

Injectable preparations may be adapted for bolus injection or continuous infusion. Such preparations are conveniently presented in unit dose or multi-dose containers which are sealed after introduction of the formulation until required for use. Alternatively, an active compound may be in powder form which is constituted with a suitable vehicle, such as sterile, pyrogen-free water, before use.

An active compound may also be formulated as long-acting depot preparations, which may be administered by intramuscular injection or by implantation, e.g., subcutaneously or intramuscularly. Depot preparations may include, for example, suitable polymeric or hydrophobic materials, or ion-exchange resins. Such long-acting formulations are particularly convenient for prophylactic use.

Formulations suitable for pulmonary administration via the buccal cavity are presented such that particles containing an active compound and desirably having a diameter in the range of 0.5 to 7 microns are delivered in the bronchial tree of the recipient.

As one possibility such formulations are in the form of finely comminuted powders which may conveniently be presented either in a pierceable capsule, suitably of, for example, gelatin, for use in an inhalation device, or alternatively as a self-propelling formulation comprising an active compound, a suitable liquid or gaseous propellant and optionally other ingredients such as a surfactant and/or a solid diluent. Suitable liquid propellants include propane and the chlorofluorocarbons, and suitable gaseous propellants include carbon dioxide. Self-propelling formulations may also be employed wherein an active compound is dispensed in the form of droplets of solution or suspension.

Such self-propelling formulations are analogous to those known in the art and may be prepared by established procedures. Suitably they are presented in a container provided with either a manually-operable or automatically functioning valve having the desired spray characteristics; advantageously the valve is of a metered type delivering a fixed volume, for example, 25 to 100 microlitres, upon each operation thereof.

As a further possibility an active compound may be in the form of a solution or suspension for use in an atomizer or nebuliser whereby an accelerated airstream or ultrasonic agitation is employed to produce a fine droplet mist for inhalation.

Formulations suitable for nasal administration include preparations generally similar to those described above for pulmonary administration. When dispensed such formulations should desirably have a particle diameter in the range 10 to 200 microns to enable retention in the nasal cavity; this may be achieved by, as appropriate, use of a powder of a suitable particle size or choice of an appropriate valve. Other suitable formulations include coarse powders having a particle diameter in the range 20 to 500 microns, for administration by rapid inhalation through the nasal passage from a container held close up to the nose, and nasal drops comprising 0.2 to 5% w/v of an active compound in aqueous or oily solution or suspension.

Pharmaceutically acceptable carriers are well known to those skilled in the art and include, but are not limited to, 0.1 M and preferably 0.05 M phosphate buffer or 0.8% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Preservatives and other additives may also be present, such as, for example, antimicrobials, antioxidants, chelating agents, inert gases and the like.

Formulations suitable for topical formulation may be provided for example as gels, creams or ointments. Such preparations may be applied e.g. to a wound or ulcer either directly spread upon the surface of the wound or ulcer or carried on a suitable support such as a bandage, gauze, mesh or the like which may be applied to and over the area to be treated.

Liquid or powder formulations may also be provided which can be sprayed or sprinkled directly onto the site to be treated, e.g. a wound or ulcer. Alternatively, a carrier such as a bandage, gauze, mesh or the like can be sprayed or sprinkle with the formulation and then applied to the site to be treated.

According to a further aspect of the invention, there is provided a process for the preparation of a pharmaceutical or veterinary composition as described above, the process comprising bringing the active compound(s) into association with the carrier, for example by admixture.

In general, the formulations are prepared by uniformly and intimately bringing into association the active agent with liquid carriers or finely divided solid carriers or both, and then if necessary shaping the product. The invention extends to methods for preparing a pharmaceutical composition comprising bringing a compound of general formula (I) in conjunction or association with a pharmaceutically or veterinarily acceptable carrier or vehicle.

### Salts/Esters

The compounds of the invention can be present as salts or esters, in particular pharmaceutically and veterinarily acceptable salts or esters.

Pharmaceutically acceptable salts of the compounds of the invention include suitable acid addition or base salts thereof. A review of suitable pharmaceutical salts may be found in Berge et al, J Pharm Sci, 66, 1-19 (1977). Salts are formed, for example with strong inorganic acids such as mineral acids, e.g. hydrohalic acids such as hydrochloride, hydrobromide and hydroiodide, sulphuric acid, phosphoric acid sulphate, bisulphate, hemisulphate, thiocyanate, persulphate and sulphonic acids; with strong organic carboxylic acids, such as alkanecarboxylic acids of 1 to 4 carbon atoms which are unsubstituted or substituted (e.g., by halogen), such as acetic acid; with saturated or unsaturated dicarboxylic acids, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid. Salts which are not pharmaceutically or veterinarily acceptable may still be valuable as intermediates.

Preferred salts include, for example, acetate, trifluoroacetate, lactate, gluconate, citrate, tartrate, maleate, malate, pantothenate, adipate, alginate, aspartate, benzoate, butyrate, digluconate, cyclopentanate, glucoheptanate, glycerophosphate, oxalate, heptanoate, hexanoate, fumarate, nicotinate, palmoate, pectinate, 3-phenylpropionate, picrate, pivalate, proprionate, tartrate, lactobionate, pivolate, camphorate, undecanoate and succinate, organic sulphonic acids such as methanesulphonate, ethanesulphonate, 2-hydroxyethane sulphonate, camphorsulphonate, 2-naphthalenesulphonate, benzenesulphonate, p-chlorobenzenesulphonate and p-toluenesulphonate; and inorganic acids such as hydrochloride, hydrobromide, hydroiodide, sulphate, bisulphate, hemisulphate, thiocyanate, persulphate, phosphoric and sulphonic acids.

Esters are formed either using organic acids or alcohols/hydroxides, depending on the functional group being esterified. Organic acids include carboxylic acids, such as alkanecarboxylic acids of 1 to 12 carbon atoms which are unsubstituted or substituted (e.g., by halogen), such as acetic acid; with saturated or unsaturated dicarboxylic acid, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid. Suitable hydroxides include inorganic hydroxides, such as sodium hydroxide, potassium hydroxide, calcium hydroxide, aluminium hydroxide. Alcohols include alkanealcohols of 1-12 carbon atoms which may be unsubstituted or substituted, e.g. by a halogen).

### Enantiomers/Tautomers

In all aspects of the present invention previously discussed, the invention includes, where appropriate all enantiomers, diastereoisomers and tautomers of the compounds of the invention. The person skilled in the art will recognise compounds that possess optical properties (one or more chiral carbon atoms) or tautomeric characteristics. The corresponding enantiomers and/or tautomers may be isolated/prepared by methods known in the art.

Enantiomers are characterised by the absolute configuration of their chiral centres and described by the R- and S-sequencing rules of Cahn, Ingold and Prelog. Such conventions are well known in the art (e.g. see ‘Advanced Organic Chemistry’, 3^{rd} edition, ed. March, J., John Wiley and Sons, New York, 1985).

Compounds of the invention containing a chiral centre may be used as a racemic mixture, an enantiomerically enriched mixture, or the racemic mixture may be separated using well-known techniques and an individual enantiomer may be used alone.

### Stereo and Geometric Isomers

Some of the compounds of the invention may exist as stereoisomers and/or geometric isomers—e.g. they may possess one or more asymmetric and/or geometric centres and so may exist in two or more stereoisomeric and/or geometric forms. The present invention contemplates the use of all the individual stereoisomers and geometric isomers of those inhibitor agents, and mixtures thereof. The terms used in the claims encompass these forms, provided said forms retain the appropriate functional activity (though not necessarily to the same degree).

The present invention also includes all suitable isotopic variations of the agent or a pharmaceutically acceptable salt thereof. An isotopic variation of an agent of the present invention or a pharmaceutically acceptable salt thereof is defined as one in which at least one atom is replaced by an atom having the same atomic number but an atomic mass different from the atomic mass usually found in nature. Examples of isotopes that can be incorporated into the agent and pharmaceutically acceptable salts thereof include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹P, ³²P, ³⁵S, ¹⁸F and ³⁶Cl, respectively. Certain isotopic variations of the agent and pharmaceutically acceptable salts thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. For example, the invention includes compounds of general formula (I) where any hydrogen atom has been replaced by a deuterium atom. Isotopic variations of the agent of the present invention and pharmaceutically acceptable salts thereof of this invention can generally be prepared by conventional procedures using appropriate isotopic variations of suitable reagents.

### Prodrugs

The invention further includes the compounds of the present invention in prodrug form, i.e. covalently bonded compounds which release the active parent drug according to general formula (I) in vivo. Such prodrugs are generally compounds of the invention wherein one or more appropriate groups have been modified such that the modification may be reversed upon administration to a human or mammalian subject. Reversion is usually performed by an enzyme naturally present in such subject, though it is possible for a second agent to be administered together with such a prodrug in order to perform the reversion in vivo. Examples of such modifications include ester (for example, any of those described above), wherein the reversion may be carried out be an esterase etc. Other such systems will be well known to those skilled in the art.

### Solvates

The present invention also includes solvate forms of the compounds of the present invention. The terms used in the claims encompass these forms.

### Polymorphs

The invention further relates to the compounds of the present invention in their various crystalline forms, polymorphic forms and (an)hydrous forms. It is well established within the pharmaceutical industry that chemical compounds may be isolated in any of such forms by slightly varying the method of purification and or isolation form the solvents used in the synthetic preparation of such compounds.

### Administration

The pharmaceutical compositions of the present invention may be adapted for rectal, nasal, intrabronchial, topical (including buccal and sublingual), vaginal or parenteral (including subcutaneous, intramuscular, intravenous, intraarterial and intradermal), intraperitoneal or intrathecal administration. Preferably the formulation is an orally administered formulation. The formulations may conveniently be presented in unit dosage form, i.e., in the form of discrete portions containing a unit dose, or a multiple or sub-unit of a unit dose. By way of example, the formulations may be in the form of tablets and sustained release capsules, and may be prepared by any method well known in the art of pharmacy.

Formulations for oral administration in the present invention may be presented as: discrete units such as capsules, gellules, drops, cachets, pills or tablets each containing a predetermined amount of the active agent; as a powder or granules; as a solution, emulsion or a suspension of the active agent in an aqueous liquid or a non-aqueous liquid; or as an oil-in-water liquid emulsion or a water-in-oil liquid emulsion; or as a bolus etc. Preferably, these compositions contain from 1 to 250 mg and more preferably from 10-100 mg, of active ingredient per dose.

For compositions for oral administration (e.g. tablets and capsules), the term “acceptable carrier” includes vehicles such as common excipients e.g. binding agents, for example syrup, acacia, gelatin, sorbitol, tragacanth, polyvinylpyrrolidone (Povidone), methylcellulose, ethylcellulose, sodium carboxymethylcellulose, hydroxypropyl-methylcellulose, sucrose and starch; fillers and carriers, for example corn starch, gelatin, lactose, sucrose, microcrystalline cellulose, kaolin, mannitol, dicalcium phosphate, sodium chloride and alginic acid; and lubricants such as magnesium stearate, sodium stearate and other metallic stearates, glycerol stearate stearic acid, silicone fluid, talc waxes, oils and colloidal silica. Flavouring agents such as peppermint, oil of wintergreen, cherry flavouring and the like can also be used. It may be desirable to add a colouring agent to make the dosage form readily identifiable. Tablets may also be coated by methods well known in the art.

A tablet may be made by compression or moulding, optionally with one or more accessory ingredients. Compressed tablets may be prepared by compressing in a suitable machine the active agent in a free flowing form such as a powder or granules, optionally mixed with a binder, lubricant, inert diluent, preservative, surface-active or dispersing agent. Moulded tablets may be made by moulding in a suitable machine a mixture of the powdered compound moistened with an inert liquid diluent. The tablets may be optionally be coated or scored and may be formulated so as to provide slow or controlled release of the active agent.

Other formulations suitable for oral administration include lozenges comprising the active agent in a flavoured base, usually sucrose and acacia or tragacanth; pastilles comprising the active agent in an inert base such as gelatin and glycerin, or sucrose and acacia; and mouthwashes comprising the active agent in a suitable liquid carrier.

Other forms of administration comprise solutions or emulsions which may be injected intravenously, intraarterially, intrathecally, subcutaneously, intradermally, intraperitoneally or intramuscularly, and which are prepared from sterile or sterilisable solutions. Injectable forms typically contain between 10-1000 mg, preferably between 10-250 mg, of active ingredient per dose.

The pharmaceutical compositions of the present invention may also be in form of suppositories, pessaries, suspensions, emulsions, lotions, ointments, creams, gels, sprays, solutions or dusting powders.

An alternative means of transdermal administration is by use of a skin patch. For example, the active ingredient can be incorporated into a cream consisting of an aqueous emulsion of polyethylene glycols or liquid paraffin. The active ingredient can also be incorporated, at a concentration of between 1 and 10% by weight, into an ointment consisting of a white wax or white soft paraffin base together with such stabilisers and preservatives as may be required.

### Dosage

A person of ordinary skill in the art can easily determine an appropriate dose of one of the instant compositions to administer to a subject without undue experimentation. Typically, a physician will determine the actual dosage which will be most suitable for an individual patient and it will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. The dosages disclosed herein are exemplary of the average case. There can of course be individual instances where higher or lower dosage ranges are merited, and such are within the scope of this invention.

In accordance with this invention, an effective amount of a compound of general formula (I) may be administered to inhibit the kinase implicated with a particular condition or disease. Of course, this dosage amount will further be modified according to the type of administration of the compound. For example, to achieve an “effective amount” for acute therapy, parenteral administration of a compound of general formula (I) is preferred. An intravenous infusion of the compound in 5% dextrose in water or normal saline, or a similar formulation with suitable excipients, is most effective, although an intramuscular bolus injection is also useful. Typically, the parenteral dose will be about 0.01 to about 100 mg/kg; preferably between 0.1 and 20 mg/kg, in a manner to maintain the concentration of drug in the plasma at a concentration effective to inhibit a kinase. The compounds may be administered one to four times daily at a level to achieve a total daily dose of about 0.4 to about 400 mg/kg/day. The precise amount of an inventive compound which is therapeutically effective, and the route by which such compound is best administered, is readily determined by one of ordinary skill in the art by comparing the blood level of the agent to the concentration required to have a therapeutic effect.

The compounds of this invention may also be administered orally to the patient, in a manner such that the concentration of drug is sufficient to achieve one or more of the therapeutic indications disclosed herein. Typically, a pharmaceutical composition containing the compound is administered at an oral dose of between about 0.1 to about 50 mg/kg in a manner consistent with the condition of the patient. Preferably the oral dose would be about 0.5 to about 20 mg/kg.

No unacceptable toxicological effects are expected when compounds of the present invention are administered in accordance with the present invention. The compounds of this invention, which may have good bioavailability, may be tested in one of several biological assays to determine the concentration of a compound which is required to have a given pharmacological effect.

### Combinations

In a particularly preferred embodiment, the one or more compounds of the invention are administered in combination with one or more other active agents, for example, existing drugs available on the market. In such cases, the compounds of the invention may be administered consecutively, simultaneously or sequentially with the one or more other active agents.

Drugs in general are more effective when used in combination. In particular, combination therapy is desirable in order to avoid an overlap of major toxicities, mechanism of action and resistance mechanism(s). Furthermore, it is also desirable to administer most drugs at their maximum tolerated doses with minimum time intervals between such doses. The major advantages of combining chemotherapeutic drugs are that it may promote additive or possible synergistic effects through biochemical interactions and also may decrease the emergence of resistance.

Beneficial combinations may be suggested by studying the inhibitory activity of the test compounds with agents known or suspected of being valuable in the treatment of a particular disorder. This procedure can also be used to determine the order of administration of the agents, i.e. before, simultaneously, or after delivery. Such scheduling may be a feature of all the active agents identified herein.

### Assay

A further aspect of the invention relates to the use of a compound as described above in an assay for identifying further candidate compounds capable of inhibiting one or more kinases, more preferably LRRK, even more preferably, LRRK2.

Preferably, the assay is a competitive binding assay.

More preferably, the competitive binding assay comprises contacting a compound of the invention with a kinase, preferably LRRK, more preferably LRRK2, and a candidate compound and detecting any change in the interaction between the compound according to the invention and the kinase.

Preferably, the candidate compound is generated by conventional SAR modification of a compound of the invention.

As used herein, the term “conventional SAR modification” refers to standard methods known in the art for varying a given compound by way of chemical derivatisation.

Thus, in one aspect, the identified compound may act as a model (for example, a template) for the development of other compounds. The compounds employed in such a test may be free in solution, affixed to a solid support, borne on a cell surface, or located intracellularly. The abolition of activity or the formation of binding complexes between the compound and the agent being tested may be measured.

The assay of the present invention may be a screen, whereby a number of agents are tested. In one aspect, the assay method of the present invention is a high through-put screen.

This invention also contemplates the use of competitive drug screening assays in which neutralising antibodies capable of binding a compound specifically compete with a test compound for binding to a compound.

Another technique for screening provides for high throughput screening (HTS) of agents having suitable binding affinity to the substances and is based upon the method described in detail in WO 84/03564.

It is expected that the assay methods of the present invention will be suitable for both small and large-scale screening of test compounds as well as in quantitative assays.

Preferably, the competitive binding assay comprises contacting a compound of the invention with a kinase in the presence of a known substrate of said kinase and detecting any change in the interaction between said kinase and said known substrate.

A further aspect of the invention provides a method of detecting the binding of a ligand to a kinase, said method comprising the steps of:
(i) contacting a ligand with a kinase in the presence of a known substrate of said kinase;
(ii) detecting any change in the interaction between said kinase and said known substrate;
   and wherein said ligand is a compound of the invention.

One aspect of the invention relates to a process comprising the steps of:
(a) performing an assay method described hereinabove;
(b) identifying one or more ligands capable of binding to a ligand binding domain; and
(c) preparing a quantity of said one or more ligands.

Another aspect of the invention provides a process comprising the steps of:
(a) performing an assay method described hereinabove;
(b) identifying one or more ligands capable of binding to a ligand binding domain; and
(c) preparing a pharmaceutical composition comprising said one or more ligands.

Another aspect of the invention provides a process comprising the steps of:
(a) performing an assay method described hereinabove;
(b) identifying one or more ligands capable of binding to a ligand binding domain;
(c) modifying said one or more ligands capable of binding to a ligand binding domain;
(d) performing the assay method described hereinabove;
(e) optionally preparing a pharmaceutical composition comprising said one or more ligands.

The invention also relates to a ligand identified by the method described hereinabove.

Yet another aspect of the invention relates to a pharmaceutical composition comprising a ligand identified by the method described hereinabove.

Another aspect of the invention relates to the use of a ligand identified by the method described hereinabove in the preparation of a pharmaceutical composition for use in the treatment of one or more disorders [insert list of disorders].

The above methods may be used to screen for a ligand useful as an inhibitor of one or more kinases.

Compounds of general formula (I) are useful both as laboratory tools and as therapeutic agents. In the laboratory certain compounds of the invention are useful in establishing whether a known or newly discovered kinase contributes a critical or at least significant biochemical function during the establishment or progression of a disease state, a process commonly referred to as ‘target validation’.

### Synthesis

Another aspect of the invention relates to a process for preparing compounds of formula I.

More specifically, the invention provides a process for preparing a compound of formula I as defined above, said process comprising converting a compound of formula II into a compound of formula I:

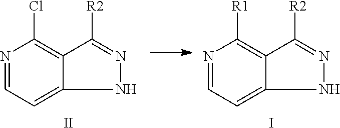

In one preferred embodiment of the invention, the process further comprises the step of preparing said compound of formula II by treating a compound of formula III with hydrazine monohydrate:

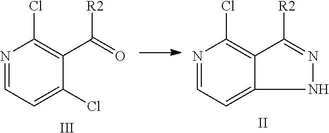

In one preferred embodiment of the invention, the process further comprises the step of preparing said compound of formula III by treating a compound of formula IV with an oxidizing agent:

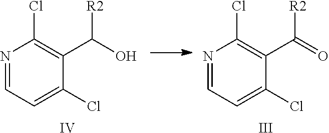

In one preferred embodiment of the invention, the process further comprises the step of preparing said compound of formula IV by treating a compound of formula V with R²—Mg—Cl:

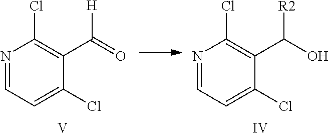

In one preferred embodiment of the invention, R¹ is —NHR³, and the process comprises reacting a compound of formula II with an amine of formula NH₂R³.

In another preferred embodiment of the invention, R¹ is an NH-containing C₄₋₇-heterocycloalkyl or an NH-containing fused aryl-C₄₋₇-heterocycloalkyl, and the process comprises reacting a compound of formula II with the NH-group of said C₄₋₇-heterocycloalkyl or fused aryl-C₄₋₇-heterocycloalkyl.

In another preferred embodiment of the invention, R¹ is selected from aryl, heteroaryl, C₄₋₇-heterocycloalkyl, fused aryl-C₄₋₇-heterocycloalkyl, —C₃₋₇ cycloalkyl and —C₁₋₆ alkyl, and said process comprises reacting a compound of formula II with X—R¹, where X is a 4,4,5,5-tetramethyl-1,3,2-dioxaborolan-2-yl group, in the presence of a coupling agent.

Preferably, the coupling agent is palladium diphenylphosphinoferrocene dichloride.

Another aspect of the invention relates to a process for preparing compounds of the invention, in accordance with the steps set forth below in Scheme 1.

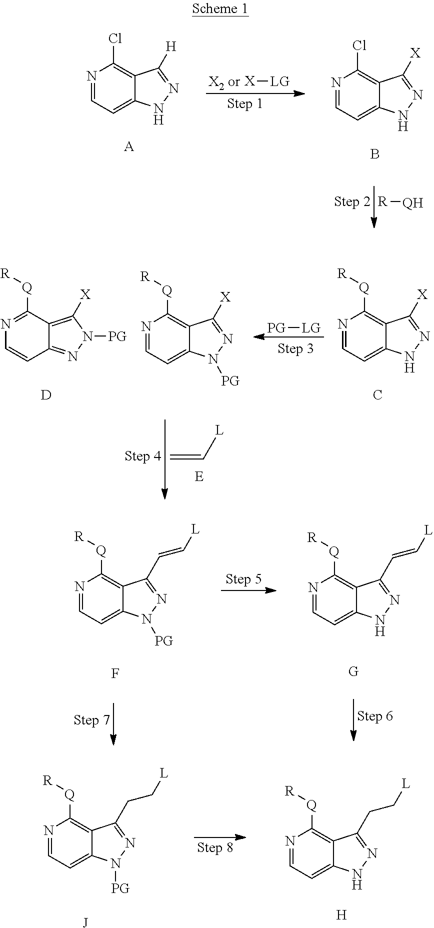

### Step 1

Step 1 describes the conversion of formula A into formula B, wherein X is a halogen, preferably bromine or iodine and LG is a leaving group such as succinimide.

The reaction is carried out in the presence of a suitable halogenating agent, such as iodine or N-bromosuccinimide, optionally in the presence of a base, such as potassium hydroxide in a suitable solvent.

Typical conditions (X=I), 1 eq. of formula A, 2 eq. of I₂, 3.7 eq of KOH in dioxane at 75° C. for 4 h; (X=Br), 1 eq. of formula A, 1 eq. of N-bromosuccinimide in acetonitrile at reflux for 3 h.

### Step 2

Step 2 describes the conversion of formula B into formula C, wherein X is a halogen, RQH can either be a primary or secondary amine, or an alcohol. The group R can optionally contain a functional group which can be manipulated at later stages in the synthetic process using standard conditions known to the skilled person.

The reaction involves nucleophilic displacement of the chloro group in formula B with a an amino group in a suitable solvent, optionally in the presence a Bronsted acid. This reaction generally requires heating, either thermally or with the use of microwave irradiation. Where RQH is an alcohol, the alcohol is deprotonated with a suitable base to the corresponding alkoxide followed by subsequent nucleophilic displacement of the chloro group in formula B.

Typical conditions (RQH=primary or secondary aliphatic amino group), 2.5 eq. of amine, 1 eq. of formula B in n-butanol, heated to 190° C. in the microwave for 20 min; (RQH=primary or secondary aromatic amino group), 2 eq. of amine, 1 eq of formula B, 3 eq of conc. HCl(aq) in n-butanol, heated to 190° C. in the microwave for 45 min; (RQH=alcohol), 4 eq. of alcohol is treated with 3.5 eq. of sodium hydride in dioxane at room temperature for 2 h prior to addition of 1 eq of formula B and subsequent heating in the microwave at 180° C. for 1.5 h.

### Step 3

Step 3 describes the conversion of formula C into formula D, wherein PG is defined as a protecting group, including but not limited to tert-butoxycarbonyl-; benzyloxycarbonyl-; benzyl-; 4-methoxybenzyl-; 2,4-dimethoxybenzyl- or trityl-; LG is defined as a leaving group, such as a halogen or tert-butylcarbonate.

The reaction involves capping of the indazole NH with a protecting group. It will be appreciated by the skilled person, that that many protecting groups can be used for this purpose (see Greene, Theodora W. and Wuts, Peter G. M. Greene's Protective Groups in Organic Synthesis. 4th Ed. (2006)). The skilled person will also appreciate that it is possible to introduce the protecting group either at N1 or N2, and the ratio may change depending on the nature of PG or the precise reaction conditions deployed. The reaction conditions will depend on the nature of the protecting group. Typical conditions (PG=4-methoxybenzyl): 1 eq of 4-methoxybenzyl chloride; 1 eq of formula C, 2 eq of potassium hydroxide is stirred in DMF at room temperature overnight.

### Step 4

Step 4 involves the conversion of formula D to formula F, wherein L is a group, such as but not limited to, aryl, substituted aryl, heteroaryl, substituted heteroaryl, an ester or an amide; X is a halogen, but preferably an iodine. The linker L can optionally contain a functional group which can be manipulated at later stages in the synthetic process using standard conditions known to the skilled person.

The reaction involves a cross coupling of a substituted vinyl derivative (formula E) with formula D in the presence of a suitable transition metal catalyst and a suitable base, preferably triethylamine and optionally additional additives, such as tetrabutyl ammonium iodide. This type of transformation is often known as a “Heck Reaction” to those skilled in the art.

Typical conditions: 1 eq. of formula D, 10 eq. of formula E, 2 eq. of tetrabutylammonium iodide, 0.2 eq. of Pd(dppf)Cl₂ in DMF:Water:triethylamine (6.25:1:1) is heated to 70° C. overnight.

### Step 5

Step 5 involves the conversion of formula F into formula G, wherein Q, PG, and L are as defined earlier.

The reaction involved removal of the protecting group from the indazole and the precise conditions will vary depending the nature of the protecting group (Greene, Theodora W. and Wuts, Peter G. M. Greene's Protective Groups in Organic Synthesis. 4th Ed. (2006).

Typical conditions (QR is a substituted amino group and PG is 4-methoxybenzyl): Formula F is treated with trifluoroacetic acid at 70° C. overnight.

### Step 6

Step 6 involves the conversion of formula G into formula H, wherein PG, L, PG, RQ- are as defined earlier.

The reaction involves hydrogenation of the double bond to the corresponding saturated compound with a hydrogen source in the presence of a suitable transition metal catalyst in a suitable solvent. It may be necessary or desirable to add a Bronsted acid (such as HCl, or acetic acid) to facilitate this reaction. The person skilled in the art will appreciate that a number of different metal catalysts can be used for this type of reaction and that it may be necessary or desirable to carry out these reactions under pressure.

Typical conditions: formula G is treated with platinum oxide in glacial acetic acid under an atmosphere of hydrogen.

### Step 7

Step 7 involves the conversion of formula F into formula J, wherein PG, L, PG, RQ- are as defined earlier.

The reaction involves hydrogenation of the double bond to the corresponding saturated compound with a hydrogen source in the presence of a suitable transition metal catalyst, such as palladium on carbon or platinum oxide in a suitable solvent, such as ethanol, ethyl acetate or dioxane. It may be necessary or desirable to add a Bronsted acid (such as HCl, or acetic acid) to facilitate this reaction. The person skilled in the art will appreciate that a number of different metal catalysts can be used for this type of reaction and that it may be necessary or desirable to carry out these reactions under pressure.

Typical conditions: formula F is treated with 10% palladium on carbon in ethyl acetate under an atmosphere of hydrogen at room temperature overnight.

### Step 8

Step 8 involves the conversion of formula J to formula H, wherein PG, L, PG, RQ- are as defined earlier.

The reaction involves removal of the protecting group from the indazole, and the precise conditions will depend on the nature of the protecting group (Greene, Theodora W. and Wuts, Peter G. M. Greene's Protective Groups in Organic Synthesis. 4th Ed. (2006).

Typical conditions (QR is a substituted amino group and PG is 4-methoxybenzyl): Formula F is treated with trifluoroacetic acid at 70° C. overnight.

### Step 9

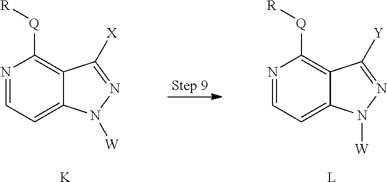

Step 9 describes the conversion of formula K into formula L wherein X and RQ are as defined previously, W can be either hydrogen or a protecting group, such as but not limited to 4-methoxybenzyl or trityl; Y can be aryl, substituted aryl, heteroaryl or substituted heteroaryl. The person skilled in the art will appreciate that where W is a protecting group, this can be removed at a later stage using standard conditions (Greene, Theodora W. and Wuts, Peter G. M. Greene's Protective Groups in Organic Synthesis. 4th Ed. (2006).

The reaction involves cross-coupling of the halide in formula K with a boronic acid or boronic ester in the presence of a transition metal catalyst in a suitable solvent. The reactions are typically carried out at elevated temperatures with either thermal or microwave heating. An inorganic base (such as sodium carbonate) is generally added to the reaction mixture. Transformations of this type are known as “Suzuki Couplings” to those skilled in the art.

Typical conditions: 1 eq. of formula K, 0.09 eq. of Pd(dppf)₂Cl₂, 1.5 eq. of the boronic acid (or boronic ester), 3.5 eq. of 2M aqueous sodium carbonate in dioxane at 90° C. for 18 h.

### Step 10-11

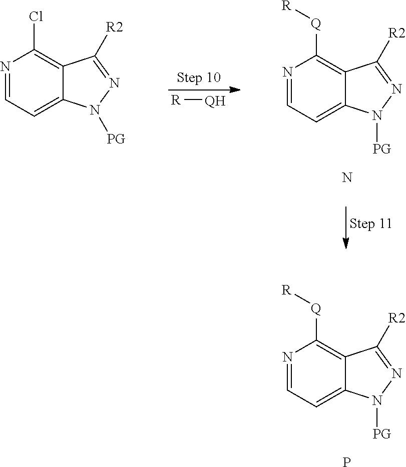

Step 10 describes the conversion of formula M into formula N, wherein R2 and RQH are as defined earlier and PG is a protecting group such as but not limited to 4-methoxybenzyl or trityl.

Where RQH is a primary or secondary amine, the reaction involves nucleophilic displacement of the chloro group in formula M with the amine. The reaction can be either carried out with or without solvent (such as but not limited to n-butanol or N-methylpyrrolidone), optionally in the presence of a Bronsted acid (such as but not limited to HCl) or an organic base (such as but not limited to N,N-diisopropylethylamine). This reaction generally requires heating, either thermally or with the use of microwave irradiation.

Alternatively, the reaction can be carried out by treatment of formula M with a primary or secondary amine in the presence or a transition metal catalyst, in the presence of a base in a suitable solvent.

Typical conditions: 1.4 equivalents of amine, 1 equivalent of formula M, 1 equivalent of cesium carbonate, 0.06 equivalents of palladium(II) acetate and 0.08 equivalents of 2,2′-bis(diphenylphosphino)-1,1′-binaphthyl (BINAP) is heated to 90° C. in 1,4-dioxane overnight.

Where RQH is an alcohol, the alcohol is deprotonated with a suitable base to the corresponding alkoxide followed by subsequent nucleophilic displacement of the chloro group in formula M. Alternatively, the reaction can be carried out by treatment of formula M with a primary or secondary alcohol in the presence or a transition metal catalyst, in the presence of a base in a suitable solvent.

Typical conditions (nucleophilic displacement): 2 eq. of alcohol is treated with 1.5 eq. of sodium hydride in dioxane at room temperature for 3 h prior to addition of 1 eq of formula B and subsequent heating in the microwave at 180° C. for 1.5 h.

Typical conditions (transition metal catalyzed): 2 equivalents of alcohol, 1 equivalent of formula M, 3 equivalents of sodium tert-butoxide, 0.06 equivalents of palladium(II) acetate and 0.08 equivalents of 2,2′-bis(diphenylphosphino)-1,1′-binaphthyl (BINAP) is heated to 100° C. in toluene overnight.

### Step 11

Step 8 involves the conversion of formula N to formula P, wherein R2, PG, RQ- are as defined earlier.

The reaction involves removal of the protecting group from the indazole, and the precise conditions will depend on the nature of the protecting group (Greene, Theodora W. and Wuts, Peter G. M. Greene's Protective Groups in Organic Synthesis. 4th Ed. (2006).

Typical conditions (QR is a substituted amino group and PG is 4-methoxybenzyl): Formula N is treated with trifluoroacetic acid (neat) at 70° C. overnight.

Typical conditions (QR is an alkoxy group and PG is trityl): Formula N is treated with trifluoroacetic acid:DCM (1:10) for 18 h at room temperature.

The invention is further described by way of the following non-limiting examples, and with reference to the following figures, wherein:

FIG. 1 shows the domain structure of LRRK1 and local mutations that have been linked to Parkinson's disease.

### EXAMPLES

### Materials and Methods

### Source and Purification of Kinases

All LRRK2 protein kinases were of human origin and were sourced from Invitrogen Corporation (Carlsbad, Calif. 92008 USA) unless otherwise indicated. The active mutant used was recombinant human, catalytic domain (amino acids 970-2527) containing a G2019S mutation, GST-tagged, expressed in insect cells (Invitrogen Cat #PV4881). The wild type used was recombinant human, catalytic domain (amino acids 970-2527) GST-tagged, expressed in insect cells (Invitrogen Cat #PV4873). The kinase dead mutant used was recombinant human, catalytic domain (amino acids 970-2527) containing a D1994A mutation, GST-tagged, expressed in insect cells (Invitrogen Cat #PM4041AE). No special measures were taken to activate any of the kinases.

### Protein Kinase Assays

All assays were carried out at room temperature (˜21° C.) and were linear with respect to time and enzyme concentration under the conditions used. Assays were performed for 180 min in a 96 well format. LRRK2 was present at a concentration of approximately 5 nM. The enzyme was diluted and assayed in 50 mM Tris-HCl pH7.5, 0.1 mM EGTA, 1 mM DTT and 10 mM MgCl₂. The concentration of magnesium chloride in the assay was 10 mM. The [γ-33P] ATP (0.4 μCi/well) was used at 134 uM for G2019S mutant and at 57 μM for the wild type kinase in order to be at Km. The peptide substrate in the assay was RLGWWRFYTLRRARQGNTKQR at 100 μM.

The assays were initiated with Mg/ATP and stopped by the addition of 25 μl/well 50% orthophosphoric acid. Reactions were harvested onto Whatman P81 Unifilter Plates (Fisher Scientific. Loughborough, LE115RG, UK. Cat #FDU-105-020U) using a Tomtec harvester. (Tomtec Hamden, Conn. 06514. USA) Plates were counted using a Perkin Elmer Top Count NX7. (Perkin Elmer, Shelton Conn. 06484-4794 USA)

IC50 values of inhibitors were determined after carrying out assays at 10 different concentrations of each compound in duplicate.

### General Procedures for Synthesis of Compounds

### Chromatography

Preparative high pressure liquid chromatography was carried out using apparatus made by Agilent. The apparatus is constructed such that the chromatography is monitored by a multi-wavelength UV detector (G1365B manufactured by Agilent) and an MM-ES+APCI mass spectrometer (G-1956A, manufactured by Agilent) connected in series, and if the appropriate criteria are met the sample is collected by an automated fraction collector (G1364B manufactured by Agilent). Collection can be triggered by any combination of UV or mass spectrometry or can be based on time. Typical conditions for the separation process are as follows: The gradient is run over a 10 minute period (gradient at start: 10% methanol and 90% water, gradient at finish: 100% methanol and 0% water; as buffer: either 0.1% trifluoroacetic acid is added to the water (low pH buffer), or ammonium bicarbonate (10 mmol/l) and 35% ammonium hydroxide (1.6 ml/l) is added to the water (high pH buffer). It will be appreciated by those skilled in the art that it may be necessary or desirable to modify the conditions for each specific compound, for example by changing the solvent composition at the start or at the end, modifying the solvents or buffers, changing the run time, changing the flow rate and/or the chromatography column.

Flash chromatography refers to silica gel chromatography and carried out using an SP4 or an Isolara 4 MPLC system (manufactured by Biotage); pre-packed silica gel cartridges (supplied by Biotage); or using conventional glass column chromatography.

### Analytical Methods

¹H Nuclear magnetic resonance (NMR) spectroscopy was carried out using an ECX400 spectrometer (manufactured by JEOL) in the stated solvent at around room temperature unless otherwise stated. In all cases. NMR data were consistent with the proposed structures. Characteristic chemical shifts (δ) are given in parts-per-million using conventional abbreviations for designation of major peaks: e.g. s, singlet; d, doublet; t, triplet; q, quartet; dd, doublet of doublets; br, broad. Mass spectra were recorded using a MM-ES+APCI mass spectrometer (G-1956A, manufactured by Agilent). Where thin layer chromatography (TLC) has been used it refers to silica gel TLC using silica gel MK6F 60 Å plates, R_{f} is the distance traveled by the compound divided by the distance traveled by the solvent on a TLC plate.

### Compound Preparation

Where the preparation of starting materials is not described, these are commercially available, known in the literature, or readily obtainable by those skilled in the art using standard procedures. Where it is stated that compounds were prepared analogously to earlier examples or intermediates, it will be appreciated by the skilled person that the reaction time, number of equivalents of reagents and temperature can be modified for each specific reaction and that it may be necessary or desirable to employ different work-up or purification techniques. Where reactions are carried out using microwave irradiation, the microwave used is an Initiator 60 supplied by Biotage. The actual power supplied varies during the course of the reaction in order to maintain a constant temperature.

### Abbreviations

DCM=Dichloromethane
DMF=N,N-Dimethylformamide
THF=Tetrahydrofuran
MeOH=Methanol
TFA=Trifluoroacetic acid
Xantphos=4,5-Bis(diphenylphosphino)-9,9-dimethylxanthene
HATU=N,N,N′,N′-Tetramethyl-O-(7-azabenzotriazol-1-yl)uronium-hexafluorophosphate
EDCI=1,3-Propanediamine, N3-(ethylcarbonimidoyl)-N1,N1-dimethyl-, hydrochloride
DCC=1,3-Dicyclohexylcarbodiimide
Pd₂(dba)₃=tris(dibenzylideneacetone)dipalladium(0)
TEA=Triethylamine
rm=Reaction mixture
rt=Room temperature
AcOH=Acetic acid
IPA=Isopropanol
DIPEA=N,N-diisopropylethylamine
TBSMSCl=Tertiarybutyldimethylsilyl chloride
MeCN=Acetonitrile
NH₃=Ammonia
EtOH=Ethanol
EtOAc=Ethyl Acetate
LCMS=Mass spectrometry directed high pressure liquid chromatography
UV=Ultraviolet
SCX=Strong cation exchange
TPAP=Tetrapropylammonium perruthenate
DMSO=Dimethylsulphoxide
BINAP=2,2′-bis(diphenylphosphino)-1,1′-binaphthyl

The structures of selected compounds of the invention are shown in the table below:

### Intermediate 1

### 1-(2,4-Dichloro-pyridin-3-yl)-ethanol

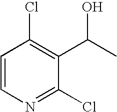

A solution of methylmagnesium chloride, 3M in THF (20.4 ml, 61.3 mmol) was added to 2,4-dichloro-pyridine-3-carbaldehyde (9.8 g, 55.7 mmol) in THF (200 ml) at −78° C. The reaction mixture was stirred at −78° C. for 30 minutes and allowed to warm to rt. The mixture was quenched with saturated ammonium chloride solution (aq) and the product was extracted with EtOAc. The organic extract was washed with brine, dried and concentrated. The crude residue was purified by flash column chromatography over silica gel (300 g) eluting with 2:1 petroleum ether:EtOAc to provide a green coloured oil (7.8 g, 73%). ¹H NMR (400 MHz, CHLOROFORM-d) δ ppm 1.67 (d, J=6.87 Hz, 3H), 5.57 (q, J=6.87 Hz, 1H), 7.29 (d, J=5.50 Hz, 1H), 8.20 (d, J=5.50 Hz, 1H).

### Intermediate 2

### 1-(2,4-Dichloro-pyridin-3-yl)-ethanone

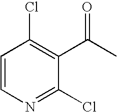

Freshly activated 4 Å molecular sieves (9.0 g) and NMO (7.1 g, 60.9 mmol) were added to a solution of Intermediate 1 (7.8 g, 40.6 mmol) in DCM (130 ml) and the mixture was stirred for 15 minutes. TPAP (403 mg, 1.15 mmol) was added and the reaction mixture was stirred for 2 hours at rt. The mixture was then filtered through Celite and the filtrate was concentrated. The crude residue was purified by flash column chromatography over silica gel (270 g) eluting with 4:1 petroleum ether:EtOAc to give a pale yellow coloured oil (6.2 g, 80%). ¹H NMR (400 MHz, CHLOROFORM-d) δ ppm 2.62 (s, 3H) 7.34 (d, J=5.50 Hz, 1H) 8.34 (d, J=5.50 Hz, 1H).

### Intermediate 3

### 4-Chloro-3-methyl-1H-pyrazolo[4,3-c]pyridine

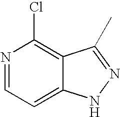

1-(2,4-Dichloro-pyridin-3-yl)-ethanone (6.2 g, 32.6 mmol) in 65% hydrazine monohydrate (45 ml) was stirred at rt overnight. The mixture was diluted with EtOAc and water. The organic extract was washed with brine, dried and concentrated to give a white solid. The crude product was purified by flash column chromatography over silica gel (200 g) eluting with 1:1 petroleum ether:EtOAc to give an off-white solid (3.5 g, 64%). ¹H NMR (400 MHz, DMSO-d₆) δ ppm 2.64 (s, 3H) 7.47 (d, J=5.95 Hz, 1H) 8.06 (d, J=5.95 Hz, 1H). m/z (ES+APCI)⁺: 168/170 [M+H]⁺.

### Intermediate 4

### 4-Chloro-1-(4-methoxy-benzyl)-3-methyl-1H-pyrazolo[4,3-]pyridine

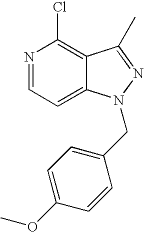

Intermediate 3 (1 g, 5.99 mmol), 4-methoxybenzylchloride (0.82 ml, 5.99 mmol) and potassium hydroxide (0.5 g, 8.98 mmol) were combined in DMF (20 ml) under nitrogen. The reaction was stirred at room temperature overnight. The reaction mixture was evaporated, the residue dissolved in EtOAc (20 ml) and partitioned with water (20 ml). The aqueous layer was extracted with EtOAc (20 ml) and then the combined organic layers were washed with brine, dried (MgSO₄) and evaporated. The crude product was purified by flash chromatography on the Biotage SP4, eluting with 0 to 60% EtOAc/petroleum ether to give a white solid (1.65 g, 96%). The product was isolated as a 4:1 mixture of N1 and N2 alkylated regioisomers: Major regioisomer: ¹H NMR (400 MHz, DMSO-d₆) δ ppm 2.64 (s, 3H), 3.70 (s, 3H), 5.52 (s, 2H), 6.87 (d, J=8.7 Hz, 2H), 7.22 (d, J=8.7 Hz, 2H), 7.77 (d, J=6.0 Hz, 1H), 8.11 (d, J=6.0 Hz, 1H). m/z (ES+APCI)⁺: 288/290. Minor regioisomer: ¹H NMR (400 MHz, DMSO-d₆) δ ppm 2.83 (s, 3H), 3.71 (s, 3H), 5.61 (s, 2H), 6.90 (d, J=8.7 Hz, 2H), 7.22 (d, J=8.7 Hz, 2H), 7.49 (d, J=6.0 Hz, 1H), 7.91 (d, J=6.4 Hz, 1H); m/z (ES+APCI)⁺: 288/290.

### Intermediate 5

### 1-(4-Methoxy-benzyl)-3-methyl-1H-pyrazolo[4,3-c]pyridine-4-carbonitrile

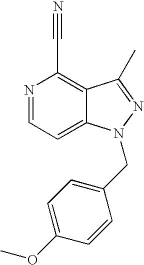

Intermediate 4 (200 mg, 0.70 mmol), zinc cyanide (81 mg, 0.70 mmol) and Pd(PPh₃)₄ (80 mg, 0.07 mmol) were combined in DMF (2.5 ml), degassed for 10 minutes and placed under an atmosphere of nitrogen. The reaction mixture was irradiated at 180° C. for 20 min in a Biotage I-60 microwave reactor. The reaction was diluted with EtOAc (5 ml) and partitioned with saturated NaHCO₃ aqueous solution (10 ml). The aqueous layer was then extracted with EtOAc (2×20 ml). The combined organic layers were washed with brine, dried (MgSO₄) and evaporated. The crude product was purified by flash chromatography on the Biotage SP4, eluting with 0 to 60% EtOAc/petroleum ether to give a white solid (148 mg, 76%). NMR data indicate a mixture of N-1 and N-2 regioisomers:

Major product: ¹H NMR (400 MHz, DMSO-d₆) δ ppm 2.70 (s, 3H), 3.70 (s, 3H), 5.59 (s, 2H), 6.85-6.89 (m, 2H), 7.22-7.26 (m, 2H), 8.11 (d, J=6.0 Hz, 1H), 8.52 (d, J=6.0 Hz, 1H); m/z (ES+APCI)⁺: 279 [M+H]⁺.

Minor product: ¹H NMR (400 MHz, DMSO-d₆) δ ppm 2.88 (s, 3H), 3.72 (s, 3H), 5.68 (s, 2H), 6.89-6.93 (m, 2H), 7.24-7.27 (m, 2H), 7.87 (d, J=6.0 Hz, 1H), 8.32 (d, J=5.9 Hz, 1H); m/z (ES+APCI)⁺: 279 [M+H]⁺.

### Intermediate 6

### 1-(4-Methoxy-benzyl)-3-methyl-1H-pyrazolo[4,3-c]pyridine-4-carboxylic acid

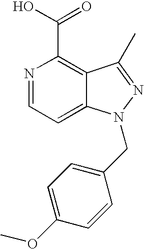

Potassium hydroxide (1.45 g, 25.9 mmol) was added to a solution of Intermediate 5 (720 mg, 2.51 mmol) in EtOH (20 ml) and H₂O (3 ml), and the mixture was refluxed for 18 h. The reaction was allowed to cool, then diluted with H₂O (100 ml) and adjusted to pH3 with concentrated HCl(aq). During extraction with EtOAc, a white solid crashed out of the aqueous phase, which was filtered and dried to give a white solid (217 mg, 28%). ¹H NMR (400 MHz, DMSO-d₆) δ ppm 2.61 (s, 3H), 3.70 (s, 3H), 5.57 (s, 2H), 6.83-6.91 (m, 2H), 7.18-7.25 (m, 2H), 7.93 (d, J=6.0 Hz, 1H), 8.34 (d, J=6.0 Hz, 1H). m/z (ES+APCI)⁺: 298 [M+H]⁺.

### Intermediate 7

### 4-[1-(4-Methoxy-benzyl)-3-methyl-1H-pyrazolo[4,3-c]pyridin-4-ylamino]-piperidine-1-carboxylic acid tert-butyl ester

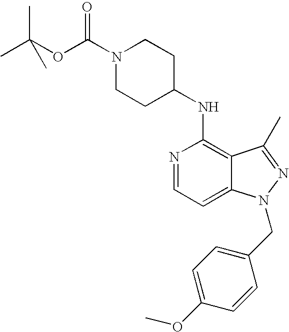

Intermediate 4 (1 g, 3.48 mmol), 4-amino-1-boc-piperidine (0.98 g, 4.88 mmol), Pd(OAc)₂ (47 mg, 0.21 mmol), BINAP (174 mg, 0.28 mmol) and cesium carbonate (3.39 g, 10.5 mmol) were combined in dioxane (20 ml). The mixture was degassed and placed under an atmosphere of nitrogen, then stirred at 90° C. for 18 h. The mixture was diluted with DCM (50 ml), partitioned with H₂O (50 ml) and the aqueous layer extracted with DCM (2×50 ml). The combined organic layers were washed with brine, dried (MgSO₄) and evaporated. The crude product was purified by flash chromatography on the Biotage SP4, eluting with 0 to 100% EtOAc/petroleum ether to give a yellow solid (950 mg, 60%). ¹H NMR (400 MHz, DMSO-d₆) δ ppm 1.40 (s, 9H), 1.44-1.57 (m, 2H), 1.85-1.93 (m, 2H), 2.56 (s, 3H), 2.74-2.93 (m, 2H), 3.69 (s, 3H), 3.88-4.00 (m, 2H), 4.18-4.28 (m, 1H), 5.32 (s, 2H), 5.68-5.73 (m, 1H), 6.79 (d, J=6.0 Hz, 1H), 6.81-6.88 (m, 2H), 7.12-7.17 (m, 2H), 7.69 (d, J=6.0 Hz, 1H). m/z (ES+APCI)⁺: 452 [M+H]⁺

### Intermediate 8

### [1-(4-Methoxy-benzyl)-3-methyl-1H-pyrazolo[4,3-c]pyridin-4-yl]-piperidin-4-yl-amine hydrochloride salt

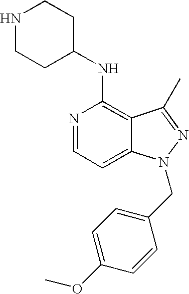

Intermediate 7 (0.92 g, 2.04 mmol) and 4M hydrochloric acid (20 ml) were combined and stirred at room temperature for 3 h. The reaction mixture was evaporated to give a white solid (0.85 g, 100%). ¹H NMR (400 MHz, DMSO-d₆) δ ppm 1.93-2.05 (m, 2H), 2.06-2.14 (m, 2H), 2.66 (s, 3H), 2.94-3.08 (m, 2H), 3.31-3.45 (m, 2H), 3.71 (s, 3H), 4.21-4.51 (m, 1H), 5.50 (s, 2H), 6.86-6.91 (m, 2H), 7.19-7.25 (m, 2H), 7.37-7.44 (m, 1H), 7.67-7.73 (m, 1H), 7.79-7.86 (m, 1H), 8.95 (br. s., 1H). m/z (ES+APCI)⁺: 352 [M+H]⁺.

### Intermediate 9

### 4-[1-(4-Methoxy-benzyl)-3-methyl-1H-pyrazolo[4,3-c]pyridin-4-ylamino]-benzoic acid

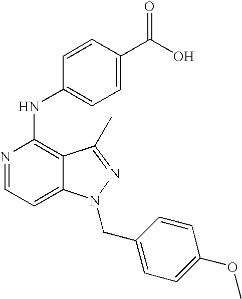

### Step 1

Intermediate 4 (1 g, 3.48 mmol), 4-amino-benzoic acid methyl ester (0.74 g, 4.88 mmol), Pd(OAc)₂ (47 mg, 0.21 mmol), BINAP (174 mg, 0.28 mmol) and cesium carbonate (3.4 g, 10.5 mmol) were combined in dioxane (20 ml). The mixture was degassed and placed under an atmosphere of nitrogen, then stirred at 90° C. for 18 h. The mixture was diluted with DCM (50 ml), partitioned with H₂O (50 ml) and the aqueous layer extracted with DCM (2×50 ml). The combined organic layers were washed with brine, dried (MgSO₄) and evaporated. The crude product was purified by flash chromatography on the Biotage SP4, eluting with 0 to 60% EtOAc/petroleum ether to give a pale yellow solid (945 mg) which was used in the next step without further purification.

### Step 2

2M NaOH (aq) (3.5 ml, 7.05 mmol) was added to the crude product of Step 1 (945 mg) in EtOH (20 ml). The reaction mixture was stirred at 70° C. for 4 h. The reaction mixture was evaporated, dissolved in H₂O (20 ml) and adjusted to pH6 with 1M HCl (aq). The precipitate was filtered and washed with H₂O. The solid was azeotroped with toluene and then acetonitrile to give a yellow solid (832 mg, 91%). NMR data indicate a mixture of N-1 and N-2 regioisomers:

Major regioisomer: ¹H NMR (400 MHz, DMSO-d₆) δ ppm 2.71 (s, 3H), 3.60 (br. s., 1H), 3.72 (s, 3H), 5.53 (s, 2H), 6.87-6.92 (m, 2H), 7.23-7.27 (m, 2H), 7.46 (d, J=6.0 Hz, 1H), 7.67 (d, J=8.2 Hz, 2H), 7.75 (d, J=6.9 Hz, 1H), 8.00 (d, J=8.2 Hz, 2H), 9.65 (br. s., 1H); m/z (ES+APCI)⁺: 389 [M+H]⁺

Minor regioisomer: ¹H NMR (400 MHz, DMSO-d₆) δ ppm 2.91 (s, 3H), 3.61 (br. s., 1H), 3.73 (s, 3H), 5.62 (s, 2H), 6.92-6.96 (m, 2H), 7.18 (d, J=7.3 Hz, 1H), 7.25-7.29 (m, 2H), 7.38 (d, J=6.9 Hz, 1H), 7.62 (d, J=8.7 Hz, 2H), 8.08 (d, J=8.7 Hz, 2H), 9.65 (br. s., 1H); m/z (ES+APCI)⁺: 389 [M+H]⁺

### Intermediate 10

### 2,4-Dichloro-pyridine-3-carbaldehyde

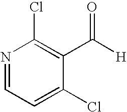

To a solution of n-butyllithium (1.6 M in hexane, 64 ml, 101 mmol) in THF (150 ml) at −78° C. was added diisopropylamine (14.3 ml, 101 mmol) dropwise. The reaction mixture was allowed to warm to 0° C. over 1 h, and then cooled down to −78° C. 2,4-Dichloropyridine (11 ml, 101 mmol) was added dropwise and the solution was stirred at −78° C. for 2.5 h. N-Formylpiperidine (11.2 ml, 101 mmol) was then added dropwise and the mixture stirred at −78° C. for a further 1.5 h. The solution was quenched at −78° C. with saturated NH₄Cl (aq) and then allowed to warm to room temperature. The reaction mixture was diluted with ethyl acetate and washed with 1M HCl (aq), the organic phase was separated, washed with saturated NaHCO₃ (aq), dried (MgSO₄) and evaporated to dryness. The crude residue was purified by flash chromatography, eluting with 0 to 20% ethyl acetate/petroleum ether gradient to give a yellow solid (9.7 g, 54%). ¹H NMR (400 MHz, DMSO-d₆) δ ppm 7.78 (d, J=5.04 Hz, 1H), 8.56 (d, J=5.50 Hz, 1H), 10.31 (s, 1H). R_{f} (20% ethyl acetate in petroleum ether)=0.70.

### Intermediate 11

### 4-Chloro-1H-pyrazolo[4,3-c]pyridine

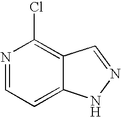

To a solution of Intermediate 11 (1.7 g, 9.7 mmol) in dimethoxyethane (12 ml) at room temperature was added hydrazine monohydrate (1.2 ml, 38.6 mmol) and the resulting mixture was stirred at 75° C. overnight. The mixture was then concentrated to dryness and the crude residue was purified by flash chromatography, eluting with 20 to 100% ethyl acetate/petroleum ether gradient to give a white solid (0.82 g, 56%). ¹H NMR (400 MHz, DMSO-d₆) δ ppm 7.60 (d, J=6.9 Hz, 1H), 8.14 (d, J=6.0 Hz, 1H), 8.32 (s, 1H); m/z (ES+APCI)⁺: 154 [M+H]⁺.

### Intermediate 12

### 4-Chloro-3-iodo-1H-pyrazolo[4,3-c]pyridine

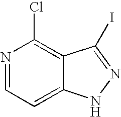

To a mixture of Intermediate 11 (5.8 g, 38 mmol) and KOH (8 g, 142 mmol) in dioxane (100 ml) at room temperature was added iodine (19 g, 76 mmol). The reaction mixture was stirred at 75° C. for 4 h, and then allowed to cool to room temperature. The solution was diluted with saturated Na₂S₂O₃ (aq), and the resulting precipitate was filtered and dried to give a yellow solid (4.1 g). The filtrate was left standing for 3 days and filtration of the resulting precipitate yielded a further 2.35 g of the product. Combined yield (6.45 g, 61%). ¹H NMR (400 MHz, DMSO-d₆) δ ppm 7.64 (d, J=6.0 Hz, 1H), 8.11 (d, J=6.0 Hz, 1H); m/z (ES+APCI)⁺: 280 [M+H]⁺.

### Intermediate 13

### 4-Chloro-3-iodo-1-(4-methoxy-benzyl)-1H-pyrazolo[4,3-c]pyridine

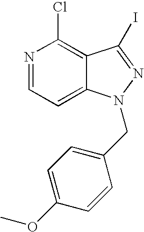

To a mixture of Intermediate 12 (1 g, 3.6 mmol) and KOH (0.3 mg, 5.4 mmol) in DMF (10 ml) at room temperature was added 4-methoxybenzyl chloride (0.5 ml, 3.6 mmol). The resulting mixture was stirred at room temperature for 2.5 h, and then evaporated to dryness. The crude residue was dissolved in EtOAc and washed with water. The organic phase was dried and purified by flash chromatography, eluting with 0 to 30% ethyl acetate/petroleum ether gradient to give a 9:1 mixture of N1:N2 regioisomers as a solid (1.3 g, 93%). Major regioisomer: ¹H NMR (400 MHz, DMSO-d₆) δ ppm 3.72 (s, 3H), 5.62 (s, 2H), 6.85-6.94 (m, 2H), 7.20-7.27 (m, 2H), 7.95 (d, J=6.0 Hz, 1H), 8.20 (d, J=6.0 Hz, 1H); m/z (ES+APCI)⁺: 400 [M+H]⁺.

### Intermediate 14

### Cyclohexyl-[3-iodo-1-(4-methoxy-benzyl)-1H-pyrazolo[4,3-c]pyridin-4-yl]-amine

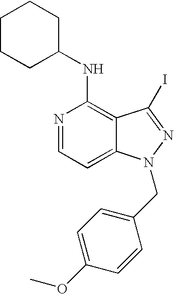

To a solution of Intermediate 13 (0.95 g, 2.4 mmol) in 1-butanol (5 ml) at room temperature was added cyclohexylamine (1.1 ml, 9.52 mmol). The resulting mixture was irradiated at 190° C. for 1 h in a Biotage I-60 microwave reactor. The reaction mixture was then evaporated to dryness and the crude residue was purified by flash chromatography eluting with 10 to 100% ethyl acetate/petroleum ether gradient to give a white solid (0.87 g, 80%) ¹H NMR (400 MHz, DMSO-d₆) δ ppm 1.24-1.50 (m, 5H), 1.50-1.63 (m, 1H), 1.63-1.80 (m, 2H), 1.86-2.03 (m, 2H), 3.72 (s, 3H), 4.02-4.15 (m, 1H), 5.43 (s, 2H), 5.95 (d, J=7.3 Hz, 1H), 6.85-6.90 (m, 2H), 6.95 (d, J=6.0 Hz, 1H), 7.15-7.24 (m, 2H), 7.76 (d, J=6.0 Hz, 1H); m/z (ES+APCI)⁺: 463 [M+H]⁺.

### Intermediate 15

### 3-Bromo-4-chloro-1H-pyrazolo[4,3-c]pyridine

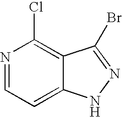

N-bromosuccinimide (1.87 g, 10.5 mmol) was added to a solution of Intermediate 11 (1.61 g, 10.5 mmol) in acetonitrile (50 ml), and the mixture was heated to reflux for 3 h. The solvents were evaporated and DCM (60 ml) was added to the crude solid and the mixture stirred at r.t. for 30 min. The beige solid was filtered off, washed with DCM, then dried under vacuum (1.98 g, 81%). ¹H NMR (400 MHz, DMSO-d₆) δ ppm 7.69 (d, J=6.0 Hz, 1H), 8.22 (d, J=6.0 Hz, 1H); m/z (ES+APCI)⁺: 232/234/236 [M+H]⁺.

### Intermediate 16

### Cyclopropyl-(2,4-dichloro-pyridin-3-yl)-methanol

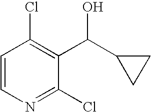

To a solution of Intermediate 10 (1.00 g, 5.68 mmol) in dry THF (100 ml), under nitrogen at −78° C. was added dropwise cyclopropyl magnesium bromide (0.5 M in THF, 12.5 ml, 6.25 mmol). After stirring at −78° C. for a further 3 h, the mixture was warmed up to −20° C., and then quenched with saturated ammonium chloride. The aqueous phase was extracted twice with ethyl acetate and the combined organic extracts washed with brine, dried (MgSO₄) and concentrated. Purification by flash chromatography using a Biotage SP4 (ethyl acetate/petroleum ether gradient) gave the product (443 mg, 36%). ¹H NMR (400 MHz, DMSO-d₅) δ ppm 0.25-0.32 (m, 1H), 0.38-0.46 (m, 1H), 0.48-0.55 (m, 1H), 0.62-0.70 (m, 1H), 1.61-1.73 (m, 1H), 4.47 (dd, J=9.2, 4.1 Hz, 1H), 5.67 (d, J=4.1 Hz, 1H), 7.62 (d, J=5.5 Hz, 1H), 8.31 (d, J=5.5 Hz, 1H); m/z (ES+APCI)⁺: 218 [M+H]⁺.

### Intermediate 17

### Cyclopropyl-(2,4-dichloro-pyridin-3-yl)-methanone

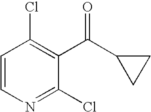

Freshly activated 4 Å molecular sieves and N-methylmorpholine-N-oxide (336 mg, 2.87 mmol) were added to a solution of Intermediate 16 (417 mg, 1.91 mmol) in DCM (10 ml), under nitrogen and stirred for 15 min. After this time, TPAP (20 mg, 0.06 mmol) was added and stirring then continued for further 3.5 h at r.t. The reaction mixture was filtered through Celite and the filtrate concentrated. Purification by flash chromatography using a Biotage SP4 (ethyl acetate/petroleum ether gradient) gave the product (298 mg, 72%). ¹H NMR (400 MHz, DMSO-d₆) δ ppm 1.19-1.33 (m, 4H), 2.43-2.52 (m, 1H), 7.81 (d, J=5.5 Hz, 1H), 8.53 (d, J=5.5 Hz, 1H); Rf=0.62 (1:1 petroleum ether/ethyl acetate).

### Intermediate 18

### 4-Chloro-3-cyclopropyl-1H-pyrazolo[4,3-c]pyridine

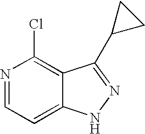

65% Hydrazine hydrate (1 ml) was added to Intermediate 17 (278 mg, 1.29 mmol) and the reaction stirred at r.t. for 19 h. The reaction mixture was partitioned between water and ethyl acetate and extracted twice with ethyl acetate. The combined organic extracts were washed with brine and dried (MgSO₄). Purification by flash chromatography using a Biotage SP4 (ethyl acetate/petroleum ether gradient) gave the product as a white solid (70 mg, 28%). ¹H NMR (400 MHz, DMSO-d₆) δ ppm 0.96-1.11 (m, 4H), 2.56-2.64 (m, 1H), 7.52 (d, J=6.0 Hz, 1H), 8.10 (d, J=6.0 Hz, 1H); m/z (ES+APCI)⁺: 194/196 [M+H]⁺.

### Intermediate 19

### 4-Cyclohexyloxy-3-iodo-1-(4-methoxy-benzyl)-1H-pyrazolo[4,3-c]pyridine

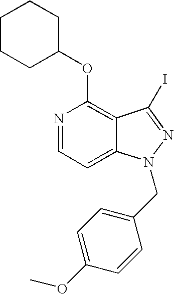

To a 2-5 ml microwave vial containing a solution of cyclohexanol (156 μl, 1.50 mmol) in dioxane (3 ml) was added NaH (60% dispersion, 45 mg, 1.13 mmol), the vessel was capped and flushed out with nitrogen and stirred for 3 h at r.t. A solution of Intermediate 13 (300 mg, 0.75 mmol) in dioxane (1 ml) was added and the vessel was irradiated at 180° C. for 1.5 h in the microwave. The solvents were evaporated, the crude mixture was partitioned between ethyl acetate and water, and the aqueous phase was extracted twice with ethyl acetate. The combined organic extracts washed with brine, dried (MgSO₄) concentrated. Purification by flash chromatography using a Biotage SP4 (ethyl acetate/petroleum ether gradient) gave a white solid (168 mg, 48%). ¹H NMR (400 MHz, DMSO-d₆) δ ppm 1.42-1.58 (m, 4H), 1.65-1.78 (m, 2H), 1.82-1.96 (m, 4H), 3.75 (s, 3H), 5.35-5.41 (m, 1H), 5.55 (s, 2H), 6.92 (d, J=9.2 Hz, 2H), 7.25 (d, J=9.2 Hz, 2H), 7.39 (d, J=6.0 Hz, 1H), 7.90 (d, J=6.4 Hz, 1H); m/z (ES+APCI)⁺: 464 [M+H]⁺.

### Intermediate 20

### (E)-3-[4-Cyclohexylamino-1-(4-methoxy-benzyl)-1H-pyrazolo[4,3-c]pyridin-3-yl]-acrylic acid methyl ester

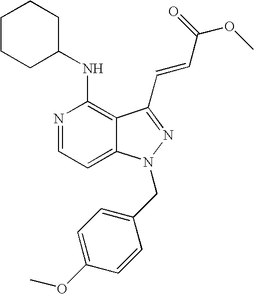

To a mixture of Intermediate 14 (3.1 g, 6.7 mmol) and tetrabutylammonium iodide (4.9 g, 13.4 mmol) in DMF/water/triethylamine (60 ml/9.2 ml/9.2 ml) at room temperature was added methyl acrylate (6 ml, 67 mmol) and Pd(dppf)Cl₂ (1.1 g, 1.34 mmol) respectively. The resulting mixture was heated at 70° C. overnight and then evaporated to dryness. The crude residue was dissolved in EtOAc and washed with water. The organic phase was dried, evaporated and purified by flash chromatography, eluting with 15 to 70% ethyl acetate/petroleum ether gradient to give a yellow solid (2 g, 71%) ¹H NMR (400 MHz, DMSO-d₆) 5 ppm 1.10-1.26 (m, 1H), 1.28-1.45 (m, 4H), 1.62 (d, J=12.4 Hz, 1H), 1.69-1.77 (m, 2H), 1.92-1.99 (m, 2H), 3.70 (s, 3H), 3.76 (s, 3H), 3.98-4.07 (m, 1H), 5.49 (s, 2H), 6.21 (d, J=7.8 Hz, 1H), 6.67 (d, J=15.6 Hz, 1H), 6.84-6.96 (m, 3H), 7.21-7.25 (m, 2H), 7.80 (d, 1H), 8.05 (d, J=15.6 Hz, 1H); m/z (ES+APCI)⁺: 421 [M+H]⁺.

### Intermediate 21

### 3-[4-Cyclohexylamino-1-(4-methoxy-benzyl)-1H-pyrazolo[4,3-c]pyridin-3-yl]-propionic acid methyl ester

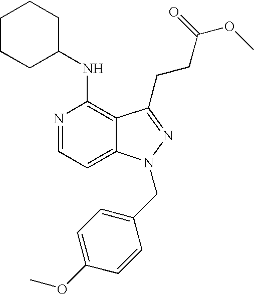

To a solution of Intermediate 20 (2 g, 4.7 mmol) in ethyl acetate (50 ml) at room temperature was added 10% Pd/C (0.4 g). The resulting mixture was stirred under hydrogen at room temperature overnight. The reaction mixture was then filtered through Celite™ and evaporated to give a gum (2 g, 100%). ¹H NMR (400 MHz, DMSO-d₆) δ ppm 1.13-1.25 (m, 1H), 1.27-1.43 (m, 4H), 1.60-1.72 (m, 1H), 1.67-1.77 (m, 2H), 1.89-1.99 (m, 2H), 2.77 (t, 2H), 3.27 (t, J=7.1 Hz, 2H), 3.57 (s, 3H), 3.69 (s, 3H), 3.99-4.08 (m, 1H), 5.32 (s, 2H), 5.63 (d, J=7.8 Hz, 1H), 6.74 (d, J=6.0 Hz, 1H), 6.81-6.86 (m, 2H), 7.10-7.15 (m, 2H), 7.69 (d, J=6.0 Hz, 1H); m/z (ES+APCI)⁺: 423 [M+H]⁺.

### Intermediate 22

### 3-(4-Cyclohexylamino-1H-pyrazolo[4,3-c]pyridin-3-yl)-propionic acid methyl ester

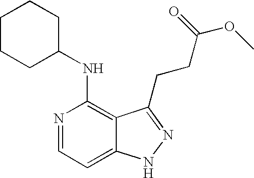

A solution of Intermediate 21 (1.37 g, 3.24 mmol) in TFA (12 ml) was stirred at 70° C. for 4 h, and then allowed to cool to room temperature overnight. 2M NaOH (aq) was added, followed by NH₃ (aq), and then the aqueous was extracted with EtOAc. The organic phase was dried (MgSO₄), evaporated and purified by flash chromatography, eluting with 50% ethyl acetate/petroleum ether to 10% methanol/ethyl acetate gradient to give a cream solid (1.0 g, 100%) ¹H NMR (400 MHz, DMSO-d₆) δ ppm 1.14-1.25 (m, 1H), 1.29-1.44 (m, 4H), 1.58-1.66 (m, 1H), 1.69-1.79 (m, 2H), 1.91-2.01 (m, 2H), 2.79 (t, 2H), 3.28 (t, J=7.1 Hz, 2H), 3.60 (s, 3H), 3.98-4.07 (m, 1H), 5.68 (br. s., 1H), 6.59 (d, J=6.0 Hz, 1H), 7.67 (d, J=6.0 Hz, 1H); m/z (ES+APCI)⁺: 303 [M+H]⁺.

### Intermediate 23

### 3-(4-Cyclohexylamino-1H-pyrazolo[4,3-c]pyridin-3-yl)-propionic acid

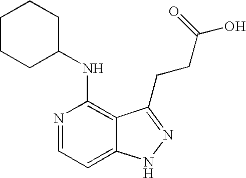

To a stirred solution of Intermediate 22 (0.98 g, 3.24 mmol) in methanol (10 ml) at room temperature was added 2M NaOH (4 ml, 8.11 mmol). The resulting mixture was stirred at room temperature overnight. Acetic acid (0.57 ml, 9.73 mmol) was then added, and the resulting precipitate was filtered and dried to give a white solid (0.7 g, 75%). ¹H NMR (400 MHz, DMSO-d₆) δ ppm 1.04-1.45 (m, 5H), 1.60-1.72 (m, 1H), 1.66-1.78 (m, 2H), 1.87-2.01 (m, 2H), 2.68 (t, J=7.1 Hz, 2H), 3.19 (t, 2H), 3.97-4.07 (m, 1H), 5.82 (br. s., 1H), 6.57 (d, J=6.0 Hz, 1H), 7.66 (d, J=6.0 Hz, 1H); m/z (ES+APCI)⁺: 289 [M+H]⁺.

### Intermediate 24

### (E)-3-[4-Cyclohexylamino-1-(4-methoxy-benzyl)-1H-pyrazolo[4,3-c]pyridin-3-yl]-N,N-dimethyl-acrylamide

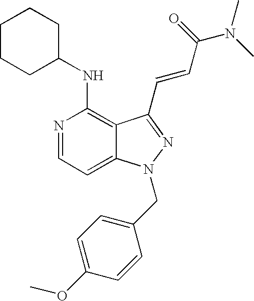

To a mixture of Intermediate 14 (0.25 g, 0.54 mmol) and tetrabutylammonium iodide (0.4 g, 1.08 mmol) in DMF/water/triethylamine (5 ml/0.8 ml/0.8 ml) at room temperature was added N,N-dimethylacrylamide (0.56 ml, 5.4 mmol) and Pd(dppf)Cl₂ (88 mg, 0.11 mmol) respectively. The resulting mixture was heated at 65° C. overnight and then evaporated to dryness. The crude residue was dissolved in EtOAc and washed with water. The organic phase was dried (MgSO₄), evaporated and purified by flash chromatography, eluting with 50 to 100% ethyl acetate/petroleum ether gradient to give a brown gum (185 mg, 79%). ¹H NMR (400 MHz, DMSO-d₆) δ ppm 1.16-1.24 (m, 1H), 1.28-1.42 (m, 4H), 1.61 (m, 1H), 1.68-1.75 (m, 2H), 1.93-1.99 (m, 2H), 2.95 (s, 3H), 3.16 (s, 3H), 3.70 (s, 3H), 4.00-4.06 (m, 1H), 5.49 (s, 2H), 5.98 (d, J=7.8 Hz, 1H), 6.85-6.90 (m, 3H), 7.18-7.23 (m, 3H), 7.77-7.80 (m, 2H); m/z (ES+APCI)⁺: 434 [M+H]⁺.

### Intermediate 25

### 3-[4-Cyclohexylamino-1-(4-methoxy-benzyl)-1H-pyrazolo[4,3-c]pyridin-3-yl]-N,N-dimethyl-propionamide

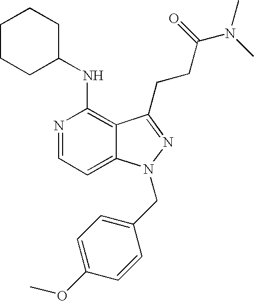

To Intermediate 24 (185 mg, 0.43 mmol) in ethyl acetate (3 ml) at room temperature was added 10% Pd/C (30 mg, 20% by wt). The resulting mixture was stirred under an atmosphere of hydrogen at room temperature overnight. The reaction mixture was then filtered through Celite™ and evaporated to give a gum (126 mg, 68%). ¹H NMR (400 MHz, DMSO-d₆) δ ppm 1.12-1.26 (m, 1H), 1.20-1.46 (m, 4H), 1.55-1.67 (m, 1H), 1.67-1.80 (m, 2H), 1.90-2.03 (m, 2H), 2.75 (t, J=6.6 Hz, 2H), 2.81 (s, 3H), 2.94 (s, 3H), 3.17 (t, J=6.6 Hz, 2H), 3.69 (s, 3H), 3.96-4.07 (m, 1H), 5.34 (s, 2H), 6.53 (d, J=7.3 Hz, 1H), 6.74 (d, J=6.4 Hz, 1H), 6.82-6.90 (m, 2H), 7.11-7.19 (m, 2H), 7.68 (d, J=6.4 Hz, 1H); m/z (ES+APCI)⁺: 436 [M+H]⁺.

### Intermediate 26

### Cyclohexyl-[1-(4-methoxy-benzyl)-3-((E)-styryl)-1H-pyrazolo[4,3-c]pyridin-4-yl]-amine

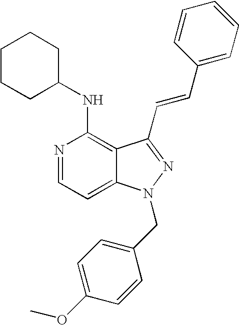

To a mixture of Intermediate 14 (0.25 g, 0.54 mmol) and tetrabutylammonium iodide (0.4 g, 1.08 mmol) in DMF/water/triethylamine (5 ml/0.8 ml/0.8 ml) at room temperature was added styrene (0.62 ml, 5.4 mmol) and Pd(dppf)Cl₂ (88 mg, 0.11 mmol) respectively. The resulting mixture was heated at 70° C. overnight and then evaporated to dryness. The crude residue was dissolved in DCM and washed with water. The organic phase was collected and dried using a phase separation cartridge and then evaporated. The crude residue was purified by flash chromatography, eluting with 20 to 40% ethyl acetate/petroleum ether gradient to give the desired product 50 mg, 63%) ¹H NMR (400 MHz, DMSO-d₆) δ ppm 1.15-1.26 (m, 1H), 1.29-1.49 (m, 4H), 1.59-1.66 (m, 1H), 1.71-1.77 (m, 2H), 1.95-2.02 (m, 2H), 3.70-3.73 (m, 3H), 4.01-4.12 (m, 1H), 5.47 (s, 2H), 6.04 (d, J=7.8 Hz, 1H), 6.83-6.92 (m, 3H), 7.19-7.25 (m, 2H), 7.29-7.36 (m, 1H), 7.39-7.45 (m, 3H), 7.63 (d, J=16.0 Hz, 1H), 7.68-7.74 (m, 2H), 7.77 (d, 1H); m/z (ES+APCI)⁺: 439 [M+H]⁺.

### Intermediate 27

### Cyclohexyl-[1-(4-methoxy-benzyl)-3-phenethyl-1H-pyrazolo[4,3-c]pyridin-4-yl]-amine

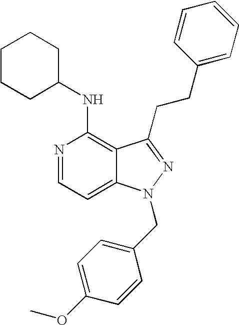

To Intermediate 26 (150 mg, 0.34 mmol) in ethyl acetate (5 ml) at room temperature was added 10% Pd/C (30 mg, 20% by wt). The resulting mixture was stirred under an atmosphere of hydrogen at room temperature over night. The reaction mixture was then filtered through Celite™ and evaporated to give a gum (140 mg, 93%). ¹H NMR (400 MHz, DMSO-d₆) δ ppm 1.12-1.26 (m, 1H), 1.20-1.43 (m, 4H), 1.57-1.66 (m, 1H), 1.67-1.77 (m, 2H), 1.90-2.01 (m, 2H), 2.98 (m, 2H), 3.29 (m, 2H), 3.71 (s, 3H), 3.97-4.06 (m, 1H), 5.36 (s, 2H), 5.67 (br. s., 1H), 6.72-6.81 (m, 1H), 6.75-6.90 (m, 2H), 7.07-7.13 (m, 2H), 7.15-7.21 (m, 1H), 7.22-7.34 (m, 4H), 7.69 (d, J=6.0 Hz, 1H); m/z (ES+APCI)⁺: 441 [M+H]⁺.

### Intermediate 28

### Cyclohexyl-[1-(4-methoxy-benzyl)-3-((E)-2-pyridin-2-yl-vinyl)-1H-pyrazolo[4,3-c]pyridin-4-yl]-amine

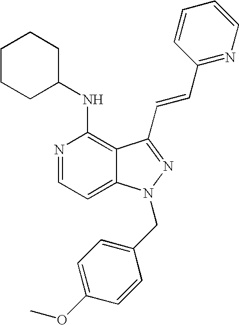

Prepared analogously to Intermediate 26 from Intermediate 14 and 2-vinylpyridine to give the desired product (200 mg, 84%). ¹H NMR (400 MHz, DMSO-d₆) δ ppm 1.15-1.27 (m, 1H), 1.28-1.45 (m, 4H), 1.57-1.64 (m, 1H), 1.69-1.76 (m, 2H), 1.93-2.03 (m, 2H), 3.70 (s, 3H), 4.00-4.09 (m, 1H), 5.48 (s, 2H), 5.95 (d, J=7.8 Hz, 1H), 6.83-6.92 (m, 3H), 7.20-7.25 (m, 2H), 7.27-7.31 (m, 1H), 7.42 (d, J=15.6 Hz, 1H), 7.71 (d, J=8.2 Hz, 1H), 7.78 (d, J=6.0 Hz, 1H), 7.80-7.85 (m, 1H), 7.95 (d, J=16.0 Hz, 1H), 8.61 (d, J=3.7 Hz, 1H); m/z (ES+APCI)⁺: 440 [M+H]⁺.

### Intermediate 29

### Cyclohexyl-[1-(4-methoxy-benzyl)-3-(2-pyridin-2-yl-ethyl)-1H-pyrazolo[4,3-c]pyridin-4-yl]-amine

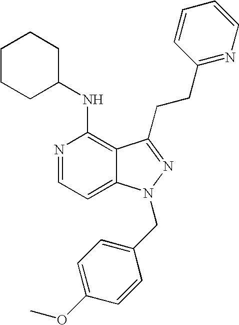

To a stirred solution of Intermediate 28 (150 mg, 0.34 mmol) in ethyl acetate (4.5 ml) at room temperature was added acetic acid (0.5 ml) and 10% Pd/C (30 mg). The resulting mixture was stirred under an atmosphere of hydrogen at room temperature for 72 h. The reaction mixture was then filtered through Celite™ and evaporated. The crude residue was dissolved in DCM and washed with 2M NaOH. The organic phase was dried and evaporated to give a brown gum (80 mg, 53%). ¹H NMR (400 MHz, DMSO-d₆) δ ppm 1.15-1.27 (m, 1H), 1.27-1.45 (m, 4H), 1.58-1.66 (m, 1H), 1.70-1.79 (m, 2H), 1.94-2.03 (m, 2H), 3.11-3.18 (m, 2H), 3.34-3.40 (m, 2H), 3.72 (s, 3H), 4.04-4.13 (m, 1H), 5.34 (s, 2H), 5.94 (d, J=7.8 Hz, 1H), 6.74 (d, J=6.0 Hz, 1H), 6.82-6.91 (m, 2H), 7.09-7.14 (m, 2H), 7.21-7.31 (m, 2H), 7.68-7.73 (m, 2H), 8.54 (d, J=4.1 Hz, 1H); m/z (ES+APCI)⁺: 442 [M+H]⁺.

### Intermediate 30

### 3-[4-Cyclohexylamino-1-(4-methoxy-benzyl)-1H-pyrazolo[4,3-c]pyridin-3-yl]-propan-1-ol

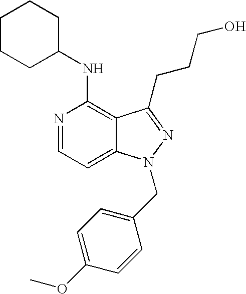

Lithium borohydride (132 mg, 6.0 mmol) was added to Intermediate 21 (0.85 g, 2.0 mmol) in THF (10 ml) at 0° C. Methanol (0.25 ml, 6.0 mmol) was then added dropwise and the resulting mixture was stirred at 0° C. for 10 mins and then allowed to warm to room temperature overnight. The reaction mixture was diluted with methanol and evaporated. 6M HCl (aq) was then added and the resulting solution stirred at 50° C. for 35 mins and then evaporated. Saturated NaHCO₃ (aq) was added and then the aqueous phase was extracted with EtOAc (×2). The combined organic phases were dried (MgSO₄) and evaporated to give the product as a gum (0.74 g, 93%) ¹H NMR (400 MHz, DMSO-d₆) δ ppm 1.13-1.27 (m, 1H), 1.27-1.40 (m, 4H), 1.57-1.65 (m, 1H), 1.67-1.84 (m, 4H), 1.90-2.01 (m, 2H), 2.98 (t, J=7.6 Hz, 2H), 3.44-3.50 (m, 2H), 3.70 (s, 3H), 3.99-4.09 (m, 1H), 4.85 (t, J=4.6 Hz, 1H), 5.34 (s, 2H), 5.79 (d, 1H), 6.74 (d, J=6.4 Hz, 1H), 6.83-6.88 (m, 2H), 7.12-7.17 (m, 2H), 7.68 (d, J=6.0 Hz, 1H); m/z (ES+APCI)⁺: 395 [M+H]⁺.

### Intermediate 31

### 3-[4-Cyclohexylamino-1-(4-methoxy-benzyl)-1H-pyrazolo[4,3-c]pyridin-3-yl]-propionic acid

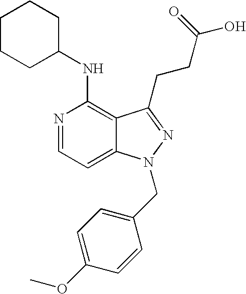

Prepared analogously to Intermediate 23 from Intermediate 21 to give the product (0.3 g, 89%). ¹H NMR (400 MHz, DMSO-d₆) δ ppm 1.14-1.43 (m, 5H), 1.56-1.63 (m, 1H), 1.67-1.75 (m, 2H), 1.90-2.02 (m, 2H), 2.44 (t, J=6.9 Hz, 2H), 3.08 (t, J=6.6 Hz, 2H), 3.69 (s, 3H), 3.93-4.01 (m, 1H), 5.31 (s, 2H), 6.66 (d, J=6.4 Hz, 1H), 6.82-6.86 (m, 2H), 7.11-7.16 (m, 2H), 7.65 (d, J=6.0 Hz, 1H); R_{f} (100% ethyl acetate)=0.10.

### Intermediate 32

### Benzoic acid N′-{3-[4-cyclohexylamino-1-(4-methoxy-benzyl)-1H-pyrazolo[4,3-c]pyridin-3-yl]-propionyl}-hydrazide

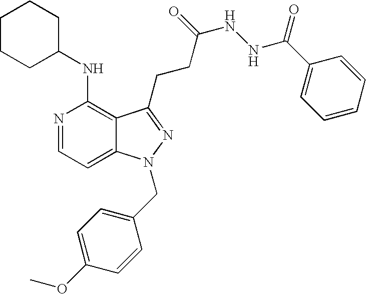

To a solution of Intermediate 31 (100 mg, 0.25 mmol) in DMF (2 ml) was added HATU (93 mg, 0.25 mmol) and N,N-diisopropylethylamine (250 μl, 1.5 mmol). Benzoic hydrazide (33 mg, 0.25 mmol) was then added and the resulting solution was left to stir at room temperature overnight. The volatiles were removed under reduced pressure and the crude product was dissolved in 10% MeOH/DCM and eluted though an Isolute-NH₂ cartridge and evaporated. The crude residue was re-dissolved in DCM and washed with water. The organic phase was separated, dried and evaporated to give the desired product (90 mg, 70%). ¹H NMR (400 MHz, DMSO-d₆) δ ppm 1.07-1.19 (m, 1H), 1.22-1.45 (m, 4H), 1.53-1.61 (m, 1H), 1.05-1.76 (m, 2H), 1.91-1.99 (m, 2H), 2.65-2.77 (m, 2H), 3.25 (t, J=6.9 Hz, 2H), 3.69 (s, 3H), 387-3.95 (m, 1H), (m, 2H), 7.54-7.60 (m, 1H), 7.65-7.69 (m, 1H), 7.82-7.87 (m, 1H), 7.95 (s, 1H), 10.08 (s, 1H), 10.33 (s, 1H); m/z (ES+APCI)⁺: 527 [M+H]⁺.

### Intermediate 33

### Cyclohexyl-{1-(4-methoxy-benzyl)-3-[2-(5-phenyl-1,3,4-oxadiazol-2-yl)-ethyl]-1H-pyrazolo[4,3-c]pyridin-4-yl}-amine

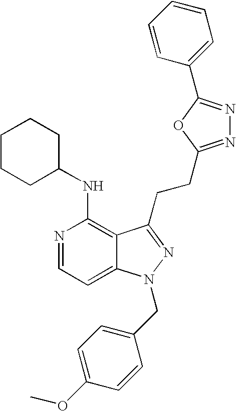

To Intermediate 32 (90 mg, 0.17 mmol) in THF (2 ml) at room temperature was added methyl-N-(triethylammoniumsulfonyl)carbamate inner salt (Burgess reagent) (51 mg, 0.21 mmol). The resulting mixture was irradiated at 100° C. for 30 mins in a Biotage I-60 microwave reactor, monitoring the reaction by LCMS. Irradiation was continued for a further 30 mins at 150° C. prior to concentration followed by flash chromatography, eluting with 20 to 100% ethyl acetate/petroleum ether gradient to give a the product as a gum (20 mg, 23%). ¹H NMR (400 MHz, CDCl₃) δ ppm 1.11-1.18 (m, 1H), 1.19-1.62 (m, 4H), 1.61-1.72 (m, 1H), 1.72-1.85 (m, 2H), 2.09-2.21 (m, 2H), 3.46-3.61 (m, 4H), 3.71 (s, 3H), 4.09-4.26 (m, 1H), 5.31 (s, 2H), 6.50 (d, J=6.4 Hz, 1H), 6.75-6.81 (m, 2H), 7.08-7.16 (m, 2H), 7.44-7.56 (m, 3H), 7.82 (d, J=6.4 Hz, 1H), 7.96-8.08 (m, 2H); m/z (ES+APCI)⁺: 509 [M+H]⁺.

### Intermediate 34

### Cyclohexyl-[1-(4-methoxy-benzyl)-3-((E)-2-pyridin-4-yl-vinyl)-1H-pyrazolo[4,3-c]pyridin-4-yl]-amine

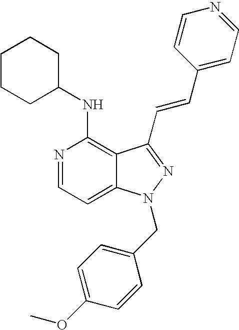

Prepared analogously to Intermediate 26 from Intermediate 14 and 4-vinylpyridine to give the product as a yellow solid (0.93 g, 65%) ¹H NMR (400 MHz, DMSO-d₆) δ ppm 1.13-1.26 (m, 1H), 1.28-1.51 (m, 4H), 1.59-1.68 (m, 1H), 1.70-1.80 (m, 2H), 1.95-2.04 (m, 2H), 3.71 (s, 3H), 4.02-4.13 (m, 1H), 5.49 (s, 2H), 6.17 (d, J=7.8 Hz, 1H), 6.86-6.92 (m, 3H), 7.16-7.25 (m, 2H), 7.37 (d, J=16.0 Hz, 1H), 7.68 (d, J=6.0 Hz, 2H), 7.78 (d, J=6.0 Hz, 1H), 7.89 (d, J=16.0 Hz, 1H), 8.59 (d, J=6.0 Hz, 2H); m/z (ES+APCI)⁺: 440 [M+H]⁺.

### Intermediate 35

### 3-(4-Cyclohexylamino-1H-pyrazolo[4,3-c]pyridin-3-yl)-propan-1-ol

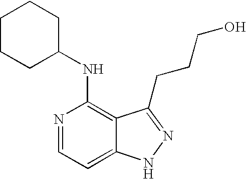

A solution of Intermediate 30 (0.74 g, 1.9 mmol) in TFA (5 ml) was stirred at 70° C. overnight. The reaction mixture was cooled to room temperature and then diluted with DCM, and saturated Na₂CO₃ (aq) was added. The organic phase was separated, filtered through a phase separation tube and evaporated. The crude residue was purified by flash chromatography, eluting with 20% ethyl acetate/petroleum ether to 10% methanol/ethyl acetate gradient to give a gum (0.47 g, 91%). ¹H NMR (400 MHz, DMSO-d₆) δ ppm 1.13-1.26 (m, 1H), 1.32-1.58 (m, 4H), 1.02-1.70 (m, 1H), 1.75-1.88 (m, 4H), 1.93-2.03 (m, 2H), 3.04-3.14 (m, 2H), 3.14-3.25 (m, 1H), 3.48 (t, J=5.7 Hz, 2H), 3.79-3.88 (m, 1H), 5.20 (br. s., 1H), 6.98 (d, 1H), 7.52-7.68 (m, 1H); m/z (ES+APCI)⁺: 275 [M+H]⁺.

### Intermediate 36

### Cyclohexyl-[3-((E)-2-pyridin-4-yl-vinyl)-1H-pyrazolo[4,3-c]pyridin-4-yl]-amine

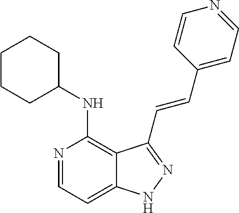

Prepared analogously to Intermediate 35 from Intermediate 34 to give the product as yellow solid (23 mg, 21%) ¹H NMR (400 MHz, MeOD) δ ppm 1.25-1.54 (m, 5H), 1.60-1.74 (m, 1H), 1.74-1.87 (m, 2H), 1.99-2.22 (m, 2H), 3.97-4.06 (m, 1H), 6.74 (d, J=6.0 Hz, 1H), 7.38 (d, J=16.0 Hz, 1H), 7.65 (d, J=6.4 Hz, 2H), 7.72 (d, J=6.0 Hz, 1H), 7.84 (d, J=16.0 Hz, 1H), 8.54 (d, J=6.4 Hz, 2H); m/z (ES+APCI)⁺: 320 [M+H]⁺.

### Intermediate 37

### Cyclohexyl-[1-(4-methoxy-benzyl)-3-(2-pyridin-4-yl-ethyl)-1H-pyrazolo[4,3-c]pyridin-4-yl]-amine

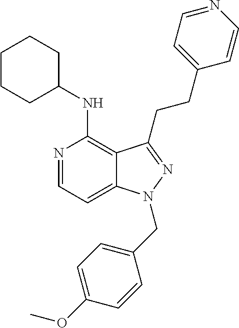

To a stirring solution of Intermediate 34 (0.53 g, 1.2 mmol) in ethanol (10 ml) at room temperature was added platinum oxide (53 mg) and 4M HCl in dioxane (0.3 ml). The resulting mixture was stirred under hydrogen at room temperature overnight. More platinum oxide (50 mg) was added and the mixture was stirred for a further 18 h at room temperature. The reaction mixture was then filtered through Celite™, evaporated and partitioned between DCM and saturated Na₂CO₃ (aq). The organic phase was collected using a phase separation tube, evaporated and then purified by flash chromatography, eluting with ethyl acetate to 20% 2M NH₃ in methanol(aq)/ethyl acetate gradient to give the desired product (0.47 mg, 88%). ¹H NMR (400 MHz, CDCl₃) δ ppm 1.10-1.38 (m, 3H), 1.38-1.60 (m, 2H), 1.60-1.83 (m, 3H), 2.08-2.37 (m, 2H), 3.11-3.21 (m, 2H), 3.21-3.32 (m, 2H), 3.78 (s, 3H), 4.11-4.26 (m, 1H), 4.72 (d, J=7.3 Hz, 1H), 5.32 (s, 2H), 6.51 (d, J=6.4 Hz, 1H), 6.79-6.88 (m, 2H), 7.04-7.11 (m, 2H), 7.15 (d, J=6.0 Hz, 2H), 7.82 (d, J=6.4 Hz, 1H), 8.51 (d, J=5.0 Hz, 2H); m/z (ES+APCI)⁺: 442 [M+H]⁺.

### Intermediate 38

### Cyclohexyl-[1-(4-methoxy-benzyl)-3-(2-piperidin-4-yl-ethyl)-1H-pyrazolo[4,3-c]pyridin-4-yl]-amine

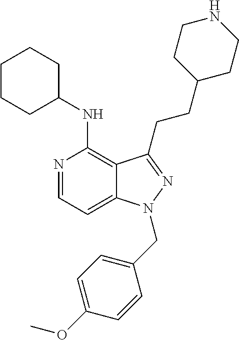

To a stirred solution of Intermediate 37 (0.47 g, 1.1 mmol) in ethanol (10 ml) at room temperature was added platinum oxide (50 mg) and 4M HCl in dioxane (0.27 ml, 1.1). The resulting mixture was stirred under hydrogen at 50° C. overnight. The crude product was filtered through Celite™, evaporated and then partitioned between DCM and saturated Na₂CO₃ (aq). The organic phase was collected, dried (MgSO₄) and evaporated to give the desired product (28 mg, 59%). ¹H NMR (400 MHz, DMSO-d₆) δ ppm 0.93-1.09 (m, 2H), 1.14-1.41 (m, 6H), 1.47-1.82 (m, 7H), 1.84-2.04 (m, 2H), 2.32-2.49 (m, 2H), 2.86-2.94 (m, 2H), 2.95-3.07 (m, 2H), 3.71 (s, 3H), 3.97-4.08 (m, 1H), 5.34 (s, 2H), 6.75 (d, J=6.0 Hz, 1H), 6.82-6.89 (m, 2H), 7.08-7.16 (m, 2H), 7.68 (d, 1H); m/z (ES+APCI)⁺: 448 [M+H]⁺.

### Intermediate 39

### Cyclohexyl-{1-(4-methoxy-benzyl)-3-[(E)-2-(3-nitro-phenyl)-vinyl]-1H-pyrazolo[4,3-c]pyridin-4-yl}-amine

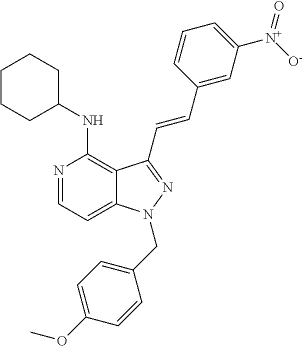

Prepared analogously to Intermediate 26 from Intermediate 14 and 3-nitrostyrene to give the product as a yellow solid (0.93 g, 59%). ¹H NMR (400 MHz, DMSO-d₆) δ ppm 1.17-1.25 (m, 1H), 1.29-1.39 (m, 2H), 1.39-1.50 (m, 2H), 1.60-1.66 (m, 1H), 171-1.78 (m, 2H), 1.96-2.02 (m, 2H), 3.70 (s, 3H), 4.04-4.12 (m, 1H), 5.48 (s, 2H), 6.19 (d, J=7.8 Hz, 1H), 6.86-6.91 (m, 3H), 7.20-7.24 (m, 2H), 7.52 (d, J=16.0 Hz, 1H), 7.71-7.74 (m, 1H), 7.77 (d, J=6.4 Hz, 1H), 7.84 (d, J=15.6 Hz, 1H), 8.13-8.16 (m, 1H), 8.19 (d, J=7.8 Hz, 1H), 8.55 (s, 1H); m/z (ES+APCI)⁺: 484 [M+H]⁺.

### Intermediate 40

### Cyclohexyl-{3-[(E)-2-(3-nitro-phenyl)-vinyl]-1H-pyrazolo[4,3-c]pyridin-4-yl}-amine

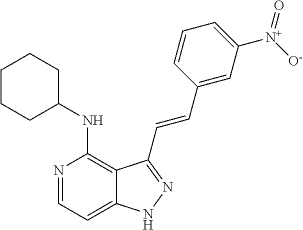

Prepared analogously to Intermediate 35 from Intermediate 39 to give the product (as a brown solid (0.48 g, 100%). ¹H NMR (400 MHz, DMSO-d₆) δ ppm 1.16-1.26 (m, 1H), 1.29-1.50 (m, 4H), 1.60-1.67 (m, 1H), 1.72-1.79 (m, 2H), 1.97-2.10 (m, 2H), 4.04-4.13 (m, 1H), 6.13 (d, J=7.8 Hz, 1H), 6.67 (d, J=6.0 Hz, 1H), 7.55 (d, J=16.0 Hz, 1H), 7.70-7.76 (m, 2H), 7.87 (d, J=16.0 Hz, 1H), 8.13-8.17 (m, 1H), 8.18-8.21 (m, 1H), 8.55 (s, 1H); m/z (ES+APCI)⁺: 364 [M+H]⁺.

### Intermediate 41

### 4-{(E)-2-[4-Cyclohexylamino-1-(4-methoxy-benzyl)-1H-pyrazolo[4,3-c]pyridin-3-yl]-vinyl}-benzoic acid methyl ester

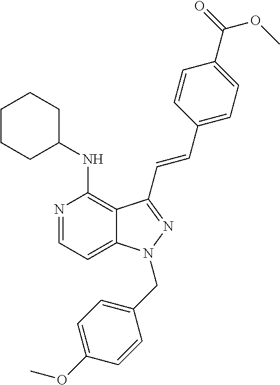

Prepared analogously to Intermediate 26 from Intermediate 14 and methyl-4-vinyl benzoate to give the desired product as a brown solid (190 mg, 70%). ¹H NMR (400 MHz, DMSO-d₆) δ ppm 1.15-1.24 (m, 1H), 1.27-1.48 (m, 4H), 1.59-1.65 (m, 1H), 1.70-1.77 (m, 2H), 1.94-2.01 (m, 2H), 3.69 (s, 3H), 3.86 (s, 3H), 4.03-4.11 (m, 1H), 5.47 (s, 2H), 6.14 (d, J=7.8 Hz, 1H), 6.85-6.90 (m, 3H), 7.19-7.23 (m, 2H), 7.45 (d, J=16.0 Hz, 1H), 7.73-7.82 (m, 2H), 7.85 (d, J=8.2 Hz, 2H), 7.98 (d, J=^{˜}8.7 Hz, 2H); m/z (ES+APCI)⁺: 497 [M+H]⁺.

### Intermediate 42

### 4-{2-[4-Cyclohexylamino-1-(4-methoxy-benzyl)-1H-pyrazolo[4,3-c]pyridin-3-yl]-ethyl}-benzoic acid methyl ester

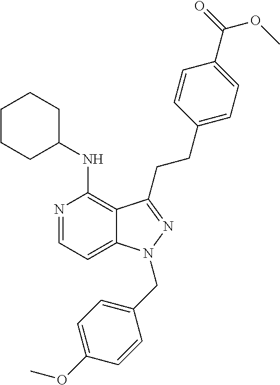

To Intermediate 41 (0.19 g, 0.38 mmol) in ethanol (5 ml) at room temperature was added 10% Pd/C (40 mg). The resulting mixture was stirred under an atmosphere of hydrogen at room temperature overnight. More 10% Pd/C (40 mg) was added and the mixture was stirred for a further 18 h at room temperature. The reaction mixture was then filtered through Celite™ and evaporated to give the product as a brown gum (185 mg, 97%). ¹H NMR (400 MHz, DMSO-d₆) δ ppm 1.12-1.27 (m, 1H), 1.27-1.42 (m, 4H), 1.57-1.65 (m, 1H), 1.66-1.76 (m, 2H), 1.92-1.99 (m, 2H), 3.05-3.11 (m, 2H), 3.34-3.41 (m, 2H), 3.70 (s, 3H), 3.83 (s, 3H), 3.98-4.06 (m, 1H), 5.32 (s, 2H), 5.61 (d, J=6.4 Hz, 1H), 6.74 (d, J=6.0 Hz, 1H), 6.77-6.82 (m, 2H), 7.01-7.06 (m, 2H), 7.34-7.38 (m, 2H), 7.68 (d, J=6.4 Hz, 1H), 7.82-7.87 (m, 2H); m/z (ES+APCI)⁺: 499 [M+H]⁺.

### Intermediate 43

### 4-[2-(4-Cyclohexylamino-1H-pyrazolo[4,3-c]pyridin-3-yl)-ethyl]-benzoic acid methyl ester

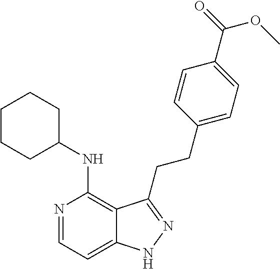

Prepared analogously to Intermediate 35 from Intermediate 42 to give the product as a brown solid (0.12 g, 86%). ¹H NMR (400 MHz, DMSO-d₆) δ ppm 1.13-1.24 (m, 1H), 1.27-1.41 (m, 4H), 1.58-1.66 (m, 1H), 1.67-1.76 (m, 2H), 1.92-2.01 (m, 2H), 3.06-3.12 (m, 2H), 3.34-3.42 (m, 2H), 3.83 (s, 3H), 3.99-4.07 (m, 1H), 5.52 (d, J=7.8 Hz, 1H), 6.58 (d, J=6.0 Hz, 1H), 7.42 (d, J=8.2 Hz, 2H), 7.67 (d, J=6.0 Hz, 1H), 7.88 (d, J=8.7 Hz, 2H); m/z (ES+APCI)⁺: 379 [M+H]⁺.

### Intermediate 44

### 4-[2-(4-Cyclohexylamino-1H-pyrazolo[4,3-c]pyridin-3-yl)-ethyl]-benzoic acid

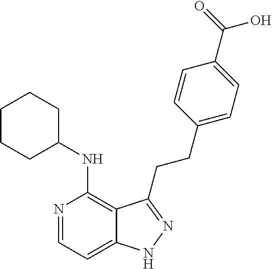

To a stirred solution of Intermediate 43 (0.1 g, 0.26 mmol) in methanol (2 ml) at room temperature was added 2M NaOH (0.33 ml, 0.66 mmol). The resulting mixture was stirred at 70° C. overnight. Acetic acid (40 μl, 0.66 mmol) was then added, and the resulting precipitate was filtered and dried to give a white solid (35 mg, 36%). ¹H NMR (400 MHz, DMSO-d₆) δ ppm 1.13-1.24 (m, 1H), 1.27-1.43 (m, 4H), 1.58-1.65 (m, 1H), 1.68-1.78 (m, 2H), 1.93-2.01 (m, 2H), 3.05-3.12 (m, 2H), 3.36-3.45 (m, 2H), 3.96-4.05 (m, 1H), 5.68 (br. s., 1H), 6.62 (d, J=6.0 Hz, 1H), 7.36-7.41 (m, 2H), 7.66 (d, J=6.0 Hz, 1H), 7.84-7.88 (m, 2H); m/z (ES+APCI)⁺: 365 [M+H]⁺.

### Intermediate 45

### 4-Chloro-3-iodo-1-trityl-1H-pyrazolo[4,3-c]pyridine

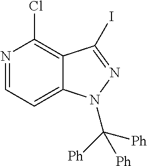

NaH (108 mg, 2.69 mmol, 60% dispersion) was added to a solution of Intermediate 12 (500 mg, 1.79 mmol) in DMF (2 ml) at 0° C. and the mixture was stirred at this temperature for 30 min. Trityl chloride (550 mg, 1.97 mmol) was then added and stirring continued at room temperature for 19 h. Water was added, and the white precipitate was filtered and washed with water. The residue was then dissolved in DCM, washed with water, followed by brine, dried (MgSO₄) and solvents evaporated to give a white solid (900 mg, 96%). ¹H NMR (400 MHz, DMSO-d₆) δ ppm 6.33 (d, J=7.8 Hz, 1H), 7.15-7.19 (m, 5H), 7.31-7.46 (m, 10H), 7.91 (d, J=6.4 Hz, 1H); Rf=0.52 (1:1, petroleum ether:ethyl acetate).

### Intermediate 46

### 4-Cyclohexyloxy-3-iodo-1-trityl-1H-pyrazolo[4,3-c]pyridine

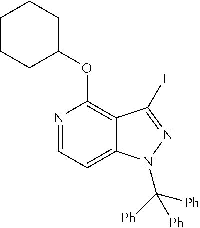

To a solution of cyclohexanol (223 μA 2.15 mmol) in dioxane (1 ml), in a 2-5 ml microwave vial was added NaH (64 mg, 1.61 mmol, 60% dispersion). The vial was sealed and the mixture was stirred for 3 h at room temperature under nitrogen. After this time Intermediate 45 (559 mg, 1.07 mmol) in dioxane (2 ml) was added and the reaction irradiated in the microwave at 180° C. for 1.5 h. The solvents were evaporated and the crude product were partitioned between ethyl acetate and water, extracted twice with ethyl acetate, the organic extracts combined, washed with brine, dried (MgSO₄) and solvents removed. Purification by flash chromatography using a Biotage SP4 (ethyl acetate/petroleum ether gradient) gave a white solid (319 mg, 51%). ¹H NMR (400 MHz, DMSO-d₆) δ ppm 1.46-1.58 (m, 4H), 1.66-1.77 (m, 2H), 1.80-1.96 (m, 4H), 5.27-5.39 (m, 1H), 5.80-5.84 (m, 1H), 7.13-7.20 (m, 5H), 7.22-7.44 (m, 10H), 7.57 (d, J=6.4 Hz, 1H); m/z (ES+APCI)⁺: 586 [M+H]⁺.

### Intermediate 47

### (E)-3-[4-Chloro-1-(4-methoxy-benzyl)-1H-pyrazolo[4,3-c]pyridin-3-yl]-acrylic acid methyl ester

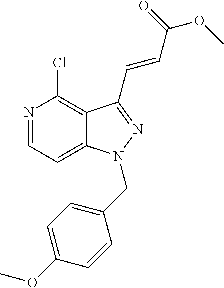

To a mixture of Intermediate 13 (2.0 g, 5.0 mmol) and tetrabutylammonium iodide (3.7 g, 10.0 mmol) in DMF/water/triethylamine (50 ml/8 ml/8 ml) at room temperature was added methyl acrylate (4.5 ml, 50 mmol) and Pd(dppf)Cl₂ (0.82 mg, 1.0 mmol) respectively. The resulting mixture was heated at 50° C. overnight and then evaporated to dryness. The crude residue was purified by flash chromatography, eluting with 20 to 100% ethyl acetate/petroleum ether gradient to give a brown solid (1.2 g, 67%) ¹H NMR (400 MHz, CDCl₃) δ ppm 3.80 (s, 3H), 3.86 (s, 3H), 5.52 (s, 2H), 6.83-6.91 (m, 2H), 6.97 (d, J=16.0 Hz, 1H), 7.12-7.24 (m, 3H), 8.13 (d, J=6.0 Hz, 1H), 8.37 (d, J=16.0 Hz, 1H); m/z (ES+APCI)⁺: 358/360 [M+H]⁺.

### Intermediate 48

### 3-[4-Chloro-1-(4-methoxy-benzyl)-1H-pyrazolo[4,3-c]pyridin-3-yl]-propionic acid methyl ester

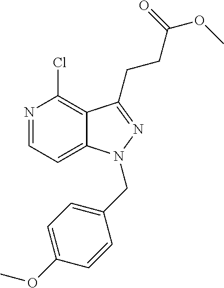

To a solution of Intermediate 47 (0.5 g, 1.4 mmol) in 1:1 mixture of 2-propanol/ethyl acetate (10 ml) at room temperature was added 5% Rh/Al₂O₃ (0.25 g). The resulting mixture was stirred under an atmosphere of hydrogen at room temperature overnight. A further 0.25 g of 5% Rh/Al₂O₃ was added and the mixture stirred for a further 18 h. The reaction mixture was then filtered through Celite™ and evaporated. The crude residue was purified by preparative LCMS (high pH buffer) to give the product as a white solid (0.15 mg, 30%). ¹H NMR (400 MHz, CDCl₃) δ ppm 2.89-2.95 (m, 2H), 3.49-3.56 (m, 2H), 3.70 (s, 3H), 3.78 (s, 3H), 5.43 (s, 2H), 6.82-6.87 (m, 2H), 7.08-7.17 (m, 3H), 8.07 (d, J=^{˜}6.0 Hz, 1H); m/z (ES+APCI)⁺: 360 362 [M+H]⁺.

### Intermediate 49

### 3-[4-Chloro-1-(4-methoxy-benzyl)-1H-pyrazolo[4,3-c]pyridin-3-yl]-propionic acid

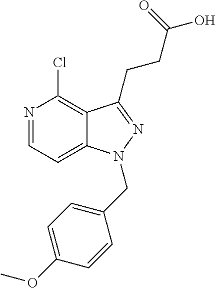

To a stirred solution of Intermediate 48 (0.15 g, 0.42 mmol) in methanol (15 ml) at room temperature was added 2M NaOH (0.52 ml, 1.0 mmol). The resulting mixture was stirred at room temperature overnight, then evaporated. The crude residue was dissolved in water, and then 2M HCl (0.52 ml, 1.0 mmol) was added. The resulting precipitate was filtered and dried to give a white solid (0.11 g, 77%). ¹H NMR (400 MHz, DMSO-d₆) δ ppm 2.77 (t, J=7.6 Hz, 2H), 329-3.39 (m, 2H), 3.71 (s, 3H), 5.55 (s, 2H), 6.84-6.90 (m, 2H), 7.16-7.26 (m, 2H), 7.76 (d, J=6.0 Hz, 1H), 8.13 (d, J=6.0 Hz, 1H); m/z (ES+APCI)⁺: 346/348 [M+H]⁺.

### Intermediate 50

### 3-[4-Chloro-1-(4-methoxy-benzyl)-1H-pyrazolo[4,3-c]pyridin-3-yl]-1-((R)-3-phenyl-piperidin-1-yl)-propan-1-one

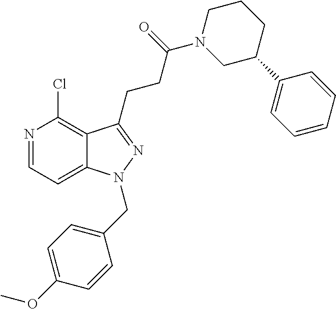

To a solution of Intermediate 49 (0.11 g, 0.32 mmol) in DMF (3 ml) was added HATU (0.13 g, 0.33 mmol) and N,N-diisopropylethylamine (332 μl, 1.9 mmol), followed by (R)-3-phenylpiperidine (52 mg, 0.32 mmol). The resulting solution was left to stir at room temperature overnight, and then evaporated. The crude product purified by flash chromatography eluting with 30-100% ethyl acetate/petroleum ether gradient to give a gum (70 mg, 45%). ¹H NMR (400 MHz, CDCl₃) δ ppm 1.53-1.78 (m, 2H), 1.79-1.88 (m, 1H), 2.00-2.26 (m, 1H), 2.51-2.75 (m, 2H), 2.84-3.11 (m, 3H), 3.36-3.65 (m, 2H), 3.77 (s, 3H), 3.88-4.06 (m, 1H), 4.67-4.88 (m, 1H), 5.35-5.54 (m, 2H), 6.72-6.95 (m, 2H), 7.05-7.35 (m, 8H), 7.99-8.14 (m, 1H); m/z (ES+APCI)⁺: 489/491 [M+H]⁺.

### Intermediate 51

### (E)-3-[4-Methoxy-1-(4-methoxy-benzyl)-1H-pyrazolo[4,3-c]pyridin-3-yl]-acrylic acid

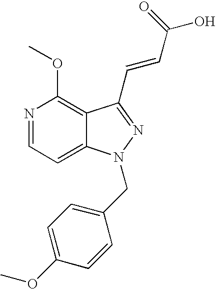

To a stirred solution of Intermediate 47 (0.5 g, 1.4 mmol) in methanol (10 ml) at room temperature was added 2M NaOH (1.75 ml, 3.5 mmol). The resulting mixture was stirred at room temperature overnight. The mixture was heated to 70° C. for a further 4 h, and evaporated. The crude residue was dissolved in water, and then 2M HCl (1.75 ml, 3.5 mmol) was added. The resulting precipitate was filtered and dried to give a brown solid (0.42 g, 87%). ¹H NMR (400 MHz, DMSO-d₆) δ ppm 3.70 (s, 3H), 4.05 (s, 3H), 5.59 (s, 2H), 6.77-6.96 (m, 3H), 7.15-7.36 (m, 2H), 7.42 (d, J=6.0 Hz, 1H), 7.84 (d, J=16.0 Hz, 1H), 7.92-7.99 (m, 1H); m/z (ES+APCI)⁺: 340 [M+H]⁺.

### Intermediate 52

### (E)-3-[4-Methoxy-1-(4-methoxy-benzyl)-1H-pyrazolo[4,3-c]pyridin-3-yl]-1-((R)-3-phenyl-piperidin-1-yl)-propenone

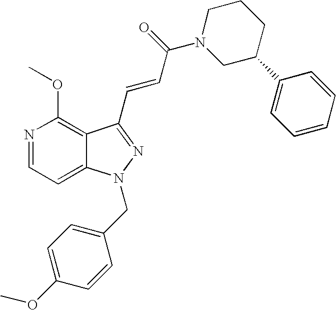

Prepared analogously to Intermediate 50 from Intermediate 51 and (R)-3-phenylpiperidine to yield a white solid (0.43 mg, 72%). ¹H NMR (400 MHz, DMSO-d₆) δ ppm 1.44-1.68 (m, 1H), 1.71-1.92 (m, 2H), 1.93-2.11 (m, 1H), 2.62-2.83 (m, 4H), 2.86-3.23 (m, 1H), 3.74 (s, 3H), 3.94-4.18 (m, 2H), 4.42 (br. s, 1H), 5.57 (s, 2H), 6.89 (d, J=8.7 Hz, 2H), 7.16-7.44 (m, 8H), 7.53-7.66 (m, 1H), 7.70-7.84 (m, 1H), 7.93 (d, J=6.0 Hz, 1H); m/z (ES+APCI)⁺: 483 [M+H]⁺.

### Intermediate 53

### 3-[4-Methoxy-1-(4-methoxy-benzyl)-1H-pyrazolo[4,3-c]pyridin-3-yl]-1-((R)-3-phenyl-piperidin-1-yl)-propan-1-one

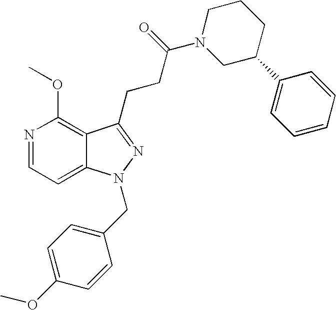

To a solution of Intermediate 52 (0.43 g, 0.89 mmol) in ethyl acetate (10 ml) at room temperature was added 10% Pd/C (90 mg). The resulting mixture was stirred under hydrogen at room temperature overnight. A further 100 mg of 10% Pd/C was added and the mixture stirred for a further 72 h. The reaction mixture was then filtered through Celite™ and evaporated to give a white foam (0.36 mg, 83%) ¹H NMR (400 MHz, DMSO-d₆) δ ppm 1.30-1.58 (m, 1H), 1.04-1.77 (m, 2H), 1.85-1.92 (m, 1H), 2.52-2.65 (m, 2H), 2.71-2.94 (m, 2H), 3.02-3.23 (m, 3H), 3.66-3.71 (m, 3H), 3.79-4.15 (m, 4H), 4.40-4.50 (m, 1H), 5.45 (d, J=10.5 Hz, 2H), 6.82-6.88 (m, 2H), 7.13-7.34 (m, 8H), 7.84 (dd, J=10.5, 6.0 Hz, 1H); m/z (ES+APCI)⁺: 485 [M+H]⁺.

### Intermediate 54

### 4-Cyclohexyloxy-3-iodo-1H-pyrazolo[4,3-c]pyridine

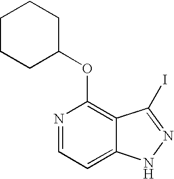

To a solution of cyclohexanol (1.5 ml, 14.3 mmol) in dioxane (28 ml) at room temperature was added sodium hydride (0.5 g, 12.5 mmol). The resulting mixture was stirred at room temperature for 1.5 h, then Intermediate 12 (1 g, 3.6 mmol) was added. The mixture was irradiated at 180° C. for 1.5 h in a Biotage I-60 microwave reactor and then evaporated to dryness. The crude residue was partitioned between DCM and H₂O, the aqueous phase was decanted and the organic phase was dry-loaded onto silica and purified by flash chromatography eluting with 20 to 100% ethyl acetate/petroleum ether gradient. The product was dissolved in 10% MeOH/EtOAc and eluted through an SCX cartridge, eluting first with 10% MeOH/EtOAc, followed by 2M/NH₃ in methanol to yield a white foam (0.84 g, 68%). ¹H NMR (400 MHz, DMSO-d₆) δ ppm 1.28-1.56 (m, 4H), 1.56-1.80 (m, 2H), 1.80-1.94 (m, 4H), 5.20-5.40 (m, 1H), 7.06-7.11 (m, 1H), 7.79-7.84 (m, 1H); m/z (ES+APCI)⁺: 344 [M+H]⁺.

### Intermediate 55

### 4-Cyclohexyloxy-3-iodo-pyrazolo[4,3-c]pyridine-1-carboxylic acid tert-butyl ester

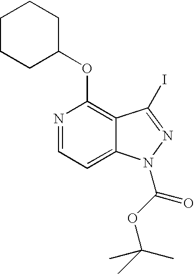

To a solution of Intermediate 54 (0.84 g, 2.45 mmol) and di-tert-butyl dicarbonate (1.38 g, 6.36 mmol) in THF (12 ml) at room temperature was added 4-(dimethylamino)pyridine (15 mg, 0.12 mmol). The resulting mixture was stirred at 70° C. for 2.5 h and then evaporated to dryness. The crude residue was partitioned between DCM and H₂O, and the organic phase collected and dried through a phase separating tube and evaporated to yield a yellow solid (1.1 g, 100%). ¹H NMR (400 MHz, DMSO-d₆) δ ppm 1.33-1.57 (m, 4H), 1.57-1.78 (m, 11H), 1.78-1.94 (m, 4H), 5.33-5.40 (m, 1H), 7.54 (d, J=6.0 Hz, 1H), 8.12 (d, J=6.0 Hz, 1H); m/z (ES+APCI)⁺: 444 [M+H]⁺.

### Intermediate 56

### 4-Cyclohexyloxy-3-((E)-2-methoxycarbonyl-vinyl)-pyrazolo[4,3-c]pyridine-1-carboxylic acid tert-butyl ester

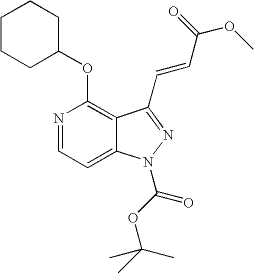

To a mixture of Intermediate 55 (0.6 g, 1.35 mmol) and tetrabutylammonium iodide (1 g, 2.7 mmol) in DMF/water/triethylamine (15 ml/2.4 ml/2.4 ml) at room temperature was added methyl acrylate (1.2 ml, 13.5 mmol) and Pd(dppf)Cl₂ (0.22 mg, 0.27 mmol) respectively. The resulting mixture was heated at 50° C. for 6 h and then evaporated to dryness. The crude residue was purified by flash chromatography, eluting with 5 to 20% ethyl acetate/petroleum ether gradient to give a white solid (80 mg, 15%) ¹H NMR (400 MHz, MeOD) δ ppm 1.40-1.67 (m, 4H), 1.68-1.92 (m, 13H), 1.99-2.13 (m, 2H), 3.84 (s, 3H), 5.21-5.49 (m, 1H), 7.17 (d, J=16.0 Hz, 1H), 7.48-7.74 (m, 1H), 7.94-8.19 (m, 2H); m/z (ES+APCI)⁺: 402 [M+H]⁺.

### Intermediate 57

### (E)-3-(4-Cyclohexyloxy-1H-pyrazolo[4,3-c]pyridin-3-yl)-acrylic acid

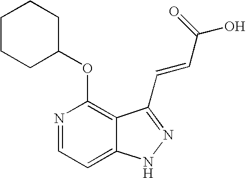

To a stirred suspension of Intermediate 56 (0.13 g, 0.32 mmol) in THF/methanol/water (2 ml/1 ml/1 ml) at room temperature was added lithium hydroxide monohydrate (41 mg, 0.97 mmol). The resulting mixture was stirred at 65° C. overnight and then evaporated to dryness. The crude residue was dissolved in water and then 1M HCl (0.7 ml) was added. The resulting precipitate was filtered and dried to give a cream solid (60 mg, 65%). ¹H NMR (400 MHz, DMSO-d₆) δ ppm 1.31-1.71 (m, 6H), 1.72-1.87 (m, 2H), 1.91-2.16 (m, 2H), 5.22-5.50 (m, 1H), 6.93 (d, J=16.0 Hz, 1H), 7.04-7.16 (m, 1H), 7.85-7.98 (m, 2H).

### Intermediate 58

### Cyclohexyl-{1-(4-methoxy-benzyl)-3-[(E)-2-(4-nitro-phenyl)-vinyl]-1H-pyrazolo[4,3-c]pyridin-4-yl}-amine

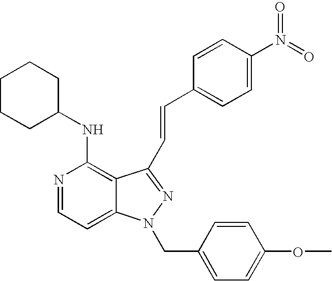

To a stirred solution of Intermediate 14 (3 g, 6.49 mmol) and tetrabutylammonium iodide (4.80 g, 13 mmol) in DMF/water/triethylamine (60 ml/9 ml/9 ml) was added 4-nitrostyrene (4.84 g, 32.4 mmol) and Pd(dppf)Cl₂ (1.06 g, 1.30 mmol). The reaction mixture was heated at 70° C. overnight under a nitrogen atmosphere. The mixture was allowed to cool to rt, concentrated and purified by column chromatography using a Biotage SP4 (petroleum ether/EtOAc gradient) gave the product as an orange solid (1.02 g). The column was further eluted with 100% EtOAc to 10-15% MeOH in EtOAc and the eluent was concentrated. The residue was diluted with EtOAc and H₂O. The organic layer was dried and concentrated. The residue was re-purified by column chromatography using a Biotage SP4 (DCM/MeOH gradient). The chromatographed product was then triturated in MeOH. The orange solid was collected by filtration, washed with MeOH and dried to yield a further 1.03 g of the product. Combined yield (2.05 g, 65% yield). ¹H NMR (400 MHz, CHLOROFORM-d) δ ppm 1.18-1.38 (m, 2H) 1.38-1.58 (m, 2H) 1.58-1.86 (m, 4H) 2.14 (d, J=8.7 Hz, 2H) 3.74-3.89 (m, 3H) 4.10-4.25 (m, 1H) 4.91 (d, J=7.8 Hz, 1H) 5.43 (s, 2H) 6.52-6.59 (m, 1H) 6.80-6.93 (m, 2H) 7.19 (d, J=7.8 Hz, 2H) 7.33-7.59 (m, 2H) 7.67 (d, J=7.8 Hz, 2H) 7.89 (d, J=5.6 Hz, 1H) 8.28 (d, J=7.8 Hz, 2H); m/z (ES+APCI)⁺: 484 [M+H]⁺.

### Intermediate 59

### Cyclohexyl-{3-[(E)-2-(4-nitro-phenyl)-vinyl]-1H-pyrazolo[4,3-c]pyridin-4-yl}-amine

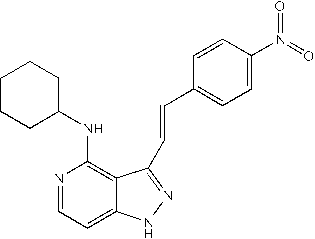

Intermediate 58 (2.05 g, 4.24 mmol) and TFA (20 ml) were combined and heated to 75° C. overnight under a nitrogen atmosphere. After cooling to rt, the solvent was evaporated, the residue was partitioned between DCM and saturated sodium carbonate (aq), and the organic layer was dried and concentrated. Purification by column chromatography using a Biotage SP4 (petroleum ether/EtOAc gradient) gave the desired product as a yellow solid (1.15 g, 75% yield). ¹H NMR (400 MHz, DMSO-d₆) δ ppm 1.12-1.25 (m, 1H) 1.25-1.52 (m, 4H) 1.63 (d, J=12.4 Hz, 1H) 1.75 (d, J=12.8 Hz, 2H) 1.94-2.07 (m, 2H) 4.02-4.13 (m, 1H) 6.13 (d, J=8.2 Hz, 1H) 6.67 (d, J=6.0 Hz, 1H) 7.54 (d, J=16.0 Hz, 1H) 7.74 (d, J=6.0 Hz, 1H) 7.86-8.05 (m, 3H) 8.26 (d, J=8.7 Hz, 2H); m/x (ES+APCI)⁺: 364 [M+H]⁺.

### Intermediate 60

### {3-[2-(4-Amino-phenyl)-ethyl]-1H-pyrazolo[4,3-c]pyridin-4-yl}-cyclohexyl-amine

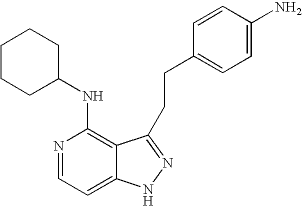

To a RB flask was added cyclohexyl Intermediate 59 (1.15 g) and 10% palladium on charcoal (115 mg) in ethanol (42 ml) and the mixture was stirred at rt under a hydrogen atmosphere for 18 hours. The reaction mixture was filtered through Celite and the filtrate was concentrated to afford the product as a pale yellow solid (1.07 g, 99% yield). ¹H NMR (400 MHz, DMSO-d₆) δ ppm 1.21 (d, J=8.7 Hz, 1H) 1.25-1.48 (m, 4H) 1.60 (d, J=11.9 Hz, 1H) 1.66-1.81 (m, 2H) 1.97 (br. s., 2H) 2.78 (dd, J=9.4, 6.6 Hz, 2H) 3.12-3.25 (m, 2H) 4.01 (br. s., 1H) 4.85 (s, 2H) 5.36 (d, J=7.8 Hz, 1H) 6.48 (m, J=8.2 Hz, 2H) 6.57 (d, J=6.0 Hz, 1H) 6.90 (m, J=8.2 Hz, 2H) 7.66 (d, J=6.0 Hz, 1H); m/z (ES+APCI)⁺: 336 [M+H]⁺.

### Intermediate 61

### 4-Chloro-3-methyl-1-trityl-1H-pyrazolo[4,3-c]pyridine

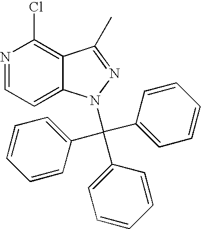

NaH (60% dispersion, 1.07 g, 27.0 mmol) was added to a solution of Intermediate 3 (3 g, 18.0 mmol) in DMF (50 ml). The suspension was stirred for 30 minutes at 0° C. Triphenylmethyl chloride (5.51 g, 20.0 mmol) was added and the reaction stirred for 18 h at rt. The mixture was quenched with water (100 ml), extracted with EtOAc (×2), and the combined organic layers were washed water (×3) followed by brine, (MgSO₄) and evaporated. Purification by flash chromatography using a Biotage SP4, eluting with 0 to 40% EtOAc/petroleum ether gave a pale yellow solid (5.2 g, 71%). The product was isolated as a 9:1 mixture of N1 (A) and N2 (B) alkylated regioisomers: m/z (ES+APCI)⁺: 410 [M+H]⁺

### Example 1

### (4-Chloro-benzyl)-(3-methyl-1H-pyrazolo[4,3-c]pyridin-4-yl)-amine

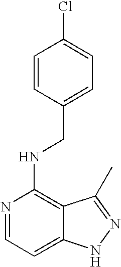

Intermediate 3 (60 mg, 0.357 mmol), 4-chlorobenzylamine (203 mg, 1.43 mmol) and 1-butanol (1 ml) were placed in a sealed microwave reactor vial. The vial was irradiated at 190° C. in a Biotage I-60 microwave reactor for 20 minutes. On cooling to it the mixture was concentrated to dryness. The residue was dissolved in DMSO (1.2 ml) and purified by preparative LCMS to give a yellow solid (53 mg, 54%). ¹H NMR (400 MHz, DMSO-d₆) δ ppm 2.62 (m, 3H) 4.67 (d, J=6.0 Hz, 2H) 6.58 (d, J=5.95 Hz, 1H) 6.82 (t, J=6.0 Hz, 1H) 7.31-7.40 (m, 4H) 7.60 (d, J=6.0 Hz, 1H). m/z (ES+APCI)⁺: 273/275 [M+H]⁺

### Example 2

### (3-Methyl-1H-pyrazolo[4,3-c]pyridin-4-yl)-(2-pyridin-2-yl-ethyl)-amine

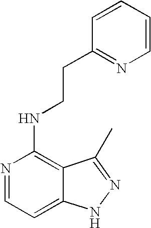

Example 2 was prepared analogously to Example 1 from Intermediate 3 and 2-Pyridin-2-yl-ethylamine to give the product (7 mg, 16%). ¹H NMR (400 MHz, DMSO-d₆) δ ppm 2.59 (s, 3H), 3.12 (t, J=7.1 Hz, 2H), 3.78-3.85 (m, 2H), 6.52 (t, J=5.5 Hz, 1H), 6.61 (d, J=6.0 Hz, 1H), 7.26-7.30 (m, 1H), 7.35 (d, J=7.8 Hz, 1H), 7.72 (d, J=6.0 Hz, 1H), 7.77 (m, 1H), 8.56 (d, J=4.1 Hz, 1H). m/z (ES+APCI)⁺: 254 [M+H]⁺.

### Example 3

### Cyclohexyl-(3-methyl-1H-pyrazolo[4,3-c]pyridin-4-yl)-amine

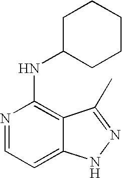

Example 3 was prepared analogously to Example 1 from Intermediate 3 and cyclohexylamine to give the product (2.5 mg, 5%). ¹H NMR (400 MHz, DMSO-d₆) δ ppm 1.17-1.47 (m, 6H), 1.62-1.70 (m, 1H), 1.71-1.83 (m, 2H), 1.95-2.05 (m, 2H), 2.60 (s, 3H), 4.01-4.11 (m, 1H), 5.53 (d, J=7.8 Hz, 1H), 6.59 (d, J=6.0 Hz, 1H), 7.69 (d, J=6.0 Hz, 1H). m/z (ES+APCI)⁺: 231 [M+H]⁺.

### Example 4

### (3-Methyl-1H-pyrazolo[4,3-c]pyridin-4-yl)-(tetrahydro-pyran-4-yl)-amine

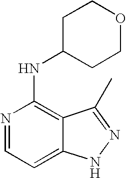

Example 4 was prepared analogously to Example 1 from Intermediate 3 and tetrahydro-pyran-4-ylamine to give the desired product as an off-white solid (27 mg, 64%). ¹H NMR (400 MHz, DMSO-d₆) δ ppm 1.57-1.71 (m, 2H), 1.86-1.95 (m, 2H), 2.59 (s, 3H), 3.37-3.46 (m, 2H), 3.84-3.93 (m, 2H), 4.19-4.30 (m, 1H), 5.62-5.69 (m, 1H), 6.58 (d, J=6.0 Hz, 1H), 7.66 (d, J=6.0 Hz, 1H). m/z (ES+APCI)⁺: 233 [M+H]⁺

### Examples 5-46

Examples 5-46 in the table below were prepared analogously to Example 1 from Intermediate 3 and the corresponding amine:

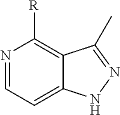

| | | | m/z | HPLC |
|---|---|---|---|---|
| Ex- | | | (ES + | retention |
| ample | R group | Name | APCI)⁺ | time (min) |
| 5 | 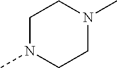 | 3-Methyl-4-(4-methyl- piperazin-1-yl)-1H- pyrazolo[4,3-c]pyridine | 232 | 1.05^{c} |
| 6 | 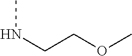 | (2-Methoxy-ethyl)-(3- methyl-1H- pyrazolo[4,3-c]pyridin- 4-yl)-amine | 207 | 0.96^{c} |
| 7 | 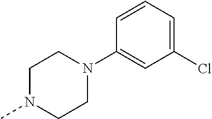 | 4-[4-(3-Chloro-phenyl)- piperazin-1-yl]-3- methyl-1H- pyrazolo[4,3-c]pyridine | 328 | 1.91^{c} |
| 8 | 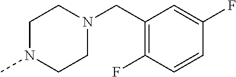 | 4-[4-(2,5-Difluoro- benzyl)-piperazin-1-yl]- 3-methyl-1H- pyrazolo[4,3-c]pyridine | 344 | 1.67^{c} |
| 9 | 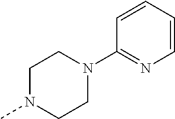 | 3-Methyl-4-(4-pyridin-2- yl-piperazin-1-yl)-1H- pyrazolo[4,3-c]pyridine | 295 | 1.43^{c} |
| 10 | 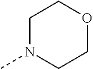 | 3-Methyl-4-morpholin- 4-yl-1H-pyrazolo[4,3- clpyridine | 219 | 1.02^{c} |
| 11 | 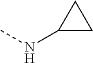 | Cyclopropyl-(3-methyl-1H-pyrazolo[4,3-c]pyridin-4-yl)-amine | 189 | 1.00^{c} |
| 12 | 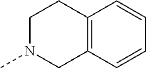 | 2-(3-Methyl-1H-pyrazolo[4,3-c]pyridin-4-yl)-1,2,3,4- tetrahydro-isoquinoline | 265 | 1.71^{c} |
| 13 | 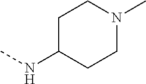 | (1-Methyl-piperidin-4-yl)-(3-methyl-1H-pyrazolo[4,3-c]pyridin-4-yl)-amine | 246 | 2.34^{a} |
| 14 | 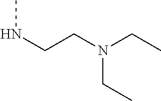 | N,N-Diethyl-N′-(3-methyl-1H- pyrazolo[4,3-c]pyridin- 4-yl)-ethane-1,2- diamine | 248 | 2.05^{a} |
| 15 | 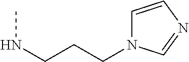 | (3-lmidazol-1-yl-propyl)-(3-methyl-1H-pyrazolo[4,3-c]pyridin-4-yl)-amine | 257 | 0.42^{b} |
| 16 | 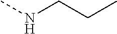 | (3-Methyl-1H-pyrazolo[4,3-c]pyridin-4-yl)-propyl-amine | 191 | 0.47^{b} |
| 17 | 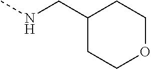 | (3-Methyl-1H- pyrazolo[4,3-c]pyridin- 4-yl)-( tetrahydro-pyran- 4-ylmethyl)-amine | 247 | 0.47^{b} |
| 18 | 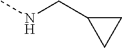 | Cyclopropylmethyl-(3-methyl-1H- pyrazolo[4,3-c]pyridin-4-yl)-amine | 203 | 2.77^{b} |
| 19 | 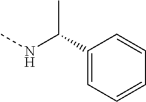 | (3-Methyl-1H-pyrazolo[4,3-c]pyridin-4-yl)-((R)-1-phenyl-ethyl)-amine | 253 | 1.72^{b} |
| 20 | 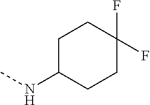 | (4,4-Difluoro-cyclohexyl)-(3-methyl-1H-pyrazolo[4,3-c]pyridin-4-yl)-amine | 267 | 1.87^{b} |
| 21 | 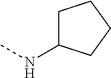 | Cyclopentyl-(3-methyl- 1H-pyrazolo[4,3- c]pyridin-4-yl)-amine | 217 | 1.85^{b} |
| 22 | 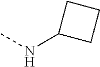 | Cyclobutyl-(3-methyl-1H-pyrazolo[4,3- c]pyridin-4-yl)-amine | 203 | 1.67^{b} |
| 23 | 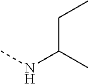 | sec-Butyl-(3-methyl- 1H-pyrazolo[4,3-c]pyridin-4-yl)-amine | 205 | 1.71^{b} |
| 24 | 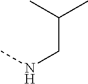 | lsobutyl-(3-methyl-1H-pyrazolo[4,3-c]pyridin-4-yl)-amine | 205 | 1.83^{b} |
| 25 | 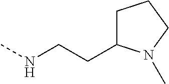 | (3-Methyl-1H-pyrazolo[4,3-c]pyridin-4-yl)-[2-(1-methyl-pyrrolidin-2-yl)-ethyl]-amine | 260 | 2.94^{a} |
| 26 | 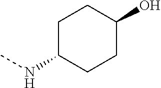 | Trans-4-(3-Methyl-1H-pyrazolo[4,3-c]pyridin-4-ylamino)- cyclohexanol | 247 | 1.49^{b} |
| 27 | 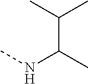 | (1,2-Dimethyl-propyl)-(3~methyl-1H-pyrazolo[4,3-c]pyridin-4-yl)-amine | 219 | 0.99^{d} |
| 28 | 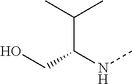 | (S)-3-Methyl-2-(3-methyl-1H- pyrazolo[4,3-c]pyridin-4-ylamino)-butan-1-ol | 235 | 1.75^{b} |
| 29 | 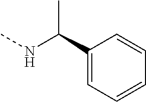 | (3-Methyl-1H-pyrazolo[4,3-c]pyridin-4-yl)-((S)-1-phenyl-ethyl)-amine | 253 | 1.12^{d} |
| 30 | 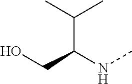 | (R)-3-Methyl-2-(3-methyl-1H- pyrazolo[4,3-c]pyridin-4-ylamino)-butan-1-ol | 235 | 1.65^{b} |
| 31 | 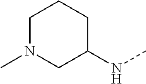 | (1-Methyl-piperidin-3-yl)-(3-methyl-1H-pyrazolo[4,3-c]pyridin-4-yl)-amine | 246 | 1.32^{c} |
| 32 | 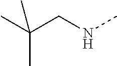 | (2,2-Dimethyl-propyl)-(3-methyl-1H-pyrazolo[4,3-c]pyridin-4-yl)-amine | 219 | 1.56^{c} |
| 33 | 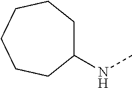 | Cycloheptyl-(3-methyl-1H-pyrazolo[4,3-c]pyridin-4-yl)-amine | 245 | 1.76^{c} |
| 34 | 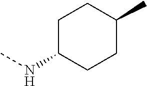 | Trans-(4-Methyl-cyclohexyl)-(3-methyl-1H-pyrazolo[4,3-c]pyridin-4-yl)-amine | 245 | 1.95^{c} |
| 35 | 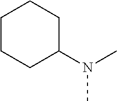 | Cyclohexyl-methyl- (3- methyl-1H- pyrazolo[4,3-c]pyridin- 4-yl)-amine | 245 | 1.71^{c} |
| 36 | 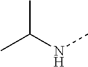 | Isopropyl-(3-methyl-1H-pyrazolo[4,3-c]pyridin-4-yl)-amine | 191 | 1.18^{c} |
| 37 | 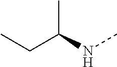 | ((R)-sec-Butyl)-(3- methyl-1H- pyrazolo[4,3-c]pyridin- 4-yl)-amine | 205 | 1.37^{c} |
| 38 | 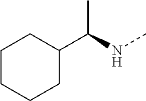 | ((R)-1-Cyclohexyl-ethyl)-(3-methyl-1H-pyrazolo[4,3-c]pyridin-4-yl)-amine | 259 | 1.85^{c} |
| 39 | 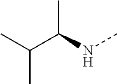 | ((R)-1,2-Dimethyl-propyl)-(3-methyl-1H-pyrazolo[4,3-c]pyridin-4-yl)-amine | 219 | 1.13^{d} |
| 40 | 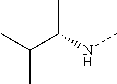 | ((S)-1,2-Dimethyl- propyl)-(3-methyl-1H- pyrazolo[4,3-c]pyridin- 4-yl)-amine | 219 | 1.51^{c} |
| 41 | 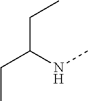 | (1-Ethyl-propyl)-(3-methyl-1H- pyrazolo[4,3-c]pyridin-4-yl)-amine | 219 | 1.12^{d} |
| 42 | 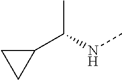 | ((S)-1-Cyclopropyl-ethyl)-(3-methyl-1H-pyrazolo[4,3-c]pyridin-4-yl)-amine | 217 | 1.42^{c} |
| 43 | 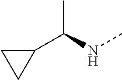 | ((R)-1-Cyclopropyl-ethyl)-(3-methyl-1H-pyrazolo[4,3-c]pyridin-4-yl)-amine | 217 | 1.41^{c} |
| 44 | 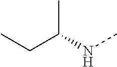 | ((S)-sec-Butyl)-(3-methyl-1H- pyrazolo[4,3-c]pyridin-4-yl)-amine | 205 | 1.36^{c} |
| 45 | 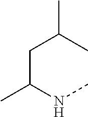 | (1,3-Dimethyl-butyl)-(3-methyl-1H- pyrazolo[4,3-c]pyridin-4-yl)-amine | 233 | 2.4^{c} |
| 46 | 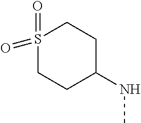 | (1,1-Dioxo-hexahydro-1λ⁶-thiopyran-4-yl)-(3- methyl-1H- pyrazolo[4,3-c]pyridin-4-yl)-amine | 2.81 | 1.02^{c} |

^{a}HPLC column: 4.6 × 50 mm (5 μm) C-18 Xbridge; flow rate: 2 ml/min; Run time: 4.6 min: Solvent A: 0.1% Ammonium Hydroxide in water, Solvent B: Methanol; Gradient-10-100% B; Gradient time: 3.5 min.
^{b}HPLC column: 4.6 × 50 mm (5 μm) C-18 Xbridge; flow rate: 2 m1/min; Run time: 4.6 min: Solvent A: 0.1% Formic acid in water, Solvent B: Methanol; Gradient-10-100% B; Gradient time: 3.5 min.
^{c}HPLC column: 4.6 × 50 mm (5 μm) C-18 Xbridge; flow rate: 3 ml/min; Run time: 3.2 min: Solvent A: 0.1% Ammonium Hydroxide in water, Solvent B: Acetonitrile; Gradient-10-100% B; Gradient time: 2.35 min.
^{d}HPLC column: 4.6 × 50 mm (5 μm) C-18 Xbridge; flow rate: 3 ml/min; Run time: 3.2 min: Solvent A: 0.1% Formic acid in water, Solvent B: Acetonitrile; Gradient-10-100% B; Gradient time: 2.35 min.

### Example 47

### 3-Methyl-4-(1-methyl-1H-pyrazol-4-yl)-1H-pyrazolo[4,3-c]pyridine

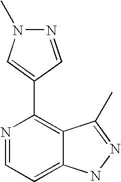

Intermediate 3 (50 mg, 0.298 mmol), 1-methyl-4-(4,4,5,5-tetramethyl-1,3,2-dioxaborolan-2-yl)-1H-pyrazole (93 mg, 0.446 mmol), palladium diphenylphosphinoferrocene dichloride (12.2 mg, 0.015 mmol), 2M aqueous sodium carbonate solution (521 □l, 1.04 mmol) and 1,4-dioxane (2 ml) were placed in a sealed microwave reactor vial. The vial was degassed and place under an atmosphere of nitrogen. The vial was irradiated at 160° C. in a Biotage I-60 microwave reactor for 20 minutes. On cooling the reaction mixture was concentrated to dryness. The residue was dissolved in MeOH and filtered through a plug of Celite. The filtrate was concentrated and the residue dissolved in DMSO (1.2 ml) prior to purification by mass-triggered preparative HPLC. A white solid was obtained (21 mg, 33%). ¹H NMR (400 MHz, DMSO-d₆) δ ppm 3.32 (s, 3H) 3.94 (s, 3H) 7.31 (d, J=5.5 Hz, 1H) 7.84 (s, 1H) 8.17 (s, 1H) 8.23 (d, J=6.0 Hz, 1H). m/z (ES+APCI)⁺: 214 [M+H]⁺

### Example 48

### 4-Furan-2-yl-3-methyl-1H-pyrazolo[4,3-c]pyridine

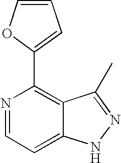

Example 48 was prepared analogously to Example 47 from Intermediate 3 and furan-2-boronic acid to give the product (34 mg, 58%). ¹H NMR (400 MHz, DMSO-d₆) δ ppm 2.37 (s, 3H), 6.43-670 (m, 1H), 7.04 (d, J=3.2 Hz, 1H), 7.26 (d, J=6.0 Hz, 1H), 7.84 (s, 1H), 8.15 (d, J=6.0 Hz, 1H). m/z (ES+APCI)⁺: 200 [M+H]⁺.

### Examples 49-58

Examples 49-58 in following table were prepared analogously to Example 47 from Intermediate 3 and the corresponding boronic acid or boronic ester:

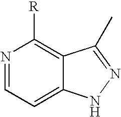

| | | | m/z | HPLC retention |
|---|---|---|---|---|
| Example | R group | Name | (ES + APCl)+ | time (min) |
| 49 | 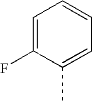 | 4-(2-Fluoro-phenyl)-3- methyl-1H-pyrazolo[4,3- c]pyridine | 228 | 2.97^{a} |
| 50 | 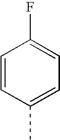 | 4-(4-Fluoro-phenyl)-3- methyl-1H-pyrazolo[4,3- c]pyridine | 228 | 3.12^{a} |
| 51 | 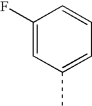 | 4-(3-Fluoro-phenyl)-3- methyl-1H-pyrazolo[4,3- c]pyridine | 228 | 3.15^{a} |
| 52 | 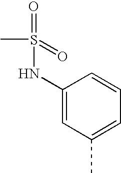 | N-[3-(3-Methyl-1H- pyrazolo[4,3-c]pyridin-4- yl)-phenyl]- methanesulfonamide | 303 | 2.62^{a} |
| 53 | 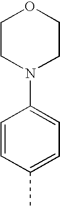 | 3-Methyl-4-(4-morpholin- 4-yl-phenyl)-1H- pyrazolo[4, 3-c]pyridine | 295 | 2.97^{a} |
| 54 | 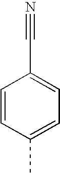 | 4-(3-Methyl-1H- pyrazolo[4,3-c]pyridin-4- yl)-benzonitrile | 235 | 2.83^{a} |
| 55 | 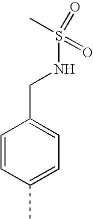 | N-[4-(3-Methyl-1H- pyrazolo[4,3-c]pyridin-4- yl)-benzyl]- methanesulfonamide | 317 | 2.53^{a} |
| 56 | 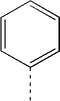 | 3-Methyl-4-phenyl-1H- pyrazolo[4,3-c]pyridine | 210 | 3.07^{a} |
| 57 | 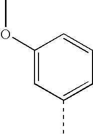 | 4-(3-Methoxy-phenyl)-3- methyl-1H-pyrazolo[4,3- c]pyridine | 240 | 3.12^{a} |
| 58 | 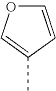 | 4-Furan-3-yl-3-methyl- 1H-pyrazolo[4,3- c]pyridine | 200 | 0.94^{c} |

^{a}HPLC column: 4.6 × 50 mm (5 μm) C-18 Xbridge; flow rate: 2ml/min; Run time: 4.6 min: Solvent A: 0.1% Ammonium Hydroxide in water, Solvent B: Methanol; Gradient - 10-100% B; Gradient time: 3.5 min.
^{c}HPLC column: 4.6 × 50 mm (5 μm) C-18 Xbridge; flow rate: 3ml/min; Run time: 3.2 min: Solvent A: 0.1% Ammonium Hydroxide in water, Solvent B: Acetonitrile; Gradient- 10-100% B; Gradient time: 2.35 min.

### Example 59

### (3-Chloro-phenyl)-(3-methyl-1H-pyrazolo[4,3-c]pyridin-4-yl)-amine

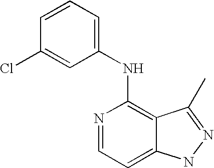

Intermediate 3 (40 mg, 0.24 mmol), 3-chloro-phenylamine (61 mg, 0.48 mmol) and concentrated hydrochloric acid (21 μl, 0.72 mmol), were dissolved in n-butanol (1.1 ml). The reaction mixture was irradiated at 190° C. for 45 minutes in a Biotage I-60 microwave reactor. The mixture was evaporated then purified by preparative LCMS (high pH buffer) to give the desired product as a white solid (49 mg, 79%). ¹H NMR (400 MHz, DMSO-d₆) δ ppm 2.72 (s, 3H), 6.93 (d, J=6.0 Hz, 1H), 6.96-7.00 (m, 1H), 7.27-7.34 (m, 1H), 7.61-7.68 (m, 1H), 7.85 (d, J=6.0 Hz, 1H), 7.92-7.96 (m, 1H), 8.21 (br. s., 1H). m/z (ES+APCI)⁺: 259/261 [M+H]⁺.

### Example 60

### 4-[5-(3-Methyl-1H-pyrazolo[4,3-c]pyridin-4-ylamino)-pyridin-3-yl]-benzonitrile

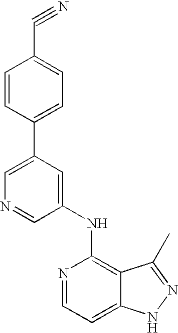

Example 60 was prepared analogously to Example 59 from Intermediate 3 and 4-(5-Amino-pyridin-3-yl)-benzonitrile to give the desired product as a white solid (4 mg, 4%). ¹H NMR (400 MHz, DMSO-d₆) δ ppm 2.81 (s, 3H), 6.99 (d, J=6.0 Hz, 1H), 7.88 (d, J=6.0 Hz, 1H), 7.98 (m, 2H), 8.04 (m, 2H), 8.37 (s, 1H), 8.53 (t, J=2.3 Hz, 1H), 8.60 (d, J=2.3 Hz, 1H), 9.07 (d, J=2.3 Hz, 1H). m/z (ES+APCI)⁺: 327 [M+H]⁺.

### Example 61

### (3-Methyl-1H-pyrazolo[4,3-c]pyridin-4-yl)-(1-methyl-1H-pyrazol-3-yl)-amine

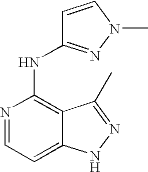

Example 61 was prepared analogously to Example 59 from Intermediate 3 and 1-methyl-1H-pyrazol-3-ylamine to give an off-white solid (16 mg, 38%). ¹H NMR (400 MHz, METHANOL-d₄) δ ppm 2.74 (s, 3H), 3.90 (s, 3H), 6.76 (d, J=6.4 Hz, 1H), 7.58-7.64 (m, 1H), 7.74 (d, J=6.0 Hz, 1H), 7.91-7.98 (m, 1H). m/z (ES+APCI)⁺: 229 [M+H]⁺

### Example 62

### (2-Fluoro-phenyl)-(3-methyl-1H-pyrazolo[4,3-c]pyridin-4-yl)-amine

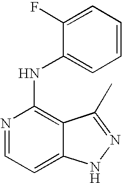

Example 62 was prepared analogously to Example 59 from Intermediate 3 and 2-fluoro-phenylamine to give a white solid (37 mg, 64%). ¹H NMR (400 MHz, DMSO-d) δ ppm 2.70 (s, 3H), 6.87 (d, J=6.0 Hz, 1H), 7.05-7.12 (m, 1H), 7.14-7.28 (m, 2H), 7.76 (d, J=6.0 Hz, 1H), 7.87 (br. s., 1H), 8.04-8.10 (m, 1H). m/z (ES+APCI)⁺: 243 [M+H]⁺

### Examples 63-91

Examples 63-91 in the following table were prepared analogously to Example 59 from Intermediate 3 and the corresponding amine:

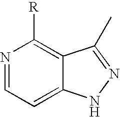

| | | | | HPLC |
|---|---|---|---|---|
| | | | m/z | retention |
| Example | R group | Name | (ES + APCI)⁺ | time (min) |
| 63 | 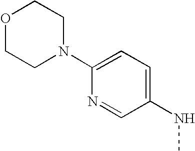 | (3-Methyl-1H- pyrazolo[4,3-c]pyridin-4- yl)-(6-morpholin-4-yl- pyridin-3-yl)-amine | 311 | 1.5^{e} |
| 64 | 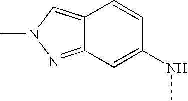 | (2-Methyl-2H-indazol-6- yl)-(3-methyl-1H- pyrazolo[4,3-c]pyridin-4- yl)-amine | 279 | 2.7^{a} |
| 65 | 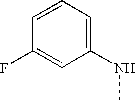 | (3-Fluoro-phenyl)-(3- methyl-1H-pyrazolo[4,3- c]pyridin-4-yl)-amine | 243 | 1.92^{e} |
| 66 | 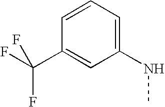 | (3-Methyl-1H- pyrazolo[4,3-c]pyridin-4- yl)-(3-trifluoromethyl- phenyl)-amine | 293 | 3.62^{a} |
| 67 | 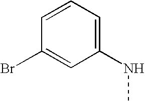 | (3-Bromo-phenyl)-(3- methyl-1H-pyrazolo[4,3- c]pyridin-4-yl)-amine | 303/305 | 3.60^{a} |
| 68 | 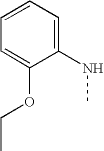 | (2-Ethoxy-phenyl)-(3- methyl-1H-pyrazolo[4,3- c]pyridin-4-yl)-amine | 269 | 3.73^{a} |
| 69 | 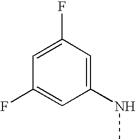 | (3,5-Difluoro-phenyl)-(3- methyl-1H-pyrazolo[4,3- c]pyridin-4-yl)-amine | 261 | 3.53^{a} |
| 70 | 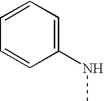 | (3-Methyl-1H- pyrazolo[4,3-c]pyridin-4- yl)-phenyl-amine | 225 | 3.13^{a} |
| 71 | 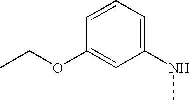 | (3-Ethoxy-phenyl)-(3- methyl-1H-pyrazolo[4,3- c]pyridin-4-yl)-amine | 269 | 1.08^{d} |
| 72 | 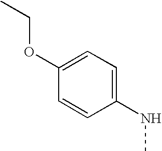 | (4-Ethoxy-phenyl)-(3- methyl-1H-pyrazolo[4,3- c]pyridin-4-yl)-amine | 269 | 1.07^{d} |
| 73 | 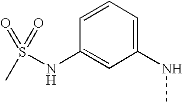 | N-[3-(3-Methyl-1H- pyrazolo[4,3-c]pyridin-4- ylamino)-phenyl]- methanesulfonamide | 318 | 0.88^{d} |
| 74 | 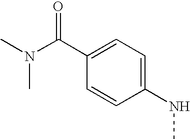 | N,N-Dimethyl-4-(3- methyl-1H-pyrazolo[4,3- c]pyridin-4-ylamino)- benzamide | 296 | 0.87^{d} |
| 75 | 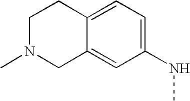 | (3-Methyl-1H- pyrazolo[4,3-c]pyridin-4- yl)-(2-methyl-1,2,3,4- tetrahydro-isoquinolin-7- yl)-amine | 294 | 0.44^{d} |
| 76 | 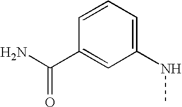 | 3-(3-Methyl-1H- pyrazolo[4,3-c]pyridin-4- ylamino)-benzamide | 268 | 0.6^{d} |
| 77 | 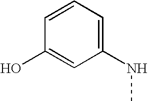 | 3-(3-Methyl-1H- pyrazolo[4,3-c]pyridin-4- ylamino)-phenol | 241 | 0.98^{c} |
| 78 | 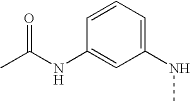 | N-[3-(3-Methyl-1H- pyrazolo[4,3-c]pyridin-4- ylamino)-phenyl]- acetamide | 282 | 1.24^{c} |
| 79 | 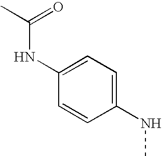 | N-[4-(3-Methyl-1H- pyrazolo[4,3-c]pyridin-4- ylamino)-phenyl]- acetamide | 282 | 0.79^{d} |
| 80 | 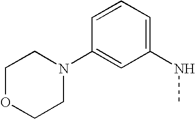 | (3-Methyl-1H- pyrazolo[4,3-c]pyridin-4- yl)-(3-morpholin-4-yl- phenyl)-amine | 310 | 1.8^{b} |
| 81 | 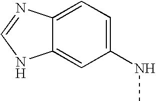 | (3H-Benzimidazol-5-yl)- (3-methyl-1H- pyrazolo[4,3-c]pyridin-4- yl)-amine | 265 | 1.12^{c} |
| 82 | 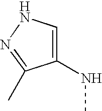 | (3-Methyl-1H- pyrazolo[4,3-c]pyridin-4- yl)-(3-methyl-1H-pyrazol- 4-yl)-amine | 229 | 0.91^{c} |
| 83 | 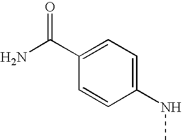 | 4-(3-Methyl-1H- pyrazolo[4,3-c]pyridin-4- ylamino)-benzamide | 268 | 1.07^{c} |
| 84 | 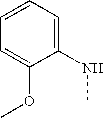 | (2-Methoxy-phenyl)-(3- methyl-1H-pyrazolo[4,3- c]pyridin-4-yl)-amine | 255 | 1.77^{c} |
| 85 | 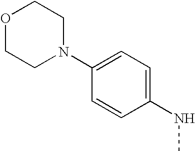 | (3-Methyl-1H- pyrazolo[4,3-c]pyridin-4- yl)-(4-morpholin-4-yl- phenyl)-amine | 310 | 1.34^{c} |
| 86 | 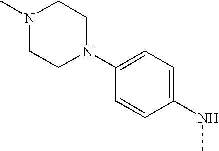 | [4-(4-Methyl-piperazin-1- yl)-phenyl]-(3-methyl-1H- pyrazolo[4,3-c]pyridin-4- yl)-amine | 323 | 1.27^{c} |
| 87 | 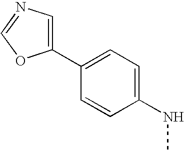 | (3-Methyl-1H- pyrazolo[4,3-c]pyridin-4- yl)-(4-oxazol-5-yl- phenyl)-amine | 292 | 1.43^{c} |
| 88 | 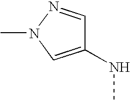 | (3-Methyl-1H- pyrazolo[4,3-c]pyridin-4- yl)-(1-methyl-1H-pyrazol- 4-yl)-amine | 229 | 1.04^{c} |
| 89 | 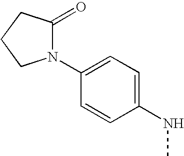 | 1-[4-(3-Methyl-1H- pyrazolo[4,3-c]pyridin-4- ylamino)-phenyl]- pyrrolidin-2-one | 308 | 1.31^{c} |
| 90 | 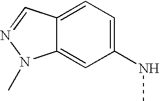 | (1-Methyl-1H-indazol-6- yl)-(3-methyl-1H- pyrazolo[4,3-c]pyridin-4- yl)-amine | 279 | 1.52^{c} |
| 91 | 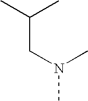 | Isobutyl-methyl-(3- methyl-1H-pyrazolo[4,3- c]pyridin-4-yl)-amine | 219 | 1.61^{c} |

^{a}HPLC column: 4.6 × 50 mm (5 μm) C-18 Xbridge; flow rate: 2 ml/min; Run time: 4.6 min: Solvent A: 0.1% Ammonium Hydroxide in water, Solvent B: Methanol; Gradient—10-100% B; Gradient time: 3.5 min.
^{b}HPLC column: 4.6 × 50 mm (5 μm) C-18 Xbridge; flow rate: 2 ml/min; Run time: 4.6 min: Solvent A: 0.1% Formic acid in water, Solvent B: Methanol; Gradient—10-100% B; Gradient time: 3.5 min.
^{c}HPLC column: 4.6 × 50 mm (5 μm) C-18 Xbridge; flow rate: 3 ml/min; Run time: 3.2 min: Solvent A: 0.1% Ammonium Hydroxide in water, Solvent B: Acetonitrile; Gradient—10-100% B; Gradient time: 2.35 min.
^{d}HPLC column: 4.6 × 50 mm (5 μm) C-18 Xbridge; flow rate: 3 ml/min; Run time: 3.2 min: Solvent A: 0.1% Formic acid in water, Solvent B: Acetonitrile; Gradient—10-100% B; Gradient time: 2.35 min.
^{e}HPLC column: 4.6 × 50 mm (5 μm) C-18 Xbridge; flow rate: 2 ml/min; Run time: 4.6 min: Solvent A: 0.1% Trifluoroacetic acid in water, Solvent B: Methanol; Gradient—10-100% B; Gradient time: 3.5 min.

### Example 92

### N,N-Dimethyl-N′-(3-methyl-1H-pyrazolo[4,3-c]pyridin-4-yl)-benzene-1,3-diamine

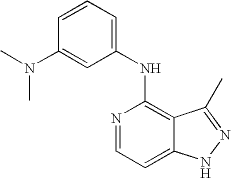

Intermediate 3 (40 mg, 0.24 mmol), N,N-dimethyl-benzene-1,3-diamine (65 mg, 0.48 mmol) and concentrated hydrochloric acid (21 μl, 0.72 mmol), were dissolved in n-butanol (1.1 ml). The reaction mixture was irradiated at 190° C. for 45 minutes in a Biotage I-60 microwave reactor. The mixture was evaporated, purified by preparative LCMS (low pH buffer) then eluted through a 0.5 gram Isolute-NH₂ cartridge with 9:1 DCM:methanol to give the free base as a white solid (15 mg, 23%). ¹H NMR (400 MHz, DMSO-d₆) δ ppm 2.71 (s, 3H), 2.89 (s, 6H), 6.35-6.40 (m, 1H), 6.84 (d, J=6.0 Hz, 1H), 7.05-7.13 (m, 3H), 7.78 (d, J=6.0 Hz, 1H), 7.83 (br. s., 1H). m/z (ES+APCI)⁺: 268 [M+H]⁺

### Examples 93-95

Examples 93-95 in the following table were prepared analogously to Example 92 from Intermediate 3 and the corresponding amine.

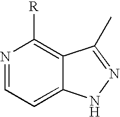

| | | | | HPLC |
|---|---|---|---|---|
| | | | m/z | retention |
| Example | R group | Name | (ES + APCI)⁺ | time (min) |
| 93 | 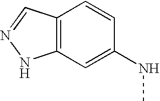 | (1H-Indazol-6-yl)-(3- methyl-1H- pyrazolo[4,3-c]pyridin- 4-yl)-amine | 265 | 1.3^{c} |
| 94 | 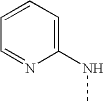 | (3-Methyl-1H- pyrazolo[4,3-c]pyridin- 4-yl)-pyridin-2-yl-amine | 226 | 1.03^{d} |
| 95 | 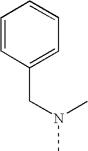 | Benzyl-methyl-(3- methyl-1H- pyrazolo[4,3-c]pyridin- 4-yl)-amine | 253 | 1.42^{d} |

^{c}HPLC column: 4.6 × 50 mm (5 μm) C-18 Xbridge; flow rate: 3 ml/min; Run time: 3.2 min: Solvent A: 0.1% Ammonium Hydroxide in water, Solvent B: Acetonitrile; Gradient—10-100% B; Gradient time: 2.35 min.
^{d}HPLC column: 4.6 × 50 mm (5 μm) C-18 Xbridge; flow rate: 3 ml/min; Run time: 3.2 min: Solvent A: 0.1% Formic acid in water, Solvent B: Acetonitrile; Gradient—10-100% B; Gradient time: 2.35 min.

### Example 96

### (3-Methyl-1H-pyrazolo[4,3-c]pyridin-4-yl)-(4-1,2,4-triazol-1-yl-phenyl)-amine hydrochloride salt

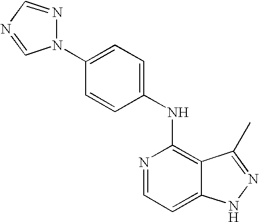

Intermediate 3 (50 mg, 0.3 mmol), 4-1,2,4-triazol-1-yl-phenylamine (95 mg, 0.60 mmol) and concentrated hydrochloric acid (27 μl, 0.39 mmol), were dissolved in n-butanol (1.1 ml). The reaction mixture was irradiated at 190° C. for 45 minutes in a Biotage I-60 microwave reactor. The mixture was evaporated, dissolved in DMSO (1 ml), filtered, washed with water and dried to give the desired product as a pale green solid (46 mg, 47%). ¹H NMR (400 MHz, 60° C., DMSO-d₆) δ ppm 2.75 (s, 3H), 7.14 (d, J=7.3 Hz, 1H), 7.56 (d, J=6.9 Hz, 1H), 7.73 (d, J=9.2 Hz, 2H), 8.02 (d, J=9.2 Hz, 2H), 8.25 (s, 1H), 9.32 (s, 1H), 9.99 (s, 1H). m/z (ES+APCI)⁺: 292 [M+H]⁺

### Example 97

### (2,4-Dimethoxy-benzyl)-(3-methyl-1H-pyrazolo[4,3-c]pyridin-4-yl)-amine

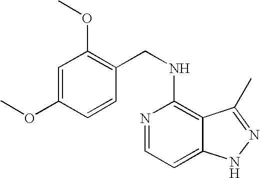

Intermediate 3 (200 mg, 1.20 mmol), 2,4-dimethoxybenzylamine (0.72 ml, 4.79 mmol) and n-butanol (2.5 ml) were combined and irradiated at 190° C. for 30 minutes in a Biotage I-60 microwave reactor. The reaction mixture was evaporated, the residue was partitioned between DCM (20 ml) and water (20 ml). The aqueous layer was extracted with DCM (20 ml) and then the combined organic layers were washed with brine (20 ml), dried (MgSO₄) and evaporated. The crude product was concentrated onto silica and purified by flash chromatography on the Biotage SP4, eluting with 0 to 100% EtOAc/petroleum ether to give the desired product as a pale yellow oil (303 mg, 85%). ¹H NMR (400 MHz, DMSO-d₆) δ ppm 2.61 (s, 3H), 3.71 (s, 3H), 3.84 (s, 3H), 4.57 (d, J=6.0 Hz, 2H), 6.37-6.46 (m, 2H), 6.52-6.59 (m, 2H), 7.04-7.12 (m, 1H), 7.60 (d, J=6.0 Hz, 1H). m/z (ES+APCI)⁺: 299 [M+H]⁺

### Example 98

### (1-Benzyl-piperidin-4-yl)-(3-methyl-1H-pyrazolo[4,3-c]pyridin-4-yl)-amine

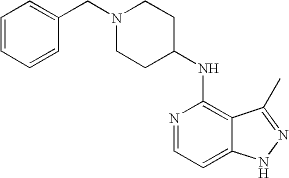

Intermediate 3 (200 mg, 1.20 mmol), 4-amino-1-benzylpiperidine (0.98 ml, 4.79 mmol) and n-butanol (2.5 ml) were combined and irradiated at 190° C. for 1.5 h in a Biotage I-60 microwave reactor. The reaction mixture was evaporated and the crude product was purified by flash chromatography on the Biotage SP4, eluting with 12 to 100% EtOAc/petroleum ether. Further purification by cation exchange chromatography using an Isolute SCX cartridge gave an off-white foam (153 mg, 40%). ¹H NMR (400 MHz, DMSO-d₆) δ ppm 1.58-1.71 (m, 2H), 1.88-1.96 (m, 2H), 2.03-2.12 (m, 2H), 2.57 (s, 3H), 2.76-2.84 (m, 2H), 3.47 (s, 2H), 3.98-4.10 (m, 1H), 5.52-5.59 (m, 1H), 6.56 (d, J=6.0 Hz, 1H), 7.22-7.35 (m, 5H), 7.64 (d, J=6.0 Hz, 1H). m/z (ES+APCI)⁺: 322 [M+H]⁺

### Example 99

### (1-Methyl-1H-indazol-5-yl)-(3-methyl-1H-pyrazolo[4,3-c]pyridin-4-yl)-amine

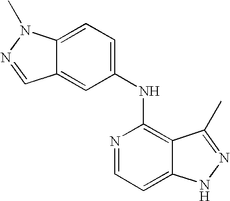

Intermediate 3 (40 mg, 0.24 mmol), 1-methyl-1H-indazol-5-amine (140 mg, 0.95 mmol) and concentrated hydrochloric acid (21 μl, 0.72 mmol) were dissolved in n-butanol (1 ml). The reaction mixture was irradiated at 190° C. for 1 h in a Biotage I-60 microwave reactor. The mixture was evaporated and then purified by preparative LCMS (high pH buffer). Further purification by cation exchange chromatography using an Isolute SCX cartridge eluting with MeOH then 2M NH₃ in MeOH, followed by anion exchange chromatography using an Isolute-NH₂ cartridge eluting with 9:1 DCM:MeOH gave an off-white solid (28 mg, 42%). ¹H NMR (400 MHz, DMSO-d₆) δ ppm 2.73 (s, 3H), 4.03 (s, 3H), 6.82 (d, J=6.0 Hz, 1H), 7.54-7.62 (m, 2H), 7.76 (d, J=6.0 Hz, 1H), 7.96-7.98 (m, 2H), 8.17 (dd, J=1.8, 0.9 Hz, 1H). m/z (ES+APCI)⁺: 279 [M+H]⁺

### Example 100

### 3-Methyl-1H-pyrazolo[4,3-c]pyridine-4-carboxylic acid cyclohexylamide

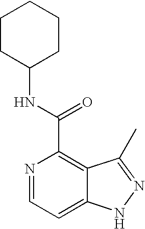

### Step 1

To a solution of cyclohexylamine (18 μl, 0.16 mmol) in DMF (2 ml) was added Intermediate 6 (70 mg, 0.24 mmol), HATU (96 mg, 0.25 mmol) and diisopropylethylamine (164 μl, 0.94 mmol), and the reaction was stirred at room temperature for 72 h. The mixture was evaporated, dissolved in a minimum amount of 10% MeOH in DCM and eluted through an Isolute-NH₂ cartridge. The filtrate was concentrated and used in the next step without purification.

### Step 2

The crude product from Step 1 (100 mg, 0.26 mmol) and trifluoroacetic acid (2 ml, 3.07 mmol) were combined and stirred at reflux for 18 h. The reaction mixture was evaporated and then purified by preparative LCMS (high pH buffer) to give the product as a white solid (28 mg, 68%). ¹H NMR (400 MHz, DMSO-d₆) δ ppm 1.08-1.22 (m, 1H), 1.26-1.42 (m, 4H), 1.56-1.65 (m, 1H), 1.68-1.78 (m, 2H), 1.80-1.91 (m, 2H), 2.63 (s, 3H), 3.75-3.88 (m, 1H), 7.57 (d, J=6.0 Hz, 1H), 8.27 (d, J=6.0 Hz, 1H), 8.46-8.53 (m, 1H). m/z (ES+APCI)⁺: 259 [M+H]⁺

### Example 101

### 1-[4-(3-Methyl-1H-pyrazolo[4,3-c]pyridin-4-ylamino)-piperidin-1-yl]-ethanone

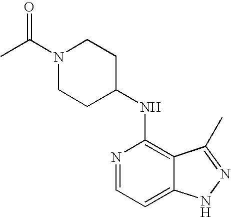

### Step 1

To a solution of Intermediate 8 (70 mg, 0.18 mmol) in DMF (2 ml) was added acetic acid (15 μl, 0.27 mmol), HATU (110 mg, 0.29 mmol) and diisopropylethylamine (188 μl, 1.08 mmol), and the reaction was stirred at room temperature for 18 h. The mixture was evaporated, dissolved in a minimum amount of 10% MeOH in DCM and eluted through an Isolute-NH₂ cartridge and the filtrate evaporated and used in the next step without further purification.

### Step 2

The crude product of Step 1 and trifluoroacetic acid (1 ml, 1.54 mmol) were combined and stirred at reflux for 18 h. The reaction mixture was evaporated and then purified by preparative LCMS (high pH buffer) to give a white solid (24 mg, 48%). ¹H NMR (400 MHz, DMSO-d₆) δ ppm 1.40-1.51 (m, 1H), 1.51-1.63 (m, 1H), 1.89-2.03 (m, 5H), 2.57 (s, 3H), 2.66-2.75 (m, 1H), 3.11-3.21 (m, 1H), 3.79-3.86 (m, 1H), 4.23-4.37 (m, 2H), 5.62-5.68 (m, 1H), 6.59 (d, J=6.0 Hz, 1H), 7.66 (d, J=6.0 Hz, 1H). m/z (ES+APCI)⁺: 274 [M+H]⁺

### Examples 102-103

Examples 102-103 in the table below were prepared analogously to Example 101

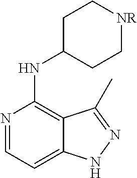

| | | | | HPLC |
|---|---|---|---|---|
| | | | m/z | retention |
| Example | R group | Name | (ES + APCI)⁺ | time (min) |
| 102 | 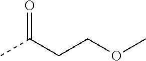 | 3-Methoxy-1-[4-(3- methyl-1H- pyrazolo[4,3-c]pyridin- 4-ylamino)-piperidin-1- yl]-propan-1-one | 318 | 1.04^{c} |
| 103 | 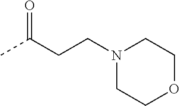 | 1-[4-(3-Methyl-1H- pyrazolo[4,3-c]pyridin- 4-ylamino)-piperidin-1- yl]-3-morpholin-4-yl- propan-1-one | 373 | 1.00^{c} |

^{c}HPLC column: 4.6 × 50 mm (5 μm) C-18 Xbridge; flow rate: 3 ml/min; Run time: 3.2 min:
Solvent A: 0.1% Ammonium Hydroxide in water, Solvent B: Acetonitrile; Gradient-10-100% B; Gradient time: 2.35 min.

### Example 104

### Cyclobutyl-[4-(3-methyl-1H-pyrazolo[4,3-c]pyridin-4-ylamino)-piperidin-1-yl]-methanone

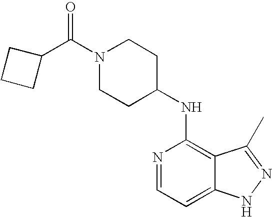

To a solution of Intermediate 8 (70 mg, 0.18 mmol) in DMF (2 ml) was added cyclobutyl acid (26 μl, 0.27 mmol), HATU (110 mg, 0.29 mmol) and diisopropylethylamine (188 μl, 1.08 mmol), and the mixture was stirred at room temperature for 18 h. The mixture was evaporated, dissolved in a minimum amount of 10% MeOH in DCM and eluted through an Isolute-NH₂ cartridge and the filtrate evaporated. The residue was combined with trifluoroacetic acid (1 ml) and stirred at reflux for 18 h. The mixture was concentrated and the residue purified by preparative LCMS (low pH buffer). The product was then eluted through an Isolute-NH₂ cartridge with 10% MeOH in DCM to a white solid (20 mg, 35%). ¹H NMR (400 MHz, DMSO-d₆) δ ppm 1.37-1.56 (m, 2H), 1.67-1.79 (m, 1H), 1.83-1.99 (m, 3H), 2.02-2.25 (m, 4H), 2.57 (s, 3H), 2.65-2.75 (m, 1H), 3.01-3.10 (m, 1H), 3.34 (s, 1H), 3.67-3.75 (m, 1H), 4.22-4.38 (m, 2H), 5.61-5.68 (m, 1H), 6.58 (d, J=6.4 Hz, 1H), 7.66 (d, J=6.0 Hz, 1H). m/z (ES+APCI)⁺: 314 [M+H]⁺.

### Example 105

### (4-Methoxy-phenyl)-[4-(3-methyl-1H-pyrazolo[4,3-c]pyridin-4-ylamino)-piperidin-1-yl]-methanone

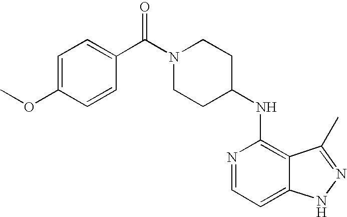

Example 105 was prepared analogously to Example 104 from Intermediate 8 and 4-methoxybenzoic acid to give the product (17 mg, 26%). ¹H NMR (400 MHz, 60° C., DMSO-d₆) δ ppm 1.56-1.68 (m, 2H), 1.95-2.04 (m, 2H), 2.60 (s, 3H), 3.05-3.14 (m, 2H), 3.81 (s, 3H), 3.96-4.15 (m, 2H), 4.31-4.42 (m, 1H), 5.54-5.60 (m, 1H), 6.59 (d, J=6.4 Hz, 1H), 6.96-7.02 (m, 2H), 7.34-7.40 (m, 2H), 7.67 (d, J=6.0 Hz, 1H). m/z (ES+APCI)⁺: 366 [M+H]⁺

### Example 106

### N-Cyclopentyl-4(3-methyl-1H-pyrazolo[4,3-c]pyridin-4-ylamino)-benzamide

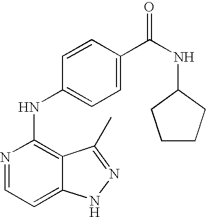

To a solution of cyclopentylamine (13 μl, 0.13 mmol) in DMF (2 ml) was added Intermediate 9 (78 mg, 0.20 mmol), HATU (81 mg, 0.21 mmol) and diisopropylethylamine (139 μl, 0.80 mmol) and the mixture was stirred at room temperature for 18 h. The mixture was evaporated, dissolved in a minimum amount of 10% MeOH in DCM and eluted through an Isolute-NH₂ cartridge and the filtrate concentrated. The residue was combined with trifluoroacetic acid (1 ml) and stirred at reflux for 18 h. The reaction mixture was evaporated and then purified by preparative LCMS (high pH buffer) to give a white solid (36 mg, 80%). ¹H NMR (400 MHz, DMSO-d₆) δ ppm 1.47-1.60 (m, 4H), 1.64-1.76 (m, 2H), 1.83-1.94 (m, 2H), 2.72 (s, 3H), 4.16-4.27 (m, 1H), 6.94 (d, J=6.0 Hz, 1H), 7.74-7.82 (m, 4H), 7.85 (d, J=6.0 Hz, 1H), 8.04-8.10 (m, 1H), 8.28 (br. s., 1H). m/z (ES+APCI)⁺: 336 [M+H]⁺

### Examples 107-113

Examples 107-113 in the table below were prepared analogously to Example 106 from Intermediate 44 and the appropriate amine:

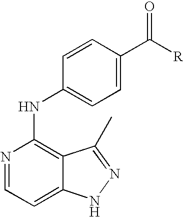

| | | | m/z | HPLC retention |
|---|---|---|---|---|
| Example | R group | Name | (ES + APCl)⁺ | time (min) |
| 107 | 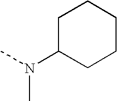 | N-Cyclohexyl-N- methyl-4-(3-methyl-1H- pyrazolo[4,3-c]pyridin- 4-ylamino)-benzamide | 364 | 2.04^{b} |
| 108 | 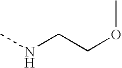 | N-(2-Methoxy-ethyl)-4- (3-methyl-1H- pyrazolo[4,3-c]pyridin- 4-ylamino)-benzamide | 326 | 1.36^{b} |
| 109 | 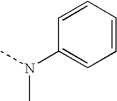 | N-Methyl-4-(3-methyl- 1H-pyrazolo[4,3- c]pyridin-4-ylamino)-N- phenyl-benzamide | 358 | 1.85^{b} |
| 110 | 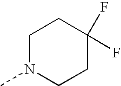 | (4,4-Difluoro-piperidin- 1-yl)-[4-(3-methyl-1H- pyrazolo[4,3-c]pyridin- 4-ylamino)-phenyl]- methanone | 372 | 1.49^{c} |
| 111 | 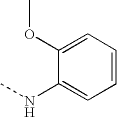 | N-(2-Methoxy-phenyl)- 4-(3-methyl-1H- pyrazolo[4,3-c]pyridin- 4-ylamino)-benzamide | 374 | 1.69^{c} |
| 112 | 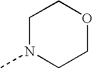 | [4-(3-Methyl-1H- pyrazolo[4,3-c]pyridin- 4-ylamino)-phenyl]- morpholin-4-yl- methanone | 338 | 1.15^{c} |
| 113 | 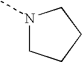 | [4-(3-Methyl-1H- pyrazolo[4,3-c]pyridin- 4-ylamino)-phenyl]- pyrrolidin-1-yl- methanone | 322 | 1.30^{c} |

^{b}HPLC column: 4.6 × 50 mm (5 μm) C-18 Xbridge; flow rate: 2 ml/min; Run time: 4.6 min: Solvent A: 0.1% Formic acid in water, Solvent B: Methanol; Gradient - 10-100% B; Gradient time: 3.5 min.
^{c}HPLC column: 4.6 × 50 mm (5 μm) C-18 Xbridge; flow rate: 3 ml/min; Run time: 3.2 min: Solvent A: 0.1% Ammonium Hydroxide in water, Solvent B: Acetonitrile; Gradient- 10-100% B; Gradient time: 2.35 min.

### Example 114

### 4-Cyclohexyloxy-3-methyl-1H-pyrazolo[4,3-c]pyridine

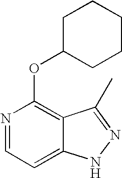

To a solution of cyclohexanol (249 μl, 2.40 mmol) in dioxane (2 ml) in a microwave vial was added sodium hydride (60% in mineral oil, 84 mg, 2.10 mmol). The mixture was allowed to stir at room temperature for 2 h. A solution of Intermediate 3 (100 mg, 0.60 mmol) in dioxane (1 ml) was added, then the reaction mixture was irradiated at 180° C. for 1.5 h in a Biotage I-60 microwave reactor. The mixture was evaporated and water (20 ml) and ethyl acetate (20 ml) were added. The layers were separated and the aqueous extracted with further ethyl acetate (20 ml). The organic layers were combined and washed with brine (20 ml), dried and evaporated. The crude product was then purified by LCMS (high pH buffer) to give the desired product as a colourless oil (37 mg, 27%). ¹H NMR (400 MHz, DMSO-d₆) δ ppm 1.39-1.55 (m, 4H), 1.57-1.68 (m, 2H), 1.69-1.81 (m, 2H), 1.87-2.00 (m, 2H), 2.55 (s, 3H), 5.17-5.38 (m, 1H), 6.96 (d, J=6.4 Hz, 1H), 7.76 (d, J=6.0 Hz, 1H). m/z (ES+APCI)⁺: 232 [M+H]⁺.

### Examples 115-117

Examples 115-117 in the table below were prepared analogously to Example 114 from Intermediate 3 and the corresponding alcohol:

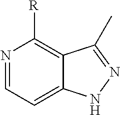

| | | | | HPLC |
|---|---|---|---|---|
| Ex- | | | m/z | retention |
| am- | | | (ES + | time |
| ple | R group | Name | APCI)⁺ | (min) |
| 115 | 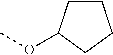 | 4-Cyclopentyloxy- 3-methyl-1H- pyrazolo[4,3-c] pyridine | 218 | 1.43^{d} |
| 116 | 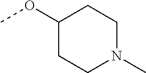 | 3-Methyl-4-(1- methyl-piperidin-4- yloxy)-1H- pyrazolo[4,3-c] pyridine | 247 | 1.18^{c} |
| 117 | 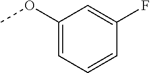 | 4-(3-Fluoro- phenoxy)- 3-methyl-1H- pyrazolo[4,3-c] pyridine | 244 | 1.65^{c} |

^{c}HPLC column: 4.6 × 50 mm (5 μm) C-18 Xbridge; flow rate: 3 ml/min; Run time: 3.2 min: Solvent A: 0.1% Ammonium Hydroxide in water, Solvent B: Acetonitrile; Gradient-10-100% B; Gradient time: 2.35 min.
^{d}HPLC column: 4.6 × 50 mm (5 μm) C-18 Xbridge; flow rate: 3 m1/rnin; Run time: 3.2 min: Solvent A: 0.1% Formic acid in water, Solvent B: Acetonitrile; Gradient-10-100% B; Gradient time: 2.35 min.

### Example 118

### 3-Methyl-4-(tetrahydro-pyran-4-yloxy)-1H-pyrazolo[4,3-c]pyridine

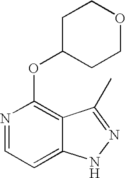

To a solution of tetrahydro-pyran-4-ol (137 μl, 1.48 mmol) in dioxane (2 ml) in a microwave vial was added sodium hydride (60% dispersion in mineral oil, 50 mg, 1.26 mmol) and the mixture was allowed to stir at room temperature for 2 h. A solution of Intermediate 3 (60 mg, 0.36 mmol) in dioxane (1 ml) was added and the reaction mixture was irradiated at 170° C. for 1.5 h in a Biotage I-60 microwave reactor. The mixture was evaporated, and partitioned between water and ethyl acetate. The layers were separated and the aqueous extracted with further ethyl acetate (20 ml). The organic layers were combined and washed with brine, dried (MgSO₄) and evaporated. The crude product was then purified by preparative LCMS (low pH buffer) then eluted through an Isolute-NH₂ cartridge with 9:1 DCM:methanol to give the desired product as a white solid (24 mg, 27%). ¹H NMR (400 MHz, DMSO-d₆) δ ppm 1.68-1.78 (m, 2H), 1.99-2.08 (m, 2H), 2.56 (s, 3H), 3.53-3.63 (m, 2H), 3.82-3.91 (m, 2H), 5.39-5.48 (m, 1H), 6.99 (d, J=6.0 Hz, 1H), 7.77 (d, J=6.0 Hz, 1H). m/z (ES+APCI)⁺: 234 [M+H]⁺

### Example 119

### 4-(1-Isopropyl-piperidin-4-yloxy)-3-methyl-1H-pyrazolo[4,3-e]pyridine

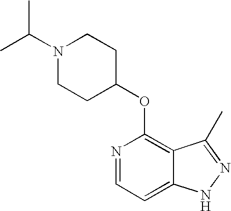

Example 119 was prepared analogously to Example 118 from Intermediate 3 and 1-Isopropyl-piperidin-4-ol to give a white solid (37 mg, 64%). ¹H NMR (400 MHz, DMSO-d₆) d ppm 0.98 (d, J=6.9 Hz, 6H), 1.70-1.80 (m, 2H), 1.91-2.00 (m, 2H), 2.37-2.45 (m, 2H), 2.55 (s, 3H), 2.65-2.75 (m, 3H), 5.22-5.31 (m, 1H), 6.96 (d, J=6.0 Hz, 1H), 7.75 (d, J=6.0 Hz, 1H). m/z (ES+APCI)⁺: 275 [M+H]⁺.

### Example 120

### 4-(4-Imidazol-1-yl-phenoxy)-3-methyl-1H-pyrazolo[4,3-c]pyridine

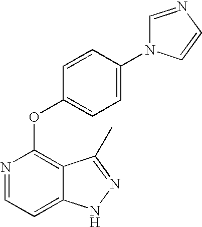

To a solution of 4-Imidazol-1-yl-phenylamine (230 mg, 1.48 mmol) in dioxane (2 ml) in a microwave vial was added sodium hydride (60% dispersion in mineral oil, 50 mg, 1.26 mmol) and the mixture was allowed to stir at room temperature for 2 h. A solution of Intermediate 3 (60 mg, 0.36 mmol) in dioxane (1 ml) was added and the reaction mixture was heated at 90° C. for 72 h. The mixture was evaporated, then water (20 ml) and ethyl acetate (20 ml) were added. The layers were separated and the aqueous extracted with further ethyl acetate (20 ml). The organic layers were combined and washed with brine, dried (MgSO₄) and evaporated. The crude product was than purified by preparative LCMS (low pH buffer) then eluted through an Isolute-NH₂ cartridge with 9:1 DCM:methanol to give the desired product as a white solid (2.3 mg, 2%). ¹H NMR (400 MHz, DMSO-d₆) δ ppm 2.66 (s, 3H), 7.12 (s, 1H), 7.16 (d, J=6.4 Hz, 1H), 7.38-7.43 (m, 2H), 7.68-7.75 (m, 3H), 7.75-7.77 (m, 1H), 8.25-8.26 (m, 1H). m/z (ES+APCI)⁺: 292 [M+H]⁺

### Example 121

### 4-((S)-sec-Butoxy)-3-methyl-1H-pyrazolo[4,3-c]pyridine

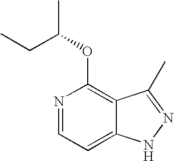

(S)-Butan-2-ol (91 μl, 0.98 mmol) was added to 1M potassium tert-butoxide in THF (1 ml, 0.98 mmol) and the mixture was stirred under a nitrogen atmosphere for 5 minutes before adding Intermediate 3 (0.041 mg, 0.25 mmol). The reaction mixture was irradiated at 120° C. for 100 minutes in a Biotage I-60 microwave reactor. The mixture was quenched with 4M hydrogen chloride in dioxane (250 μl) and then diluted with H₂O (10 ml). The mixture was extracted twice with DCM (20 ml) and the combined organic layers were evaporated. The crude product was purified preparative LCMS (high pH buffer) to give the desired product as an off-white solid (19 mg, 38%). ¹H NMR (400 MHz, DMSO-d₆) δ ppm 0.95 (t, J=7.4 Hz, 3H), 1.32 (d, J=6.0 Hz, 3H), 1.62-1.80 (m, 2H), 2.53 (s, 3H), 5.23-5.33 (m, 1H), 6.96 (d, J=6.0 Hz, 1H), 7.76 (d, J=6.0 Hz, 1H). m/z (ES+APCI)⁺: 206 [M+H]⁺.

### Examples 122-123

Examples 122-123 in the following table were prepared analogously to Example 121 from Intermediate 3 and the corresponding alcohol:

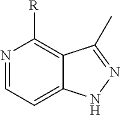

| | | | m/z | HPLC |
|---|---|---|---|---|
| Ex- | | | (ES + | retention |
| ample | R group | Name | APCI)⁺ | time (min) |
| 122 | 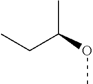 | 4-((R)-sec-Butoxy)-3- methyl-1H- pyrazolo[4,3-c]pyridine | 206 | 1.68^{c} |
| 123 | 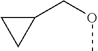 | 4-Cyclopropylmethoxy- 3-methyl-1H- pyrazolo[4,3-c]pyridine | 204 | 1.55^{c} |

^{c}HPLC column: 4.6 × 50 mm (5 μm) C-18 Xbridge; flow rate: 3 ml/min; Run time: 3.2 min: Solvent A: 0.1% Ammonium Hydroxide in water, Solvent B: Acetonitrile; Gradient-10-100% B; Gradient time: 2.35 min.

### Example 124

### 4-Ethoxy-3-methyl-1H-pyrazolo[4,3-c]pyridine

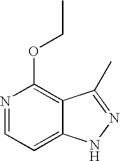

Concentrated KOH (aq) (0.5 ml) was added to a solution of Intermediate 3 (100 mg, 0.60 mmol) in ethanol (1 ml). The reaction mixture was irradiated at 120° C. for 1 h in a Biotage I-60 microwave reactor. The mixture was evaporated, diluted with H₂O (5 ml) and neutralised with dilute hydrochloric acid. The aqueous was then extracted with DCM (30 ml) twice, the combined organic layers were dried (MgSO₄), and evaporated. The crude product was purified by preparative LCMS. (high pH buffer) to give the desired product as a white solid (28 mg, 26%). ¹H NMR (400 MHz, DMSO-d₆) δ ppm 1.38 (t, J=7.1 Hz, 3H), 2.54 (s, 3H), 4.45 (q, J=7.2 Hz, 2H), 6.99 (d, J=6.0 Hz, 1H), 7.77 (d, J=6.0 Hz, 1H). m/z (ES-1-APCI)⁺: 178 [M+H]⁺.

### Example 125

### 4-Benzyl-3-methyl-1H-pyrazolo[4,3-c]pyridine

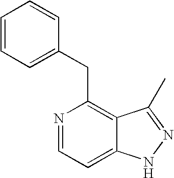

### Step 1

A 0.5M solution of benzyl zinc bromide in THF (2.1 ml, 1.05 mmol) was added to Intermediate 4 (150 mg, 0.52 mmol) and Pd(PPh₃)₄ (30 mg, 0.03 mmol) in THF (2 ml) under nitrogen at room temperature, and the resulting mixture was stirred at 60° C. overnight. The mixture was quenched with saturated NH₄Cl (aq) (10 ml) and extracted twice with EtOAc (20 ml). The combined organic layers were washed with brine (20 ml), dried (MgSO₄) and evaporated. The crude product was purified by flash chromatography on the Biotage SP4, eluting with 0 to 60% EtOAc/petroleum ether to give a yellow oil (78 mg) which was used in the next step without further purification.

### Step 2

The product of Step 1 (170 mg) and trifluoroacetic acid (3 ml, 4.61 mmol) were combined and stirred at reflux for 2.5 h. The reaction mixture was evaporated, the residue dissolved in EtOAc (20 ml) and partitioned with saturated NaHCO₃ (aq) (20 ml). The aqueous layer was extracted with EtOAc (10 ml), the combined organic layers were washed with brine, dried (MgSO₄) and evaporated. The crude product was purified by prep LCMS (high pH buffer) to give the desired product as a white solid (30 mg, 38%). ¹H NMR (400 MHz, DMSO-d₆) δ ppm 2.53 (s, 3H), 4.46 (s, 2H), 7.14-7.20 (m, 3H), 7.23-7.29 (m, 2H), 7.32 (d, J=6.0 Hz, 1H), 8.19 (d, J=6.0 Hz, 1H). m/z (ES+APCI)⁺: 224 [M+H]⁺.

### Examples 126-127

Examples 126-127 in the following table were prepared analogously to Example 125

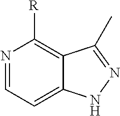

| | | | m/z | HPLC |
|---|---|---|---|---|
| Ex- | | | (ES + | retention |
| ample | R group | Name | APCI)⁺ | time (min) |
| 126 | 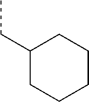 | 4-Cyclohexylmethyl-3- methyl-1H- pyrazolo[4,3-c]pyridine | 230 | 1.66^{c} |
| 127 | 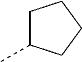 | 4-Cyclopentyl-3- methyl-1H- pyrazolo[4,3-c]pyridine | 202 | 1.48^{c} |

^{c}HPLC column: 4.6 × 50 mm (5 μm) C-18 Xbridge; flow rate: 3 ml/min; Run time: 3.2 min: Solvent A: 0.1% Ammonium Hydroxide in water, Solvent B: Acetonitrile; Gradient-10-100% B; Gradient time: 2.35 min.

### Example 128

### 3-Methyl-4-(3-methyl-butyl)-1H-pyrazolo[4,3-c]pyridine

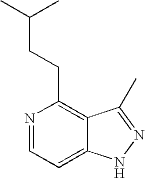

### Step 1

A 0.5M solution of 3-methylbutyl zinc bromide in THF (1.4 ml, 0.70 mmol) was added to Intermediate 4 (100 mg, 0.35 mmol) and Pd(PPh₃)₄ (20 mg, 0.02 mmol) in THF (2 ml) under nitrogen at room temperature. The reaction was stirred at 60° C. overnight. The mixture was quenched with saturated ammonium chloride aqueous solution (10 ml) and extracted twice with EtOAc (20 ml). The combined organic layers were washed with brine, dried (MgSO₄) and evaporated. The crude product was purified by flash chromatography on the Biotage SP4, eluting with 0 to 60% EtOAc/petroleum ether to give the desired product as a yellow oil which was used in Step 2 without further purification.

### Step 2

The product of step 1 (80 mg) and trifluoroacetic acid (2 ml, 3.07 mmol) were combined and stirred at reflux for 3 h. The crude product preparative LCMS (low pH buffer) than eluted through an Isolute-NH₂ cartridge with 9:1 DCM:methanol to give a white solid (8.4 mg, 12% over 2 steps). ¹H NMR (400 MHz, DMSO-d₆) δ ppm 0.96 (d, J=6.4 Hz, 6H), 1.56-1.62 (m, 2H), 1.63° 1.73 (m, 1H), 2.64 (s, 3H), 3.04-3.10 (m, 2H), 7.23 (d, J=6.0 Hz, 1H), 8.12 (d, J=6.0 Hz, 1H). m/z (ES+APCI)⁺: 204 [M+H]⁺

### Example 129

### 3-Methyl-4-oxazol-2-yl-1H-pyrazolo[4,3-c]pyridine

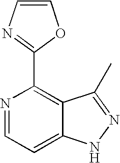

### Step 1

A solution of Intermediate 4 (80 mg, 0.28 mmol) in toluene (2 ml) was degassed for 10 minutes and placed under an atmosphere of nitrogen. 2-(Tri-n-butylstannyl) oxazole (70 μl, 0.33 mmol) and Pd(PPh₃)₄ (16 mg, 0.014 mmol) were added. The reaction vessel was evacuated backfilled with nitrogen, twice then stirred at reflux for 18 h. The mixture was evaporated, dry loaded onto silica and purified by flash chromatography on the Biotage SP4, eluting with 10 to 100% EtOAc/petroleum etherto give an orange oil (64 mg) which was used in Step 2 without further purification.

### Step 2

The product of Step 1 (62 mg, 0.19 mmol) and trifluoroacetic acid (0.5 ml, 0.77 mmol) were combined and stirred at reflux for 18 h. The reaction mixture was evaporated and then purified by preparative LCMS (high pH buffer) to give a white solid (19 mg, 34%). ¹H NMR (400 MHz, DMSO-d₆) δ ppm 2.70 (s, 3H), 7.56 (s, 1H), 7.61 (d, J=6.0 Hz, 1H), 8.38 (s, 1H), 8.40 (d, J=6.0 Hz, 1H). m/z (ES+APCI)⁺: 201 [M+H]⁺.

### Examples 130-131

Examples 130-131 in the following table were prepared analogously to Example 129:

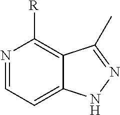

| | | | m/z | HPLC |
|---|---|---|---|---|
| Ex- | | | (ES + | retention |
| ample | R group | Name | APCI)⁺ | time (min) |
| 130 | 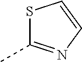 | 3-Methyl-4-thiazol-2-yl- 1H-pyrazolo[4,3- c]pyridine | 217 | 1.31^{c} |
| 131 | 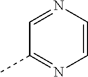 | 3-Methyl-4-pyrazin-2- yl-1 H-pyrazolo[4,3- c]pyridine | 212 | 0.92^{c} |

^{c}HPLC column: 4.6 × 50 mm (5 μm) C-18 Xbridge; flow rate: 3 ml/min; Run time: 3.2 min: Solvent A: 0.1% Ammonium Hydroxide in water, Solvent B: Acetonitrile; Gradient-10-100% B; Gradient time: 2.35min.

### Example 132

### (3-Fluoro-phenyl)-(1H-pyrazolo[4,3-c]pyridin-4-yl)-amine

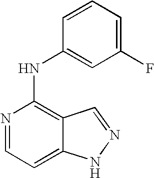

Intermediate 11 (30 mg, 0.20 mmol), 3-fluoroaniline (28 μl, 0.29 mmol) and conc. HCl (aq) (18 μl, 0.59 mmol) were combined in n-butanol (0.5 ml) and heated in the microwave at 190° C. for 1 h. The solvents were evaporated to dryness and the crude mixture purified by preparative LCMS to give a white solid (21 mg, 47%). ¹H NMR (400 MHz, DMSO-d₆) δ ppm 6.79 (td, J=8.4, 2.5 Hz, 1H), 7.01 (d, J=7.3 Hz, 1H), 7.33-7.41 (m, 1H), 7.62 (d, J=7.3 Hz, 1H), 7.95 (d, J=6.0 Hz, 1H), 8.16 (dt, J=12.6, 2.4 Hz, 1H), 8.50 (s, 1H), 9.53 (s, 1H); m/z (ES+APCI)⁺: 229 [M+H]⁺.

### Example 133

### Cyclohexyl-(1H-pyrazolo[4,3-c]pyridin-4-yl)-amine

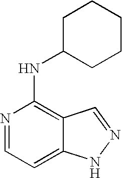

Intermediate 11 (30 mg, 0.20 mmol), cyclohexylamine (89 μl, 0.29 mmol) and conc. HCl (18 μl, 0.59 mmol) were combined in n-butanol (0.5 ml) and heated in the microwave at 190° C. for 1 h. The solvents were evaporated to dryness and the crude mixture purified by preparative LCMS to give a white solid (11 mg, 26%). ¹H NMR (400 MHz, DMSO-d₆) δ ppm 1.14-1.44 (m, 5H), 1.63-1.71 (m, 1H), 1.73-1.83 (m, 2H), 1.96-2.06 (m, 2H), 4.04 (m, 1H), 6.61 (d, J=6.0 Hz, 1H), 7.01 (d, J=7.8 Hz, 1H), 7.70 (d, J=6.0 Hz, 1H), 8.24 (s, 1H); m/z (ES+APCI)⁺: 217 [M+H]⁺.

### Example 134

### (3-Bromo-1H-pyrazolo[4,3-c]pyridin-4-yl)-cyclohexyl-amine

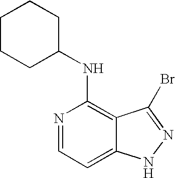

Intermediate 15 (1.9 g, 8.19 mmol) and cyclohexylamine (3.73 ml, 32.8 mmol) were combined in n-butanol (30 ml) and heated in the microwave for 1 h at 190° C. The solvents were evaporated and the crude product purified by flash chromatography using a Biotage SP4 (ethyl acetate/petroleum ether gradient) to give a white solid (1.51 g, 63%). ¹H NMR (400 MHz, DMSO-d₆) δ ppm 1.24-1.48 (m, 5H), 1.58-1.66 (m, 1H), 1.68-1.79 (m, 2H), 1.97-2.07 (m, 2H), 4.05-4.14 (m, 1H), 5.85 (d, J=7.3 Hz, 1H), 6.72 (d, J=6.0 Hz, 1H), 7.78 (d, 1H); m/z (ES+APCI)⁺: 295/297 [M+H]⁺.

### Example 135

### Cyclohexyl-(3-pyridin-3-yl-1H-pyrazolo[4,3-c]pyridin-4-yl)-amine

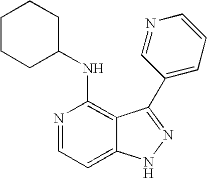

Example 134 (50 mg, 0.17 mmol), Pd(dppf)Cl₂ (14 mg, 0.02 mmol), 3-pyridineboronic acid (31 mg, 0.26 mmol) and 2 M sodium carbonate (aq) (298 μl, 0.60 mmol) were combined in dioxane (2 ml), the solvent degassed and the vial flushed out with nitrogen. The reaction mixture was then heated to 90° C. for 18 h. The solvents were evaporated and the crude residue re-dissolved in 9:1 DCM:methanol and filtered through a plug of silica, eluting with 9:1 DCM:methanol. The solvents were evaporated and the crude product purified by preparative LCMS to give a beige solid (0.8 mg, 22%). ¹H NMR (400 MHz, DMSO-d₆) δ ppm 1.13-1.27 (m, 3H), 1.27-1.42 (m, 2H), 1.51-1.65 (m, 3H), 1.89-1.98 (m, 2H), 3.99-4.09 (m, 1H), 5.02 (d, J=7.8 Hz, 1H), 6.79 (d, J=6.0 Hz, 1H), 7.63 (dd, J=7.8, 5.5 Hz, 1H), 7.84 (d, J=6.4 Hz, 1H), 8.12 (dt, J=8.0, 1.9 Hz, 1H), 8.74 (dd, J=5.0, 1.8 Hz, 1H), 8.91 (s, 1H); m/z (ES+APCI)⁺: 294 [M+H]⁺.

### Example 136

### Cyclohexyl-(3-phenyl-1H-pyrazolo[4,3-c]pyridin-4-yl)-amine

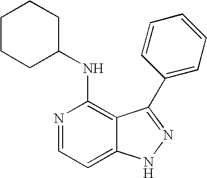

Example 134 (100 mg, 0.34 mmol), Pd(PPh₃)₄ (118 mg, 0.1 mmol), phenyl boronic acid (62 mg, 0.51 mmol) and 2 M sodium carbonate (aq) (340 μl, 0.68 mmol) were combined in a mixture of toluene (2.5 ml) and methanol (0.5 ml), the reaction content degassed and then heated to 65° C., under nitrogen for 18 h. The solvents were evaporated and the crude residue re-dissolved in 9:1 DCM:methanol and filtered through a plug of silica, eluting with 9:1 DCM:methanol. The solvents were evaporated and the crude product purified by preparative LCMS to give a white solid (7 mg, 8%). NMR (400 MHz, DMSO-d₆) δ ppm 1.08-1.28 (m, 3H), 1.29-1.41 (m, 2H), 6.73 (d, J=6.0 Hz, 1H), 7.54-7.64 (m, 3H), 7.66-7.70 (m, 2H), 7.81 (d, J=6.0 Hz, 1H); m/z (ES+APCI)⁺: 293 [M+H]⁺.

### Example 137

### N-[3-(4-Cyclohexylamino-1H-pyrazolo[4,3-c]pyridin-3-yl)-phenyl]-acetamide

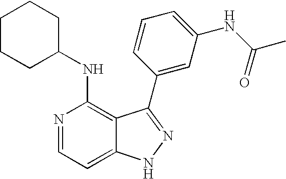

Example 134 (50 mg, 0.17 mmol), Pd(dppf)Cl₂ (14 mg, 0.02 mmol), 3-acetamidophenylboronic acid (46 mg, 0.26 mmol) and 2 M sodium carbonate (aq) (298 μl, 0.60 mmol) were combined in dioxane (2 ml), the solvent degassed and the vial flushed out with nitrogen. The reaction mixture was then heated to 90° C. for 18 h. The solvents were evaporated and the crude residue re-dissolved in 9:1 DCM:methanol and filtered through a plug of silica, eluting with 9:1 DCM:methanol. The solvents were evaporated and the crude product purified by preparative LCMS to give a brown solid (7 mg, 12%). ¹H NMR (400 MHz, DMSO-d₆) δ ppm 1.12-1.27 (m, 3H), 1.27-1.40 (m, 2H), 1.47-1.58 (m, 3H), 1.84-1.93 (m, 2H), 2.11 (s, 3H), 3.98-4.07 (m, 1H), 5.10 (d, J=7.8 Hz, 1H), 6.74 (d, J=6.0 Hz, 1H), 7.32 (d, J=7.8 Hz, 1H), 7.52 (t, J=7.8 Hz, 1H), 7.64 (d, J=9.2 Hz, 1H), 7.80 (d, J=6.0 Hz, 1H), 8.05 (s, 1H), 10.19 (s, 1H); m/z (ES+APCI)⁺: 350 [M+H]⁺.

### Example 138

### Cyclohexyl-(3-cyclopropyl-1H-pyrazolo[4,3-c]pyridin-4-yl)-amine

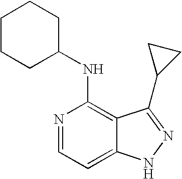

Intermediate 18 (30 mg, 0.16 mmol) and cyclohexylamine (71 μl, 0.62 mmol) were combined in n-butanol (0.5 ml) and heated in the microwave for 1 h at 190° C. The solvents were evaporated and the crude product purified by preparative LCMS to give a white solid (7 mg, 18%). ¹H NMR (400 MHz, DMSO-d₆) δ ppm 0.84-0.92 (m, 2H), 1.71-1.81 (m, 2H), 1.97-2.06 (m, 2H), 2.30-2.38 (m, 1H), 4.05-4.15 (m, 1H), 5.73 (d, J=8.2 Hz, 1H), 6.58 (d, J=6.4 Hz, 1H), 7.69 (d, J=6.0 Hz, 1H); m/z (ES+APCI)⁺: 257 [M+H]⁺.

### Example 139

### 4-(4-Cyclohexylamino-1H-pyrazolo[4,3-c]pyridin-3-yl)-N-methyl-benzamide

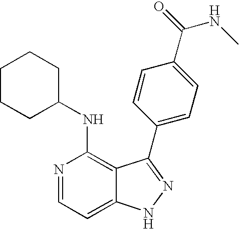

Intermediate 14 (50 mg, 0.11 mmol), Pd(dppf)Cl₂ (9 mg, 0.01 mmol), N-methyl-4-(4,4,5,5-tetramethyl-1,3,2-dioxaborolan-2-yl)benzamide (42 mg, 0.16 mmol) and 2 M aqueous sodium carbonate (189 μl, 0.38 mmol) were combined in dioxane (1 ml), the reaction mixture degassed and the vial flushed with nitrogen. The reaction mixture was then heated to 90° C. for 18 h. After heating to 90° C. for 18 h, the mixture was partitioned between DCM and water, the organic phase was collected using a phase separation cartridge and concentrated. The crude residue was treated with TFA (1 ml) at 60° C. for 19 h. The mixture was concentrated and the crude product purified by preparative LCMS (low pH buffer). The resulting salt was re-dissolved in methanol and eluted through an Isolute-NH₂ cartridge and solvents evaporated to give the final product (17 mg, 45%). ¹H NMR (400 MHz, DMSO-d₆) δ ppm 1.10-1.27 (m, 3H), 1.30-1.42 (m, 2H), 1.52-1.64 (m, 3H), 1.91-1.98 (m, 2H), 2.87 (d, J=4.6 Hz, 3H), 4.01-4.09 (m, 1H), 4.99 (d, J=7.8 Hz, 1H), 6.77 (d, J=6.0 Hz, 1H), 7.77-7.84 (m, 3H), 8.06 (d, J=8.7 Hz, 2H), 8.61-8.65 (m, 1H); m/z (ES+APCI)⁺: 350 [M+H]⁺.

### Examples 140-154

Examples 140-154 in the following table were prepared analogously to Example 139 from Intermediate 14 and the appropriate boronic acid or boronic ester:

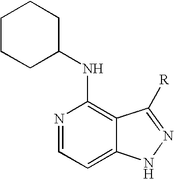

| | | | m/z | HPLC retention |
|---|---|---|---|---|
| Example | R group | Name | (ES + APCl)⁺ | time (min)^{a,b} |
| 140 | 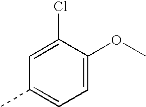 | [3-(3-Chloro-4- methoxy-phenyl)- 1H-pyrazolo[4,3- c]pyridin-4-yl]- cyclohexyl-amine | 357 | 3.84^{a} |
| 141 | 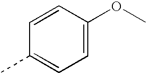 | Cyclohexyl- [3-(4- methoxy-phenyl)- 1H-pyrazolo[4,3- c]pyridin-4-yl]- amine | 323 | 3.72^{a} |
| 142 | 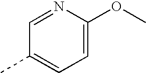 | Cyclohexyl-[3-(6- methoxy-pyridin- 3-yl)-1H- pyrazolo[4,3- c]pyridin-4-yl]- amine | 324 | 3.64^{c} |
| 143 | 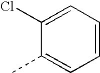 | [3-(2-Chloro- phenyl)-1H- pyrazolo[4,3- c]pyridin-4-yl]- cyclohexyl-amine | 327 | 3.66^{a} |
| 144 | 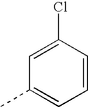 | [3-(3-Chloro- phenyl)-1H- pyrazolo[4,3- c]pyridin-4-yl]- cyclohexyl-amine | 327 | 2.16^{b} |
| 145 | 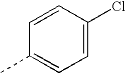 | [3-(4-Chloro- phenyl)-1H- pyrazolo[4,3- c]pyridin-4-yl]- cyclohexyl-amine | 327 | 3.94^{a} |
| 146 | 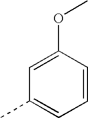 | Cyclohexyl-[3-(3- methoxy-phenyl)- 1H-pyrazolo[4,3- c]pyridin-4-yl]- amine | 323 | 3.74^{a} |
| 147 | 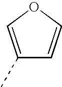 | Cyclohexyl-(3- furan-2-yl-1H- pyrazolo[4,3- c]pyridin-4-yl)- amine | 283 | 3.54a |
| 148 | 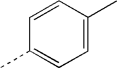 | Cyclohexyl-(3-p- tolyl-1H- pyrazolo[4,3- c]pyridin-4-yl)- amine | 307 | 3.86^{a} |
| 149 | 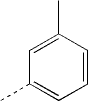 | Cyclohexyl-(3-m- tolyl-1H- pyrazolo[4,3- c]pyridin-4-yl)- amine | 307 | 3.87^{a} |
| 150 | 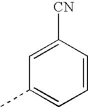 | 3-(4- Cyclohexylamino- 1H-pyrazolo[4,3- c]-pyridin-3-yl)- benzonitrile | 318 | 3.52^{a} |
| 151 | 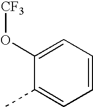 | Cyclohexyl-[3-(2- trifluoromethoxy- phenyl)-1H- pyrazolo[4,3- c]pyridin-4-yl]- amine | 377 | 3.72^{a} |
| 152 | 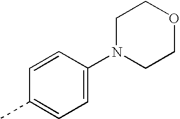 | Cyclohexyl-[3-(4- morpholin-4-yl- phenyl)-1H- pyrazolo[4,3- clpyridin-4-yl]- amine | 378 | 3.60^{a} |
| 153 | 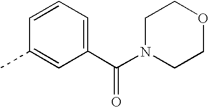 | [3-(4- Cyclohexylamino- 1H-pyrazolo[4,3- c]pyridin-3-yl)- phenyl]- morpholin-4-yl- methanone | 406 | 3.29^{a} |
| 154 | 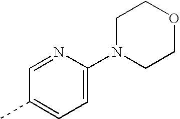 | Cyclohexyl-[3-(6- morphon-4-yl- pyridin-3-yl)-1H- pyrazolo[4,3- clpyridin-4-yl]- amine | 379 | 3.47^{a} |

^{a}HPLC column: 4.6 × 50 mm (5 μm) C-18 Xbridge; flow rate: 2 ml/min; Run time: 4.6 min: Solvent A: 0.1% Ammonium Hydroxide in water, Solvent B: Methanol; Gradient - 10-100% B; Gradient time: 3.5 min.
^{b}HPLC column: 4.6 × 50 mm (5 μm) C-18 Xbridge; flow rate: 3 ml/min; Run time: 3.2 min: Solvent A: 0.1% Ammonium Hydroxide in water Solvent B: Acetonitrile; Gradient- 10-100%B; Gradient time: 2.35 min.

### Example 155

### Cyclohexyl-[1-(4-methoxy-benzyl)-3-(3-pyrazol-1-yl-phenyl)-1H-pyrazolo[4,3-c]pyridin-4-yl]-amine

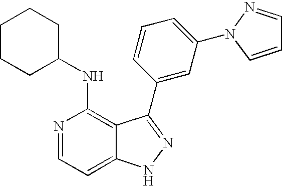

Intermediate 14 (50 mg, 0.11 mmol), Pd(dppf)Cl₂ (9 mg, 0.01 mmol), 1-[3-(4,4,5,5-tetramethyl-1,3,2-dioxaborolan-2-yl)phenyl]-1H-pyrazole (44 mg, 0.16 mmol) and 2 M aqueous sodium carbonate (189 μl, 0.38 mmol) were combined in dioxane (1 ml), the reaction mixture degassed and the vial flushed out with nitrogen. After heating to 90° C. for 18 h, the mixture was partitioned between DCM and water, the organic phase was collected using a phase separation cartridge and concentrated. The crude residue was treated with TFA (1 ml) at 60° C. for 19 h. Concentration under reduced pressure followed by purification by preparative LCMS (high pH buffer) gave the product (19 mg, 49%). ¹H NMR (400 MHz, DMSO-d₆) δ ppm 1.09-1.21 (m, 3H), 1.25-1.36 (m, 2H), 1.44-1.53 (m, 3H), 1.83-1.91 (m, 2H), 4.02-4.08 (m, 1H), 5.10 (d, J=7.3 Hz, 1H), 6.62 (d, J=1.8 Hz, 1H), 6.78 (d, J=6.0 Hz, 1H), 7.59-7.64 (m, 1H), 7.74 (t, J=7.8 Hz, 1H), 7.81-7.85 (m, 2H), 8.04 (d, J=9.2 Hz, 1H), 8.20 (s, 1H), 8.68 (d, J=2.3 Hz, 1H); m/z (ES+APCI)⁺: 359 [M+H]⁺.

### Examples 156-166

Examples 156-166 in the table below were prepared analogously to Example 155 from Intermediate 14 and the appropriate boronic acid or boronic ester:

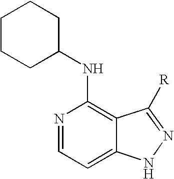

| | | | m/z | HPLC retention |
|---|---|---|---|---|
| Example | R group | Name | (ES + APCl)⁺ | time (min)* |
| 156 | 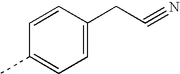 | [4-(4- Cyclohexylamino- 1H-pyrazolo[4,3- c]pyridin-3-yl)- phenyl]- acetonitrile | 332 | 1.77 |
| 157 | 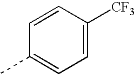 | Cyclohexyl-[1-(4- methoxy-benzyl)- 3-(4- trifluoromethyl- phenyl)-1H- pyrazolo[4,3- c]pyridin-4-yl]- amine | 361 | 2.20 |
| 158 | 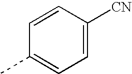 | 4-[4- Cyclohexylamino- 1-(4-methoxy- benzyl)-1H- pyrazolo[4,3- c]pyridin-3-yl]- benzonitrile | 318 | 1.87 |
| 159 | 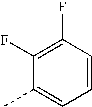 | Cyclohexyl-[3- (2,3-difluoro- phenyl)-1-(4- methoxy-benzyl)- 1H-pyrazolo[4,3- c]pyridin-4-yl]- amine | 329 | 1.97 |
| 160 | 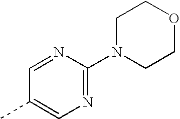 | Cyclohexyl-[3-(2- morpholin-4-yl- pyrimidin-5-yl)- 1H-pyrazolo[4,3- c]pyridin-4-yl]- amine | 380 | 1.73 |
| 161 | 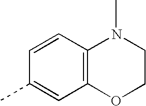 | Cyclohexyl-[3-(4- methyl-3,4- dihydro-2H-1,4- benzoxazin-7-yl)- 1H-pyrazolo[4,3- c]pyridin-4-yl]- amine | 364 | 1.94 |
| 162 | 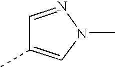 | Cyclohexyl-[3-(1- methyl-1H- pyrazol-4-yl)-1H- pyrazolo[4,3- c]pyridin-4-yl]- amine | 297 | 1.49 |
| 163 | 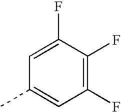 | Cyclohexyl-[3- (3,4,5-trifluoro- phenyl)-1H- pyrazolo[4,3- c]pyridin-4-yl]- amine | 347 | 2.20 |
| 164 | 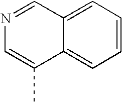 | Cyclohexyl-(3- isoquinolin-4-yl- 1H-pyrazolo[4,3- c]pyridin-4-yl)- amine | 344 | 1.74 |
| 165 | 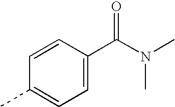 | 4-(4- Cyclohexylamino- 1H-pyrazolo[4,3- c]pyridin-3-yl)- N,N-dimethyl- benzamide | 364 | 1.60 |
| 166 | 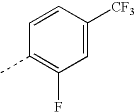 | Cyclohexyl-[3-(2- fluoro-4- trifluoromethyl- phenyl)-1H- pyrazolo[4,3- c]pyridin-4-yl]- amine | 379 | 222 |

*HPLC column: 4.6 × 50 mm (5 μm) C-18 Xbridge; flow rate: 3 ml/min; Run time: 3.2 min: Solvent A: 0.1% Ammonium Hydroxide in water Solvent B: Acetonitrile; Gradient - 10-100% B; Gradient time: 2.35 min.

### Example 167

### 3-Furan-3-yl-1H-pyrazolo[4,3-c]pyridin-4-ol

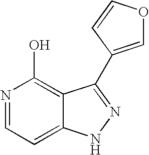

Intermediate 19 (50 mg, 0.11 mmol), Pd(dppf)Cl₂ (9 mg, 0.01 mmol), furan-3-boronic acid (18 mg, 0.16 mmol) and 2 M aqueous sodium carbonate (189 μl, 0.38 mmol) were combined in dioxane (1 ml), the reaction mixture was degassed and heated to 90° C., under nitrogen for 3 days. The mixture was partitioned between DCM and water, the organic phase was collected using a phase separation cartridge and concentrated. The crude residue was treated with TFA (1 ml) at 60° C. for 18 h. Concentration followed by purification by preparative LCMS yielded the title compound (3 mg, 14%). ¹H NMR (400 MHz, DMSO-d₆) δ ppm 6.45 (d, J=6.4 Hz, 1H), 7.09 (br. s., 1H), 7.13-7.22 (m, 1H), 7.77 (s, 1H), 8.87 (s, 1H), 11.00 (br. s., 1H); m/z (ES+APCI)⁺: 202 [M+H]⁺.

### Example 168

### 3-(4-Cyclohexylamino-1H-pyrazolo[4,3-c]pyridin-3-yl)-1-(4,4-difluoro-piperidin-1-yl)-propan-1-one

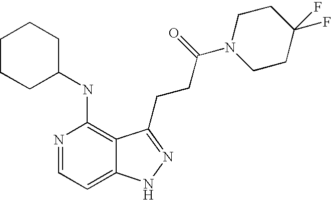

To a solution of Intermediate 23 (35 mg, 0.12 mmol) in DMF (1.5 ml) was added HATU (48 mg, 0.13 mmol) and N,N-diisopropylethylamine (126 μl, 0.73 mmol). 4,4-difluoro piperidine (19 μl, 0.18 mmol) was then added and the resulting solution was left to stir at room temperature overnight. The volatiles were removed under reduced pressure and the crude product was re-dissolved in 10% MeOH/DCM and eluted though an Isolute-NH₂ cartridge. The crude product purified by flash chromatography eluting with 10% MeOH/DCM to give a yellow gum (28 mg, 61%). ¹H NMR (400 MHz, DMSO-d₆) δ ppm 1.14-1.26 (m, 1H), 1.30-1.46 (m, 4H), 1.60-1.72 (m, 1H), 1.71-2.01 (m, 8H), 2.87 (t, 2H), 3.21 (t, J=6.4 Hz, 2H), 3.49-3.60 (m, 4H), 4.01 (br. s., 1H), 6.67 (br. s., 1H), 7.62 (d, J=6.0 Hz, 1H); m/z (ES+APCI)⁺: 392 [M+H]⁺.

### Examples 169-171

Examples 169-171 were prepared analogously to Example 167, (the general structure is shown below followed by the tabulated examples).

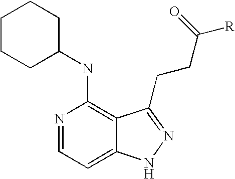

| | | | m/z | HPLC retention |
|---|---|---|---|---|
| Example | R group | Name | (ES + APCl)⁺ | time (min)* |
| 169 | 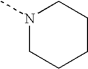 | 3-(4-Cyclohexylamino-1H- pyrazolo[4,3-c]pyridin-3-yl)-1- piperidin-1-yl-propan-1-one | 356 | 1.76 |
| 170 | 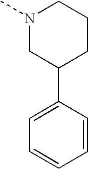 | 3-(4-Cyclohexylamino-1H- pyrazolo[4,3-c]pyridin-3-yl)-1- (3-phenyl-piperidin-1-yl)- propan-1-one | 432 | 2.09 |
| 171 | 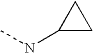 | 3-(4-Cyclohexylamino-1H- pyrazolo[4,3-c]pyridin-3-yl)-N- cyclopropyl-propionamide | 328 | 1.48 |

*HPLC column: 4.6 × 50 mm (5 μm) C-18 Xbridge; flow rate: 3 ml/min; Run time: 3.2 min: Solvent A: 0.1% Ammonium Hydroxide in water Solvent B: Acetonitrile; Gradient - 10-100% B; Gradient time: 2.35 min.

### Example 172

### 3-(4-Cyclohexylamino-1H-pyrazolo[4,3-c]pyridin-3-yl)-1-((R)-3-phenyl-piperidin-1-yl)-propan-1-one

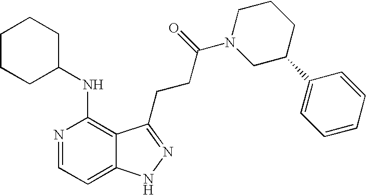

To a solution of Intermediate 23 (35 mg, 0.12 mmol) in DMF (1.5 ml) at room temperature was added HATU (48 mg, 0.13 mmol) and N,N-diisopropylethylamine (126 μl, 0.73 mmol). (R)-3-Phenylpiperidine (20 mg, 0.12 mmol) was then added, and the resulting solution was left to stir at room temperature overnight. The volatiles were removed under reduced pressure and the crude product was re-dissolved in 10% 1.5 MeOH/DCM and eluted though an Isolute-NH₂ cartridge. The solvents were removed and the crude product purified by preparative LCMS (high pH buffer) to give a white solid (4 mg, 8%). ¹H NMR (400 MHz, DMSO-d₆) δ ppm 1.13-1.43 (m, 6H), 1.50-1.76 (m, 5H), 1.70-1.87 (m, 1H), 1.97 (br. s., 2H), 2.32-2.48 (m, 1H), 2.52-2.68 (m, 1H), 2.68-2.84 (m, 2H), 2.94-3.12 (m, 1H), 3.12-3.31 (m, 2H), 3.77-3.97 (m, 1H), 3.97-4.10 (m, 1H), 4.38-4.53 (m, 1H), 6.13-6.40 (m, 1H), 6.51-6.61 (m, 1H), 7.08-7.35 (m, 5H), 7.61-7.71 (m, 1H); m/z (ES+APCI)⁺: 432 [M+H]⁺.

### Examples 173-209

Examples 173-209 in the table below were prepared analogously to Example 172 from Intermediate 23 and the appropriate amine:

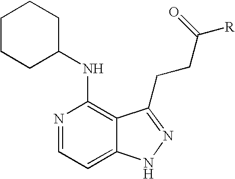

| | | | | HPLC |
|---|---|---|---|---|
| | | | | retention |
| | | | m/z | time |
| Example | R group | Name | (ES + APCI)⁺ | (min)* |
| 173 | 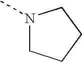 | 3-(4-Cyclohexylamino-1H- pyrazolo[4,3-c]pyridin-3-yl)- 1-pyrrolidin-1-yl-propan-1- one | 342 | 1.56 |
| 174 | 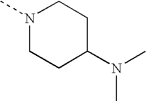 | 3-(4-Cyclohexylamino-1H- pyrazolo[4,3-c]pyridin-3-yl)- 1-(4-dimethylamino- piperidin-1-yl)-propan-1- one | 399 | 1.46 |
| 175 | 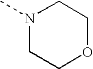 | 3-(4-Cyclohexylamino-1H- pyrazolo[4,3-c]pyridin-3-yl)- 1-morpholin-4-yl-propan-1- one | 358 | 1.46 |
| 176 | 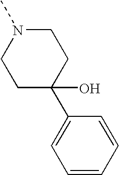 | 3-(4-Cyclohexylamino-1H- pyrazolo[4,3-c]pyridin-3-yl)- 1-(4-hydroxy-4-phenyl- piperidin-1-yl)-propan-1- one | 448 | 1.69 |
| 177 | 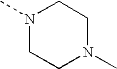 | 3-(4-Cyclohexylamino-1H- pyrazolo[4,3-c]pyridin-3-yl)- 1-(4-methyl-piperazin-1-yl)- propan-1-one | 371 | 1.40 |
| 178 | 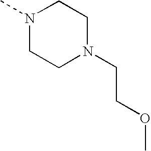 | 3-(4-Cyclohexylamino-1H- pyrazolo[4,3-c]pyridin-3-yl)- 1-[4-(2-methoxy-ethyl)- piperazin-1-yl]-propan-1- one | 415 | 1.44 |
| 179 | 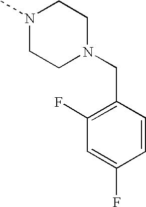 | 3-(4-Cyclohexylamino-1H- pyrazolo[4,3-c]pyridin-3-yl)- 1-[4-(2,4-difluoro-benzyl)- piperazin-1-yl]-propan-1- one | 483 | 1.94 |
| 180 | 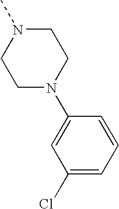 | 1-[4-(3-Chloro-phenyl)- piperazin-1-yl]-3-(4- cyclohexylamino-1H- pyrazolo[4,3-c]pyridin-3-yl)- propan-1-one | 467 | 2.12 |
| 181 | 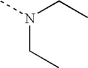 | 3-(4-Cyclohexylamino-1H- pyrazolo[4,3-c]pyridin-3-yl)- N,N-diethyl-propionamide | 344 | 1.71 |
| 182 | 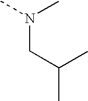 | 3-(4-Cyclohexylamino-1H- pyrazolo[4,3-c]pyridin-3-yl)- N-isobutyl-N-methyl- propionamide | 358 | 1.87 |
| 183 | 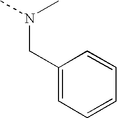 | N-Benzyl-3-(4- cyclohexylamino-1H- pyrazolo[4,3-c]pyridin-3-yl)- N-methyl-propionamide | 392 | 1.89 |
| 184 | 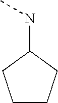 | 3-(4-Cyclohexylamino-1H- pyrazolo[4,3-c]pyridin-3-yl)- N-cyclopentyl- propionamide | 356 | 1.73 |
| 185 | 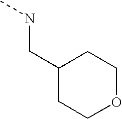 | 3-(4-Cyclohexylamino-1H- pyrazolo[4,3-c]pyridin-3-yl)- N-(tetrahydro-pyran-4- ylmethyl)-propionamide | 386 | 1.43 |
| 186 | 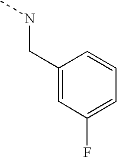 | 3-(4-Cyclohexylamino-1H- pyrazolo[4,3-c]pyridin-3-yl)- N-(3-fluoro-benzyl)- propionamide | 396 | 1.79 |
| 187 | 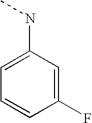 | 3-(4-Cyclohexylamino-1H- pyrazolo[4,3-c]pyridin-3-yl)- N-(3-fluoro-phenyl)- propionamide | 382 | 1.93 |
| 188 | 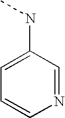 | 3-(4-Cyclohexylamino-1H- pyrazolo[4,3-c]pyridin-3-yl)- N-pyridin-3-yl- propion amide | 365 | 1.52 |
| 189 | 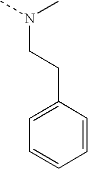 | 3-(4-Cyclohexylamino-1H- pyrazolo[4,3-c]pyridin-3-yl)- N-methyl-N-phenethyl- propionamide | 406 | 1.96 |
| 190 | 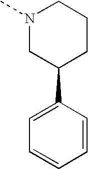 | 3-(4-Cyclohexylamino-1H- pyrazolo[4,3-c]pyridin-3-yl)- 1-((S)-3-phenyl-piperidin-1- yl)-propan-1-one | 432 | 2.09 |
| 191 | 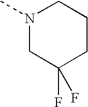 | 3-(4-Cyclohexylamino-1H- pyrazolo[4,3-c]pyridin-3-yl) 1-(3,3-difluoro-piperidin-1- yl)-propan-1-one | 392 | 1.82 |
| 192 | 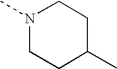 | 3-(4-Cyclohexylamino-1H- pyrazolo[4,3-c]pyridin-3-yl)- 1-(4-methyl-piperidin-1-yl)- propan-1-one | 370 | 1.93 |
| 193 | 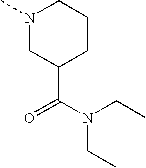 | 1-[3-(4-Cyclohexylamino- 1H-pyrazolo[4,3-c]pyridin- 3-yl)-propionyl]-piperidine- 3-carboxylic acid diethylamide | 455 | 1.64 |
| 194 | 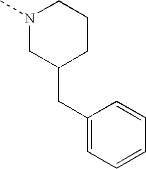 | 1-(3-Benzyl-piperidin-1-yl)- 3-(4-cyclohexylamino-1H- pyrazolo[4,3-c]pyridin-3-yl)- propan-1-one | 446 | 2.19 |
| 195 | 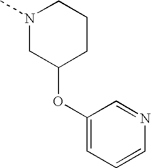 | 3-(4-Cyclohexylamino-1H- pyrazolo[4,3-c]pyridin-3-yl)- 1-[3-(pyridin-3-yloxy)- piperidin-1-yl]propan-1- one | 449 | 1.63 |
| 196 | 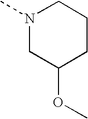 | 3-(4-Cyclohexylamino-1H- pyrazolo[4,3-c]pyridin-3-yl)- 1-(3-methoxy-piperidin-1- yl)-propan-1-one | 386 | 1.61 |
| 197 | 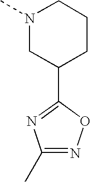 | 3-(4-Cyclohexylamino-1H- pyrazolo[4,3-c]pyridin-3-yl)- 1-[3-(3-methyl-1,2,4- oxadiazol-5-yl)-piperidin-1- yl]-propan-1-one | 438 | 1.67 |
| 198 | 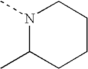 | 3-(4-Cyclohexylamino-1H- pyrazolo[4,3-c]pyridin-3-yl)- 1-(2-methyl-piperidin-1-yl)- propan-1-one | 370 | 1.86 |
| 199 | 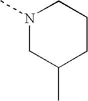 | 3-(4-Cyclohexylamino-1H- pyrazolo[4,3-c]pyridin-3-yl)- 1-(3-methyl-piperidin-1-yl)- propan-1-one | 370 | 1.88 |
| 200 | 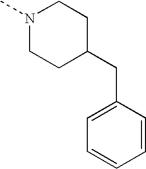 | 1-(3-Benzyl-piperidin-1-yl)- 3-(4-cyclohexylamino-1H- pyrazolo[4,3-c]pyridin-3-yl)- propan-1-one | 446 | 2.17 |
| 201 | 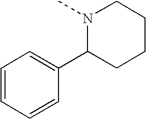 | 3-(4-Cyclohexylamino-1H- pyrazolo[4,3-c]pyridin-3-yl)- 1-(2-phenyl-piperidin-1-yl)- propan-1-one | 432 | 2.12 |
| 202 | 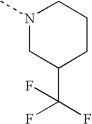 | 3-(4-Cyclohexylamino-1H- pyrazolo[4,3-c]pyridin-3-yl)- 1-(3-trifluoromethyl- piperidin-1-yl)-propan-1- one | 424 | 1.94 |
| 203 | 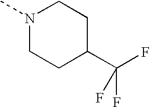 | 3-(4-Cyclohexylamino-1H- pyrazolo[4,3-c]pyridin-3-yl)- 1-(4-trifluoromethyl- piperidin-1-yl)-propan-1- one | 424 | 1.88 |
| 204 | 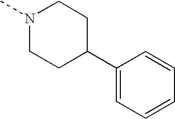 | 3-(4-Cyclohexylamino-1H- pyrazolo[4,3-c]pyridin-3-yl)- 1-(4-phenyl-piperidin-1-yl)- propan-1-one | 432 | 2.04 |
| 205 | 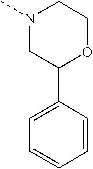 | 3-(4-Cyclohexylamino-1H- pyrazolo[4,3-c]pyridin-3-yl)- 1-(2-phenyl-morpholin-4- yl)-propan-1-one | 434 | 1.90 |
| 206 | 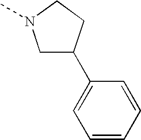 | 3-(4-Cyclohexylamino-1H- pyrazolo[4,3-c]pyridin-3-yl)- 1-(3-phenyl-pyrrolidin-1-yl)- propan-1-one | 418 | 1.94 |
| 207 | 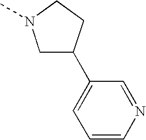 | 3-(4-Cyclohexylamino-1H- pyrazolo[4,3-c]pyridin-3-yl)- 1-(3-pyridin-3-yl-pyrrolidin- 1-yl)-propan-1-one | 419 | 1.50 |
| 208 | 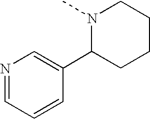 | 3-(4-Cyclohexylamino-1H- pyrazolo[4,3-c]pyridin-3-yl)- 1-(3,4,5,6-tetrahydro-2H- [2,3′]bipyridinyl-1-yl)- propan-1-one | 433 | 1.67 |
| 209 | 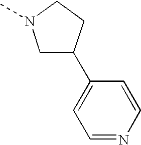 | 3-(4-Cyclohexylamino-1H- pyrazolo[4,3-c]pyridin-3-yl)- 1-(3-pyridin-4-yl-pyrrolidin- 1-yl)-propan-1-one | 419 | 1.48 |

*HPLC column: 4.6 × 50 mm (5 μm) C-18 Xbridge; flow rate: 3 ml/min; Run time: 3.2 min: Solvent A: 0.1% Ammonium Hydroxide in water Solvent B: Acetonitrile; Gradient—10-100% B; Gradient time: 2.35 min.

### Example 210

### 3-(4-Cyclohexylamino-1H-pyrazolo[4,3-c]pyridin-3-yl)-N-methyl-propionamide

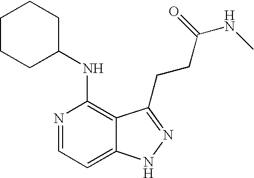

Intermediate 22 (80 mg, 0.26 mmol) was added to an excess of methylamine (33% in ethanol, 1 ml) at room temperature. The resulting mixture was irradiated at 150° C. for 1 h in a Biotage I-60 microwave reactor. The reaction mixture was then evaporated to dryness and the crude product purified by preparative LCMS (high pH buffer) to give the desired product (20 mg, 25%) ¹H NMR (400 MHz, CDCl₃) δ ppm 1.21-1.51 (m, 5H), 1.58-1.71 (m, 1H), 1.71-1.84 (m, 2H), 2.03-2.32 (m, 2H), 2.72 (t, J=6.9 Hz, 2H), 2.79 (d, J=5.0 Hz, 3H), 3.32 (t, J=7.1 Hz, 2H), 4.08-4.19 (m, 1H), 5.87 (br. s., 1H), 5.95-6.08 (m, 1H), 6.57 (d, J=6.0 Hz, 1H), 7.76-7.81 (m, 1H); m/z (ES+APCI)⁺: 302 [M+H]⁺.

### Example 211

### 3-(4-Cyclohexylamino-1H-pyrazolo[4,3-c]pyridin-3-yl)-N-(2-methoxy-ethyl)-propionamide

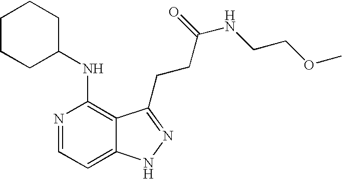

To a solution of Intermediate 22 (80 mg, 0.26 mmol) in ethanol (0.7 ml) at room temperature was added an excess of 2-methoxyethylamine (0.3 ml). The resulting mixture was irradiated at 150° C. for 1 h in a Biotage I-60 microwave reactor. Irradiation was continued at 190° C. for a further 30 mins, then the reaction mixture was evaporated to dryness and the crude product purified by mass triggered preparative LCMS (high pH buffer) to give the desired product (17 mg, 19%) ¹H NMR (400 MHz, MeOD) δ ppm 1.12-1.38 (m, 1H), 1.38-1.61 (m, 4H), 1.61-1.76 (m, 1H), 1.76-1.91 (m, 2H), 2.03-2.15 (m, 2H), 2.67 (t, J=7.1 Hz, 2H), 3.21 (s, 3H), 3.27 (t, J=7.1 Hz, 2H), 3.29-3.35 (m, 4H), 3.87-3.98 (m, 1H), 6.66 (d, J=6.0 Hz, 1H), 7.61 (d, 1H); m/z (ES+APCI)⁺: 346 [M+H]⁺.

### Example 212

### 3-(4-Cyclohexylamino-1H-pyrazolo[4,3-c]pyridin-3-yl)-N,N-dimethyl-propionamide

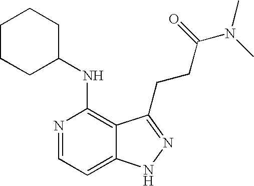

A solution of Intermediate 25 (115 mg, 0.27 mmol) in TFA (2 ml) was stirred at 70° C. for 4 h, and then allowed to cool to room temperature overnight. 2M NaOH (aq) was added and then the aqueous was extracted with EtOAc, dried (MgSO₄) and evaporated. The crude residue was purified by flash chromatography, eluting with 50% ethyl acetate/petroleum ether to 10% methanol/ethyl acetate gradient as a solid (84 mg, 100%). ¹H NMR (400 MHz, DMSO-d₆) δ ppm 1.15-1.28 (m, 1H), 1.31-1.52 (m, 4H), 1.60-1.68 (m, 1H), 1.72-1.81 (m, 2H), 1.95-2.05 (m, 2H), 2.76-2.88 (m, 5H), 2.96 (s, 3H), 3.18 (t, J=6.2 Hz, 2H), 3.87-3.97 (m, 1H), 6.79 (d, J=6.4 Hz, 1H), 7.60 (d, J=6.4 Hz, 1H).

### Example 213

### Cyclohexyl-(3-phenethyl-1H-pyrazolo[4,3-c]pyridin-4-yl)-amine

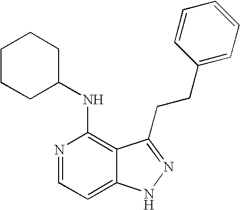

A solution of Intermediate 27 (140 mg, 0.32 mmol) in TFA (2 ml) was stirred at 70° C. for 2 h, and then allowed to cool to room temperature overnight. NH₃ (aq) was added slowly and then the aqueous was extracted with DCM, dried and evaporated. The crude residue was purified by mass triggered preparative LCMS (high Ph buffer) to give an off-white solid (36 mg, 36%) ¹H NMR (400 MHz, DMSO-d₆) δ ppm 1.15-1.24 (m, 1H), 1.32-1.41 (m, 4H), 1.58-1.65 (m, 1H), 1.68-1.76 (m, 2H), 1.94-2.02 (m, 2H), 2.97-3.03 (m, 2H), 3.27-3.30 (m, 2H), 4.03 (br. s., 1H), 5.47 (d, J=7.8 Hz, 1H), 6.58 (d, J=6.0 Hz, 1H), 7.17-7.22 (m, 1H), 7.26-7.32 (m, 4H), 7.68 (d, J=6.0 Hz, 1H); m/z (ES+APCI)⁺: 321 [M+H]⁺.

### Example 214

### Cyclohexyl-[3-(2-pyridin-2-yl-ethyl)-1H-pyrazolo[4,3-c]pyridin-4-yl]-amine

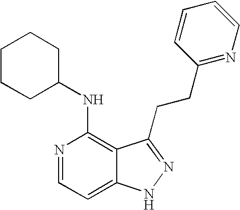

A solution of Intermediate 29 (80 mg, 0.32 mmol) in TFA (1.5 ml) was stirred at 70° C. for 24 h. The mixture was quenched by addition of ice, followed by 2M NaOH (aq) and NH₃ (aq). The aqueous was then extracted with DCM, dried and evaporated. The crude residue was purified by preparative LCMS (high pH buffer) to give the product as brown foam (6 mg, 10%). ¹H NMR (400 MHz, DMSO-d₆) δ ppm 1.17-1.26 (m, 1H), 1.30-1.44 (m, 4H), 1.60-1.66 (m, 1H), 1.71-1.78 (m, 2H), 1.98-2.04 (m, 2H), 3.11-3.17 (m, 2H), 3.33-3.40 (m, 2H), 4.07-4.14 (m, 1H), 5.90 (d, J=7.8 Hz, 1H), 6.57 (d, J=6.0 Hz, 1H), 7.23-7.27 (m, 1H), 7.32 (d, J=7.8 Hz, 1H), 7.66-7.74 (m, 2H), 8.55 (d, J=4.1 Hz, 1H). m/z (ES+APCI)⁺: 322 [M+H]⁺.

### Example 215

### Cyclohexyl-{3-[3-((S)-3-phenyl-piperidin-1-yl)-propyl]-1H-pyrazolo[4,3-c]pyridin-4-yl}-amine

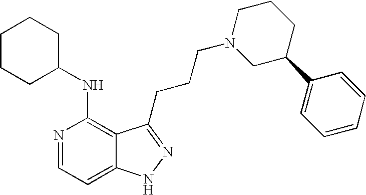

To Intermediate 35 (58 mg, 0.21 mmol) in acetonitrile (1 ml) at room temperature was added triphenylphosphine (83 mg, 0.32 mmol) and tetrabromomethane (105 mg, 0.32 mmol). The resulting mixture was stirred at room temperature for 2 hours. (S)-3-phenylpiperidine was added to this mixture and the resulting solution was irradiated at 100° C. for 30 mins in a Biotage I-60 microwave reactor. The reaction mixture was partitioned between water and DCM, and the organic phase was separated, dried and evaporated. The crude product purified by preparative LCMS (high pH buffer) to give the product as a gum (2 mg, 2%). ¹H NMR (400 MHz, DMSO-d₆) δ ppm 1.05-1.21 (m, 1H), 1.28-1.47 (m, 2H), 1.47-1.57 (m, 1H), 1.58-1.78 (m, 4H), 1.79-2.01 (m, 5H), 2.06-2.27 (m, 2H), 2.75-3.05 (m, 1H), 3.05-3.26 (m, 6H), 3.41-3.71 (m, 3H), 3.82-4.11 (m, 1H), 6.84 (br. s., 1H), 7.19-7.49 (m, 5H), 7.63 (d, J=6.0 Hz, 1H); m/z (ES+APCI)⁺: 418 [M+H]⁺.

### Example 216

### Cyclohexyl-{3-[2-(5-phenyl-1,3,4-oxadiazol-2-yl)-ethyl]-1H-pyrazolo[4,3-c]pyridin-4-yl}-amine

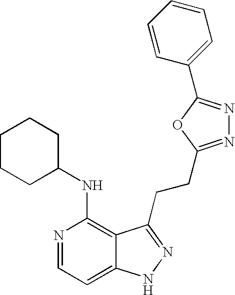

Prepared analogously to Intermediate 35 from Intermediate 33 to give the product as a white solid (4 mg, 26%). ¹H NMR (400 MHz, DMSO-d₆) δ ppm 1.13-1.25 (m, 1H), 1.28-1.45 (m, 4H), 1.58-1.65 (m, 1H), 1.69-1.77 (m, 2H), 1.94-2.02 (m, 2H), 3.40 (t, J=7.3 Hz, 2H), 3.58 (t, J=7.1 Hz, 2H), 4.01-4.10 (m, 1H), 5.73 (d, J=7.8 Hz, 1H), 6.59 (d, J=6.0 Hz, 1H), 7.56-7.65 (m, 3H), 7.69 (d, J=6.0 Hz, 1H), 7.92-7.97

### Example 217

### Cyclohexyl-[3-(2-piperidin-4-yl-ethyl)-1H-pyrazolo[4,3-c]pyridin-4-yl]-amine

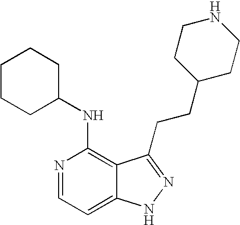

To a stirred solution of Intermediate 36 (23 mg, 0.07 mmol) in acetic acid (2 ml) at room temperature was added platinum oxide (10 mg). The resulting mixture was stirred under an atmosphere of hydrogen at room temperature overnight. The reaction mixture was then filtered through Celite™ and evaporated. The crude product was purified by preparative LCMS (high pH buffer) to give the desired product (4 mg, 17%). ¹H NMR (400 MHz, CDCl₃) δ ppm 1.10-1.36 (m, 3H), 1.36-1.61 (m, 5H), 1.62-1.91 (m, 7H), 2.00-2.21 (m, 2H), 2.66-2.87 (m, 2H), 2.87-3.14 (m, 2H), 3.14-3.39 (m, 2H), 4.10-4.24 (m, 1H), 4.72 (d, J=7.8 Hz, 1H), 6.60 (d, J=6.0 Hz, 1H), 7.86 (d, J=6.0 Hz, 1H); m/z (ES+APCI)⁺: 328 [M+H]⁺.

### Example 218

### Cyclohexyl-[3-(2-pyridin-4-yl-ethyl)-1H-pyrazolo[4,3-c]pyridin-4-yl]-amine

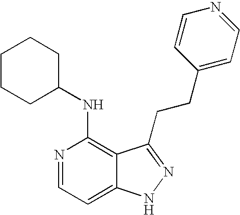

Prepared analogously to Intermediate 35 from Intermediate 37 to give the product as a white solid (4 mg, 26%). ¹H NMR (400 MHz, DMSO-d₆) δ ppm 1.10-1.24 (m, 1H), 1.24-1.50 (m, 4H), 1.50-1.66 (m, 1H), 1.66-1.81 (m, 2H), 1.89-2.04 (m, 2H), 2.98-3.19 (m, 2H), 3.19-3.59 (m, 2H), 3.96-4.10 (m, 1H), 5.54 (d, J=7.8 Hz, 1H), 6.57 (d, J=6.0 Hz, 1H), 7.29 (d, J=6.0 Hz, 2H), 7.66 (d, J=6.0 Hz, 1H), 8.42-8.48 (m, 2H); m/z (ES+APCI)⁺: 322 [M+H]⁺.

### Example 219

### 1-{4-[2-(4-Cyclohexylamino-1H-pyrazolo[4,3-c]pyridin-3-yl)-ethyl]piperidin-1-yl}-ethanone

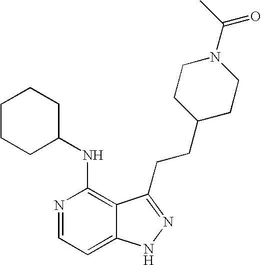

To a stirred solution of Intermediate 38 (45 mg, 0.1 mmol) and triethylamine (21 μl, 0.15 mmol) in DCM (2 ml) at 0° C. was added acetyl chloride (8 μl). The resulting mixture was allowed to warm to room temperature over 2 h. Water was added and the organic phase was collected using a phase separating cartridge and evaporated. TFA (1.5 ml) was added to the crude residue and the resulting solution was stirred at 70° C. overnight. The reaction mixture was evaporated and then partitioned between DCM, and saturated NaHCO₃ (aq). The organic phase was separated and dried using a phase separation tube and then evaporated. The crude residue was purified by preparative LCMS (high pH buffer) to give a white solid (25 mg, 67%) ¹H NMR (400 MHz, DMSO-d₆) δ ppm 0.92-1.31 (m, 3H), 1.34-1.47 (m, 2H), 1.50-1.89 (m, 10H), 1.94-2.02 (m, 5H), 2.93-3.04 (m, 1H), 3.16 (t, J=7.3 Hz, 2H), 3.29 (br. s., 1H), 3.75-3.90 (m, 2H), 4.31-4.39 (m, 1H), 6.98 (d, J=7.3 Hz, 1H), 7.57 (d, J=6.9 Hz, 1H); m/z (ES+APCI)⁺: 370 [M+H]⁺.

### Example 220

### Cyclohexyl-{3-[2-(1-methanesulfonyl-piperidin-4-yl)-ethyl]-1H-pyrazolo[4,3-c]pyridin-4-yl}-amine

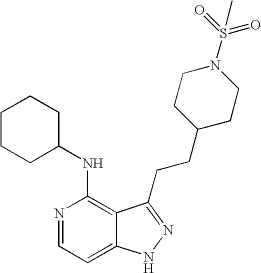

To a stirred solution of Intermediate 38 (45 mg, 0.1 mmol) and triethylamine (21 μl, 0.15 mmol) in DCM (2 ml) at 0° C. was added methanesulfonyl chloride (8.6 μl). The resulting mixture was allowed to warm to room temperature over 2 h. Water was added and the organic phase was collected using a phase separating tube and evaporated. TFA (1.5 ml) was added to the crude residue and the resulting solution was stirred at 70° C. overnight. The reaction mixture was evaporated and then partitioned between DCM, and saturated NaHCO₃ (aq). The organic phase was separated and dried using a phase separation cartridge and then evaporated. The crude residue was purified by preparative LCMS (high pH buffer) to give a white solid (11 mg, 26%) ¹H NMR (400 MHz, DMSO-d₆) δ ppm 1.15-1.27 (m, 3H), 1.28-1.46 (m, 5H), 1.58-1.69 (m, 3H), 1.69-1.79 (m, 2H), 1.80-1.88 (m, 2H), 1.92-2.02 (m, 2H), 2.60-2.69 (m, 2H), 2.83 (s, 3H), 2.99-3.07 (m, 2H), 3.50-3.57 (m, 2H), 3.97-4.06 (m, 1H), 5.44 (br. s., 1H), 6.59 (d, J=6.0 Hz, 1H), 7.66 (d, J=6.0 Hz, 1H); m/z (ES+APCI)⁺: 406 [M+H]⁺.

### Example 221

### {3-[2-(3-Amino-phenyl)-ethyl]-1H-pyrazolo[4,3-c]pyridin-4-yl}-cyclohexyl-amine

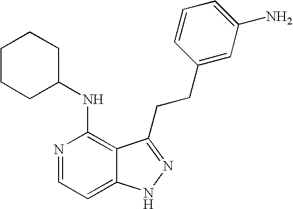

To Intermediate 40 (0.48 g, 1.3 mmol) in ethanol (10 ml) at room temperature was added 10% Pd/C (90 mg). The resulting mixture was stirred under an atmosphere of hydrogen at room temperature overnight. The reaction mixture was then filtered through Celite™ and evaporated. The crude residue was then purified by flash chromatography, eluting with ethyl acetate to 10% MeOH/ethyl acetate gradient to give a gum (0.21 g, 47%). ¹H NMR (400 MHz, DMSO-d₆) δ ppm 1.14-1.25 (m, 1H), 1.25-1.47 (m, 4H), 1.55-1.65 (m, 1H), 1.65-1.77 (m, 2H), 1.93-2.04 (m, 2H), 2.78-2.85 (m, 2H), 3.15-3.27 (m, 2H), 3.97-4.10 (m, 1H), 4.96 (s, 2H), 5.38 (d, J=7.8 Hz, 1H), 6.38-6.48 (m, 3H), 6.58 (d, J=6.0 Hz, 1H), 6.93 (t, J=7.8 Hz, 1H), 7.67 (d, J=6.4 Hz, 1H); m/z (ES+APCI)⁺: 336 [M+H]⁺.

### Example 222

### N-{3-[2-(4-Cyclohexylamino-1H-pyrazolo[4,3-c]pyridin-3-yl)-ethyl]-phenyl}-acetamide

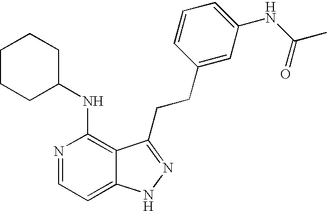

To a solution of acetic acid (7 μl, 0.12 mmol) in DMF (1 ml) at room temperature was added HATU (48 mg, 0.12 mmol) and N,N-diisopropylethylamine (125 μl, 0.72 mmol). Example 221 (40 mg, 0.12 mmol) was then added and the resulting solution was left to stir at room temperature overnight. The reaction mixture was diluted with DCM and washed with saturated NaHCO₃ (aq). The organic phase was separated and dried using a phase separation cartridge and the crude product purified by preparative LCMS (low pH buffer) to give the product as a white solid (4.5 mg, 10%). ¹H NMR (400 MHz, DMSO-d₆) δ ppm 1.13-1.26 (m, 1H), 1.28-1.42 (m, 4H), 1.57-1.66 (m, 1H), 3.31 (m, 2H), 3.97-4.07 (m, 1H), 5.42 (d, J=7.8 Hz, 1H), 6.58 (d, J=6.0 Hz, 1H), 6.93 (d, J=7.8 Hz, 1H), 7.20 (t, J=7.8 Hz, 1H), 7.41 (d, J=7.8 Hz, 1H), 7.49 (s, 1H), 7.67 (d, J=6.0 Hz, 1H), 9.87 (s, 1H); m/z (ES+APCI)⁺: 378 [M+H]⁺.

### Examples 223-228

Examples 223-228 in the table below were prepared analogously to Example 222:

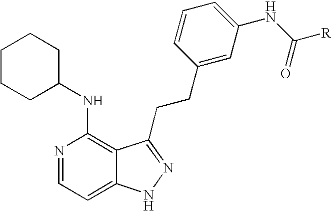

| | | | | HPLC |
|---|---|---|---|---|
| | | | | retention |
| | | | m/z | time |
| Example | R group | Name | (ES + APCl)⁺ | (min)* |
| 223 | 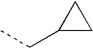 | N-{3-[2-(4- Cyclohexylamino-1H- pyrazolo[4,3-c]pyridin-3-yl)- ethyl]-phenyl}-2- cyclopropyl-acetamide | 418 | 1.88 |
| 224 | 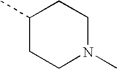 | 1-Methyl-piperidine-4- carboxylic acid {3-[2-(4- cyclohexylamino-1H- pyrazolo[4, 3-c]pyridin-3-yl)- ethyl]-phenyl}-amide | 461 | 1.70 |
| 225 | 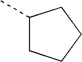 | Cyclopentanecarboxylic acid {3-[2-(4- cyclohexylamino-1H- pyrazolo[4,3-c]pyridin-3-yl)- ethyl]-phenyl}-amide | 432 | 2.02 |
| 226 | 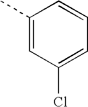 | 3-Chloro-N-{3-[2-(4- cyclohexylamino-1H- pyrazolo[4,3-c]pyridin-3-yl)- ethyl]-phenyl}-benzamide | 474 | 2.16 |
| 227 | 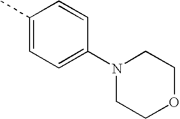 | N-{3-[2-(4- Cyclohexylamino-1H- pyrazolo[4,3-c]pyridin-3-yl)- ethyl]-phenyl}-4-morpholin- 4-yl-benzamide | 525 | 1.92 |
| 228 | 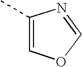 | Oxazole-4-carboxylic acid {3-[2-(4-cyclohexylamino- 1H-pyrazolo[4,3-c]pyridin- 3-yl)-ethyl]-phenyl}-amide | 431 | 1.78 |

*HPLC column: 4.6 × 50 mm (5 μm) C-18 Xbridge; flow rate: 3 ml/min; Run time: 3.2 min: Solvent A: 0.1% Ammonium Hydroxide in water Solvent B: Acetonitrile; Gradient - 10-100% B; Gradient time: 2.35 min.

### Example 229

### {4-[2-(4-Cyclohexylamino-1H-pyrazolo[4,3-c]pyridin-3-yl)-ethyl]phenyl}-(4-methyl-piperidin-1-yl)-methanone

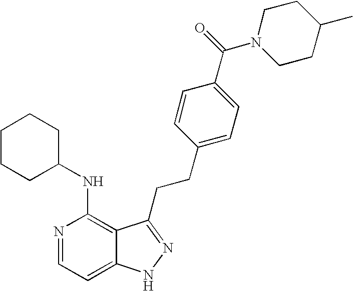

Prepared analogously to Example 172 from Intermediate 44 to give the desired product as a white solid (16 mg, 43%). ¹H NMR (400 MHz, DMSO-d₆) δ ppm 0.92 (d, J=6.0 Hz, 3H), 0.98-1.25 (m, 3H), 1.26-1.51 (m, 4H), 1.51-1.79 (m, 6H), 1.92-2.02 (m, 2H), 2.72 (br. s., 1H), 2.97-3.11 (m, 3H), 3.27-3.31 (m, 2H), 3.57 (br. s., 1H), 3.98-4.09 (m, 1H), 4.42 (br. s., 1H), 5.53 (d, J=7.8 Hz, 1H), 6.58 (d, J=6.0 Hz, 1H), 7.25-7.35 (m, 4H), 7.67 (d, J=6.0 Hz, 1H); m/z (ES+APCI)⁺: 446 [M+H]⁺.

### Example 230

### 1-(4-Cyclohexylamino-1H-pyrazolo[4,3-c]pyridin-3-yl)-pyrrolidin-2-one

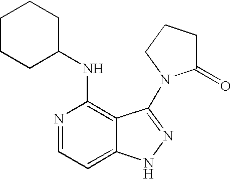

To a mixture of Intermediate 14 (100 mg, 0.2 mmol), copper iodide (10 mg, 0.054 mmol), N,N-dimethylethylenediamine (12 μl, 0.1 mmol) and potassium carbonate (45 mg, 0.32 mmol) in DMF (5 ml) at room temperature was added pyrrolidinone (25 μl, 0.32 mmol). The resulting mixture was irradiated at 150° C. for 30 mins in a Biotage I-60 microwave reactor. The reaction mixture was then partitioned between water and DCM, and the organic phase was collected, dried and evaporated. TFA (1 ml) was added to the crude residue and the resulting solution was stirred at 70° C. overnight. The reaction mixture was evaporated and then partitioned between DCM, and saturated Na₂CO₃ (aq). The organic phase was separated, dried and then evaporated. The crude residue was purified by preparative LCMS (high pH buffer) to give the product as a white solid (13 mg, 20%). ¹H NMR (400 MHz, DMSO-d₆) δ ppm 1.14-1.42 (m, 5H), 1.52-1.63 (m, 1H), 1.63-1.79 (m, 2H), 1.89-2.01 (m, 2H), 2.17 (quin, J=7.6 Hz, 2H), 2.59 (t, J=8.0 Hz, 2H), 3.89-4.03 (m, 3H), 6.33 (d, J=7.3 Hz, 1H), 6.60 (d, J=6.0 Hz, 1H), 7.71 (d, J=6.0 Hz, 1H); m/z (ES+APCI)⁺: 300 [M+H]⁺.

### Example 231

### 3-(4-Cyclohexylamino-1H-pyrazolo[4,3-c]pyridin-3-yl)-propan-1-ol

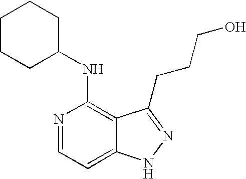

A solution of Intermediate 30 (0.74 g, 1.9 mmol) in TFA (5 ml) was stirred at 70° C. overnight. The reaction mixture was cooled to room temperature and then diluted with DCM, and saturated Na₂CO₃ (aq) was added. The organic phase was separated, filtered through a phase separation cartridge and evaporated. The crude residue was purified by flash chromatography, eluting with 20% ethyl acetate/petroleum ether to 10% methanol/ethyl acetate gradient to give a gum (0.47 g, 91%). ¹H NMR (400 MHz, DMSO-d₆) δ ppm 1.13-1.26 (m, 1H), 1.32-1.58 (m, 4H), 1.02-1.70 (m, 1H), 175-1.88 (m, 4H), 1.93-2.03 (m, 2H), 3.04-3.14 (m, 2H), 3.14-3.25 (m, 1H), 3.48 (t, J=5.7 Hz, 2H), 3.79-3.88 (m, 1H), 5.20 (br. s., 1H), 6.98 (d, 1H), 7.52-7.68 (m, 1H); m/z (ES+APCI)⁺: 275 [M+H]⁺.

### Example 232

### 4-[2-(4-Cyclohexylamino-1H-pyrazolo[4,3-c]pyridin-3-yl)-ethyl]-benzoic acid methyl ester

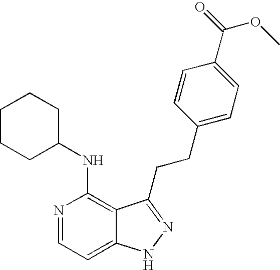

Prepared analogously to Intermediate 35 from Intermediate 42 to give the product (as a brown solid) (0.12 g, 86%). 1H NMR (400 MHz, DMSO-d₆) δ ppm 1.13-1.24 (m, 1H), 1.27-1.41 (m, 4H), 1.58-1.66 (m, 1H), 1.07-1.76 (m, 2H), 1.92-2.01 (m, 2H), 3.06-3.12 (m, 2H), 3.34-3.42 (m, 2H), 3.83 (s, 3H), 3.99-4.07 (m, 1H), 5.52 (d, J=7.8 Hz, 1H), 6.58 (d, J=6.0 Hz, 1H), 7.42 (d, J=8.2 Hz, 2H), 7.67 (d, J=6.0 Hz, 1H), 7.88 (d, J=8.7 Hz, 2H); m/z (ES+APCI)⁺: 3790 [M+H]⁺.

### Example 233

### Cyclohexyl-{3-[1-(2-morpholin-4-yl-ethyl)-1H-pyrazol-4-yl]-1H-pyrazolo[4,3-c]pyridin-4-yl}-amine

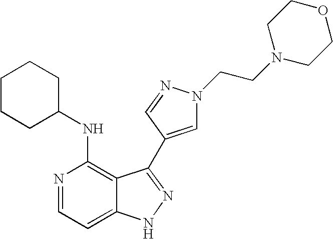

Example 233 was prepared analogously to Example 155 from Intermediate 14 and 1-(2-morpholinoethyl)-1H-pyrazole-4-boronic acid pinacol ester to give the product as a brown solid (20 mg, 39%). ¹H NMR (400 MHz, DMSO-d₆) δ ppm 1.16-1.29 (m, 3H), 1.32-1.44 (m, 2H), 1.54-1.67 (m, 3H), 1.94-2.02 (m, 2H), 2.42-2.52 (m, 4H), 2.82 (t, J=6.6 Hz, 2H), 3.57-3.63 (m, 4H), 3.98-4.06 (m, 1H), 4.38 (t, J=6.4 Hz, 2H), 5.19 (d, J=6.9 Hz, 1H), 6.71 (d, J=6.0 Hz, 1H), 7.74-7.79 (m, 2H), 8.17 (s, 1H); m/z (ES+APCI)⁺: 396 [M+H]⁺.

### Examples 234-235

Examples 234-235 in the following table were prepared analogously to Example 139 from Intermediate 14 and the appropriate boronic acid or boronic ester (the general structure is shown below followed by the tabulated examples).

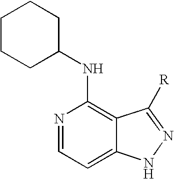

| | | | m/z | HPLC retention |
|---|---|---|---|---|
| Example | R group | Name | (ES + APCl)⁺ | time (min)* |
| 234 | 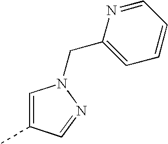 | Cyclohexyl-[3-(1- pyridin-2- ylmethyl-1H- pyrazol-4-yl)-1H- pyrazolo[4,3- clpyridin-4-yl]- amine | 374 | 1.53 |
| 235 | 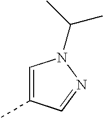 | Cyclohexyl-[3-(1- isopropyl-1H- pyrazol-4-yl)-1H- pyrazolo[4,3- cipyridin-4-yl]- amine | 325 | 1.69 |

HPLC column: 4.6 × 50 mm (5 μm) C-18 Xbridge; flow rate: 3 ml/min; Run time: 3.2 min: Solvent A: 0.1% Ammonium Hydroxide in water Solvent B: Acetonitrile; Gradient - 10-100% B; Gradient time: 2.35 min.

### Example 236

### 4-Cyclohexyloxy-3-furan-3-yl-1H-pyrazolo[4,3-c]pyridine

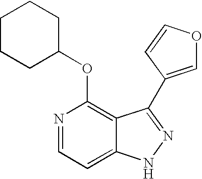

### Step 1

### 4-Cyclohexyloxy-3-furan-3-yl-1-trityl-1H-pyrazolo[4,3-c]pyridine

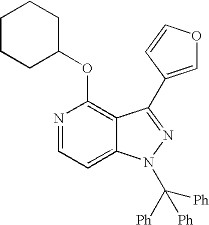

Intermediate 46 (96 mg, 0.16 mmol), Pd(dppf)Cl₂ (13 mg, 0.02 mmol), furan-3-boronic acid 28 mg, 0.25 mmol) and 2 M sodium carbonate (287 μl, 0.58 mmol) were combined in dioxane (1 ml), the solution degassed with nitrogen and then heated to 90° C. for 18 h. After evaporating the solvents, the crude material was re-dissolved in 1:1 DCM:MeOH, then dry loaded onto silica and purified by flash chromatography using a Biotage SP4 (ethyl acetate/petroleum ether gradient) to give a white solid (69 mg, 80%). ¹H NMR (400 MHz, DMSO-d₆) δ ppm 1.23-1.49 (m, 3H), 1.55-1.66 (m, 3H), 1.80 (m, J=8.7, 4.1 Hz, 2H), 2.11-2.19 (m, 2H), 5.22-5.29 (m, 1H), 5.78 (d, J=6.0 Hz, 1H), 6.79 (d, J=1.8 Hz, 1H), 7.14-7.26 (m, 5H), 7.27-7.44 (m, 10H), 7.59 (d, J=6.4 Hz, 1H), 7.76-7.78 (m, 1H), 8.27-8.38 (m, 1H); m/z (ES+APCI)⁺: 526 [M+H]⁺.

### Step 2

The product of step 1 (42 mg, 0.08 mmol) was dissolved in 2:8 TFA:DCM mixture and stirred at room temperature for 4 h. The solvents were evaporated and the crude material purified by preparative LCMS (high pH buffer) to give a white solid (2.8 mg, 12%). ¹H NMR (400 MHz, DMSO-d₆) δ ppm 1.41 (t, J=1.0 Hz, 3H), 1.54-1.68 (m, 3H), 1.76-1.85 (m, 2H), 2.12-2.21 (m, 2H), 5.20-5.41 (m, 1H), 7.08 (d, J=0.9 Hz, 1H), 7.11 (d, J=6.0 Hz, 1H), 7.81-7.82 (m, 1H), 7.89 (d, J=6.4 Hz, 1H), 8.38-8.39 (m, 1H); m/z (ES+APCI)⁺: 284 [M+H]⁺.

### Example 237

### 4-Cyclohexyloxy-3-pyrrolidin-1-yl-1H-pyrazolo[4,3-c]pyridine

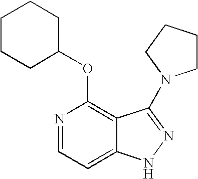

### Step 1

### 4-Cyclohexyloxy-3-pyrrolidin-1-yl-1-trityl-1H-pyrazolo[4,3-c]pyridine

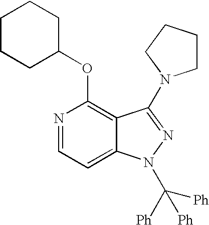

Intermediate 46 (203 mg, 0.35 mmol), pyrrolidine (327 μl, 3.99 mmol), Pd₂(dba)₃ (32 mg, 0.03 mmol), xantphos (12 mg, 0.02 mmol) and sodium t-butoxide (50 mg, 0.52 mmol) were combined in dioxane (3 ml). The solvent was degassed, the vial flushed out with nitrogen and the solution heated to 90° C. for 18 h. The solvents were evaporated, the crude material re-dissolved in 1:9 MeOH:DCM, directly dry loaded onto silica and purified by flash chromatography using a Biotage SP4 (ethyl acetate/petroleum ether gradient) to give a yellow solid (142 mg, 78%). ¹H NMR (400 MHz, DMSO-d₆) δ ppm 1.26-1.46 (m, 4H), 1.47-1.63 (m, 3H), 1.72-1.81 (m, 2H), 1.84-1.95 (m, 3H), 2.02-2.10 (m, 2H), 3.39-3.44 (m, 4H), 5.17-5.24 (m, 1H), 5.63 (d, J=6.0 Hz, 1H), 7.17-7.26 (m, 5H), 7.29-7.38 (m, 10H), 7.45 (d, J=6.4 Hz, 1H); m/z (ES+APCI)⁺: 529 [M+H]⁺.

### Step 2

The product of step 1 (80 mg, 0.15 mmol) was dissolved in 1:9 TFA:DCM mixture and stirred at room temperature for 1.5 h. The solvents were evaporated and the crude material purified by flash chromatography using a Biotage Isolera 4 (ethyl acetate/petroleum ether gradient). The material was then further purified by preparative LCMS (high pH buffer) to give the product (1.5 mg, 3%). ¹H NMR (400 MHz, DMSO-d₆) δ ppm 1.24-1.67 (m, 8H), 1.71-1.83 (m, 2H), 1.86-1.98 (m, 4H), 2.02-2.13 (m, 2H), 3.35-3.41 (m, 2H), 5.13-5.33 (m, 1H), 6.86 (d, J=6.0 Hz, 1H), 7.73 (d, J=6.0

### Examples 238-246

Examples 238-246 in the table below were prepared analogously to Example 168 from Intermediate 23 and the corresponding amine.

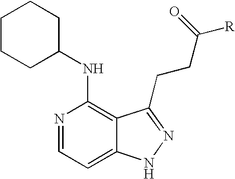

| | | | | HPLC |
|---|---|---|---|---|
| | | | m/z | retention |
| Example | R | Name | (ES + APCl)⁺ | time (min)* |
| 238 | 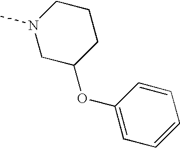 | 3-(4- Cyclohexylamino-1H- pyrazolo[4,3- c]pyridin-3-yl)-1-(3- phenoxy-piperidin-1- yl)-propan-1-one | 448 | 2.04 |
| 239 | 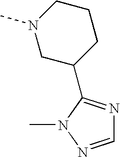 | 3-(4- Cyclohexylamino-1H- pyrazolo[4,3- c]pyridin-3-yl)-1-[3-(2- methyl-2H-1,2,4- triazol-3-yl)-piperidin- 1-yl]-propan-1-one | 437 | 1.33 |
| 240 | 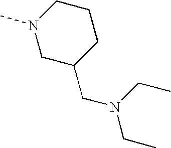 | 3-(4- Cyclohexylamino-1H- pyrazolo[4,3- c]pyridin-3-yl)-1-(3- diethylaminomethyl- piperidin-1-yl)- propan-1-one | 441 | 2.04 |
| 241 | 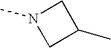 | 3-(4- Cyclohexylamino-1H- pyrazolo[4,3- c]pyridin-3-yl)-1-(3- methyl-azetidin-1-yl)- propan-1-one | 342 | 1.54 |
| 242 | 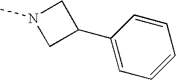 | 3-(4- Cyclohexylamino-1H- pyrazolo[4,3- c]pyridin-3-yl)-1-(3- phenyl-azetidin-1-yl)- propan-1-one | 404 | 1.80 |
| 243 | 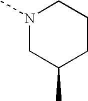 | 3-(4- Cyclohexylamino-1H- pyrazolo[4,3- c]pyridin-3-yl)-1-((R)- 3-methyl-piperidin-1- yl)-propan-1-one | 370 | 1.88 |
| 244 | 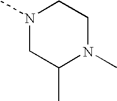 | 3-(4- Cyclohexylamino-1H- pyrazolo[4,3- c]pyridin-3-yl)-1-(3,4- dimethyl-piperazin-1- yl)-propan-1-one | 385 | 1.44 |
| 245 | 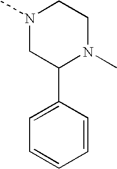 | 3-(4- Cyclohexylamino-1H- pyrazolo[4,3- c]pyridin-3-yl)-1-(4- methyl-3-phenyl- piperazin-1-yl)- propan-1-one | 447 | 1.82 |
| 246 | 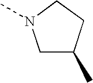 | 3-(4- Cyclohexyla mino-1H- pyrazolo[4,3- c]pyridin-3-yl)-1-((R)- 3-methyl-pyrrolidin-1- yl)-propan-1-one | 356 | 1.69 |

*HPLC column: 4.6 × 50 mm (5 μm) C-18 Xbridge; flow rate: 3 ml/min; Run time: 3.2 min: Solvent A: 0.1% Ammonium Hydroxide in water Solvent B: Acetonitrile; Gradient - 10-100% B; Gradient time: 2.35 min.

### Examples 247-256

Examples 247-256 in the table below were prepared analogously to Example 229, from Intermediate 44 and the appropriate amine.

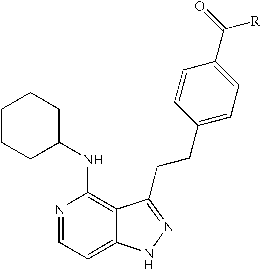

| | | | | HPLC |
|---|---|---|---|---|
| | | | m/z | retention |
| Example | R group | Name | (ES + APCl)⁺ | time (min)* |
| 247 | 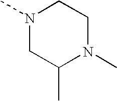 | {4-[2-(4- Cyclohexylamino-1H- pyrazolo[4,3-c]pyridin-3- yl)-ethyl]phenyl)-(3,4- dimethyl-piperazin-1-yl)- methanone | 461 | 1.60 |
| 248 | 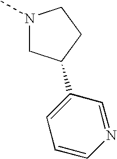 | {4-[2-(4- Cyclohexylamino-1H- pyrazolo[4,3-c]pyridin-3- yl)-ethyl]-phenyl}-((R)-3- pyridin-3-yl-pyrrolidin-1- yl)-methanone | 495 | 1.66 |
| 249 | 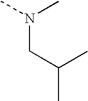 | 4-[2-(4-Cyclohexylamino- 1H-pyrazolo[4,3- c]pyridin-3-yl)-ethyl]-N- isobutyl-N-methyl- benzamide | 434 | 2.00 |
| 250 | 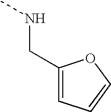 | 4-[2-(4-Cyclohexylamino- 1H-pyrazolo[4,3- c]pyridin-3-yl)-ethyl]-N- furan-2-ylmethyl- benzamide | 444 | 1.83 |
| 251 | 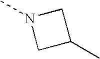 | {4-[2-(4- Cyclohexylamino-1H- pyrazolo[4,3-c]pyridin-3- yl)-ethyl]-phenyl}-(3- methyl-azetidin-1-yl)- methanone | 418 | 1.79 |
| 252 | 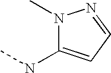 | 4-[2-(4-Cyclohexylamino- 1H-pyrazolo[4,3- c]pyridin-3-yl)-ethyl]-N-(2- methyl-2H-pyrazol-3-yl)- benzamide | 444 | 1.63 |
| 253 | 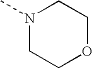 | {4-[2-(4- Cyclohexylamino-1H- pyrazolo[4,3-c]pyridin-3- yl)-ethyl]-phenyl}- morpholin-4-yl- methanone | 434 | 1.64 |
| 254 | 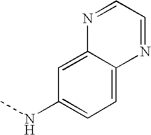 | 4-[2-(4-Cyclohexylamino- 1H-pyrazolo[4,3- c]pyridin-3-yl)-ethyl]-N- quinoxalin-6-yl- benzamide | 492 | 1.78 |
| 255 | 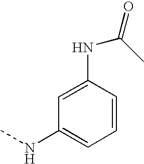 | N-(3-Acetylamino- phenyl)-4-[2-(4- cyclohexylamino-1H- pyrazolo[4,3-c]pyridin-3- yl)-ethyl]-benzamide | 497 | 1.68 |
| 256 | 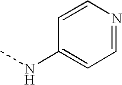 | 4-[2-(4-Cyclohexylamino- 1H-pyrazolo[4,3- c]pyridin-3-yl)-ethyl]-N- pyridin-4-yl-benzamide | 441 | 1.72 |

*HPLC column: 4.6 × 50 mm (5 μm) C-18 Xbridge; flow rate: 3 ml/min; Run time: 3.2 min: Solvent A: 0.1% Ammonium Hydroxide in water Solvent B: Acetonitrile; Gradient - 10-100% B; Gradient time: 2.35 min.

### Example 257

### 3-(4-Hydroxy-1H-pyrazolo[4,3-c]pyridin-3-yl)-1-((R)-3-phenyl-piperidin-1-yl)-propan-1-one

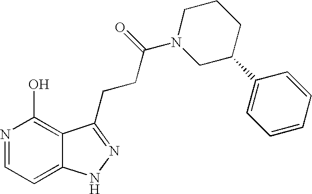

A solution of Intermediate 50 (70 mg, 0.14 mmol) in TFA (1 ml) was stirred at 50° C. for 4 h, and then allowed to cool to room temperature overnight. A further 1 ml of TFA was added, and mixture heated at 60° C. overnight, then evaporated. The crude residue was partitioned between saturated Na₂CO₃ (aq) and DCM. The organic phase was collected and dried (MgSO₄) using a phase separation tube and then evaporated. The crude residue was purified by preparative LCMS (high pH buffer) to give the product as a white solid (9 mg, 17%). ¹H NMR (400 MHz, CDCl₃) δ ppm 1.47-1.89 (m, 3H), 1.96-2.08 (m, 1H), 2.41-2.70 (m, 2H), 2.85-3.14 (m, 3H), 3.24-3.53 (m, 2H), 3.99 (t, J=15.3 Hz, 1H), 4.06-4.42 (m, 1H), 4.73 (t, J=13.7 Hz, 1H), 6.48 (t, J=6.9 Hz, 1H), 6.82-711 (m, 1H), 7.11-7.36 (m, 5H), 10.20 (br. s., 1H); m/z (ES+APCI)⁺: 351 [M+H]⁺.

### Example 258

### (E)-3-(4-Methoxy-1H-pyrazolo[4,3-c]pyridin-3-yl)-1-((R)-3-phenyl-piperidin-1-yl)-propenone

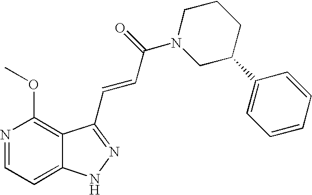

A solution of Intermediate 52 (0.1 g, 0.4 mmol) in TFA (1.5 ml) was stirred at 70° C. overnight. The reaction mixture was cooled to room temperature and then evaporated. The crude residue was re-dissolved in DCM and eluted though an Isolute-NH₂ cartridge. The solvents were removed and the crude product purified by mass triggered preparative LCMS (high pH buffer to yield a white solid (30 mg, 20%). ¹H NMR (400 MHz, DMSO-d₆) δ ppm 1.46-1.61 (m, 1H), 1.72-1.90 (m, 2H), 1.90-1.98 (m, 1H), 2.61-2.83 (m, 2H), 3.17-3.29 (m, 1H), 3.82-4.25 (m, 4H), 4.50-4.63 (m, 1H), 7.15 (m, 1H), 7.21-7.37 (m, 5H), 7.63 (m, 1H), 7.79 (m, 1H), 7.90

### Example 259

### (E)-3-(4-Methoxy-1H-pyrazolo[4,3-c]pyridin-3-yl)-1-((R)-3-methyl-piperidin-1-yl)-propenone

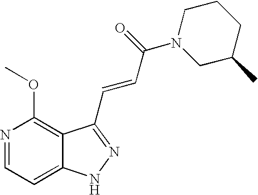

To a solution of Intermediate 51 (0.16 g, 0.47 mmol) in DMF (1.5 ml) was added HATU (0.19 g, 0.49 mmol) and N,N-diisopropylethylamine (492 μl, 2.83 mmol), followed by (R)-3-methylpiperidine (56 mg, 0.56 mmol). The resulting solution was left to stir at room temperature overnight, and then evaporated. The crude residue was re-dissolved in DCM and eluted though an Isolute-NH₂ cartridge. The solvents were removed and the crude product purified by flash chromatography eluting with 50-70% ethyl acetate/petroleum ether gradient to give a gum. TFA (1.5 ml) was then added and the resulting mixture stirred at 65° C. overnight and then evaporated. The crude residue was re-dissolved in DCM and eluted though an Isolute-NH₂ cartridge. The solvents were removed and the crude product purified by mass triggered preparative LCMS (high pH buffer to yield a white solid (30 mg, 21%). ¹H NMR (400 MHz, MeOD) δ ppm 0.77-1.08 (m, 3H), 1.20-1.38 (m, 1H), 1.40-2.09 (m, 4H), 2.38-3.27 (m, 2H), 4.00-4.29 (m, 4H), 4.29-4.54 (m, 1H), 7.08 (d, J=6.4 Hz, 1H), 7.67 (dd, J=15.6, 2.7 Hz, 1H), 7.80-7.98 (m, 2H); m/z (ES+APCI)⁺: 301 [M+H]⁺.

### Example 260

### 3-(4-Methoxy-1H-pyrazolo[4,3-c]pyridin-3-yl)-1-((R)-3-phenyl-piperidin-1-yl)-propan-1-one

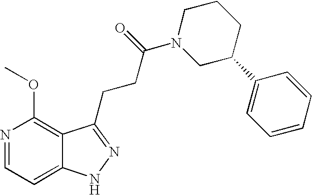

A solution of Intermediate 53 (0.15 g, 0.31 mmol) in TFA (1.5 ml) was stirred at 60° C. overnight. The reaction mixture was cooled to room temperature, evaporated, and the crude residue was re-dissolved in DCM and eluted through an SCX cartridge, eluting first with DCM, followed by 2M/NH₃ in methanol to yield a white solid (80 mg, 71%). ¹H NMR (400 MHz, DMSO-d₆) δ ppm 1.36-1.56 (m, 1H), 1.65-1.79 (m, 2H), 1.86-1.93 (m, 1H), 2.51-2.60 (m, 1H), 2.61-2.85 (m, 3H), 3.02-3.20 (m, 3H), 3.70-4.10 (m, 4H), 4.40-4.51 (m, 1H), 7.03 (dd, J=8.7, 6.0 Hz, 1H), 7.19-7.34 (m, 5H), 7.80 (dd, J=10.5, 00 Hz, 1H); m/z (ES+APCI)⁺: 365 [M+H]⁺.

### Example 261

### (E)-3-(4-Cyclohexyloxy-1H-pyrazolo[4,3-c]pyridin-3-yl)-1-((R)-3-phenyl-piperidin-1-yl)-propenone

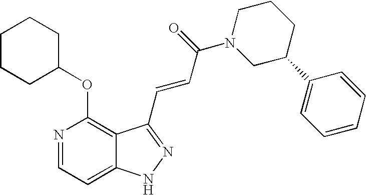

Prepared analogously to Example 172 from Intermediate 57 and (R)-3-phenylpiperidine to give the desired product as a white solid (2.2 mg, 2%) ¹H NMR (400 MHz, MeOD) δ ppm 1.38-1.60 (m, 4H), 1.61-1.77 (m, 3H), 1.84-2.14 (m, 6H), 2.71-2.90 (m, 2H), 3.25-3.43 (m, 2H), 4.25-4.37 (m, 1H), 4.71 (t, J=13.3 Hz, 1H), 5.21-5.37 (m, 1H), 7.01-7.13 (m, 1H), 7.18-7.27 (m, 1H), 7.29-7.37 (m, 4H), 7.58-7.76 (m, 1H), 7.85 (t, J=6.9 Hz, 1H), 8.06 (dd. J=15.6, 10.5 Hz, 1H); m/z (ES+APCI)⁺: 431 [M+H]⁺.

### Example 262

### N-{4-[2-(4-Cyclohexylamino-1H-pyrazolo[4,3-c]pyridin-3-yl)-ethyl]-phenyl}-acetamide

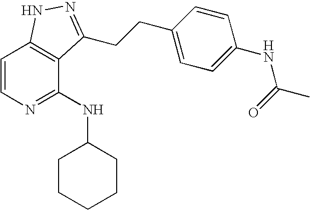

To a chromacol tube was added acetic acid (7 mg, 0.12 mmol) and HATU (48 mg, 0.125 mmol) in DMF (0.5 ml). The reaction mixture was stirred for 5 minutes followed by the addition of Intermediate 60 (40 mg, 0.12 mmol) and DIPEA (125 μl, 0.72 mmol) in DMF (0.5 ml), and the resulting mixture was allowed to stir at rt overnight. The mixture was diluted with DCM and saturated sodium bicarbonate (aq), and the organic layer was dried and concentrated. The residue was purified by mass triggered preparative HPLC (high pH buffer) to give the desired product as a white solid (26 mg, 58%). ¹H NMR (400 MHz, DMSO-d₆) δ ppm 1.13-1.24 (m, 1H) 1.24-1.41 (m, 4H) 1.60 (d, J=11.9 Hz, 1H) 1.08-1.77 (m, 2H) 1.91-2.08 (m, 5H) 2.88-2.97 (m, 2H) 3.22-3.31 (m, 2H) 4.01 (br. s., 1H) 5.43 (d, J=7.8 Hz, 1H) 6.58 (d, J=6.0 Hz, 1H) 7.17 (m, J=8.2 Hz, 2H) 7.47 (m, J=8.7 Hz, 2H) 7.66 (d, J=6.0 Hz, 1H) 9.9 (s, 1H); m/z (ES+APCI)⁺: 378 [M+H]⁺.

### Examples 263-276

Examples 263-276 in the table below were prepared analogously to Example 262 from Intermediate 60 and the appropriate carboxylic acid.

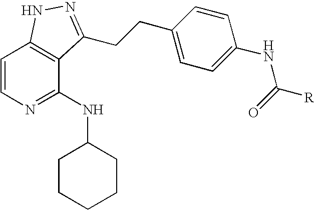

| | | | | HPLC |
|---|---|---|---|---|
| | | | m/z | retention |
| Example | R group | Name | (ES + APCl)⁺ | time (min)* |
| 263 | 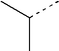 | N-{4-[2-(4- Cyclohexylamino-1H- pyrazolo[4,3-c]pyridin- 3-yl)-ethyl]phenyl}- isobutyramide | 1.87 | 406 |
| 264 | 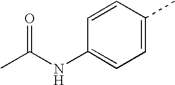 | 4-Acetylamino-N-{4-[2- (4-cyclohexylamino- 1H-pyrazolo[4,3- c]pyridin-3-yl)-ethyl]- phenyl}-benzamide | 1.70 | 497 |
| 265 | 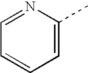 | Pyridine-2-carboxylic acid {4-[2-(4- cyclohexylamino-1H- pyrazolo[4,3-c]pyridin- 3-yl)-ethyl]-phenyl}- amide | 2.01 | 441 |
| 266 | 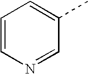 | N-{4-[2-(4- Cyclohexylamino-1H- pyrazolo[4,3-c]pyridin- 3-yl)-ethyl]-phenyl}- nicotinamide | 1.69 | 441 |
| 267 | 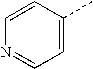 | N-{4-[2-(4- Cyclohexylamino-1H- pyrazolo[4,3-c]pyridin- 3-yl)-ethyl]-phenyl}- isonicotinamide | 1.70 | 441 |
| 268 | 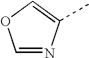 | Oxazole-4-carboxylic acid {4-[2-(4- cyclohexylamino-1H- pyrazolo[4,3-c]pyridin- 3-yl)-ethyl]-phenyl}- amide | 1.78 | 431 |
| 269 | 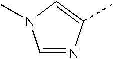 | 1-Methyl-1H-imidazole- 4-carboxylic acid {4-[2- (4-cyclohexylamino- 1H-pyrazolo[4,3- c]pyridin-3-yl)-ethyl]- phenyl}-amide | 1.65 | 444 |
| 270 | 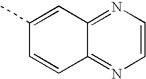 | Quinoxaline-6- carboxylic acid {4-[2-(4- cyclohexylamino-1H- pyrazolo[4,3-c]pyridin- 3-yl)-ethyl]-phenyl}- amide | 1.80 | 492 |
| 271 | 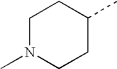 | 1-Methyl-piperidine-4- carboxylic acid {4-[2-(4- cyclohexylamino-1H- pyrazolo[4,3-c]pyridin- 3-yl)-ethyl]-phenyl}- amide | 1.68 | 461 |
| 272 | 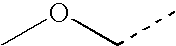 | N-{4-[2-(4- Cyclohexylamino-1H- pyrazolo[4,3-c]pyridin- 3-yl)-ethyl]-phenyl}-2- methoxy-acetamide | 1.73 | 408 |
| 273 | 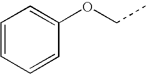 | N-{4-[2-(4- Cyclohexylamino-1H- pyrazolo[4,3-c]pyridin- 3-yl)-ethyl]-phenyl}-2- phenoxy-acetamide | 2.05 | 470 |
| 274 | 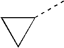 | Cyclopropanecarboxylic acid {4-[2-(4- cyclohexylamino-1H- pyrazolo[4,3-c]pyridin- 3-yl)-ethyl]phenyl}- amide | 1.79 | 404 |
| 275 | 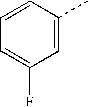 | N-{4-[2-(4- Cyclohexylamino-1H- pyrazolo[4,3-c]pyridin- 3-yl)-ethylpphenyl}-3- fluoro-benzamide | 2.05 | 458 |
| 276 | 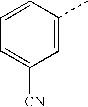 | 3-Cyano-N-{4-[2-(4- cyclohexylamino-1H- pyrazolo[4,3-c]pyridin- 3-yl)-ethyl]-phenyl}- benzamide | 1.96 | 465 |

*HPLC column: 4.6 × 5 mm (5 μm) C-18 Xbridge; flow rate: 3 ml/min; Run time: 3.2 min: Solvent A: 0.1% Ammonium Hydroxide in water Solvent B: Acetonitrile; Gradient - 10-100% B; Gradient time: 2.35 min.

### Examples 277-293

Examples 277-293 in the following table were prepared analogously to Example 114 from Intermediate 3 and the corresponding alcohol.

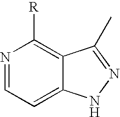

| | | | | HPLC |
|---|---|---|---|---|
| | | | m/z | retention |
| Example | R group | Name | (ES + APCI)⁺ | time (min) |
| 277 | 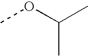 | 4-Isopropoxy-3-methyl- 1H-pyrazolo[4,3- c]pyridine | 192 | 1.51^{c} |
| 278 | 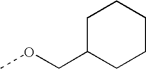 | 4-Cyclohexylmethoxy- 3-methyl-1H- pyrazolo[4,3-c]pyridine | 246 | 2.07^{c} |
| 279 | 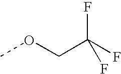 | 3-Methyl-4-(2,2,2- trifluoro-ethoxy)-1H- pyrazolo[4,3-c]pyridine | 232 | 1.64^{c} |
| 280 | 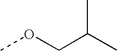 | 4-Isobutoxy-3-methyl- 1H-pyrazolo[4,3- c]pyridine | 206 | 1.69^{c} |
| 281 | 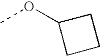 | 4-Cyclobutoxy-3- methyl-1H- pyrazolo[4,3-c]pyridine | 204 | 1.60^{c} |
| 282 | 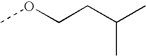 | 3-Methyl-4-(3-methyl- butoxy)-1H- pyrazolo[4,3-c]pyridine | 220 | 1.86^{c} |
| 283 | 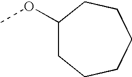 | 4-Cycloheptyloxy-3- methyl-1H- pyrazolo[4,3-c]pyridine | 246 | 2.08^{c} |
| 284 | 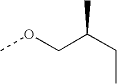 | 3-Methyl-4-((S)-2- methyl-butoxy)-1H- pyrazolo[4,3-c]pyridine | 220 | 1.85^{c} |
| 285 | 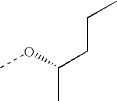 | 3-Methyl-4-((S)-1- methyl-butoxy)-1H- pyrazolo[4,3-c]pyridine | 220 | 1.87^{c} |
| 286 | 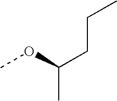 | 3-Methyl-4-((R)-1- methyl-butoxy)-1H- pyrazolo[4,3-c]pyridine | 220 | 1.87^{c} |
| 287 | 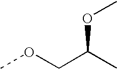 | 4-((S)-2-Methoxy- propoxy)-3-methyl-1H- pyrazolo[4,3-c]pyridine | 222 | 1.32^{c} |
| 288 | 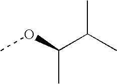 | 4-((R)-1,2-Dimethyl- propoxy)-3-methyl-1H- pyrazolo[4,3-c]pyridine | 220 | 1.83^{c} |
| 289 | 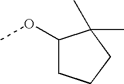 | 4-(2,2-Dimethyl- cyclopentyloxy)-3- methyl-1H- pyrazolo[4,3-c]pyridine | 246 | 2.01^{c} |
| 290 | 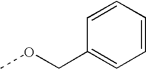 | 4-Benzyloxy-3-methyl- 1H-pyrazolo[4,3- c]pyridine | 240 | 1.73^{c} |
| 291 | 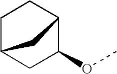 | 4-exo- Bicyclo[2.2.1]hept-2- yloxy)-3-methyl-1H- pyrazolo[4,3-c]pyridine (Racemic) | 244 | 1.94^{c} |
| 292 | 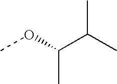 | 4-((S)-1,2-Dimethyl- propoxy)-3-methyl-1H- pyrazolo[4,3-c]pyridine | 220 | 1.83^{c} |
| 293 | 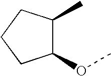 | Cis-3-Methyl-4-(2- methyl-cyclopentyloxy)- 1H-pyrazolo[4,3- c]pyridine (Racemic) | 232 | 1 .63^{c} |

^{c}HPLC column: 4.6 × 50 mm (5 μm) C-18 Xbridge; flow rate: 3 ml/min; Run time: 3.2 min: Solvent A: 0.1% Ammonium Hydroxide in water, Solvent B: Acetonitrile; Gradient—10-100% B; Gradient time: 2.35 min.
^{d}HPLC column: 4.6 × 50 mm (5 μm) C-18 Xbridge; flow rate: 3 ml/min; Run time: 3.2 min: Solvent A: 0.1% Formic acid in water, Solvent B: Acetonitrile; Gradient—10-100% B; Gradient time: 2.35 min.

### Example 294

### Trans-3-Methyl-4-(4-methyl-cyclohexyloxy)-1H-pyrazolo[4,3-c]pyridine

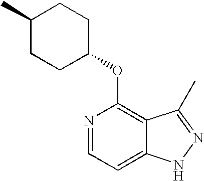

Intermediate 61 (100 mg, 0.24 mmol), trans-4-methylcyclohexanol (61 μl, 0.49 mmol), Pd(OAc)₂ (3.3 mg, 0.015 mmol), BINAP (12 mg, 0.02 mmol) and sodium tert-butoxide (70 mg, 0.73 mmol) were combined in toluene (3 ml). The mixture was degassed and placed under an atmosphere of nitrogen, then stirred at 100° C. for 18 h. The mixture was diluted with DCM, washed with H₂O, the organic layer was recovered using a phase separation cartridge, dried (MgSO₄) and evaporated. The crude product was dissolved in trifluoroacetic acid (0.2 ml, 2.70 mmol) in DCM (2 ml) and stirred at rt for 18 h. The reaction mixture was evaporated and then purified by cation exchange chromatography using an Isolute SCX cartridge. The crude product was then purified by preparative LCMS (high pH buffer) to give the product as a white solid (17 mg, 28%). ¹H NMR (400 MHz, DMSO-d₆) δ ppm 0.91 (d, J=6.9 Hz, 3H), 1.04-1.16 (m, 2H), 1.40-1.52 (m, 3H), 1.70-1.78 (m, 2H), 2.09-2.16 (m, 2H), 2.51 (s, 3H), 5.01-5.19 (m, 1H), 6.95 (d, J=6.0 Hz, 1H), 7.75 (d, J=6.0 Hz, 1H); m/z (ES+APCI)⁺: 246 [M+H]⁺.

### Examples 295-300

Examples 295-300 in the table below were prepared analogously to Example 294 from Intermediate 4 and the corresponding alcohol.

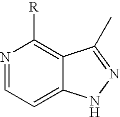

| | | | | HPLC |
|---|---|---|---|---|
| | | | m/z | retention |
| Example | R group | Name | (ES + APCI)⁺ | time (min) |
| 295 | 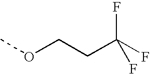 | 3-Methyl-4-(3,3,3- trifluoro-propoxy)-1H- pyrazolo[4,3-c]pyridine | 246 | 1.57^{c} |
| 296 | 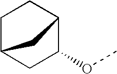 | 4-endo- Bicyclo[2.2.1]hept-2- yloxy)-3-methyl-1H- pyrazolo[4,3-c]pyridine (Racemic) | 244 | 1.93^{c} |
| 297 | 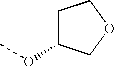 | 3-Methyl-4-[(R)- (tetrahydro-furan-3- yl)oxy]-1H- pyrazolo[4,3-c]pyridine | 220 | 1.14^{c} |
| 298 | 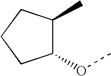 | Trans-3-Methyl-4-(2- methyl-cyclopentyloxy)- 1H-pyrazolo[4,3- c]pyridine (Racemic) | 232 | 1.92^{c} |
| 299 | 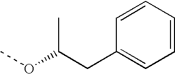 | 3-Methyl-4-((R)- 1- methyl-2-phenyl ethoxy)-1H- pyrazolo[4,3-c]pyridine | 268 | 1.89^{c} |
| 300 | 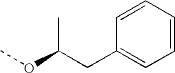 | 3-Methyl-4-((S)-1- methyl-2-phenyl- ethoxy)-1H- pyrazolo[4,3-c]pyridine | 268 | 1.93^{c} |

^{c}HPLC column: 4.6 × 50 mm (5 μm) C-18 Xbridge; flow rate: 3 ml/min; Run time: 3.2 min: Solvent A: 0.1% Ammonium Hydroxide in water, Solvent B: Acetonitrile; Gradient—10-100% B; Gradient time: 2.35 min.

### Examples 301-392

Examples 301-392 in the table below were prepared analogously to procedures described earlier, either by nucleophilic displacement of the 4-chloro group of Intermediate 3 with the appropriate amine (c.f. Example 1 or Example 59);
OR

Nucleophilic displacement of the 4-chloro group of Intermediate 4 with the corresponding amine (c.f. Intermediate 14), followed by removal of the protecting group (c.f. Intermediate 35);
OR

Palladium catalyzed amination of Intermediate 4 with the corresponding amine (c.f. Intermediate 7 and Intermediate 9, Step 1) followed by removal of the protecting group.

The person skilled in the art will appreciate that it may be necessary or desirable to modify the conditions for each specific compound, such as changing the number or equivalents of reagents, changing the solvent, changing the temperature, changing the reaction time. In the case of palladium catalysed reactions, using a different palladium salt, ligand or base. It may also be necessary or desirable to employ different work-up or purification techniques.

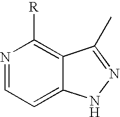

| | | | | HPLC |
|---|---|---|---|---|
| | | | | retention |
| | | | m/z | time |
| Example | R group | Name | (ES + APCI)⁺ | (min)* |
| 301 | 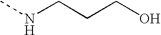 | 3-(3-Methyl-1H- pyrazolo[4,3-c]pyridin- 4-ylamino)-propan-1-ol | 207 | 0.79 |
| 302 | 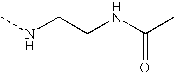 | N-[2-(3-Methyl-1H- pyrazolo[4,3-c]pyridin- 4-ylamino)-ethyl]- acetamide | 234 | 0.76 |
| 303 | 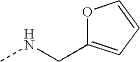 | Furan-2-ylmethyl-(3- methyl-1H- pyrazolo[4,3-c]pyridin- 4-yl)-amine | 229 | 1.27 |
| 304 | 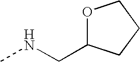 | (3-Methyl-1H- pyrazolo[4,3-c]pyridin- 4-yl)-(tetrahydro-furan- 2-ylmethyl)-amine | 233 | 1.10 |
| 305 | 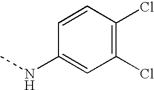 | (3,4-Dichloro-phenyl)- (3-methyl-1H- pyrazolo[4,3-c]pyridin- 4-yl)-amine | 293 | 1.94 |
| 306 | 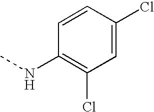 | (2,4-Dichloro-phenyl)- (3-methyl-1H- pyrazolo[4,3-c]pyridin- 4-yl)-amine | 293 | 2.12 |
| 307 | 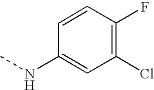 | (3-Chloro-4-fluoro- phenyl)-(3-methyl-1H- pyrazolo[4,3-c]pyridin- 4-yl)-amine | 277 | 1.76 |
| 308 | 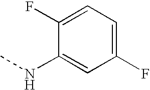 | (2,5-Difluoro-phenyl)- (3-methyl-1H- pyrazolo[4,3-c]pyridin- 4-yl)-amine | 261 | 1.75 |
| 309 | 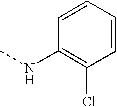 | (2-Chloro-phenyl)-(3- methyl-1H- pyrazolo[4,3-c]pyridin- 4-yl)-amine | 259 | 1.82 |
| 310 | 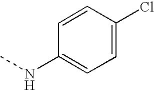 | (4-Chloro-phenyl)-(3- methyl-1H- pyrazolo[4,3-c]pyridin- 4-yl)-amine | 259 | 1.73 |
| 311 | 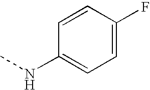 | (4-Fluoro-phenyl)-(3- methyl-1H- pyrazolo[4,3-c]pyridin- 4-yl)-amine | 243 | 1.51 |
| 312 | 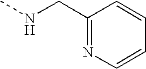 | (3-Methyl-1H- pyrazolo[4,3-c]pyridin- 4-yl)-pyridin-2- ylmethyl-amine | 240 | 1.11 |
| 313 | 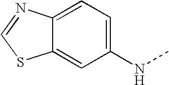 | Benzothiazol-6-yl-(3- methyl-1H- pyrazolo[4,3-c]pyridin- 4-yl)-amine | 282 | 1.33 |
| 314 | 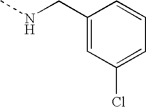 | (3-Chloro-benzyl)-(3- methyl-1H- pyrazolo[4,3-c]pyridin- 4-yl)-amine | 273 | 1.63 |
| 315 | 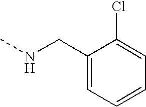 | (2-Chloro-benzyl)-(3- methyl-1H- pyrazolo[4,3-c]pyridin- 4-yl)-amine | 273 | 1.65 |
| 316 | 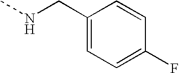 | (4-Fluoro-benzyl)-(3- methyl-1H- pyrazolo[4,3-c]pyridin- 4-yl)-amine | 257 | 1.52 |
| 317 | 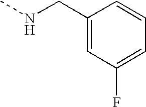 | (3-Fluoro-benzyl)-(3- methyl-1H- pyrazolo[4,3-c]pyridin- 4-yl)-amine | 257 | 1.51 |
| 318 | 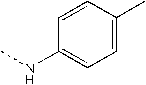 | (4-Methyl-phenyl)-(3- methyl-1H- pyrazolo[4,3-c]pyridin- 4-yl)-amine | 239 | 1.61 |
| 319 | 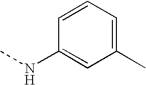 | (3-Methyl-phenyl)-(3- methyl-1H- pyrazolo[4,3-c]pyridin- 4-yl)-amine | 239 | 1.63 |
| 320 | 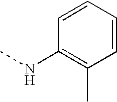 | (2-Methyl-phenyl)-(3- methyl-1H- pyrazolo[4,3-c]pyridin- 4-yl)-amine | 239 | 1.50 |
| 321 | 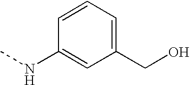 | [3-(3-Methyl-1H- pyrazolo[4,3-c]pyridin- 4-ylamino)-phenyl]- methanol | 255 | 1.14 |
| 322 | 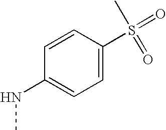 | (4-Methanesulfonyl- phenyl)-(3-methyl-1H- pyrazolo[4,3-c]pyridin- 4-yl)-amine | 303 | 1.23 |
| 323 | 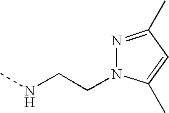 | [2-(3,5-Dimethyl- pyrazol-1-yl)-ethyl]-(3- methyl-1H- pyrazolo[4,3-c]pyridin- 4-yl)-amine | 271 | 1.25 |
| 324 | 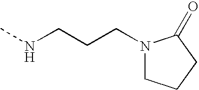 | 1-[3-(3-Methyl-1H- pyrazolo[4,3-c]pyridin- 4-ylamino)-propyl]- pyrrolidin-2-one | 274 | 1.01 |
| 325 | 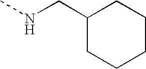 | Cyclohexylmethyl-(3- methyl-1H- pyrazolo[4,3-c]pyridin- 4-yl)-amine | 245 | 1.69 |
| 326 | 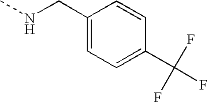 | (3-Methyl-1H- pyrazolo[4,3-c]pyridin- 4-yl)-(4-trifluoromethyl- benzyl)-amine | 307 | 1.74 |
| 327 | 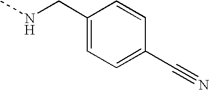 | 4-[(3-Methyl-1H- pyrazolo[4,3-c]pyridin- 4-ylamino)-methyl]- benzonitrile | 264 | 1.37 |
| 328 | 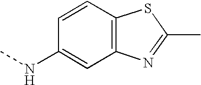 | (2-Methyl- benzothiazol-5-yl)-(3- methyl-1H- pyrazolo[4,3-c]pyridin- 4-yl)-amine | 296 | 1.45 |
| 329 | 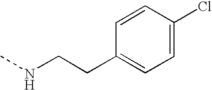 | [2-(4-Chloro-phenyl)- ethyl]-(3-methyl-1H- pyrazolo[4,3-c]pyridin- 4-yl)-amine | 287 | 1.70 |
| 330 | 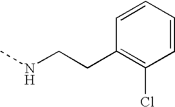 | [2-(2-Chloro-phenyl)- ethyl]-(3-methyl-1H- pyrazolo[4,3-c]pyridin- 4-yl)-amine | 287 | 1.68 |
| 331 | 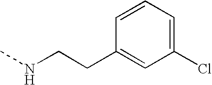 | [2-(3-Chloro-phenyl)- ethyl]-(3-methyl-1H- pyrazolo[4,3-c]pyridin- 4-yl)-amine | 287 | 1.69 |
| 332 | 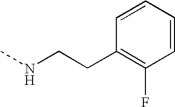 | [2-(2-Fluoro-phenyl)- ethyl]-(3-methyl-1H- pyrazolo[4,3-c]pyridin- 4-yl)-amine | 271 | 1.56 |
| 333 | 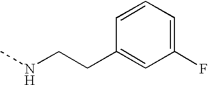 | [2-(3-Fluoro-phenyl)- ethyl]-(3-methyl-1H- pyrazolo[4,3-c]pyridin- 4-yl)-amine | 271 | 1.59 |
| 334 | 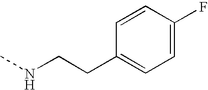 | [2-(4-Fluoro-phenyl)- ethyl]-(3-methyl-1H- pyrazolo[4,3-c]pyridin- 4-yl)-amine | 271 | 1.57 |
| 335 | 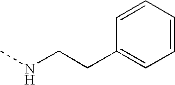 | (3-Methyl-1H- pyrazolo[4,3-c]pyridin- 4-yl)-phenethyl-amine | 253 | 1.54 |
| 336 | 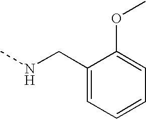 | (2-Methoxy-benzyl)-(3- methyl-1H- pyrazolo[4,3-c]pyridin- 4-yl)-amine | 269 | 1.51 |
| 337 | 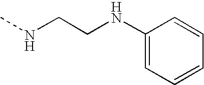 | N-(3-Methyl-1H- pyrazolo[4,3-c]pyridin- 4-yl)-N′-phenyl-ethane- 1,2-diamine | 268 | 1.47 |
| 338 | 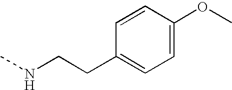 | [2-(4-Methoxy-phenyl)- ethyl]-(3-methyl-1H- pyrazolo[4,3-c]pyridin- 4-yl)-amine | 283 | 1.15 |
| 339 | 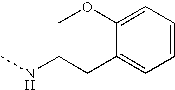 | [2-(2-Methoxy-phenyl)- ethyl]-(3-methyl-1H- pyrazolo[4,3-c]pyridin- 4-yl)-amine | 283 | 0.97 |
| 340 | 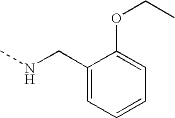 | (2-Ethoxy-benzyl)-(3- methyl-1H- pyrazolo[4,3-c]pyridin- 4-yl)-amine | 283 | 1.28 |
| 341 | 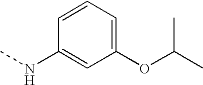 | (3-Isopropoxy-phenyl)- (3-methyl-1H- pyrazolo[4,3-c]pyridin- 4-yl)-amine | 283 | 1.49 |
| 342 | 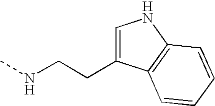 | [2-(1H-Indol-3-yl)- ethyl]-(3-methyl-1H- pyrazolo[4,3-c]pyridin- 4-yl)-amine | 292 | 1.47 |
| 343 | 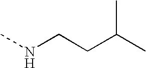 | (3-Methyl-butyl)-(3- methyl-1H- pyrazolo[4,3-c]pyridin- 4-yl)-amine | 219 | 1.52 |
| 344 | 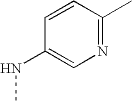 | (3-Methyl-1H- pyrazolo[4,3-c]pyridin- 4-yl)-(6-methyl-pyridin- 3-yl)-amine | 240 | 1.15 |
| 345 | 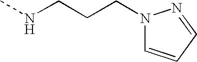 | (3-Methyl-1H- pyrazolo[4,3-c]pyridin- 4-yl)-(3-pyrazol-1-yl- propyl)-amine | 257 | 1.11 |
| 346 | 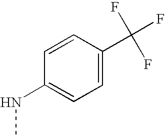 | (3-Methyl-1H- pyrazolo[4,3-c]pyridin- 4-yl)-(4-trifluoromethyl- phenyl)-amine | 293 | 1.85 |
| 347 | 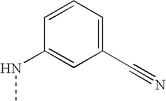 | 3-(3-Methyl-1H- pyrazolo[4,3-c]pyridin- 4-ylamino)-benzonitrile | 250 | 1.47 |
| 348 | 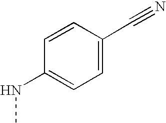 | 4-(3-Methyl-1H- pyrazolo[4,3-c]pyridin- 4-ylamino)-benzonitrile | 250 | 1.45 |
| 349 | 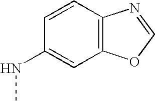 | Benzoxazol-6-yl-(3- methyl-1H- pyrazolo[4,3-c]pyridin- 4-yl)-amine | 266 | 1.23 |
| 350 | 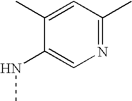 | (4,6-Dimethyl-pyridin- 3-yl)-(3-methyl-1H- pyrazolo[4,3-c]pyridin- 4-yl)-amine | 254 | 1.16 |
| 351 | 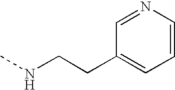 | (3-Methyl-1H- pyrazolo[4,3-c]pyridin- 4-yl)-(2-pyridin-3-yl- ethyl)-amine | 254 | 1.07 |
| 352 | 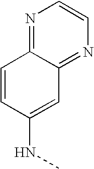 | (3-Methyl-1H- pyrazolo[4,3-c]pyridin- 4-yl)-quinoxalin-6-yl- amine | 277 | 1.18 |
| 353 | 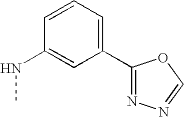 | (3-Methyl-1H- pyrazolo[4,3-c]pyridin- 4-yl)-(3-1,3,4- oxadiazol-2-yl-phenyl)- amine | 293 | 1.27 |
| 354 | 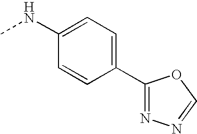 | (3-Methyl-1H- pyrazolo[4,3-c]pyridin- 4-yl)-(4-1,3,4- oxadiazol-2-yl-phenyl)- amine | 293 | 1.23 |
| 355 | 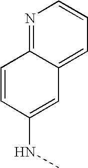 | (3-Methyl-1H- pyrazolo[4,3-c]pyridin- 4-yl)-quinolin-6-yl- amine | 276 | 1.29 |
| 356 | 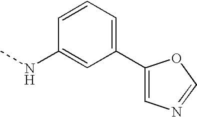 | (3-Methyl-1H- pyrazolo[4,3-c]pyridin- 4-yl)-(3-oxazol-5-yl- phenyl)-amine | 292 | 1.41 |
| 357 | 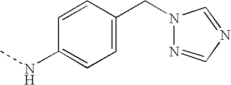 | (3-Methyl-1H- pyrazolo[4,3-c]pyridin- 4-yl)-(4-1,2,4-triazol-1- ylmethyl-phenyl)- amine | 306 | 1.13 |
| 358 | 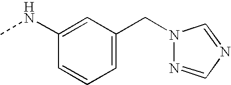 | (3-Methyl-1H- pyrazolo[4,3-c]pyridin- 4-yl)-(3-1,2,4-triazol-1- ylmethyl-phenyl)- amine | 306 | 1.17 |
| 359 | 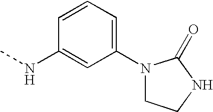 | 1-[3-(3-Methyl-1H- pyrazolo[4,3-c]pyridin- 4-ylamino)-phenyl]- imidazolidin-2-one | 309 | 1.17 |
| 360 | 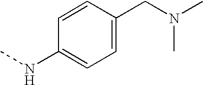 | (4- Dimethylaminomethyl- phenyl)-(3-methyl-1H- pyrazolo[4,3-c]pyridin- 4-yl)-amine | 282 | 1.38 |
| 361 | 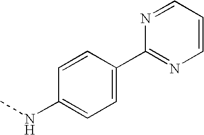 | (3-Methyl-1H- pyrazolo[4,3-c]pyridin- 4-yl)-(4-pyrimidin-2-yl- phenyl)-amine | 303 | 1.40 |
| 362 | 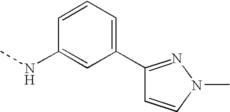 | (3-Methyl-1H- pyrazolo[4,3-c]pyridin- 4-yl)-[3-(1-methyl-1H- pyrazol-3-yl)-phenyl]- amine | 305 | 1.43 |
| 363 | 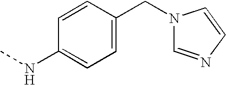 | (4-Imidazol-1-ylmethyl- phenyl)-(3-methyl-1H- pyrazolo[4,3-c]pyridin- 4-yl)-amine | 305 | 1.41 |
| 364 | 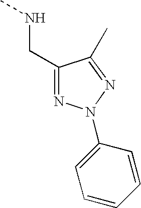 | (5-Methyl-2-phenyl-2H- 1,2,3-triazol-4- ylmethyl)-(3-methyl- 1H-pyrazolo[4,3- c]pyridin-4-yl)-amine | 320 | 1.70 |
| 365 | 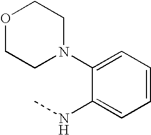 | (3-Methyl-1H- pyrazolo[4,3-c]pyridin- 4-yl)-(2-morpholin-4-yl- phenyl)-amine | 310 | 1.68 |
| 366 | 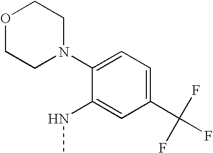 | (5-Trifluoromethyl-2- morpholin-4-yl-phenyl)- (3-methyl-1H- pyrazolo[4,3-c]pyridin- 4-yl)-amine | 378 | 1.97 |
| 367 | 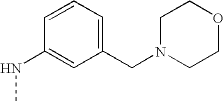 | (3-Methyl-1H- pyrazolo[4,3-c]pyridin- 4-yl)-(3-morpholin-4- ylmethyl-phenyl)- amine | 324 | 1.34 |
| 368 | 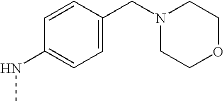 | (3-Methyl-1H- pyrazol[4,3-c]pyridin- 4-yl)-(4-morpholin-4- ylmethyl-phenyl)- amine | 324 | 1.29 |
| 369 | 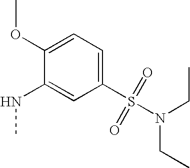 | N,N-Diethyl-4- methoxy-3-(3-methyl- 1H-pyrazolo[4,3- c]pyridin-4-ylamino)- benzenesulfonamide | 390 | 1.82 |
| 370 | 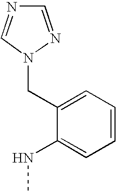 | (3-Methyl-1H- pyrazolo[4,3-c]pyridin- 4-yl)-(2-1,2,4-triazol-1- ylmethyl-phenyl)- amine | 306 | 1.12 |
| 371 | 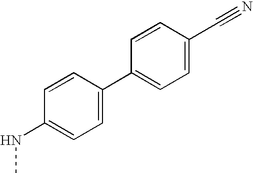 | 4′-(3-Methyl-1H- pyrazol[4,3-c]pyridin- 4-ylamino)-biphenyl-4- carbonitrile | 326 | 1.82 |
| 372 | 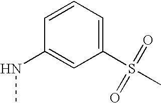 | (3-Methanesulfonyl- phenyl)-(3-methyl-1H- pyrazolo[4,3-c]pyridin- 4-yl)-amine | 303 | 1.23 |
| 373 | 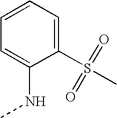 | (2-Methanesulfonyl- phenyl)-(3-methyl-1H- pyrazolo[4,3-c]pyridin- 4-yl)-amine | 303 | 1.39 |
| 374 | 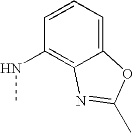 | (2-Methyl-benzoxazol- 4-yl)-(3-methyl-1H- pyrazolo[4,3-c]pyridin- 4-yl)-amine | 280 | 1.64 |
| 375 | 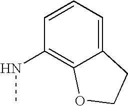 | (2,3-Dihydro- benzofuran-7-yl)-(3- methyl-1H- pyrazolo[4,3-c]pyridin- 4-yl)-amine | 267 | 1.58 |
| 376 | 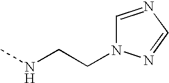 | (3-Methyl-1H- pyrazolo[4,3-c]pyridin- 4-yl)-(2-1,2,4-triazol-1- yl-ethyl)-amine | 244 | 0.81 |
| 377 | 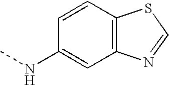 | Benzothiazol-5-yl-(3- methyl-1H- pyrazolo[4,3-c]pyridin- 4-yl)-amine | 282 | 1.34 |
| 378 | 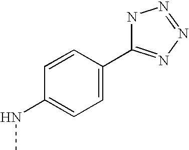 | (3-Methyl-1H- pyrazolo[4,3-c]pyridin- 4-yl)-[4-(1H-tetrazol-5- yl)-phenyl]-amine | 293 | 0.50 |
| 379 | 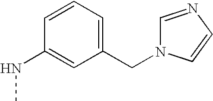 | (3-Imidazol-1-ylmethyl- phenyl)-(3-methyl-1H- pyrazolo[4,3-c]pyridin- 4-yl)-amine | 306 | 1.08 |
| 380 | 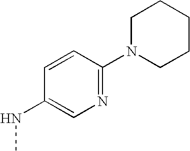 | (3-Methyl-1H- pyrazolo[4,3-c]pyridin- 4-yl)-(3,4,5,6- tetrahydro-2H- [1,2′]bipyridinyl-5′-yl)- amine | 309 | 1.07 |
| 381 | 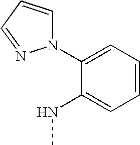 | (3-Methyl-1H- pyrazolo[4,3-c]pyridin- 4-yl)-(2-pyrazol-1-yl- phenyl)-amine | 291 | 1.27 |
| 382 | 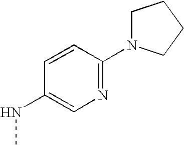 | (3-Methyl-1H- pyrazolo[4,3-c]pyridin- 4-yl)-(6-pyrrolidin-1-yl- pyridin-3-yl)-amine | 295 | 1.15 |
| 383 | 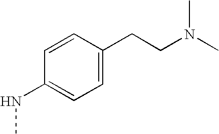 | [4-(2-Dimethylamino- ethyl)-phenyl]-(3- methyl-1H- pyrazolo[4,3-c]pyridin- 4-yl)-amine | 296 | 1.57 |
| 384 | 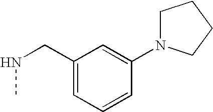 | (3-Methyl-1H- pyrazolo[4,3-c]pyridin- 4-yl)-(3-pyrrolidin-1-yl- benzyl)-amine | 308 | 1.78 |
| 385 | 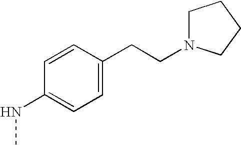 | (3-Methyl-1H- pyrazolo[4,3-c]pyridin- 4-yl)-[4-(2-pyrrolidin-1- yl-ethyl)-phenyl]-amine | 322 | 1.36 |
| 386 | 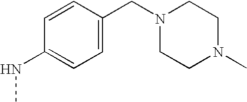 | [4-(4-Methyl- piperazin-1-ylmethyl)- phenyl]-(3-methyl-1H- pyrazolo[4,3-c]pyridin- 4-yl)-amine | 337 | 1.20 |
| 387 | 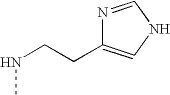 | [2-(1H-Imidazol-4-yl)- ethyl]-(3-methyl-1H- pyrazolo[4,3-c]pyridin- 4-yl)-amine | 243 | 0.85 |
| 388 | 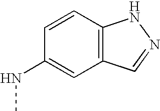 | (1H-Indazol-5-yl)-(3- methyl-1H- pyrazolo[4,3-c]pyridin- 4-yl)-amine | 265 | 1.09 |
| 389 | 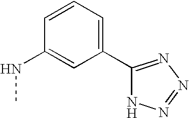 | (3-Methyl-1H- pyrazolo[4,3-c]pyridin- 4-yl)-[3-(1H-tetrazol-5- yl)-phenyl]-amine | 293 | 0.33 |
| 390 | 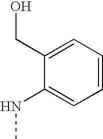 | [2-(3-Methyl-1H- pyrazolo[4,3-c]pyridin- 4-ylamino)-phenyl]- methanol | 255 | 1.23 |
| 391 | 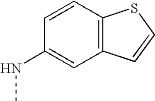 | Benzo[b]thiophen-5-yl- (3-ethyl-1H- pyrazolo[4,3-c]pyridin- 4-yl)-amine | 281 | 1.72 |
| 392 | 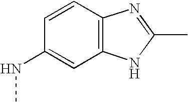 | (2-Methyl-3H- benzoimidazol-5-yl)-(3- methyl-1H- pyrazolo[4,3-c]pyridin- 4-yl)-amine | 279 | 1.01 |

*HPLC column: 4.6 × 50 mm (5 μm) C-18 Xbridge; flow rate: 3 ml/min; Run time: 3.2 min: Solvent A: 0.1% Ammonium Hydroxide in water, Solvent B: Acetonitrile; Gradient—10-100% B; Gradient time: 2.35 min.

### Results

### LRRK2 Potency

Potency scores for selected compounds of the invention against LRRK2 are shown in Table 1.

### Kinase Selectivity Data

Kinase selectivity data of representative compounds is shown in Table 2. Values are expressed as percentage inhibition of the each specific kinase at 1 μM inhibitor concentration.

Various modifications and variations of the described aspects of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the relevant fields are intended to be within the scope of the following claims.

### REFERENCES

1 Paisan-Ruiz, C., Jain, S., Evans, E. W., Gilks, W. P., Simon, J., van der Brug, M., Lopez de Munain, A., Aparicio, S., Gil, A. M., Khan, N., Johnson, J., Martinez, J. R., Nicholl, D., Carrera, I. M., Pena, A. S., de Silva, R., Lees, A., Marti-Masso, J. F., Perez-Tur, J., Wood, N. W. and Singleton, A. B. (2004) Cloning of the gene containing mutations that cause PARK8-linked Parkinson's disease. Neuron. 44, 595-600
2 Mata, I. F., Wedemeyer, W. J., Farrer, M. J., Taylor, J. P. and Gallo, K. A. (2006) LRRK2 in Parkinson's disease: protein domains and functional insights. Trends Neurosci. 29, 286-293
3 Taylor, J. P., Mata, I. F. and Farrer, M. J. (2006) LRRK2: a common pathway for parkinsonism, pathogenesis and prevention? Trends Mol Med. 12, 76-82
4 Farrer, M., Stone, J., Mata, I. F., Lincoln, S., Kachergus, J., Hulihan, M., Strain, K. J. and Maraganore, D. M. (2005) LRRK2 mutations in Parkinson disease. Neurology. 65, 738-740
5 Zabetian, C. P., Samii, A., Mosley, A. D., Roberts, J. W., Leis, B. C., Yearout, D., Raskind, W. H. and Griffith, A. (2005) A clinic-based study of the LRRK2 gene in Parkinson disease yields new mutations. Neurology. 65, 741-744
6 Bosgraaf, L. and Van Haastert, P. J. (2003) Roc, a Ras/GTPase domain in complex proteins. Biochim Biophys Acta. 1643, 5-10
7 Marin, I. (2006) The Parkinson disease gene LRRK2: evolutionary and structural insights. Mol Biol Evol. 23, 2423-2433
8 Manning, G., Whyte, D. B., Martinez, R., Hunter, T. and Sudarsanam, S. (2002) The protein kinase complement of the human genome. Science. 298, 1912-1934
9 West, A. B., Moore, D. J., Biskup, S., Bugayenko, A., Smith, W. W, Ross, C. A., Dawson, V. L. and Dawson, T. M. (2005) Parkinson's disease-associated mutations in leucine-rich repeat kinase 2 augment kinase activity. Proc Natl Acad Sci USA. 102, 16842-16847
10 Greggio, E., Jain, S., Kingsbury, A., Bandopadhyay, R., Lewis, P., Kaganovich, A., van der Brug, M. P., Beilina, A., Blackinton, J., Thomas, K. J., Ahmad, R., Miller, D. W, Kesavapany, S., Singleton, A., Lees, A., Harvey, R. J., Harvey, K. and Cookson, M. R. (2006) Kinase activity is required for the toxic effects of mutant LRRK2/dardarin. Neurobiol Dis. 23, 329-341
11 Jaleel, M., Nichols, R. J., Beak, M., Campbell, D. G., Gillardon, F., Knebel, A. and Alessi, D. R. (2007) LRRK2 phosphorylates moesin at threonine-558: characterization of how Parkinson's disease mutants affect kinase activity. Biochem J. 405, 307-317
12 Goldberg, J. M., Bosgraaf, L., Van Haastert, P. J. and Smith, J. L. (2002) Identification of four candidate cGMP targets in Dictyostelium. Proc Natl Acad Sci USA. 99, 6749-6754
13 Bosgraaf, L., Russcher, H., Smith, J. L., Wessels, D., Soil, D. R. and Van Haastert, P. J. (2002) A novel cGMP signalling pathway mediating myosin phosphorylation and chemotaxis in Dictyostelium. Embo J. 21, 4560-4570
14 Cohen, P. and Knebel, A. (2006) KESTREL: a powerful method for identifying the physiological substrates of protein kinases. Biochem J. 393, 1-6
15 Bretscher, A., Edwards, K. and Fehon, R. G. (2002) ERM proteins and merlin: integrators at the cell cortex. Nat Rev Mol Cell Biol. 3, 586-599
16 Polesello, C. and Payre, F. (2004) Small is beautiful: what flies tell us about ERM protein function in development. Trends Cell Biol. 14, 294-302
17 Nichols, R. J., Dzamko, N., Hutti, J. E., Cantley, L. C., Deak, M., Moran, J., Bamborough, P., Reith, A. D. and Alessi, D. R. (2009) Substrate specificity and inhibitors of LRRK2, a protein kinase mutated in Parkinson's disease. Biochem J. 424, 47-60

TABLE 1
Potency scores for selected compounds in the invention

| | |
|---|---|
| Example 1 | * |
| Example 2 | * |
| Example 3 | ** |
| Example 4 | ** |
| Example 5 | * |
| Example 6 | * |
| Example 7 | * |
| Example 8 | * |
| Example 9 | * |
| Example 10 | * |
| Example 11 | * |
| Example 12 | * |
| Example 13 | ** |
| Example 14 | * |
| Example 15 | ** |
| Example 16 | ** |
| Example 17 | * |
| Example 18 | * |
| Example 19 | * |
| Example 20 | ** |
| Example 21 | ** |
| Example 22 | ** |
| Example 23 | ** |
| Example 24 | ** |
| Example 25 | * |
| Example 26 | ** |
| Example 27 | ** |
| Example 28 | * |
| Example 29 | * |
| Example 30 | * |
| Example 31 | * |
| Example 32 | * |
| Example 33 | ** |
| Example 34 | ** |
| Example 35 | * |
| Example 36 | ** |
| Example 37 | * |
| Example 38 | * |
| Example 39 | ** |
| Example 40 | ** |
| Example 41 | ** |
| Example 42 | ** |
| Example 43 | * |
| Example 44 | ** |
| Example 45 | * |
| Example 46 | ** |
| Example 47 | * |
| Example 48 | ** |
| Example 49 | ** |
| Example 50 | * |
| Example 51 | * |
| Example 52 | * |
| Example 53 | * |
| Example 54 | * |
| Example 55 | ** |
| Example 56 | * |
| Example 57 | * |
| Example 58 | * |
| Example 59 | ** |
| Example 60 | ** |
| Example 61 | ** |
| Example 62 | ** |
| Example 63 | ** |
| Example 64 | * |
| Example 65 | ** |
| Example 66 | ** |
| Example 67 | ** |
| Example 68 | ** |
| Example 69 | ** |
| Example 70 | ** |
| Example 71 | * |
| Example 72 | ** |
| Example 73 | ** |
| Example 74 | ** |
| Example 75 | ** |
| Example 76 | ** |
| Example 77 | ** |
| Example 78 | * |
| Example 79 | ** |
| Example 80 | * |
| Example 81 | ** |
| Example 82 | * |
| Example 83 | ** |
| Example 84 | ** |
| Example 85 | ** |
| Example 86 | * |
| Example 87 | ** |
| Example 88 | ** |
| Example 89 | ** |
| Example 90 | ** |
| Example 91 | * |
| Example 92 | * |
| Example 93 | ** |
| Example 94 | * |
| Example 95 | * |
| Example 96 | ** |
| Example 97 | * |
| Example 98 | * |
| Example 99 | ** |
| Example 100 | * |
| Example 101 | ** |
| Example 102 | * |
| Example 103 | * |
| Example 104 | * |
| Example 105 | * |
| Example 106 | * |
| Example 107 | * |
| Example 108 | * |
| Example 109 | * |
| Example 110 | ** |
| Example 111 | * |
| Example 112 | ** |
| Example 113 | ** |
| Example 114 | *** |
| Example 115 | *** |
| Example 116 | * |
| Example 117 | ** |
| Example 118 | ** |
| Example 119 | * |
| Example 120 | ** |
| Example 121 | *** |
| Example 122 | *** |
| Example 123 | ** |
| Example 124 | ** |
| Example 125 | * |
| Example 126 | * |
| Example 127 | ** |
| Example 128 | * |
| Example 129 | * |
| Example 130 | ** |
| Example 131 | * |
| Example 132 | ** |
| Example 133 | * |
| Example 134 | *** |
| Example 135 | ** |
| Example 136 | ** |
| Example 137 | * |
| Example 138 | ** |
| Example 139 | * |
| Example 140 | ** |
| Example 141 | ** |
| Example 142 | * |
| Example 143 | ** |
| Example 144 | ** |
| Example 145 | ** |
| Example 146 | ** |
| Example 147 | ** |
| Example 148 | ** |
| Example 149 | ** |
| Example 150 | ** |
| Example 151 | * |
| Example 152 | ** |
| Example 153 | * |
| Example 154 | ** |
| Example 155 | ** |
| Example 156 | ** |
| Example 157 | * |
| Example 158 | * |
| Example 159 | * |
| Example 160 | ** |
| Example 161 | ** |
| Example 162 | ** |
| Example 163 | * |
| Example 164 | * |
| Example 165 | * |
| Example 166 | * |
| Example 167 | ** |
| Example 168 | ** |
| Example 169 | ** |
| Example 170 | ** |
| Example 171 | * |
| Example 172 | *** |
| Example 173 | ** |
| Example 174 | * |
| Example 175 | ** |
| Example 176 | ** |
| Example 177 | ** |
| Example 178 | ** |
| Example 179 | ** |
| Example 180 | ** |
| Example 181 | ** |
| Example 182 | ** |
| Example 183 | ** |
| Example 184 | ** |
| Example 185 | * |
| Example 186 | * |
| Example 187 | * |
| Example 188 | * |
| Example 189 | * |
| Example 190 | ** |
| Example 191 | *** |
| Example 192 | ** |
| Example 193 | ** |
| Example 194 | *** |
| Example 195 | ** |
| Example 196 | ** |
| Example 197 | ** |
| Example 198 | ** |
| Example 199 | *** |
| Example 200 | ** |
| Example 201 | ** |
| Example 202 | ** |
| Example 203 | ** |
| Example 204 | *** |
| Example 205 | ** |
| Example 206 | *** |
| Example 207 | *** |
| Example 208 | *** |
| Example 209 | *** |
| Example 210 | * |
| Example 211 | * |
| Example 212 | ** |
| Example 213 | *** |
| Example 214 | *** |
| Example 215 | * |
| Example 216 | ** |
| Example 217 | * |
| Example 218 | ** |
| Example 219 | * |
| Example 220 | ** |
| Example 221 | *** |
| Example 222 | *** |
| Example 223 | *** |
| Example 224 | ** |
| Example 225 | *** |
| Example 226 | *** |
| Example 227 | *** |
| Example 228 | *** |
| Example 229 | *** |
| Example 230 | * |
| Example 231 | * |
| Example 232 | ** |
| Example 233 | ** |
| Example 234 | ** |
| Example 235 | *** |
| Example 236 | *** |
| Example 237 | ** |
| Example 238 | ** |
| Example 239 | ** |
| Example 240 | ** |
| Example 241 | *** |
| Example 242 | ** |
| Example 243 | *** |
| Example 244 | ** |
| Example 245 | ** |
| Example 246 | *** |
| Example 247 | *** |
| Example 248 | *** |
| Example 249 | *** |
| Example 250 | *** |
| Example 251 | *** |
| Example 252 | ** |
| Example 253 | *** |
| Example 254 | *** |
| Example 255 | *** |
| Example 256 | *** |
| Example 257 | * |
| Example 259 | *** |
| Example 260 | ** |
| Example 261 | *** |
| Example 262 | *** |
| Example 263 | ** |
| Example 264 | *** |
| Example 265 | *** |
| Example 266 | *** |
| Example 267 | *** |
| Example 268 | *** |
| Example 269 | *** |
| Example 270 | *** |
| Example 271 | ** |
| Example 272 | *** |
| Example 273 | *** |
| Example 274 | *** |
| Example 275 | *** |
| Example 276 | *** |
| Example 277 | *** |
| Example 278 | ** |
| Example 279 | ** |
| Example 280 | *** |
| Example 281 | ** |
| Example 282 | ** |
| Example 283 | *** |
| Example 284 | *** |
| Example 285 | ** |
| Example 286 | ** |
| Example 287 | * |
| Example 288 | *** |
| Example 289 | ** |
| Example 290 | ** |
| Example 291 | ** |
| Example 292 | ** |
| Example 293 | ** |
| Example 294 | *** |
| Example 295 | ** |
| Example 296 | *** |
| Example 297 | ** |
| Example 298 | *** |
| Example 299 | *** |
| Example 300 | ** |
| Example 301 | * |
| Example 302 | * |
| Example 303 | * |
| Example 304 | * |
| Example 305 | ** |
| Example 306 | ** |
| Example 307 | ** |
| Example 308 | ** |
| Example 309 | ** |
| Example 310 | ** |
| Example 311 | ** |
| Example 312 | * |
| Example 313 | ** |
| Example 314 | * |
| Example 315 | ** |
| Example 316 | * |
| Example 317 | * |
| Example 318 | *** |
| Example 319 | ** |
| Example 320 | ** |
| Example 321 | ** |
| Example 322 | * |
| Example 323 | * |
| Example 324 | * |
| Example 325 | * |
| Example 326 | * |
| Example 327 | * |
| Example 328 | * |
| Example 329 | ** |
| Example 330 | *** |
| Example 331 | ** |
| Example 332 | ** |
| Example 333 | ** |
| Example 334 | ** |
| Example 335 | ** |
| Example 336 | * |
| Example 337 | * |
| Example 338 | * |
| Example 339 | * |
| Example 340 | * |
| Example 341 | * |
| Example 342 | * |
| Example 343 | * |
| Example 344 | * |
| Example 345 | * |
| Example 346 | ** |
| Example 347 | ** |
| Example 348 | * |
| Example 349 | ** |
| Example 350 | * |
| Example 351 | ** |
| Example 352 | * |
| Example 353 | * |

*** = LRRK2 IC50 <100 nM
** = LRRK2 IC50 between 100 nM and 1 μM
* = LRRK2 IC50 between 1 μM and 10 μM

TABLE 2
Kinase selectivity data of representative compounds

| | Example | Example | | Example | Example |
|---|---|---|---|---|---|
| Kinase | 121 | 172 | Kinase | 121 | 172 |
| AMPK | 29 | 0 | PIM1 | 15 | 0 |
| BRSK2 | 13 | 0 | PKA | 26 | 5 |
| BTK | 0 | 12 | PKBa | 19 | 0 |
| CAMK1 | 5 | 14 | PKBb | 3 | 0 |
| CAMKKb | 12 | 21 | PKCa | 2 | 0 |
| CDK2- | 61 | 0 | PKD1 | 29 | 1 |
| Cyclin A | | | | | |
| CHK1 | 10 | 0 | PLK1 | 19 | 3 |
| CHK2 | 13 | 40 | PRAK | 0 | 0 |
| CK1 | 31 | 6 | PRK2 | 39 | 16 |
| CK2 | 0 | 3 | ROCK 2 | 57 | 7 |
| CSK | 6 | 11 | S6K1 | 0 | 14 |
| DYRK1A | 27 | 27 | SGK1 | 23 | 1 |
| EPH-B3 | 6 | 3 | SmMLCK | 22 | 18 |
| ERK1 | 1 | 0 | Src | 0 | 6 |
| ERK2 | 0 | 9 | SRPK1 | 21 | 17 |
| FGF-R1 | 36 | 0 | SYK | 0 | 4 |
| GSK3b | 25 | 5 | TBK1 | 6 | 14 |
| HIPK2 | 0 | 0 | Aurora B | 0 | 15 |
| IKKb | 15 | 22 | EF2K | 0 | 0 |
| IKKe | 19 | 17 | IGF-1R | 46 | 18 |
| IRR | 22 | 15 | PKCz | 0 | 10 |
| JNK1 | 7 | 17 | Aurora A | 46 | 0 |
| JNK2 | 0 | 22 | EPH-A2 | 8 | 0 |
| Lck | 0 | 25 | GCK | 68 | 19 |
| MARK3 | 12 | 18 | HER4 | 0 | 10 |
| MELK | 61 | 5 | IR | 8 | 4 |
| MKK1 | 47 | 17 | IRAK4 | 12 | 0 |
| MNK1 | 1 | 0 | JAK2 | 47 | 12 |
| MNK2 | 7 | 0 | LKB1 | 42 | 8 |
| MSK1 | 48 | 1 | MEKK1 | 0 | 7 |
| MST2 | 55 | 10 | MINK1 | 17 | 18 |
| MST4 | 23 | 10 | MLK1 | 19 | 13 |
| NEK2a | 0 | 5 | MLK3 | 23 | 29 |
| NEK6 | 6 | 1 | NUAK1 | 59 | 14 |
| p38a MAPK | 0 | 6 | RIPK2 | 1 | 0 |
| PAK4 | 26 | 24 | TAK1 | 72 | 9 |
| PDK1 | 13 | 0 | TrkA | 26 | 5 |
| PHK | 7 | 26 | TTK | 30 | 14 |

Data are expressed as percentage inhibition of each specific kinase at 1 μM inhibitor concentration

1. A compound of formula I, or a pharmaceutically acceptable salt or ester thereof, 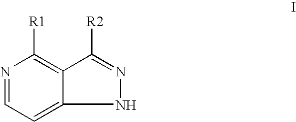
wherein:
R¹ is selected from:
aryl;
heteroaryl;
—NHR³;
fused aryl-C₄₋₇-heterocycloalkyl;
—CONR⁴R⁵;
—NHCOR⁶;
—C₃₋₇-cycloalkyl;
—NR³R⁶;
OR³;
OH;
NR⁴R⁵; and
—C₁₋₆ alkyl optionally substituted with a substituent selected from R¹¹ and a group A;
wherein said aryl, heteroaryl, fused aryl-C₄₋₇-heterocycloalkyl and C₄₋₇-heterocycloalkyl are each optionally substituted with one or more substituents selected from C₁₋₆-alkyl, C₃₋₇-cycloalkyl, heteroaryl, C₄₋₇-heterocycloalkyl, aryl and a group A, and said C₁₋₆-alkyl, C₃₋₇-cycloalkyl, heteroaryl, C₄₋₇-heterocycloalkyl, and aryl substituents are in turn each optionally substituted with one or more groups selected from R¹¹ and a group A;
R² is selected from hydrogen, aryl, C₁₋₆-alkyl, C₂₋₆-alkenyl, C₃₋₇-cycloalkyl, heteroaryl, C₄₋₇ heterocycloalkyl, fused aryl-C₄₋₇-heterocycloalkyl and halogen, wherein said C₁₋₆-alkyl, C₂₋₆-alkenyl, aryl, heteroaryl, fused aryl-C₄₋₇-heterocycloalkyl and C₄₋₇-heterocycloalkyl are each optionally substituted with one or more substituents selected from R¹¹ and A;
each R³ is selected from aryl, heteroaryl, C₄₋₇-heterocycloalkyl, C₃₋₇-cycloalkyl, fused aryl-C₄₋₇-heterocycloalkyl and C₁₋₆-alkyl, each of which is optionally substituted with one or more substituents selected from R¹¹ and A;
R⁴ and R⁵ are each independently selected from hydrogen, C₃₋₇-cycloalkyl, C₁₋₆-alkyl-C₃₋₇-cycloalkyl, aryl, heteroaryl, C₁₋₆-alkyl and a C₃₋₆-heterocycloalkyl ring optionally further containing one or more groups selected from oxygen, sulfur, nitrogen and CO, and optionally substituted by one or more R¹⁰ groups, wherein each C₁₋₆-alkyl, heteroaryl and aryl is optionally substituted by one or more substituents selected from C₁₋₆-alkyl, halogen, cyano, hydroxyl, aryl, halo-substituted aryl, heteroaryl, —NR⁸R⁹, —NR⁶R⁷, NR⁷(CO)R⁶, —NR⁷COOR⁶, —NR⁷(SO₂)R⁶, —COOR⁶, —CONR⁸R⁹, OR⁶, —SO₂R⁶ and a C₃₋₆-heterocycloalkyl ring optionally further containing one or more groups selected from oxygen, sulfur, nitrogen and CO and optionally substituted by one or more or R¹⁰ groups; or
R⁴ and R⁵ together with the N to which they are attached form a C₃₋₆-heterocycloalkyl ring optionally further containing one or more groups selected from oxygen, sulfur, nitrogen and CO, wherein said C₃₋₆-heterocycloalkyl ring is saturated or unsaturated and is optionally substituted with one or more groups selected from A, NR⁸R⁹ and R¹⁰;
each R⁶ is independently selected from C₁₋₆-alkyl, C₃₋₇ cycloalkyl, C₄₋₇-heterocycloalkyl, aryl and heteroaryl, each of which is optionally substituted by one or more substituents selected from R¹⁰, R¹¹ and A;
each R⁷ is selected from hydrogen, C₁₋₆-alkyl and C₃₋₇-cycloalkyl, wherein said C₁₋₆-alkyl is optionally substituted by one or more halogens;
each of R⁸ and R⁹ is independently selected from hydrogen and C₁₋₆-alkyl, wherein said C₁₋₆-alkyl group is optionally substituted by one or more halogens; or
R⁸ and R⁹ together with the N to which they are attached form a C₄₋₆-heterocycloalkyl ring optionally further containing one or more heteroatoms selected from oxygen and sulfur, wherein said C₄₋₆-heterocycloalkyl ring is optionally substituted by one or more R¹⁰ groups; and
each R¹⁰ is selected from C₃₋₇-cycloalkyl, aryl, heteroaryl, O-heteroaryl, aralkyl and C₁₋₆-alkyl, each of which is optionally substituted by one or more A groups, wherein where R¹⁰ is C₁₋₆-alkyl and two or more R¹⁰ groups are attached to the same carbon atom, the R¹⁰ groups may be linked to form a spiroalkyl group; and
each R¹¹ is independently selected from C₁₋₆-alkyl, C₃₋₇-cycloalkyl, C₁₋₆-alkyl-C₃₋₇-cycloalkyl, C₁₋₆-alkyl-heteroaryl, C₄₋₇-heterocycloalkyl, aryl and heteroaryl, each of which is optionally substituted with one or more substituents selected from A; and
A is selected from halogen, —NR⁴SO₂R⁵, —CN, —OR⁶, —NR⁴R⁵, —NR⁷R¹¹, hydroxyl, —CF₃, —CONR⁴R⁵, —NR⁴COR⁵, —NR⁷(CO)NR⁴R⁵, —NO₂, —CO₂H, —CO₂R⁶, —SO₂R⁶, —SO₂NR⁴R⁵, —NR⁴COR⁵, —NR⁴COOR⁵, C₁₋₆-alkyl, aryl and —COR⁶.

2. A compound according to claim 1 wherein R² is selected from:
hydrogen;
halogen, more preferably bromine;
aryl optionally substituted by one or more substituents selected from R¹¹ and A;
C₁₋₆-alkyl optionally substituted by one or more substituents selected from R¹¹ and A;
C₂₋₆-alkenyl optionally substituted by one or more A substituents;
C₃₋₇-cycloalkyl;
heteroaryl optionally substituted by one or more substituents selected from R¹¹ and A;
C₄₋₇-heterocycloalkyl; and
fused aryl-C₄₋₇-heterocycloalkyl.

3. A compound according to claim 1 wherein R² is selected from:
aryl optionally substituted by one or more substituents selected from —NR⁴R⁵, —NR⁴COR⁵, —CONR⁴R⁵, OR⁶, halogen, optionally substituted C₁₋₆-alkyl, CN, C₄₋₇-heterocycloalkyl and heteroaryl;
C₁₋₆-alkyl optionally substituted by one or more substituents selected from —NR⁴COR⁵, —CONR⁴R⁵, —NR⁴R⁵, OR⁶, optionally substituted aryl, optionally substituted heteroaryl and C₄₋₇-heterocycloalkyl;
C₂₋₆-alkenyl optionally substituted by one or more —CONR⁴R⁵ substituents;
C₃₋₇-cycloalkyl, more preferably cyclopropyl;
heteroaryl optionally substituted by one or more substituents selected from —NR⁴R⁵, C₄₋₇-heterocycloalkyl, C₁₋₆-alkyl, C₃₋₇-cycloalkyl, C₁₋₆-alkyl-C₃₋₇-cycloalkyl and OR⁶;
C₄₋₇-heterocycloalkyl; and
fused aryl-C₄₋₇-heterocycloalkyl.

4. A compound according to claim 1 wherein R² is selected from:
a phenyl group optionally substituted by one or more substituents selected from —NHCO—C₁₋₆-alkyl, —CONHC₁₋₆-alkyl, CO—(N-morpholinyl), Cl, F, —OC₁₋₆-alkyl, —CONMe₂, OCF₃, CN, CF₃, C₁₋₆-alkyl-(A), N-morpholinyl and pyrazolyl;
a heteroaryl group selected from pyridinyl, quinolinyl, pyrazoyl, furanyl and pyrimidinyl, each of which may be optionally substituted by one or more substituents selected from C₁₋₆-alkyl, aralkyl, OC₁₋₆-alkyl, N-morpholinyl;
a C₁₋₆-alkyl group optionally substituted by one or more substituents selected from —CONR⁴R⁵, phenyl, pyridinyl and oxadiazolyl and piperidinyl, wherein said phenyl, pyridinyl and oxadiazolyl and piperidinyl groups are each optionally further substituted by one or more —NR⁴COR⁵, —CONR⁴R⁵, COR⁶, SO₂R⁶ or aryl groups.

5. A compound according to claim 4 wherein each —CONR⁴R⁵ group is independently selected from:
—CO(N-morpholinyl), —CO(N-piperidinyl), —CO(N-pyrrolidinyl), —CO—(N-piperazinyl), each of which may be optionally further substituted by one or more substituents selected from aryl, heteroaryl, —OR⁶, CF₃, aralkyl, —NR⁴COR⁵—CONR⁴R⁵, —NR⁴R⁵, halogen, C₁₋₆-alkyl; and
—CON(C₁₋₆-alkyl)₂, CONH(C₁₋₆-alkyl), CON(C₁₋₆-alkyl)(aralkyl), CONH(C₃₋₇-cycloalkyl), —CONH(aryl), —CONH(heteroaryl), wherein said C₁₋₆-alkyl, aralkyl, aryl and heteroaryl groups are each optionally further substituted by one or more R¹¹ or A groups.

6. A compound according to claim 1 wherein R² is a C₁₋₆-alkyl group optionally substituted by one or more substituents selected from —NR⁴COR⁵, —CONR⁴R⁵, —NR⁴R⁵, OR⁶, C₄₋₇-heterocycloalkyl, heteroaryl and aryl, wherein said aryl group is optionally substituted by one or more substituents selected from —NR⁴COR⁵ and —CONR⁴R⁵.

7. A compound according to claim 1 wherein R² is selected from —CH₂CH₂CO—NR⁴R⁵, C₁₋₆-alkyl, C₃₋₇ cycloalkyl and a heteroaryl selected from furanyl and pyrazolyl, wherein said furanyl and pyrazolyl groups may be optionally substituted by one or more substituents selected from C₁₋₆-alkyl, C₃₋₇-cycloalkyl and C₁₋₆-alkyl-C₃₋₇-cycloalkyl.

8. A compound according to claim 7 wherein R² is selected from Me, 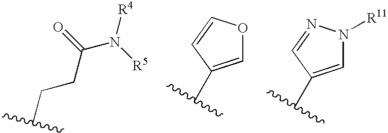
wherein R⁴ and R⁵ together with the N to which they are attached form a C₃₋₆-heterocycloalkyl ring optionally further containing one or more groups selected from oxygen, sulfur, nitrogen and CO, wherein said C₃₋₆-heterocycloalkyl ring is saturated or unsaturated and is optionally substituted with one or more groups selected from A, NR⁸R⁹ and R¹⁰.

9. A compound according to claim 1 wherein R² is an unsubstituted C₁₋₆-alkyl group, more preferably methyl.

10. A compound according to claim 1 wherein R¹ is selected from:
—NHR³;
aryl;
heteroaryl;
C₄₋₇-heterocycloalkyl;
fused aryl-C₄₋₇-heterocycloalkyl;
—C₃₋₇-cycloalkyl;
—NR³R⁶;
OR³;
NR⁴R⁵; and
—C₁₋₆ alkyl optionally substituted with a substituent selected from R¹¹ and a group A;
wherein said aryl, heteroaryl, fused aryl-C₄₋₇-heterocycloalkyl and C₄₋₇-heterocycloalkyl are each optionally substituted with one or more substituents selected from C₁₋₆-alkyl, C₃₋₇-cycloalkyl, heteroaryl, C₄₋₇-heterocycloalkyl, aryl and a group A, and said C₁₋₆-alkyl, C₃₋₇-cycloalkyl, heteroaryl, C₄₋₇-heterocycloalkyl, and aryl substituents are in turn each optionally substituted with one or more groups selected from R¹¹ and a group A.

11. A compound according to claim 1 wherein R¹ is —NHR³, wherein R³ is selected from C₁₋₆-alkyl, C₃₋₇-cycloalkyl, C₄₋₇-heterocycloalkyl and aryl, each of which may be optionally substituted by one or more with one or more substituents selected from R¹¹ and A.

12. A compound according to claim 11 wherein R¹ is —NHR³ and R³ is selected from:
C₁₋₆-alkyl, optionally substituted by one or more —OR⁶, NR⁴COR⁵, heteroaryl, aryl, C₄₋₇-heterocycloalkyl, and C₃₋₇-cycloalkyl groups, wherein said aryl and heteroaryl groups are each independently optionally further substituted by one or more groups selected from CF₃, halogen, C₁₋₆-alkyl, —OR⁶ and —NR⁴R⁵;
a phenyl group optionally substituted by one or more substituents selected from —OR⁶, NR⁴COR⁵, —CONR⁴R⁵, aryl, —NR⁴R⁵, C₁₋₆-alkyl-heteroaryl, heteroaryl, halogen, —SO₂R⁶, CN, CF₃, C₁₋₆-alkyl, —SO₂NR⁴R⁵, —NR⁴SO₂R⁵, wherein said C₁₋₆-alkyl, heteroaryl and aryl groups are each independently optionally further substituted by one or more groups selected from CN, CF₃, halogen, C₁₋₆-alkyl, —OR⁶ and —NR⁴R⁵;
a heteroaryl group optionally substituted by one or more substituents selected from aryl, C₁₋₆-alkyl, and —NR⁴R⁵, wherein said aryl group is optionally further substituted by one or more A groups;
a C₄₋₇-heterocycloalkyl optionally substituted by one or more —COR^{E} groups;
a C₃₋₇-cycloalkyl group optionally substituted by one or more halogen or C₁₋₆-alkyl groups.

13. A compound according to claim 1 wherein R¹ is —OR³, wherein R³ is selected from C₁₋₆-alkyl, C₃₋₇-cycloalkyl, C₄₋₇-heterocycloalkyl and aryl, each of which may be optionally substituted by one or more with one or more substituents selected from R¹¹ and A.

14. A compound according to claim 13 wherein R¹ is —OR³, wherein R³ is C₁₋₆-alkyl, C₃₋₇-cycloalkyl or C₄₋₇-heterocycloalkyl, each of which may be optionally substituted by one or more A substituents.

15. A compound according to claim 1 wherein R¹ is aryl or heteroaryl, each of which may be optionally substituted by one or more with one or more substituents selected from R¹¹ and A.

16. A compound according to claim 1 wherein R¹ is —NH—C₃₋₇-cycloalkyl or NH—C₄₋₇-heterocycloalkyl, each of which may be optionally substituted by one or more A substituents.

17. A compound according to claim 1 wherein R³ is cyclohexyl or tetrahydropyranyl, each of which may be optionally substituted by one or more A substituents.

18. A compound according to claim 1 wherein R¹ is selected from the following:

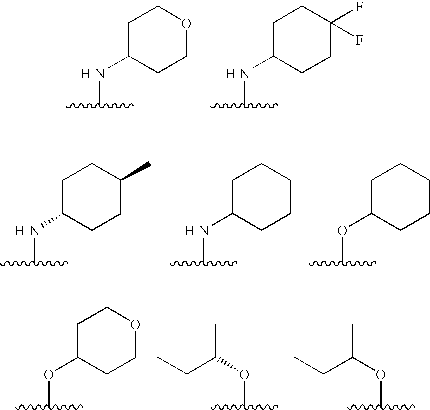

19. A compound according to claim 18 wherein R¹ is —NH-cyclohexyl.

20. A compound according to claim 1 wherein R¹ is —NHR³ and R² is an unsubstituted C₁₋₆-alkyl group, more preferably methyl.

21. A compound according to claim 1 wherein R¹ is —NHR³ and R² is a C₁₋₆-alkyl group substituted by one or more —CONR⁴R⁵ groups.

22. A compound according to claim 1 wherein R¹ is —NHR³ and R² is an aryl or heteroaryl group, each of which may be optionally substituted by one or more substituents selected from C₄₋₇-heterocycloalkyl, C₁₋₆-alkyl, C₃₋₇-cycloalkyl, C₁₋₆-alkyl-C₃₋₇-cycloalkyl and OR⁶.

23. A compound according to claim 1 wherein R¹ is —OR³ and R² is a C₁₋₆-alkyl group, more preferably methyl.

24. A compound according to claim 1 wherein R¹ is selected from: 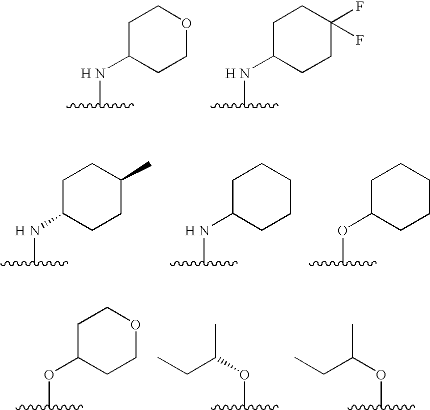
and R² is selected from Me 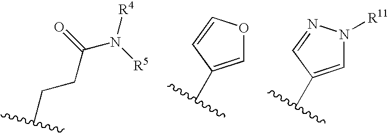
wherein R⁴ and R⁵ together with the N to which they are attached form a C₃₋₆-heterocycloalkyl ring optionally further containing one or more groups selected from oxygen, sulfur, nitrogen and CO, wherein said C₃₋₆-heterocycloalkyl ring is saturated or unsaturated and is optionally substituted with one or more groups selected from A, NR⁸R⁹ and R¹⁰.

25. A compound according to claim 1 which is selected from the following:

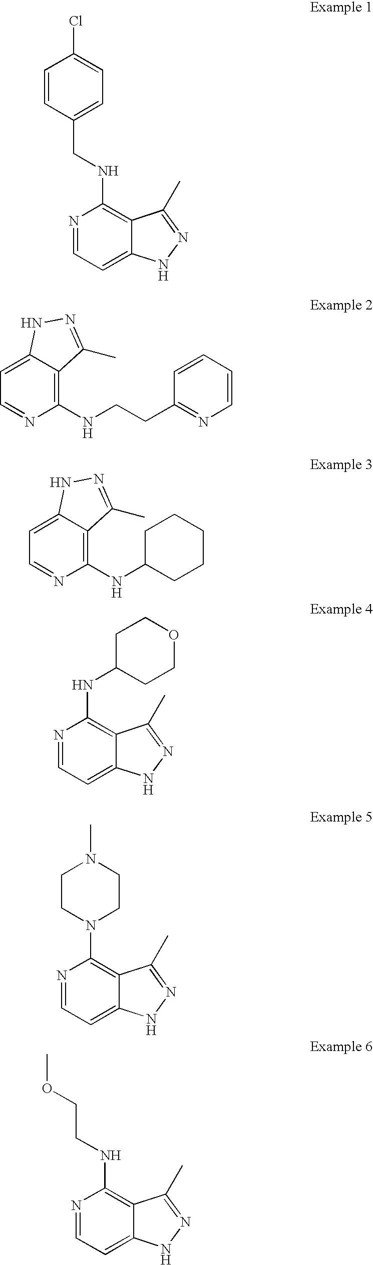

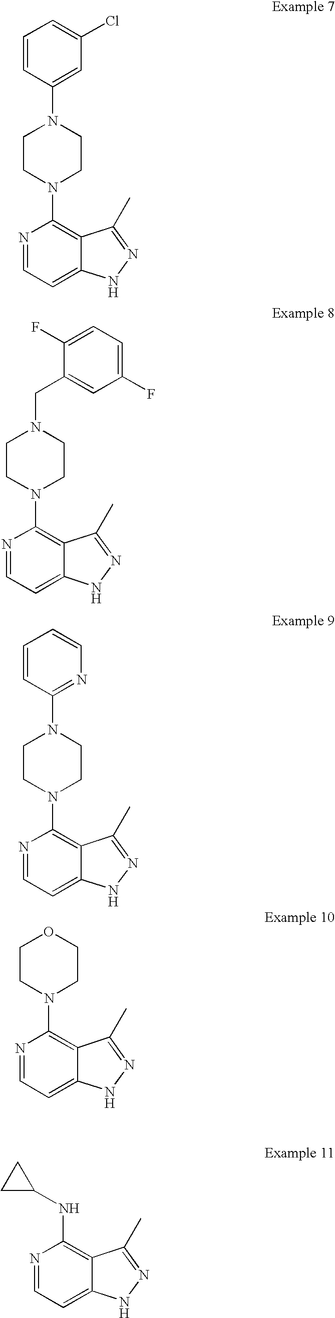

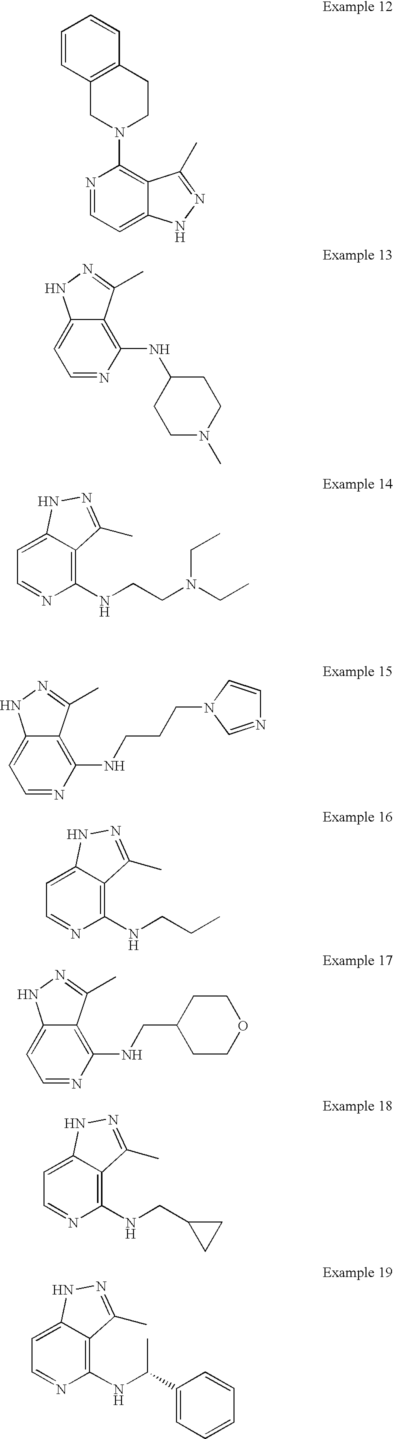

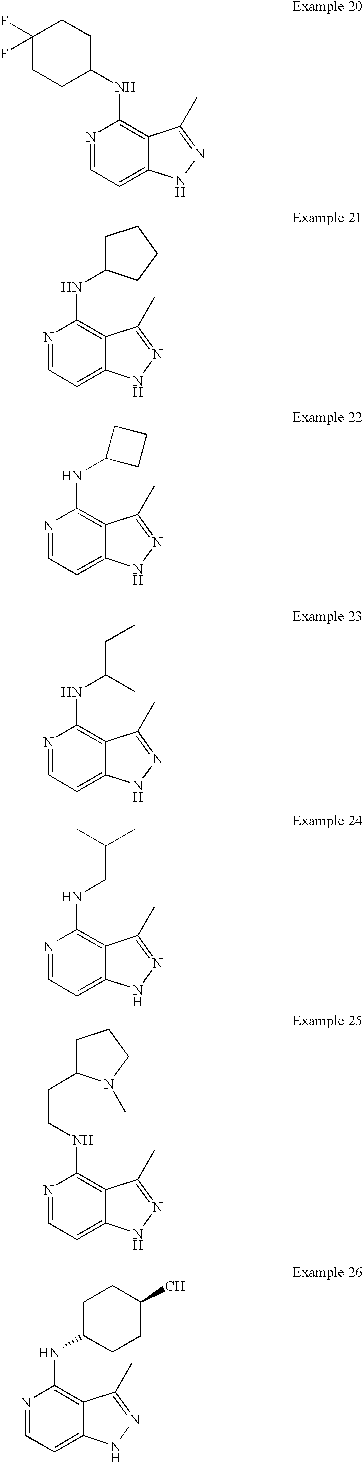

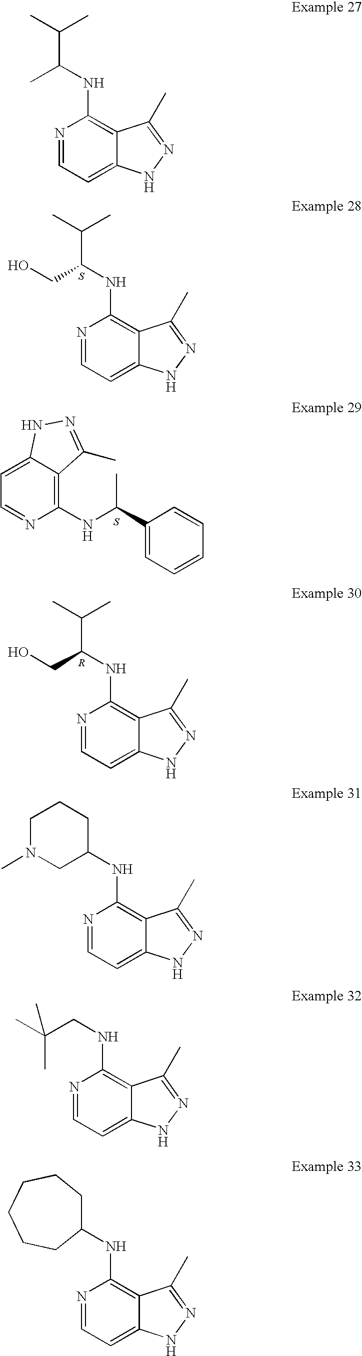

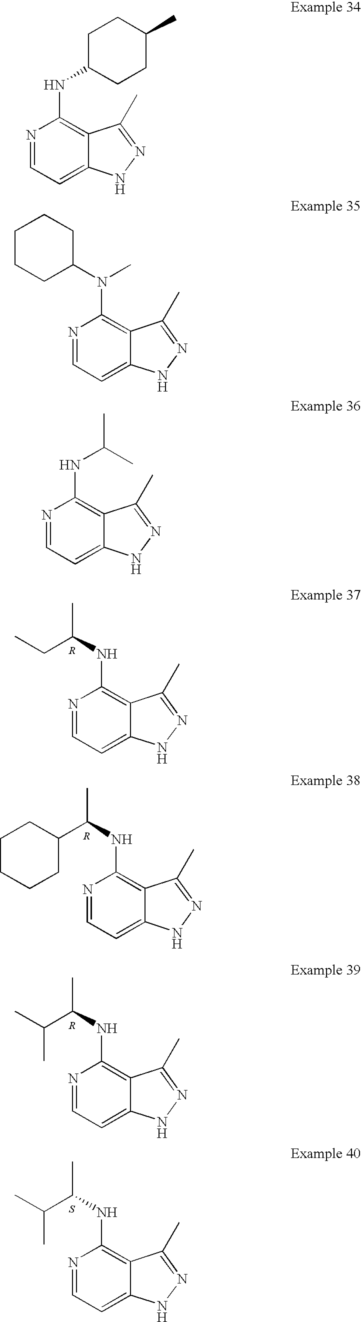

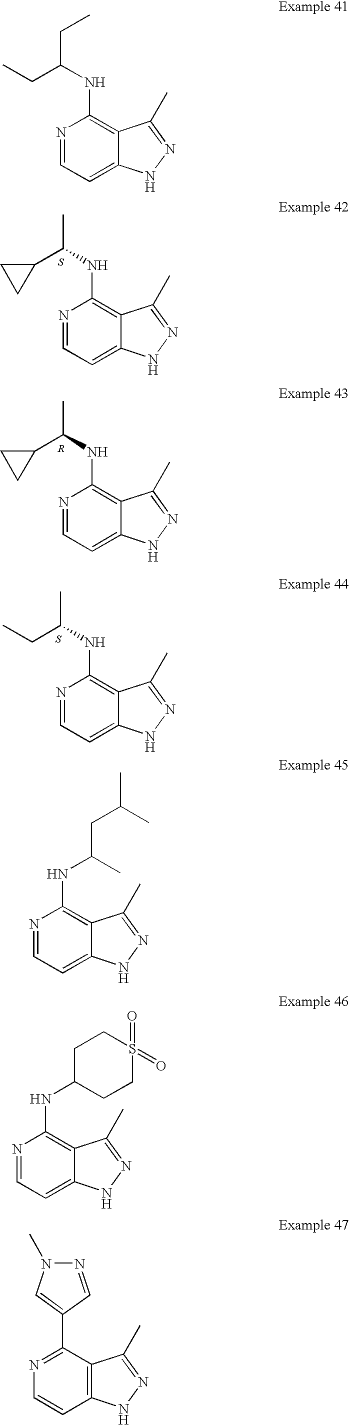

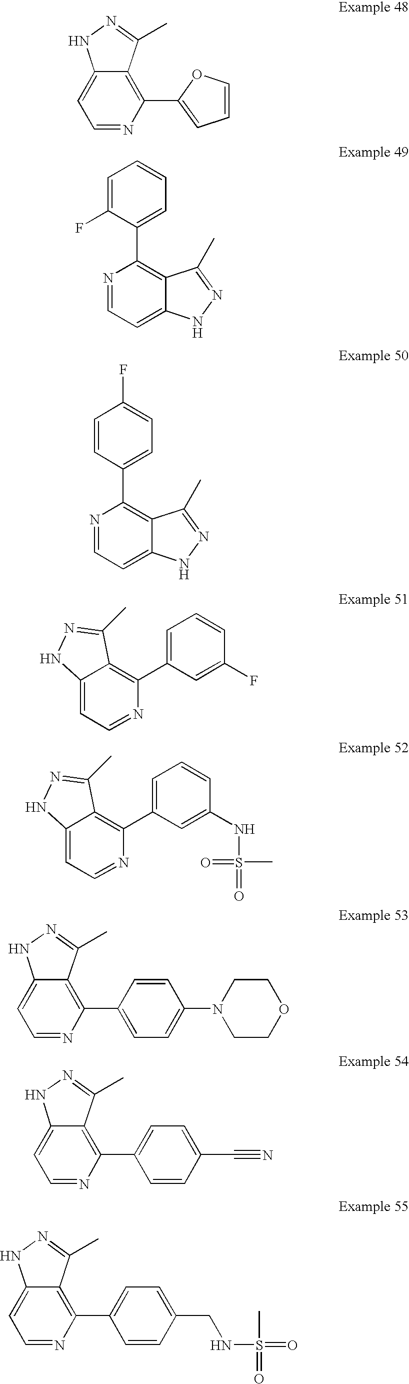

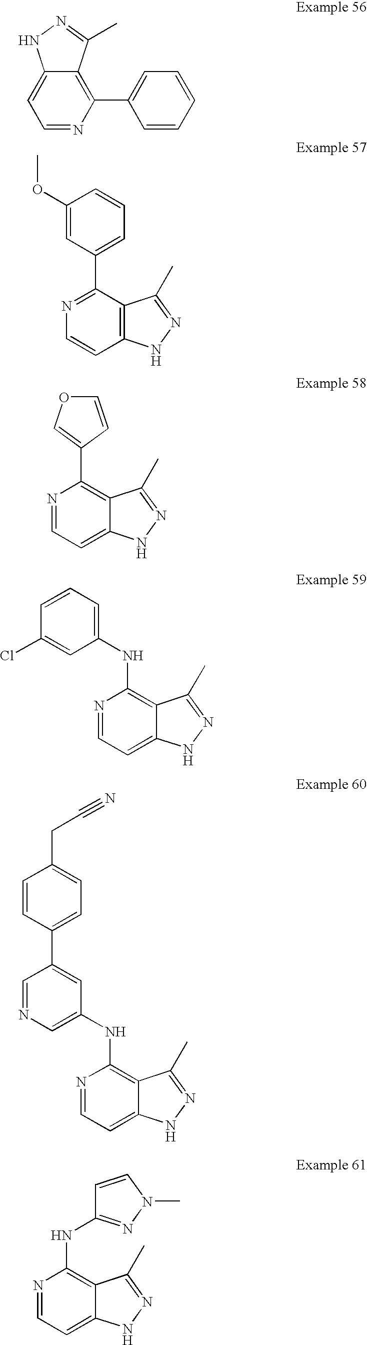

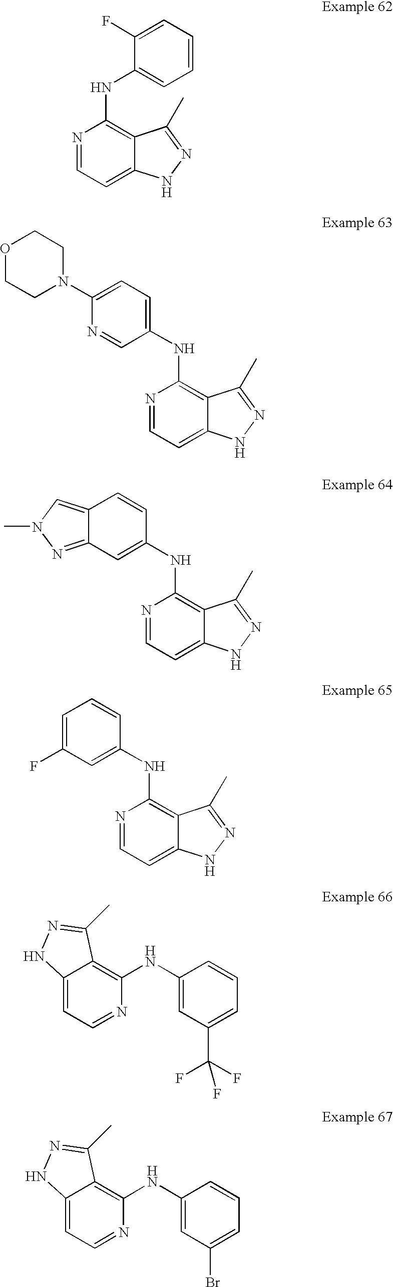

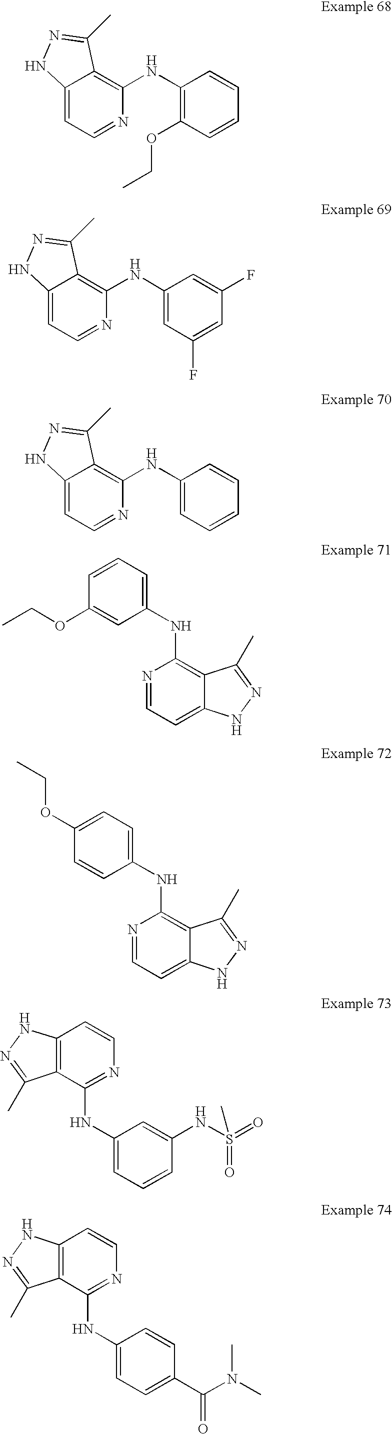

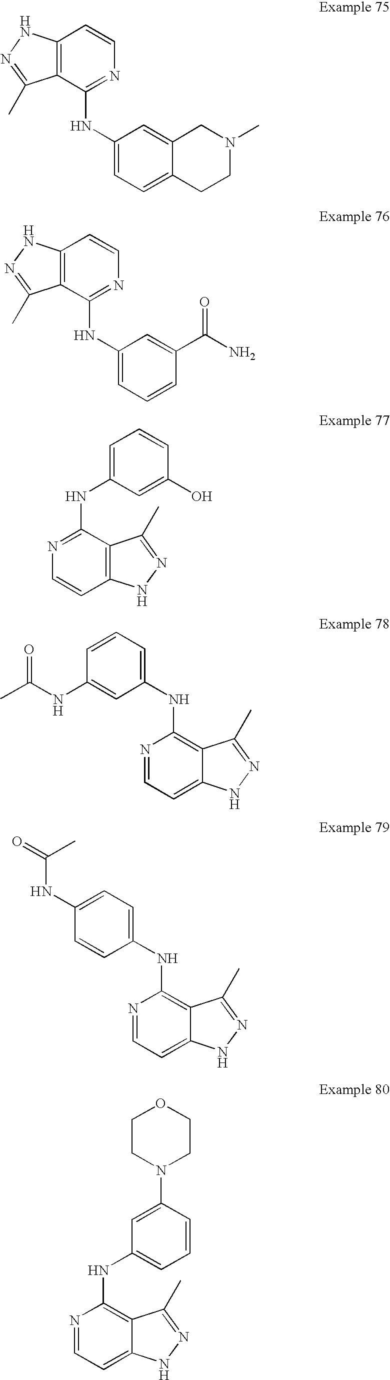

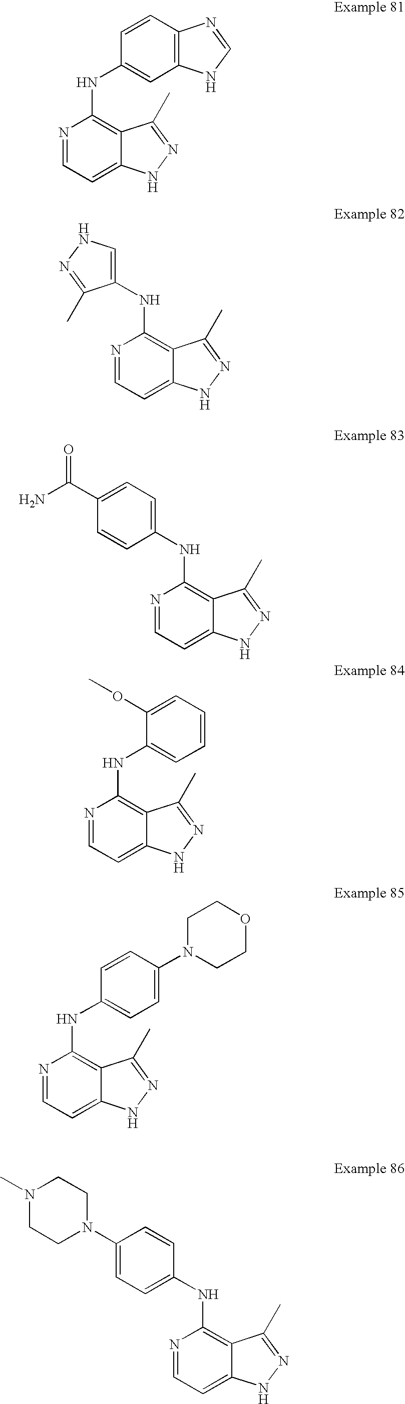

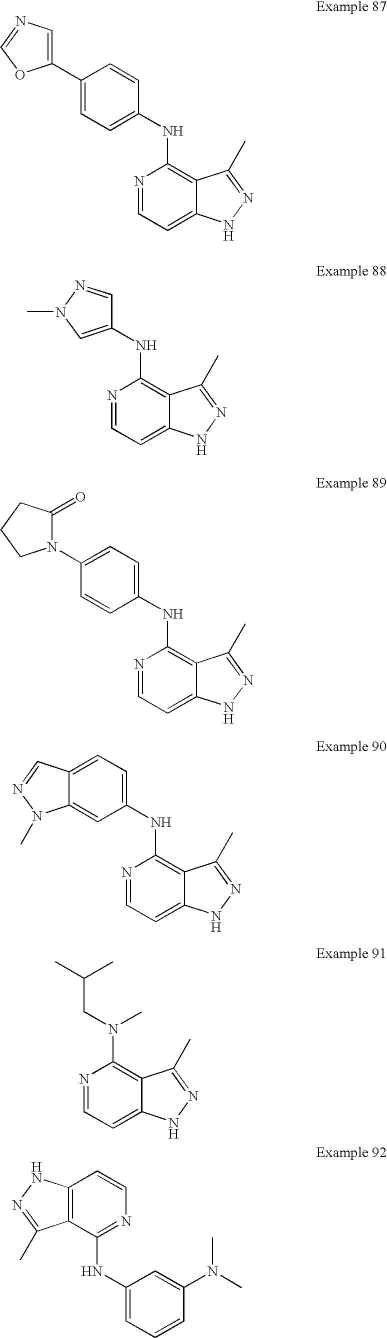

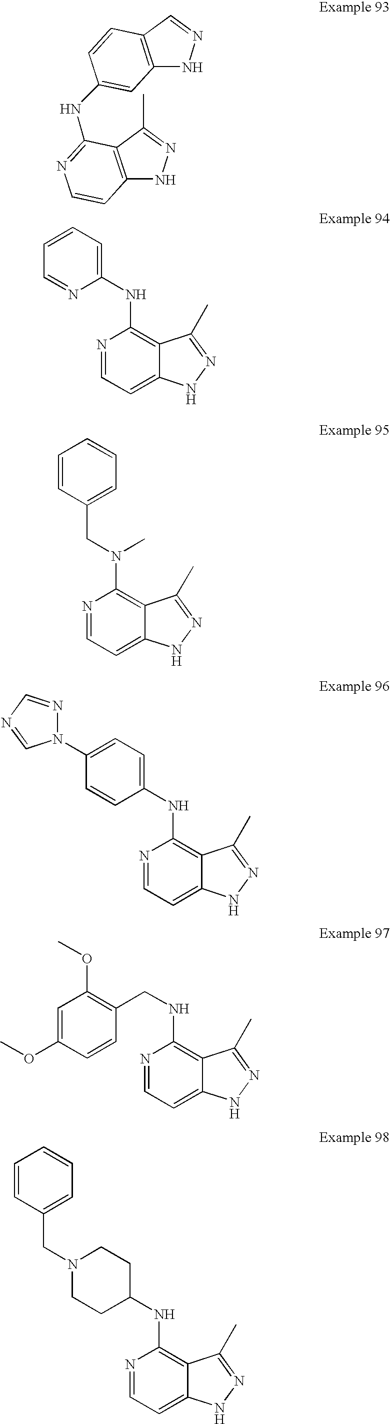

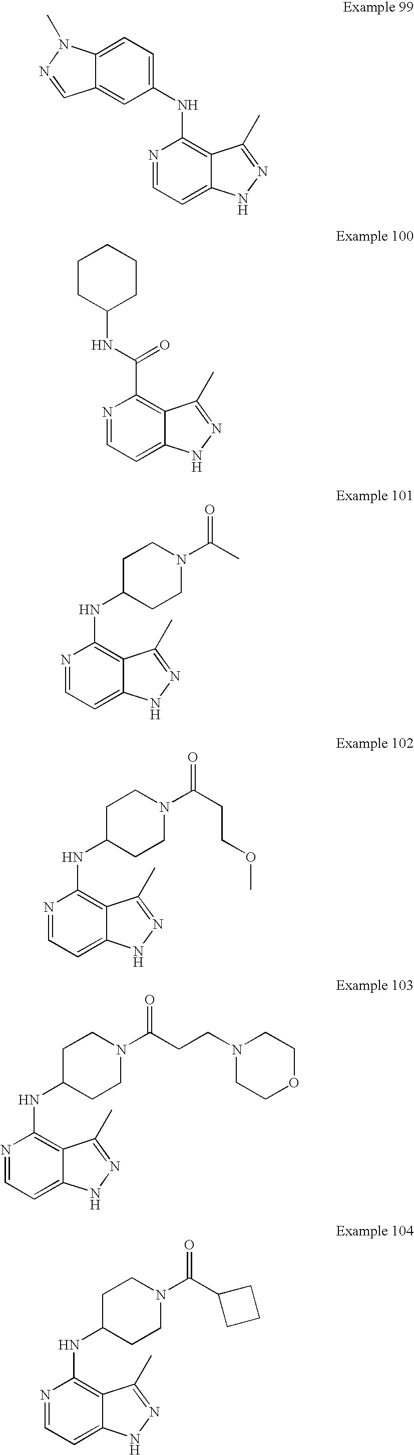

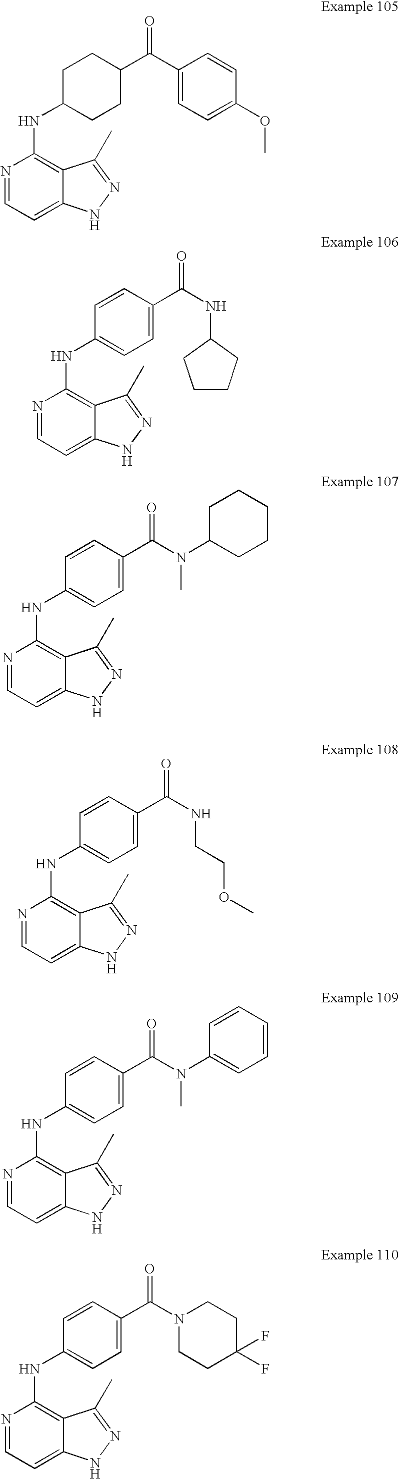

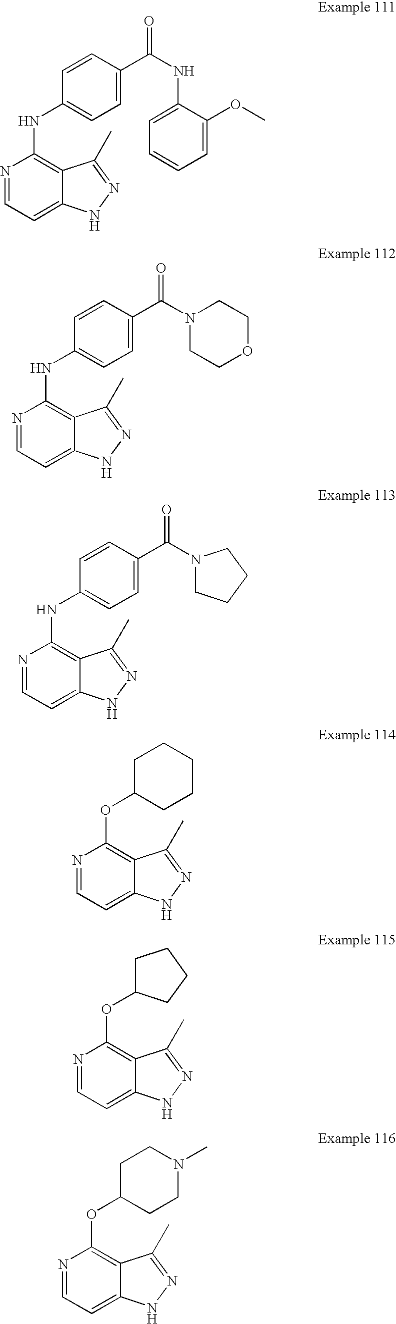

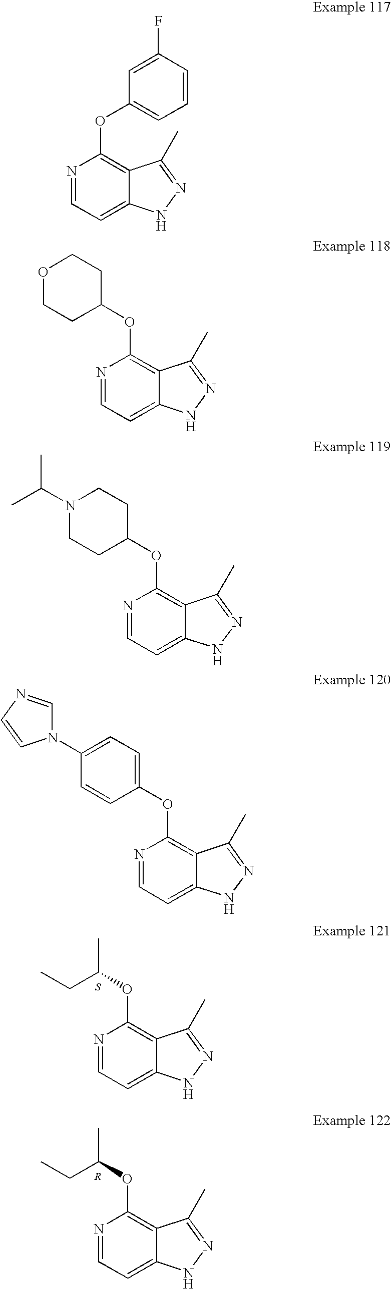

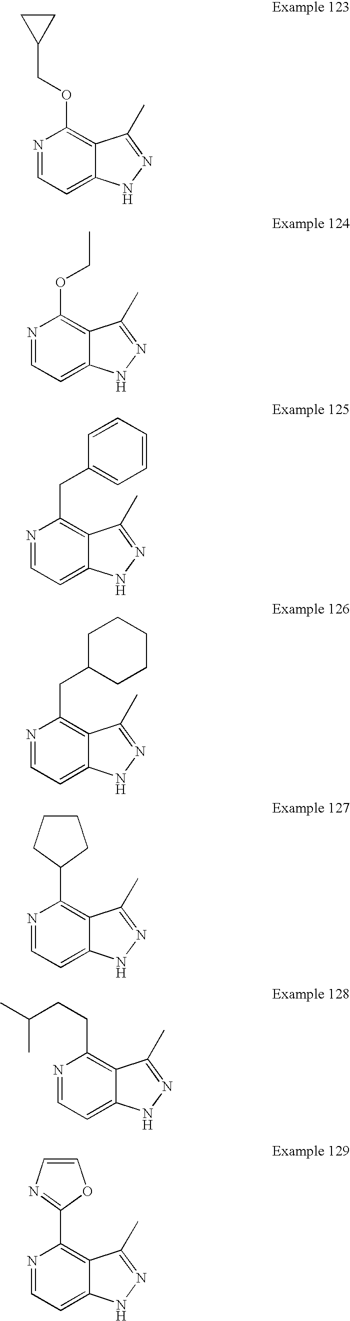

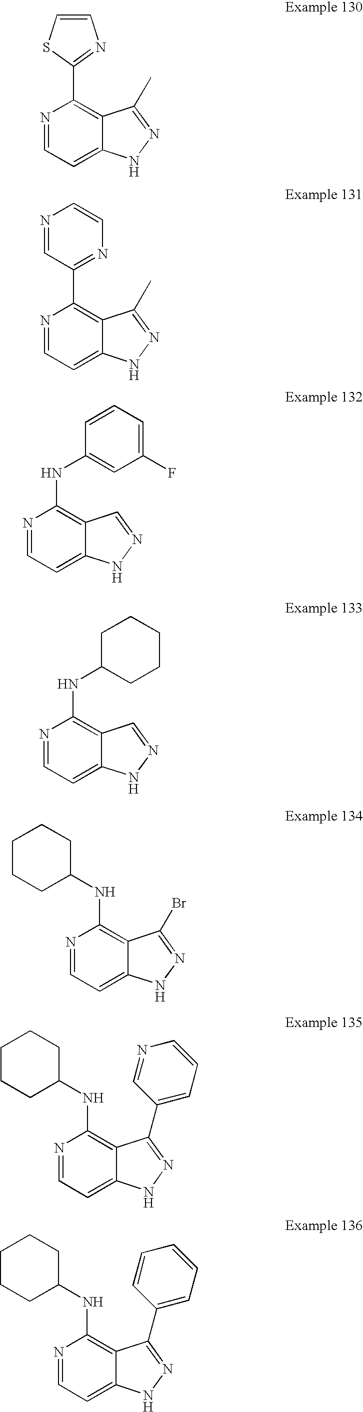

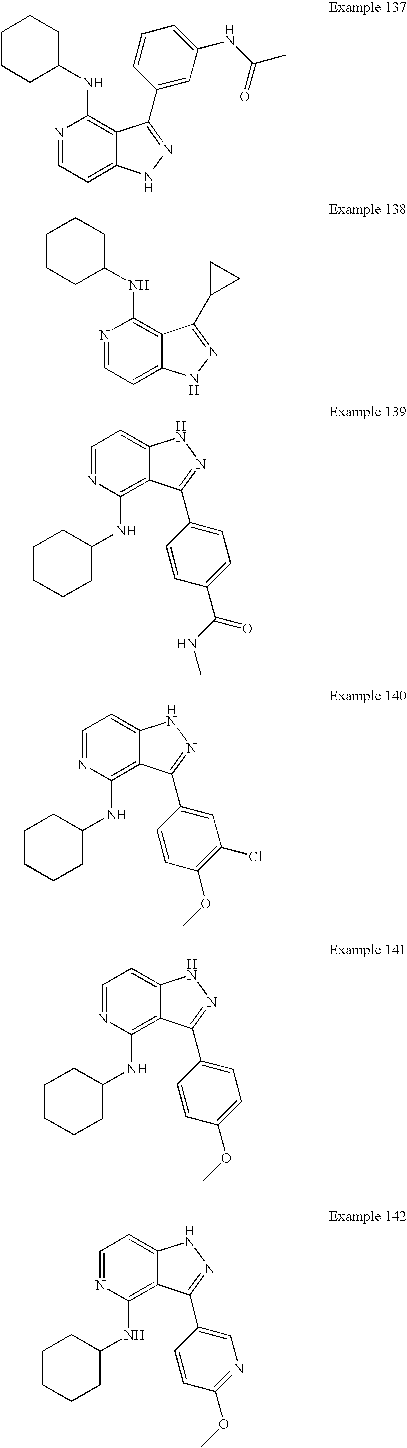

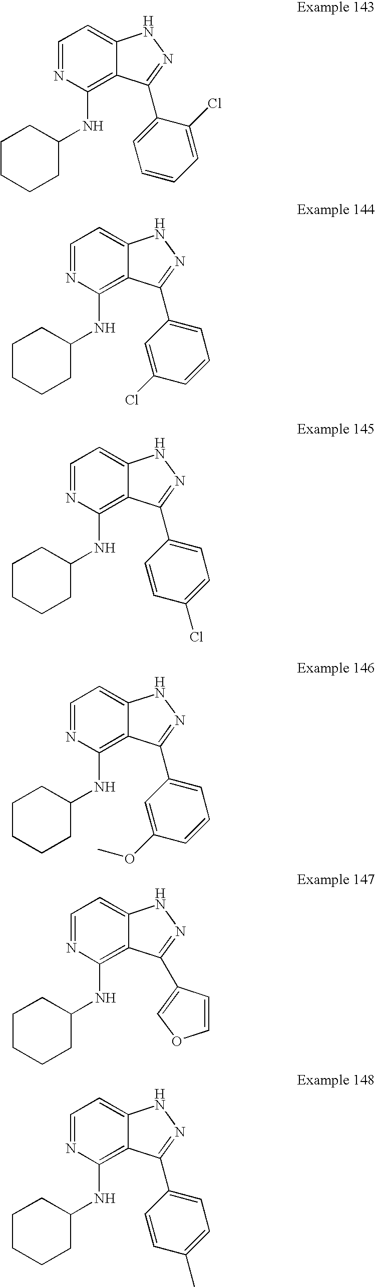

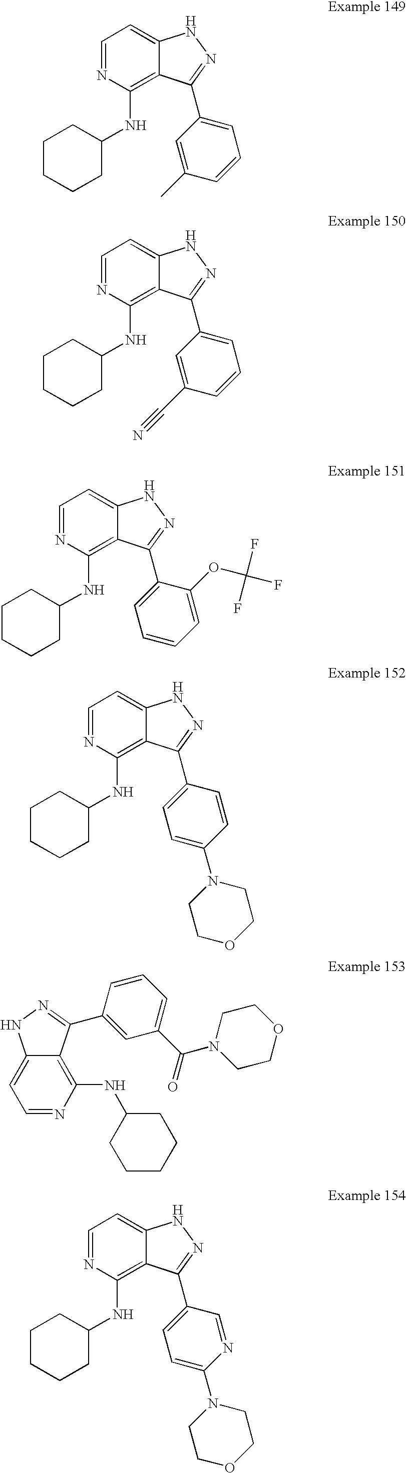

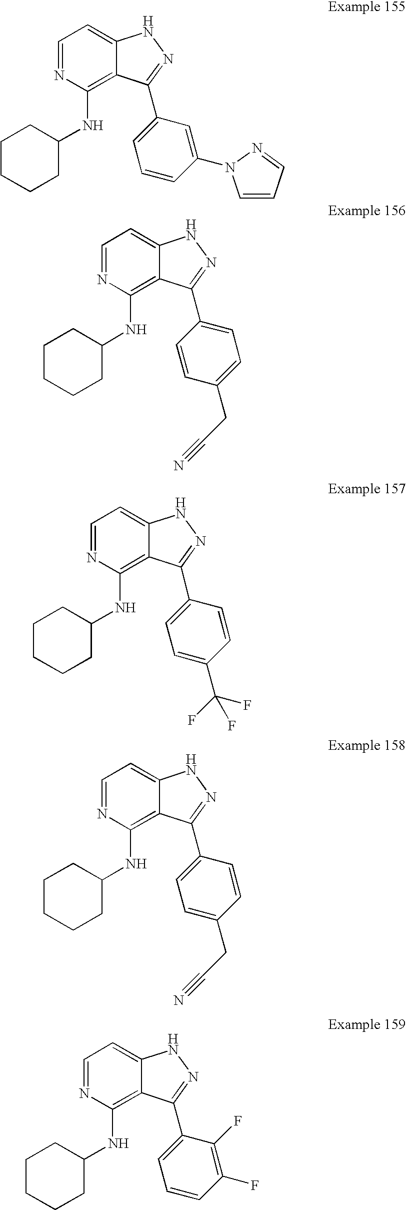

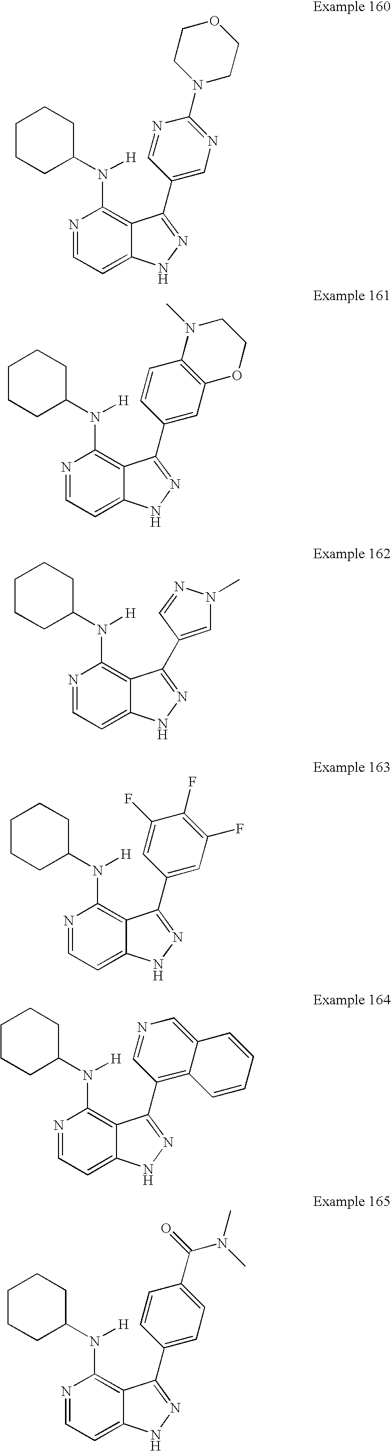

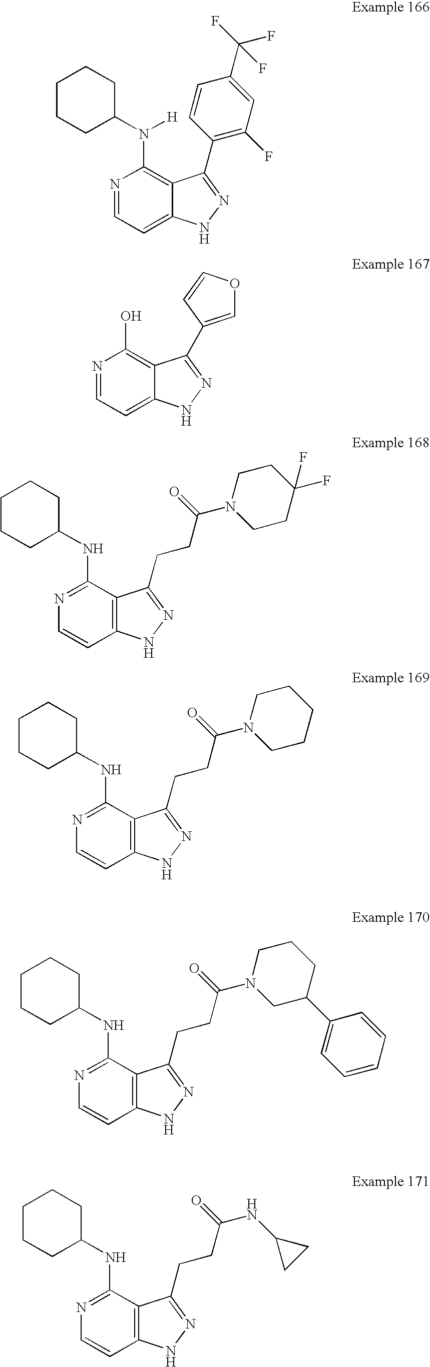

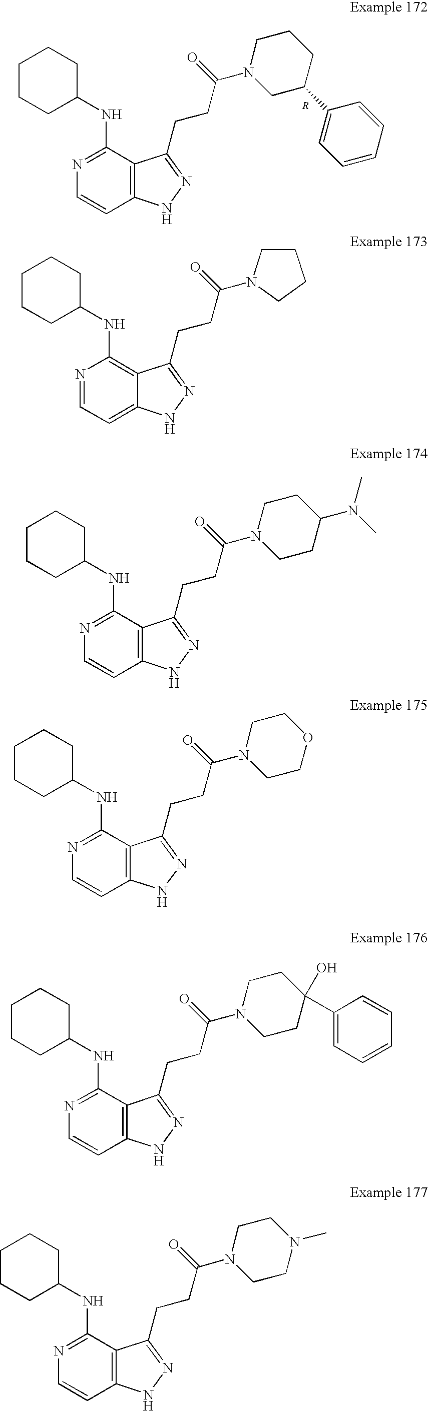

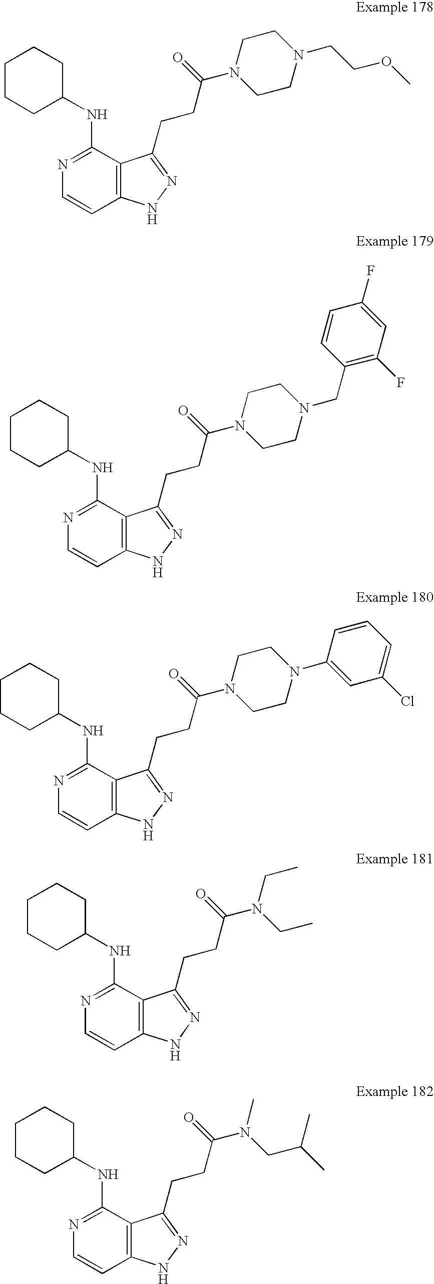

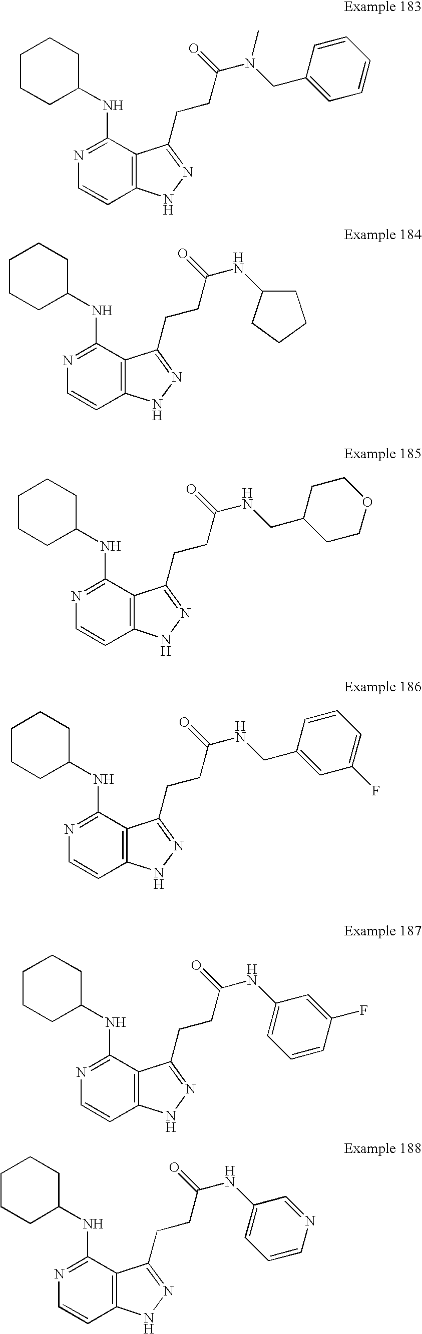

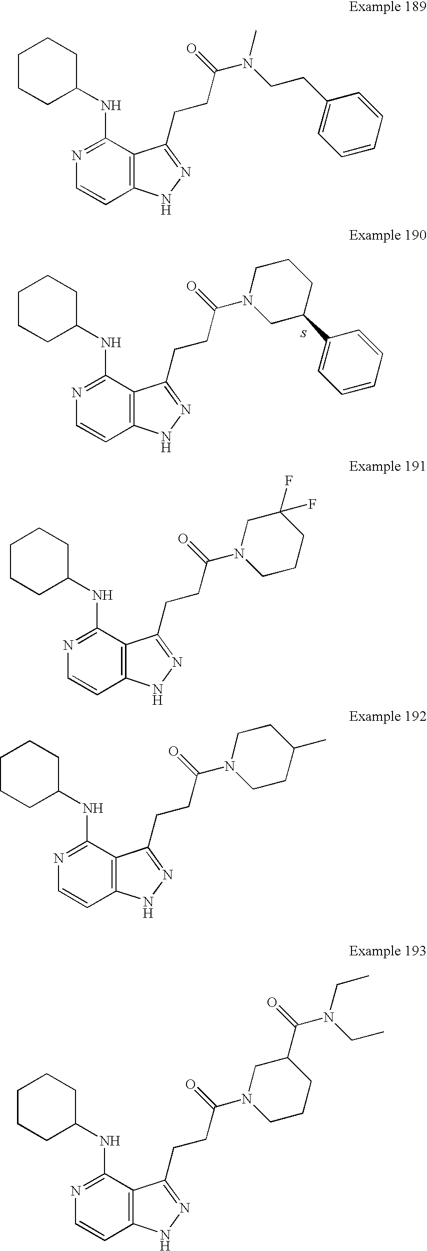

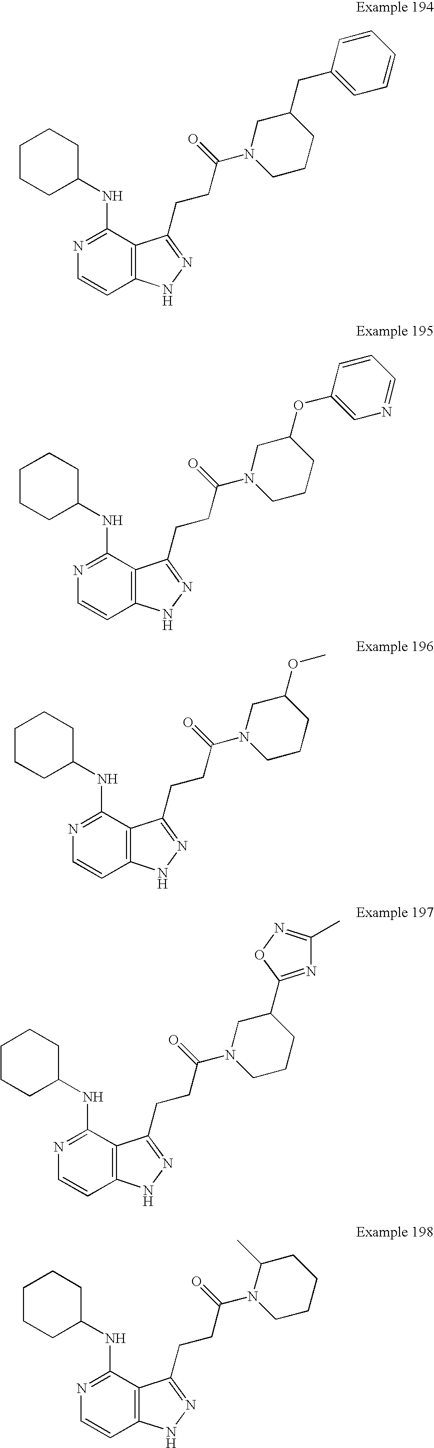

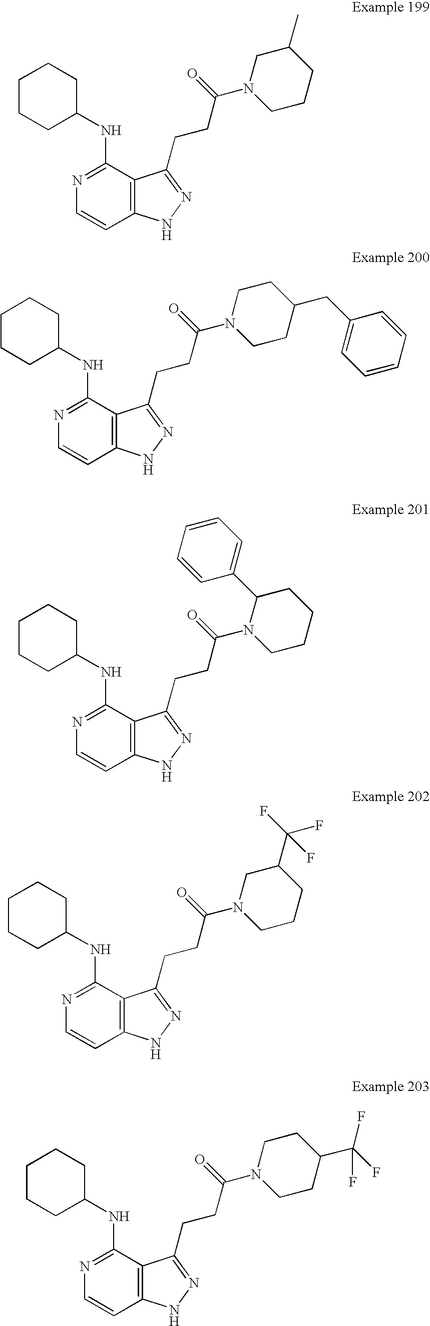

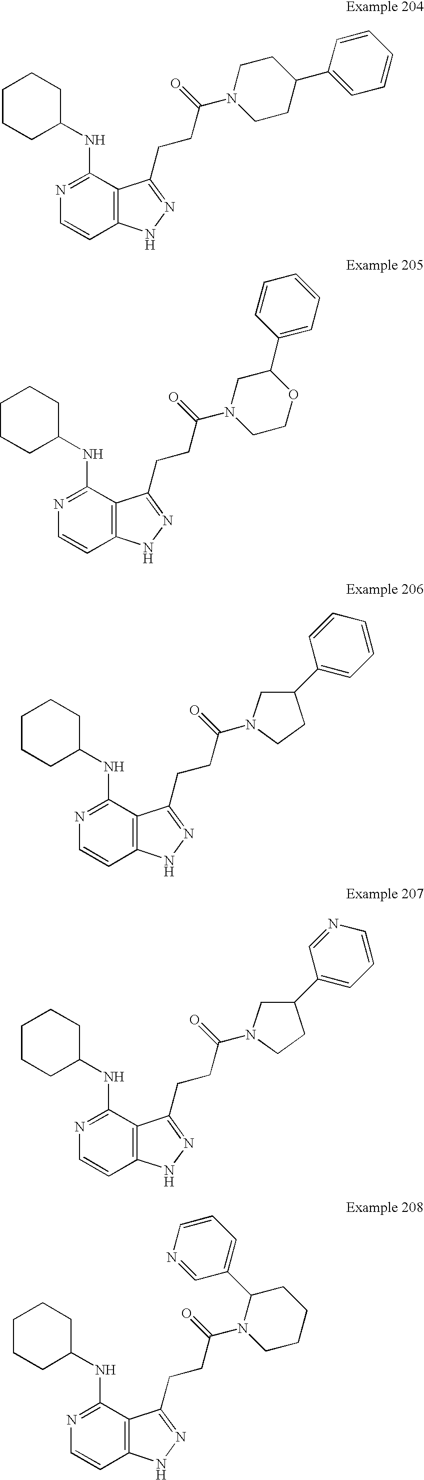

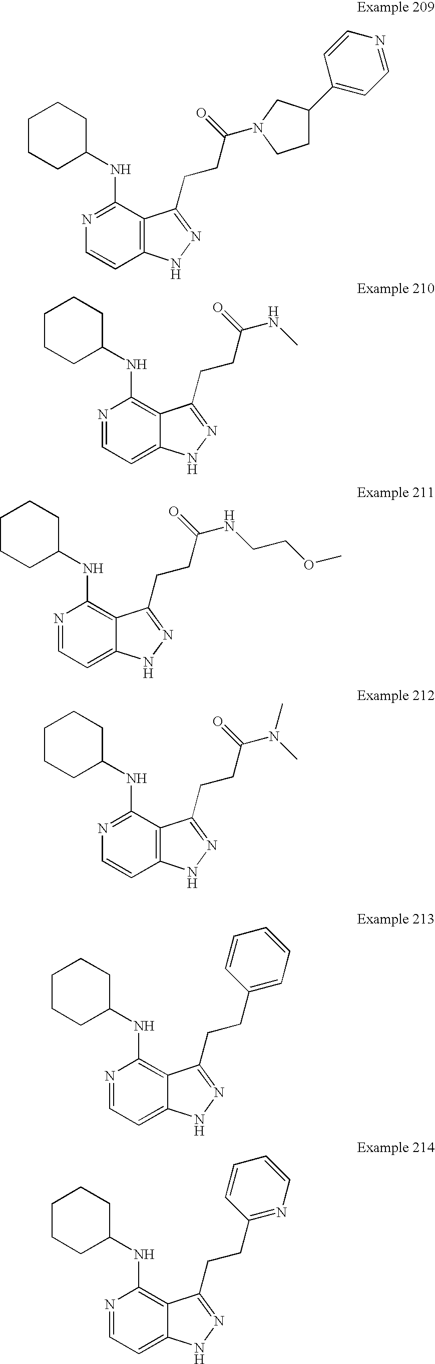

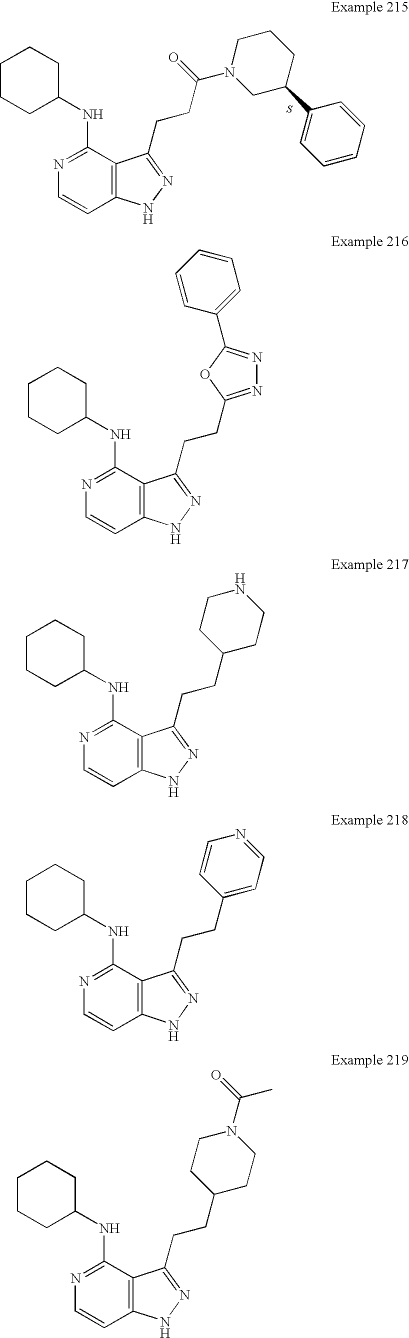

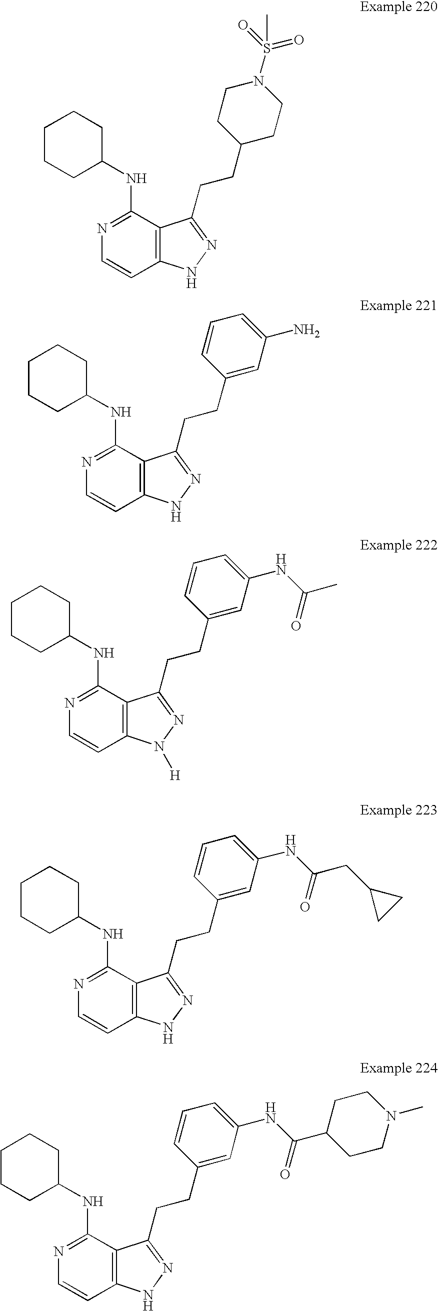

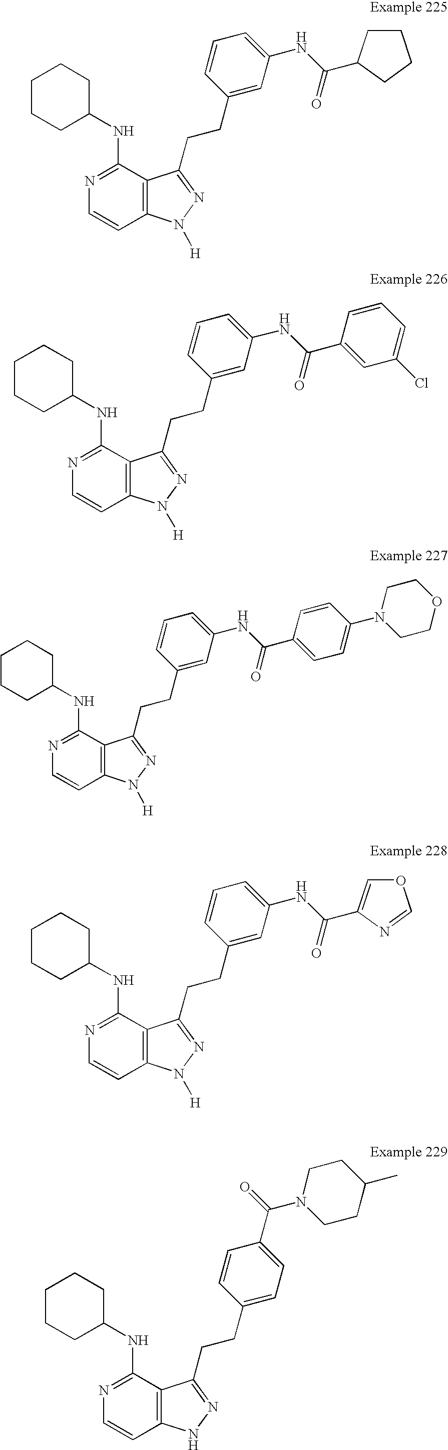

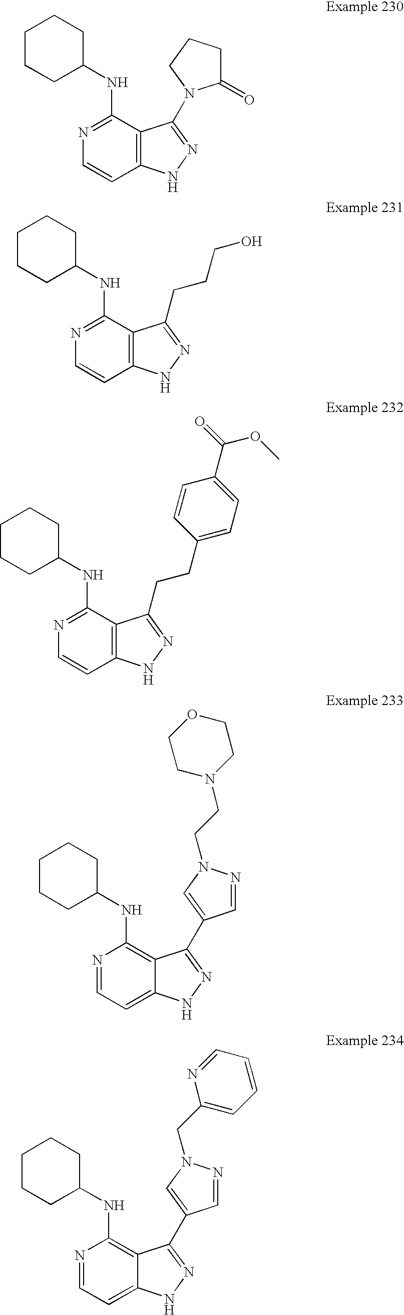

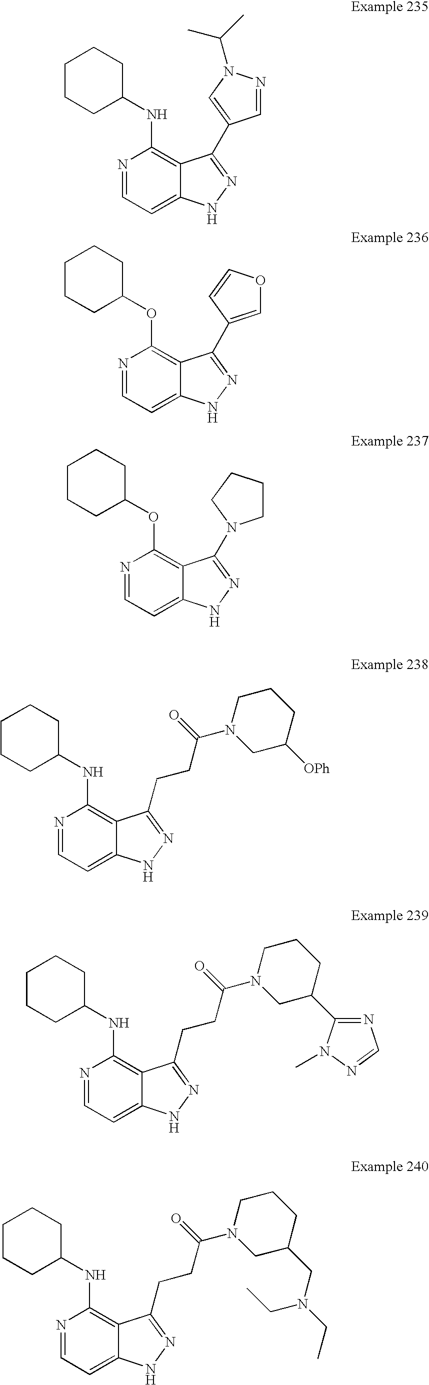

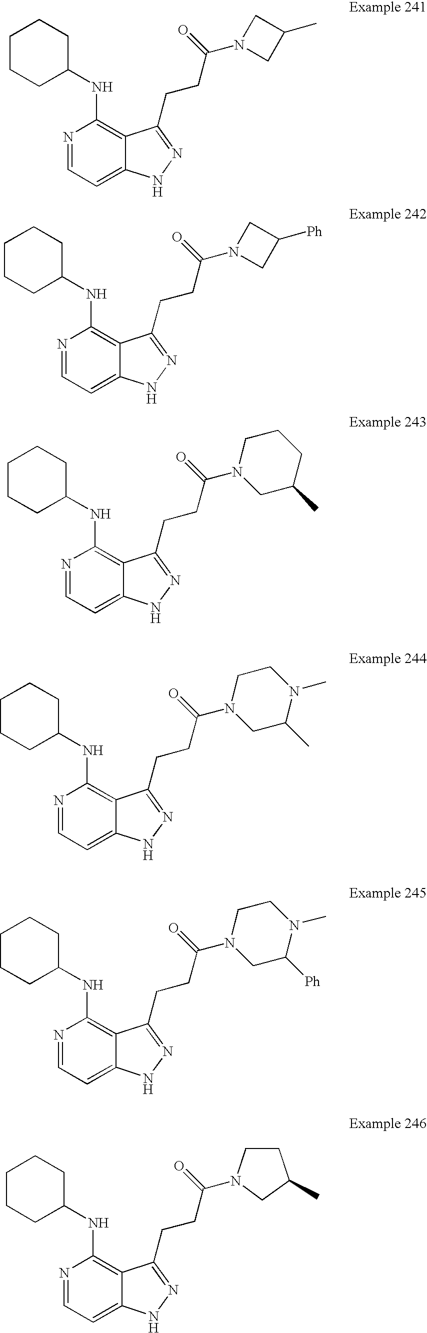

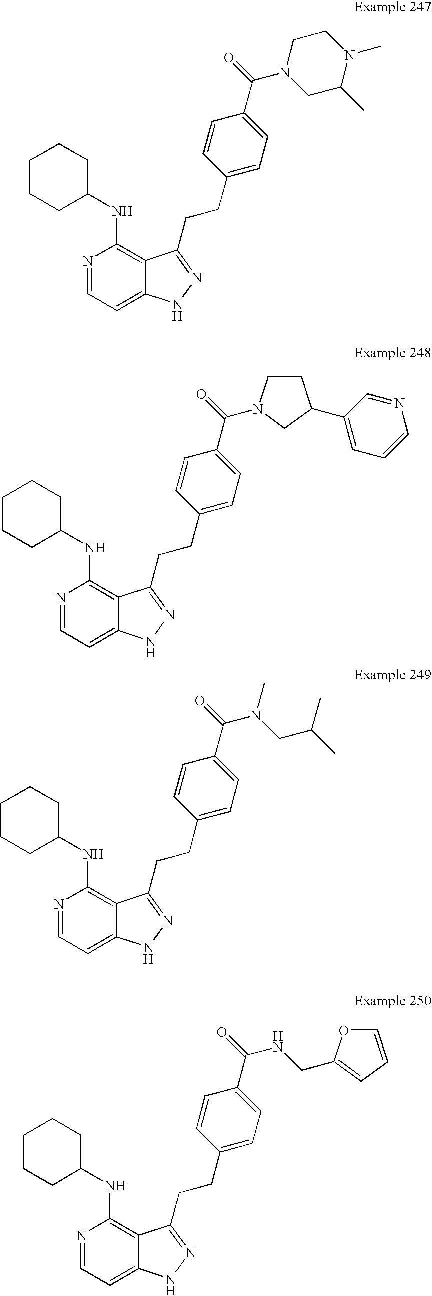

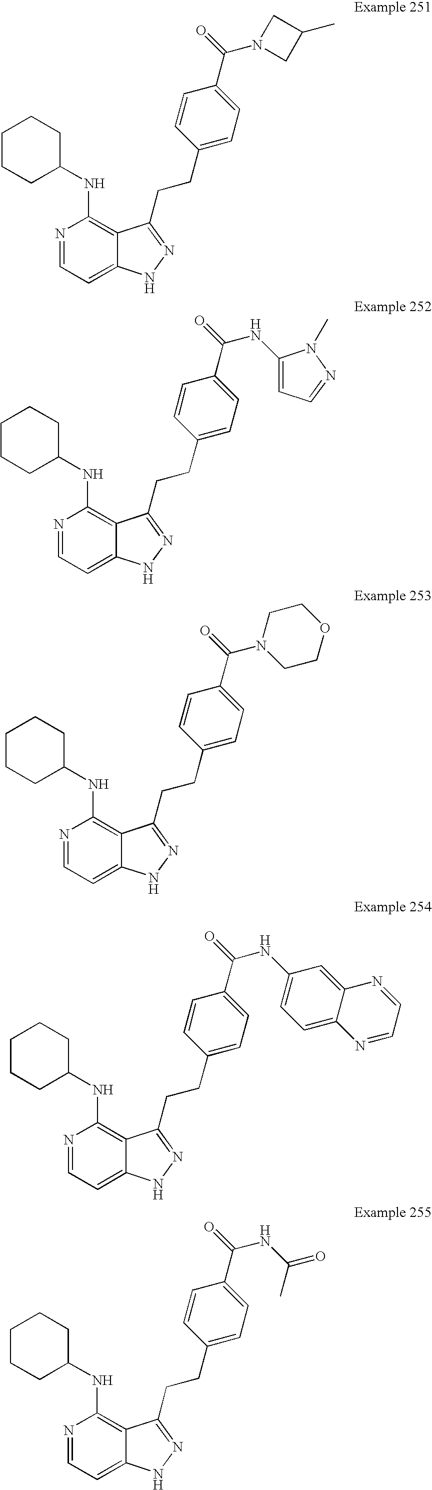

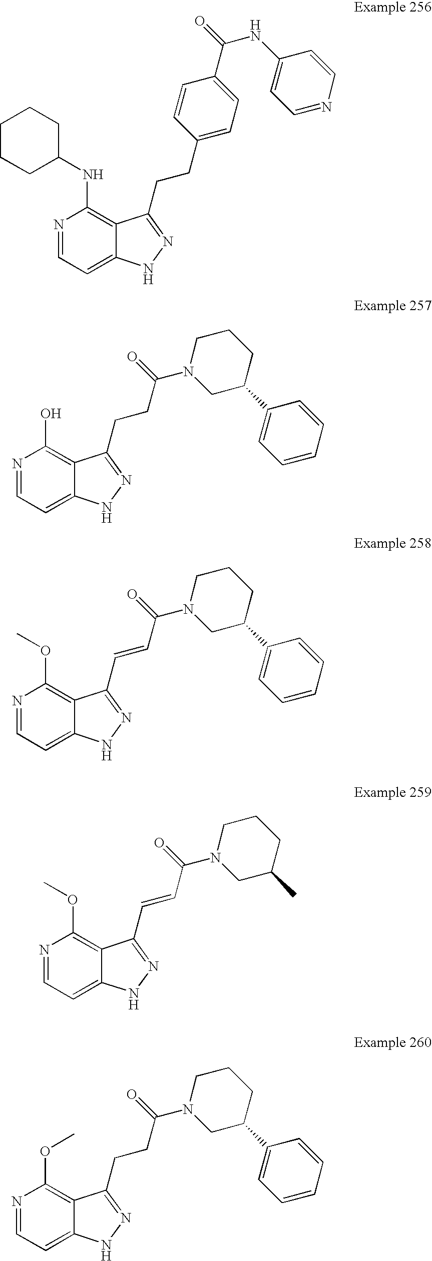

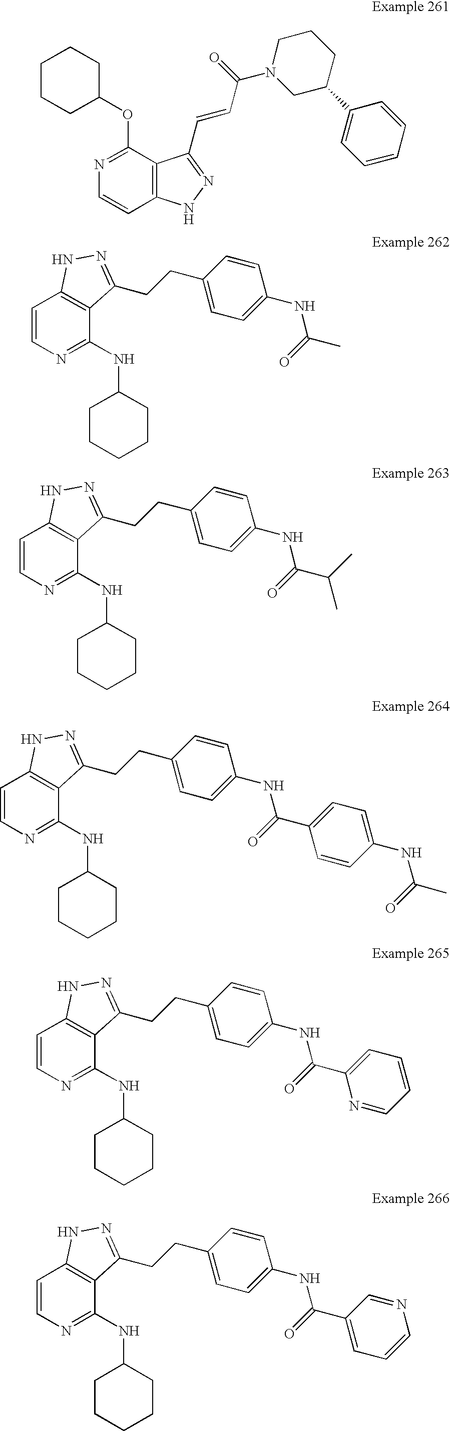

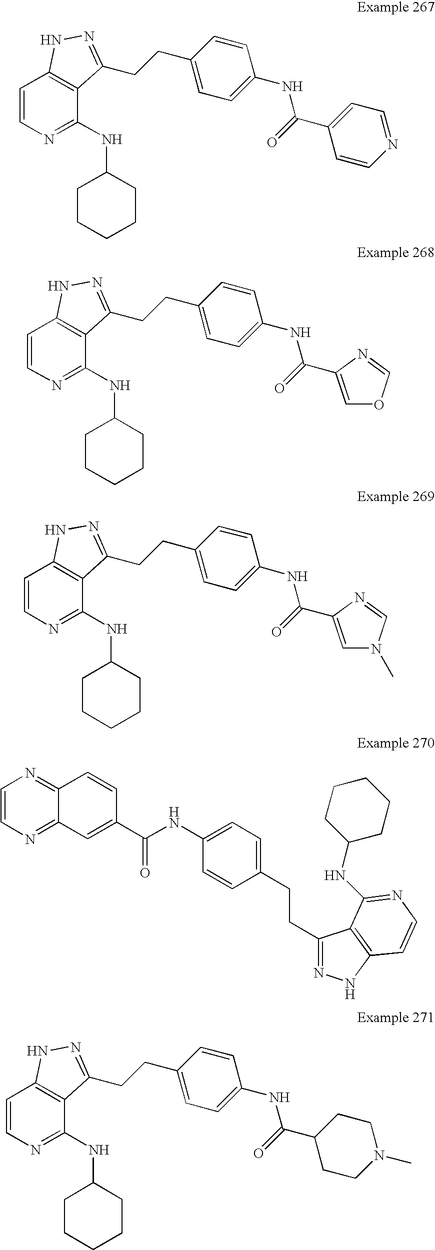

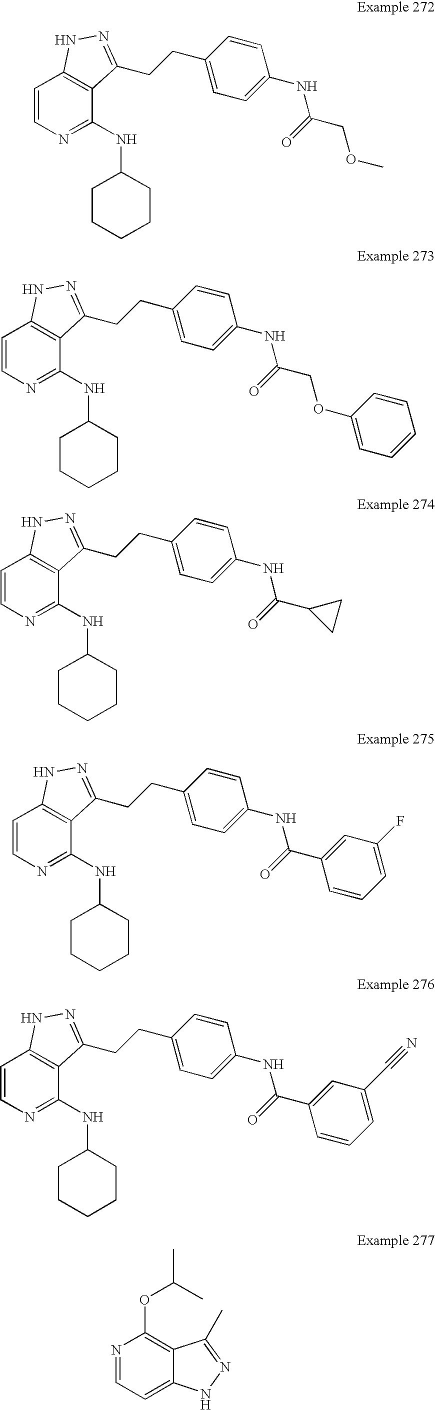

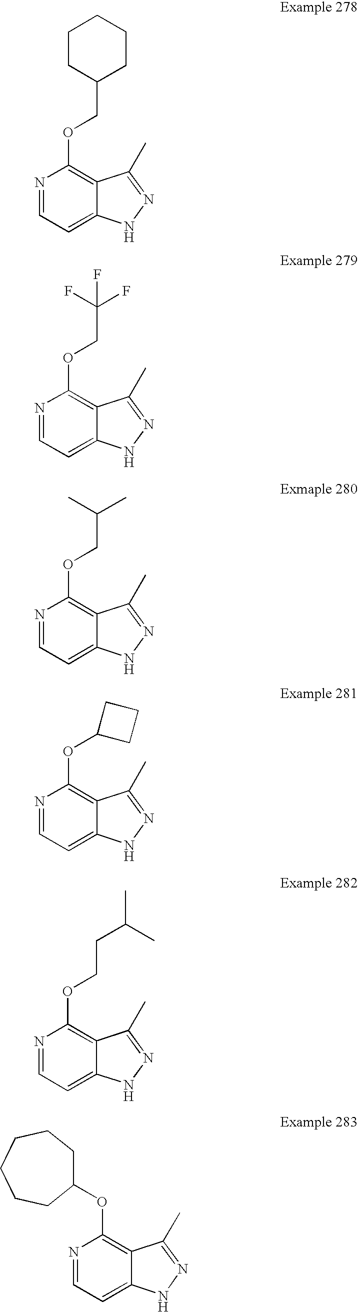

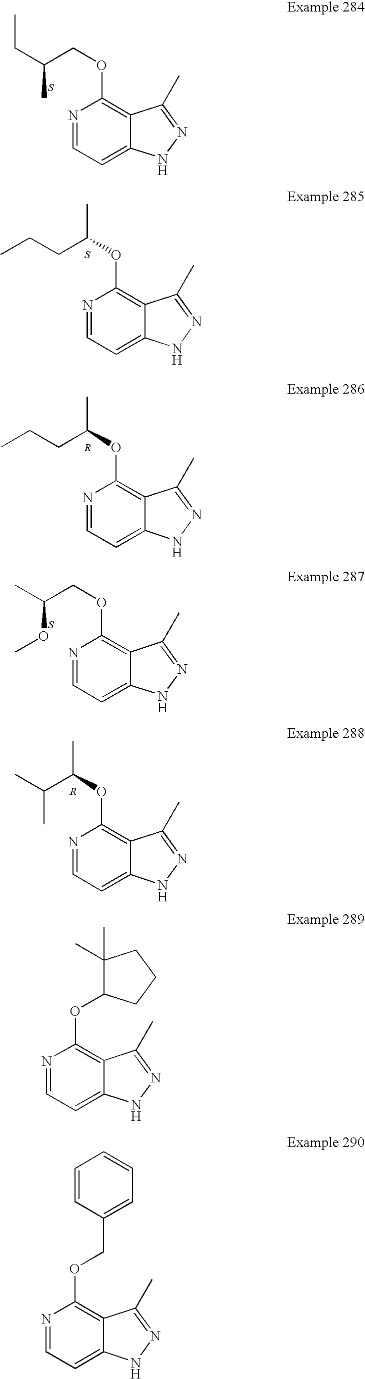

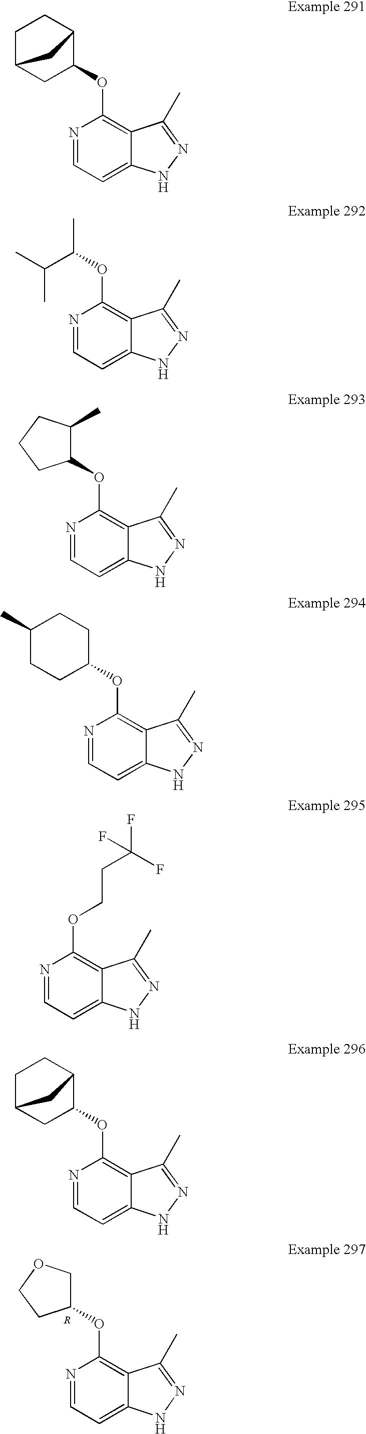

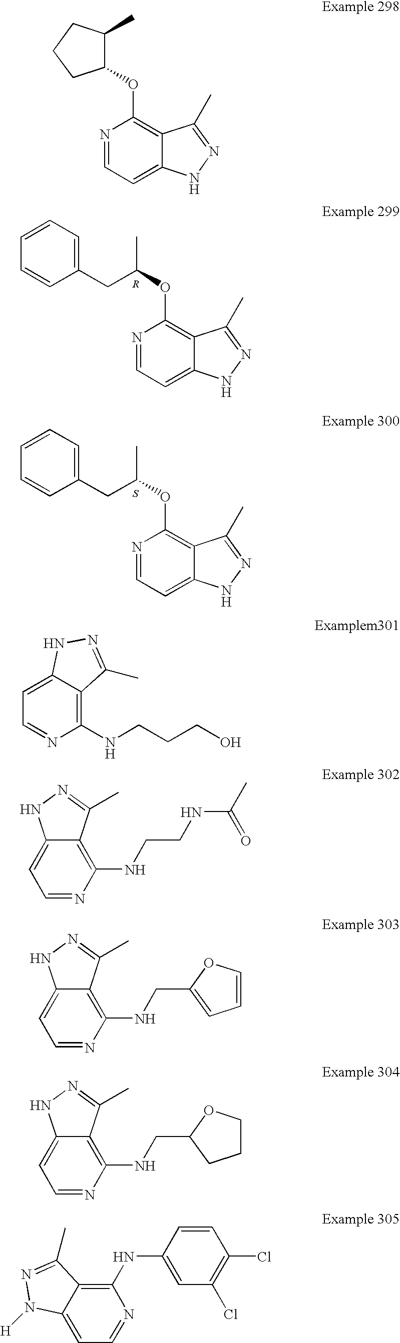

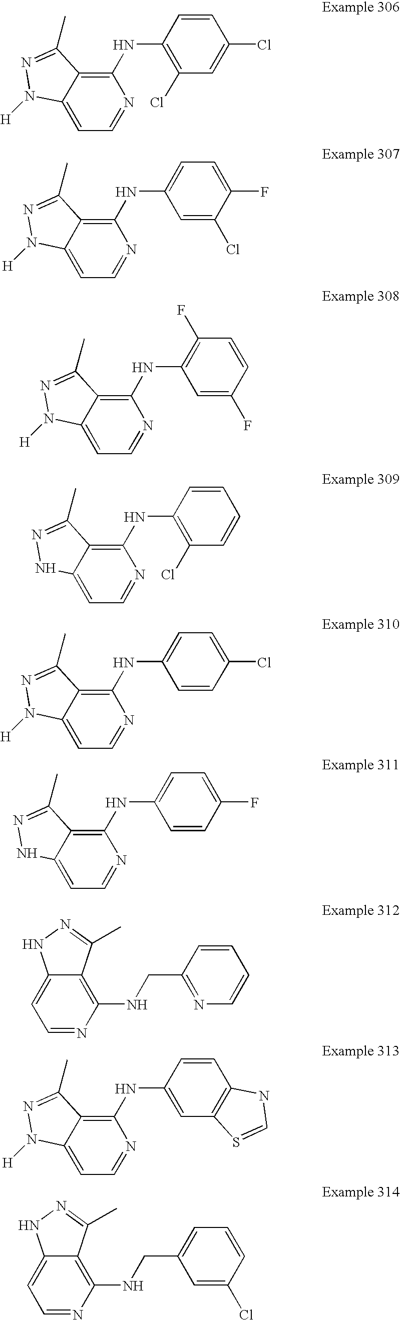

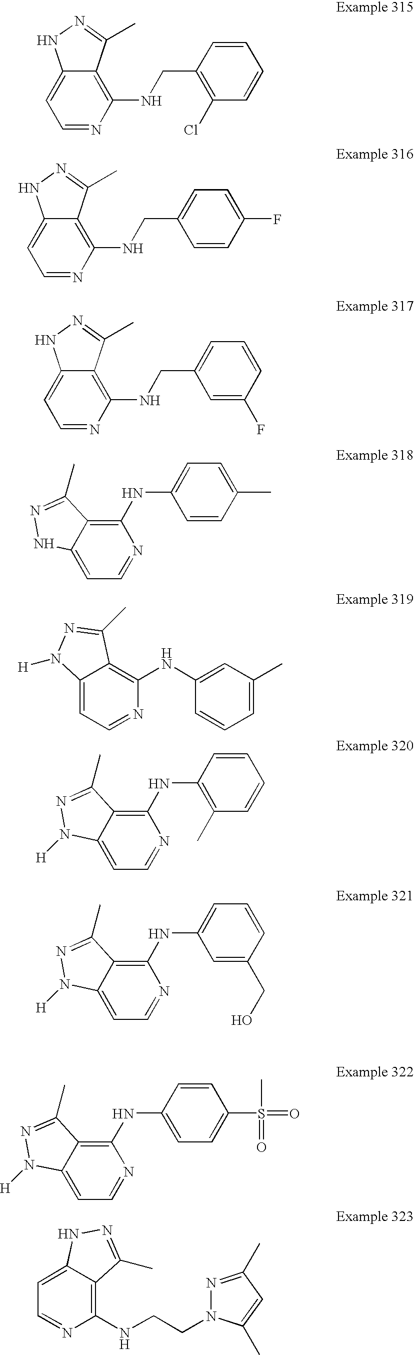

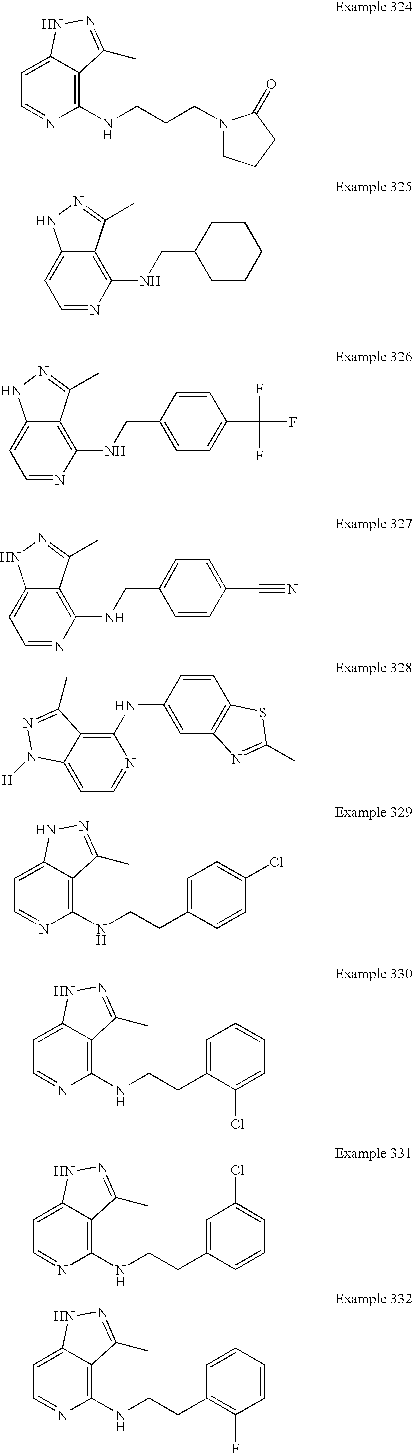

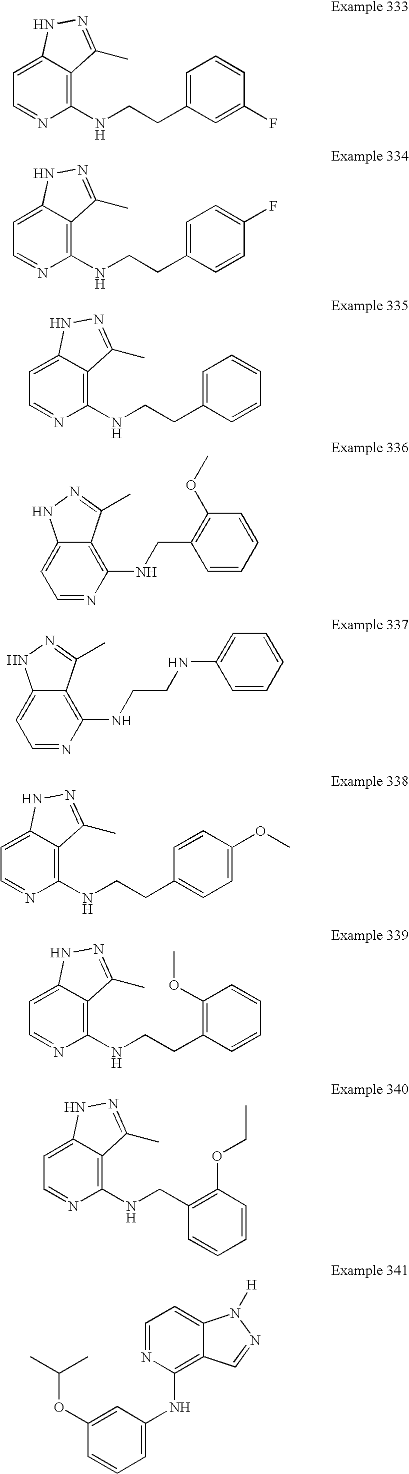

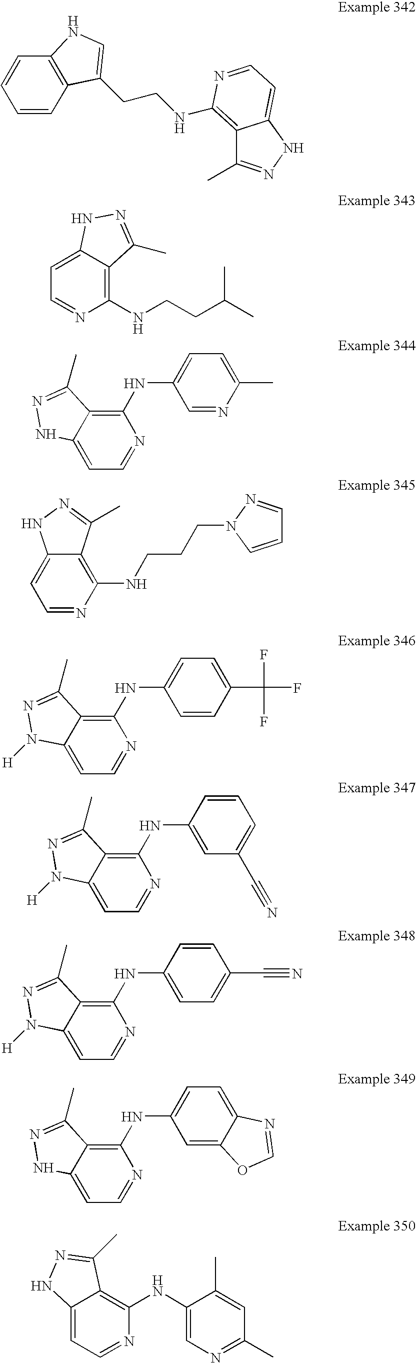

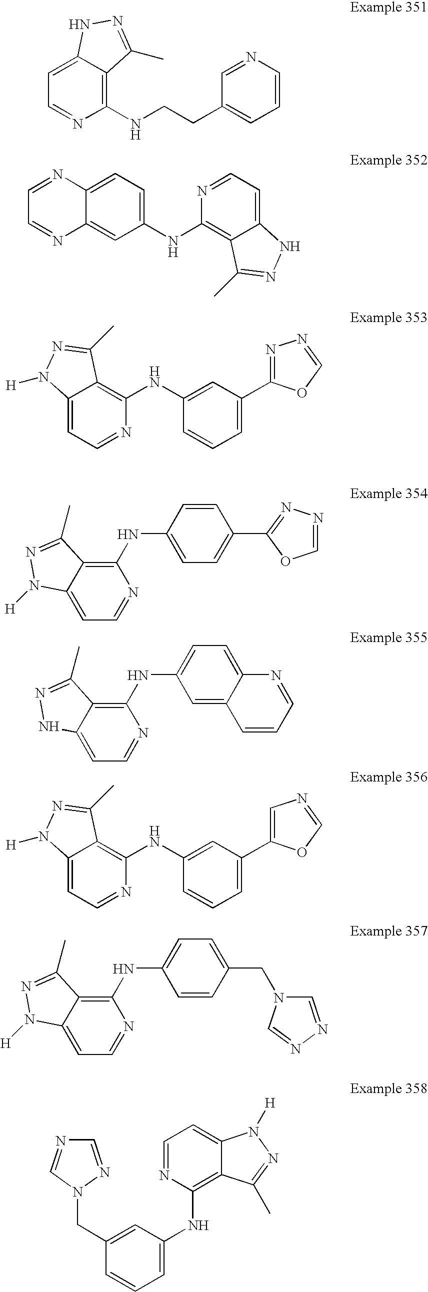

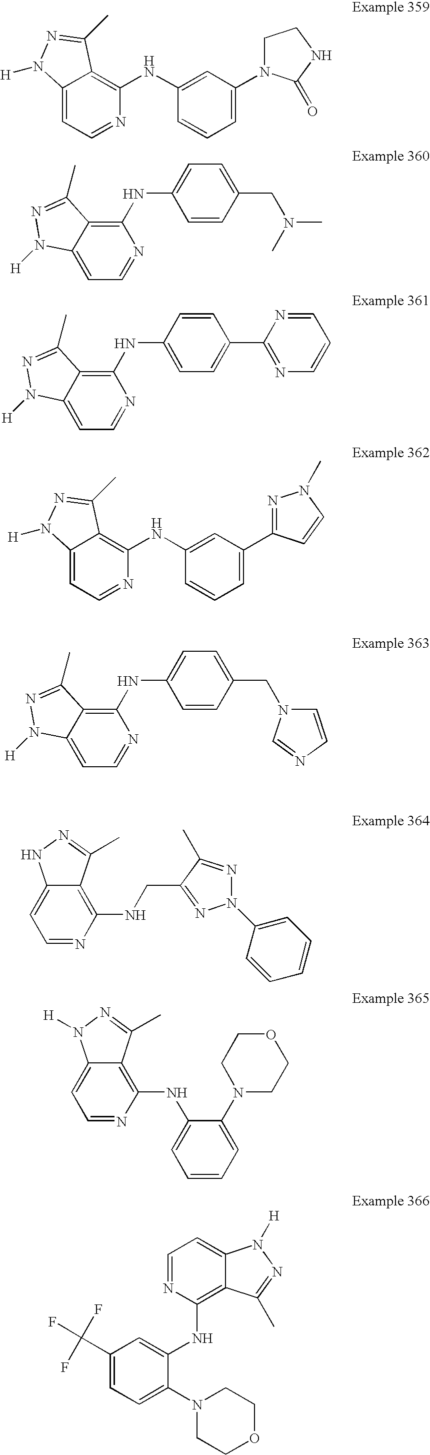

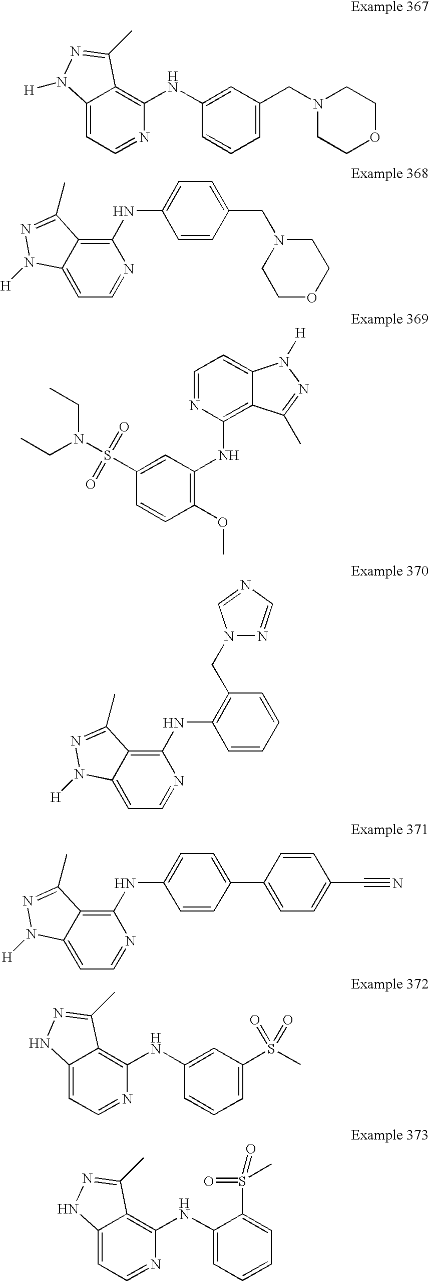

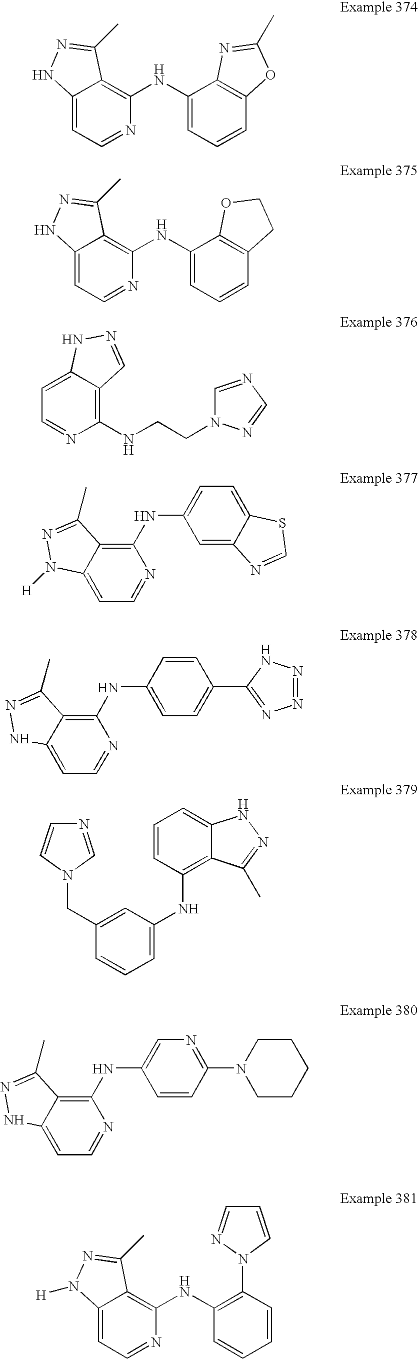

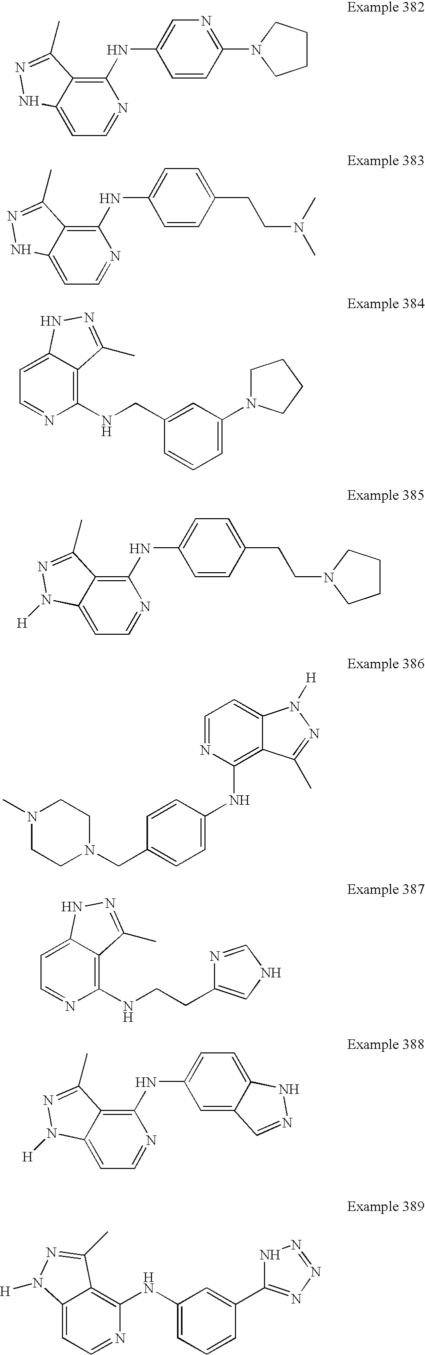

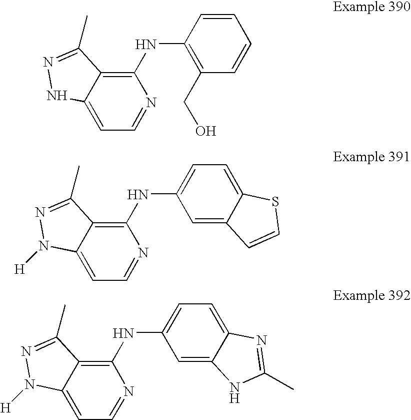

26. A pharmaceutical composition comprising a compound according to claim 1 and a pharmaceutically acceptable carrier, diluent or excipient.

27. A compound according to claim 1 for use in medicine.

28. A compound according to claim 1 for use in treating a disorder selected from cancer and neurodegenerative diseases.

29. A method for treating or preventing a disorder selected from cancer and neurodegenerative diseases in a subject, comprising administering to the subject a compound of claim 1.

30. A method for the prevention or treatment of a disorder caused by, associated with or accompanied by abnormal kinase activity, preferably abnormal LRRK2 activity in a subject, comprising administering to the subject a compound of claim 1.

31. A method of treating a mammal having a disease state alleviated by the inhibition of LRRK2, wherein the method comprises administering to a mammal a therapeutically effective amount of a compound according to claim 1.

32. Use of a compound according to claim 1 in an assay for identifying further candidate compounds capable of inhibiting LRRK, more preferably LRRK2.

33. A process for preparing a compound of formula I as defined in claim 1, said process comprising converting a compound of formula II into a compound of formula I:

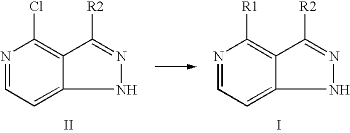

34. A process according to claim 33 which further comprises the step of preparing said compound of formula II by treating a compound of formula III with hydrazine monohydrate:

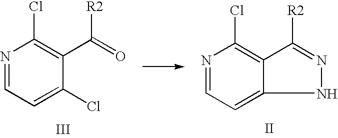

35. A process according to claim 34 which further comprises the step of preparing said compound of formula III by treating a compound of formula IV with an oxidizing agent:

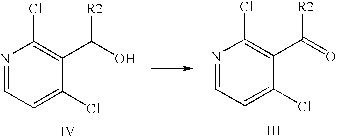

36. A process according to claim 35 which further comprises the step of preparing said compound of formula IV by treating a compound of formula V with R²—Mg—Cl:

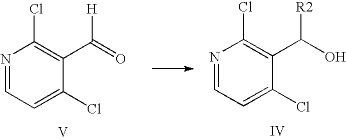

37. A process according to claim 33 where R¹ is —NHR³, and said process comprises reacting a compound of formula II with an amine of formula NH₂R³.

38. A process according to claim 33 where R¹ is an NH-containing C₄₋₇-heterocycloalkyl or an NH-containing fused aryl-C₄₋₇-heterocycloalkyl, and said process comprises reacting a compound of formula II with the NH-group of said C₄₋₇-heterocycloalkyl or fused aryl-C₄₋₇-heterocycloalkyl.

39. A process according to claim 33 wherein R¹ is selected from aryl, heteroaryl, C₄₋₇-heterocycloalkyl, fused aryl-C₄₋₇-heterocycloalkyl, —C₃₋₇ cycloalkyl and —C₁₋₆ alkyl, and said process comprises reacting a compound of formula II with X—R¹, where X is a 4,4,5,5-tetramethyl-1,3,2-dioxaborolan-2-yl group, in the presence of a coupling agent.

40. A process according to claim 39 wherein the coupling agent is palladium diphenylphosphinoferrocene dichloride.

41. A combination comprising a compound according to claim 1 and a further therapeutic agent.

42. A pharmaceutical composition according to claim 26 which further comprises a second therapeutic agent.
